SEQUENCE LISTING (161 sequences)

SEQ ID NO: 1 - DNA, 22 bp, Homo sapiens
  source 1..22: mol_type = genomic DNA
caacggatttggtcgtattggg

SEQ ID NO: 2 - DNA, 25 bp, Homo sapiens
  source 1..25: mol_type = genomic DNA
tgatggcaacaatatccactttacc

SEQ ID NO: 3 - DNA, 21 bp, Homo sapiens
  source 1..21: mol_type = genomic DNA
cgcctggtcaccagggctgct

SEQ ID NO: 4 - DNA, 22 bp, Homo sapiens
  source 1..22: mol_type = genomic DNA
caaattcaacctatgacagagg

SEQ ID NO: 5 - skipped

SEQ ID NO: 6 - protein, 4 aa, Homo sapiens
ATLE

SEQ ID NO: 7 - protein, 6 aa, Homo sapiens
RRERQQ

SEQ ID NO: 8 - DNA, 12 bp, Homo sapiens
  source 1..12: mol_type = genomic DNA
gcaacccttgag

SEQ ID NO: 9 - DNA, 12 bp, Homo sapiens
  source 1..12: mol_type = genomic DNA
gccctgtcctgg

SEQ ID NO: 10 - DNA, 55 bp, Homo sapiens
  source 1..55: mol_type = genomic DNA
caaggcctgctatccctagaacccacgctctcaaattcaacctatgacagaggca

SEQ ID NO: 11 - DNA, 54 bp, Homo sapiens
  source 1..54: mol_type = genomic DNA
caaacgaaggtacacgagtgggcattctgtgactcggtacttccctttaggccc

SEQ ID NO: 12 - protein, 3144 aa, Homo sapiens
MATLEKLMKAFESLKSFQQQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 13 - DNA, 9435 bp, Homo sapiens
  source 1..9435: mol_type = genomic DNA
atggcgaccctggaaaagctgatgaaggccttcgagtccctcaagtccttccagcagcagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccaaagaaagaactttcagctaccaagaaagaccgtgtgaatcattgtctgacaatatgtgaaaacatagtggcacagtctgtcagaaattctccagaatttcagaaacttctgggcatcgctatggaactttttctgctgtgcagtgatgacgcagagtcagatgtcaggatggtggctgacgaatgcctcaacaaagttatcaaagctttgatggattctaatcttccaaggttacagctcgagctctataaggaaattaaaaagaatggtgcccctcggagtttgcgtgctgccctgtggaggtttgctgagctggctcacctggttcggcctcagaaatgcaggccttacctggtgaaccttctgccgtgcctgactcgaacaagcaagagacccgaagaatcagtccaggagaccttggctgcagctgttcccaaaattatggcttcttttggcaattttgcaaatgacaatgaaattaaggttttgttaaaggccttcatagcgaacctgaagtcaagctcccccaccattcggcggacagcggctggatcagcagtgagcatctgccagcactcaagaaggacacaatatttctatagttggctactaaatgtgctcttaggcttactcgttcctgtcgaggatgaacactccactctgctgattcttggcgtgctgctcaccctgaggtatttggtgcccttgctgcagcagcaggtcaaggacacaagcctgaaaggcagcttcggagtgacaaggaaagaaatggaagtctctccttctgcagagcagcttgtccaggtttatgaactgacgttacatcatacacagcaccaagaccacaatgttgtgaccggagccctggagctgttgcagcagctcttcagaacgcctccacccgagcttctgcaaaccctgaccgcagtcgggggcattgggcagctcaccgctgctaaggaggagtctggtggccgaagccgtagtgggagtattgtggaacttatagctggagggggttcctcatgcagccctgtcctttcaagaaaacaaaaaggcaaagtgctcttaggagaagaagaagccttggaggatgactctgaatcgagatcggatgtcagcagctctgccttaacagcctcagtgaaggatgagatcagtggagagctggctgcttcttcaggggtttccactccagggtcagcaggtcatgacatcatcacagaacagccacggtcacagcacacactgcaggcggactcagtggatctggccagctgtgacttgacaagctctgccactgatggggatgaggaggatatcttgagccacagctccagccaggtcagcgccgtcccatctgaccctgccatggacctgaatgatgggacccaggcctcgtcgcccatcagcgacagctcccagaccaccaccgaagggcctgattcagctgttaccccttcagacagttctgaaattgtgttagacggtaccgacaaccagtatttgggcctgcagattggacagccccaggatgaagatgaggaagccacaggtattcttcctgatgaagcctcggaggccttcaggaactcttccatggcccttcaacaggcacatttattgaaaaacatgagtcactgcaggcagccttctgacagcagtgttgataaatttgtgttgagagatgaagctactgaaccgggtgatcaagaaaacaagccttgccgcatcaaaggtgacattggacagtccactgatgatgactctgcacctcttgtccattgtgtccgccttttatctgcttcgtttttgctaacagggggaaaaaatgtgctggttccggacagggatgtgagggtcagcgtgaaggccctggccctcagctgtgtgggagcagctgtggccctccacccggaatctttcttcagcaaactctataaagttcctcttgacaccacggaataccctgaggaacagtatgtctcagacatcttgaactacatcgatcatggagacccacaggttcgaggagccactgccattctctgtgggaccctcatctgctccatcctcagcaggtcccgcttccacgtgggagattggatgggcaccattagaaccctcacaggaaatacattttctttggcggattgcattcctttgctgcggaaaacactgaaggatgagtcttctgttacttgcaagttagcttgtacagctgtgaggaactgtgtcatgagtctctgcagcagcagctacagtgagttaggactgcagctgatcatcgatgtgctgactctgaggaacagttcctattggctggtgaggacagagcttctggaaacccttgcagagattgacttcaggctggtgagctttttggaggcaaaagcagaaaacttacacagaggggctcatcattatacagggcttttaaaactgcaagaacgagtgctcaataatgttgtcatccatttgcttggagatgaagaccccagggtgcgacatgttgccgcagcatcactaattaggcttgtcccaaagctgttttataaatgtgaccaaggacaagctgatccagtagtggccgtggcaagagatcaaagcagtgtttacctgaaacttctcatgcatgagacgcagcctccatctcatttctccgtcagcacaataaccagaatatatagaggctataacctactaccaagcataacagacgtcactatggaaaataacctttcaagagttattgcagcagtttctcatgaactaatcacatcaaccaccagagcactcacatttggatgctgtgaagctttgtgtcttctttccactgccttcccagtttgcatttggagtttaggttggcactgtggagtgcctccactgagtgcctcagatgagtctaggaagagctgtaccgttgggatggccacaatgattctgaccctgctctcgtcagcttggttcccattggatctctcagcccatcaagatgctttgattttggccggaaacttgcttgcagccagtgctcccaaatctctgagaagttcatgggcctctgaagaagaagccaacccagcagccaccaagcaagaggaggtctggccagccctgggggaccgggccctggtgcccatggtggagcagctcttctctcacctgctgaaggtgattaacatttgtgcccacgtcctggatgacgtggctcctggacccgcaataaaggcagccttgccttctctaacaaaccccccttctctaagtcccatccgacgaaaggggaaggagaaagaaccaggagaacaagcatctgtaccgttgagtcccaagaaaggcagtgaggccagtgcagcttctagacaatctgatacctcaggtcctgttacaacaagtaaatcctcatcactggggagtttctatcatcttccttcatacctcaaactgcatgatgtcctgaaagctacacacgctaactacaaggtcacgctggatcttcagaacagcacggaaaagtttggagggtttctccgctcagccttggatgttctttctcagatactagagctggccacactgcaggacattgggaagtgtgttgaagagatcctaggatacctgaaatcctgctttagtcgagaaccaatgatggcaactgtttgtgttcaacaattgttgaagactctctttggcacaaacttggcctcccagtttgatggcttatcttccaaccccagcaagtcacaaggccgagcacagcgccttggctcctccagtgtgaggccaggcttgtaccactactgcttcatggccccgtacacccacttcacccaggccctcgctgacgccagcctgaggaacatggtgcaggcggagcaggagaacgacacctcgggatggtttgatgtcctccagaaagtgtctacccagttgaagacaaacctcacgagtgtcacaaagaaccgtgcagataagaatgctattcataatcacattcgtttgtttgaacctcttgttataaaagctttaaaacagtacacgactacaacatgtgtgcagttacagaagcaggttttagatttgctggcgcagctggttcagttacgggttaattactgtcttctggattcagatcaggtgtttattggctttgtattgaaacagtttgaatacattgaagtgggccagttcagggaatcagaggcaatcattccaaacatctttttcttcttggtattactatcttatgaacgctatcattcaaaacagatcattggaattcctaaaatcattcagctctgtgatggcatcatggccagtggaaggaaggctgtgacacatgccataccggctctgcagcccatagtccacgacctctttgtattaagaggaacaaataaagctgatgcaggaaaagagcttgaaacccaaaaagaggtggtggtgtcaatgttactgagactcatccagtaccatcaggtgttggagatgttcattcttgtcctgcagcagtgccacaaggagaatgaagacaagtggaagcgactgtctcgacagatagctgacatcatcctcccaatgttagccaaacagcagatgcacattgactctcatgaagcccttggagtgttaaatacattatttgagattttggccccttcctccctccgtccggtagacatgcttttacggagtatgttcgtcactccaaacacaatggcgtccgtgagcactgttcaactgtggatatcgggaattctggccattttgagggttctgatttcccagtcaactgaagatattgttctttctcgtattcaggagctctccttctctccgtatttaatctcctgtacagtaattaataggttaagagatggggacagtacttcaacgctagaagaacacagtgaagggaaacaaataaagaatttgccagaagaaacattttcaaggtttctattacaactggttggtattcttttagaagacattgttacaaaacagctgaaggtggaaatgagtgagcagcaacatactttctattgccaggaactaggcacactgctaatgtgtctgatccacatcttcaagtctggaatgttccggagaatcacagcagctgccactaggctgttccgcagtgatggctgtggcggcagtttctacaccctggacagcttgaacttgcgggctcgttccatgatcaccacccacccggccctggtgctgctctggtgtcagatactgctgcttgtcaaccacaccgactaccgctggtgggcagaagtgcagcagaccccgaaaagacacagtctgtccagcacaaagttacttagtccccagatgtctggagaagaggaggattctgacttggcagccaaacttggaatgtgcaatagagaaatagtacgaagaggggctctcattctcttctgtgattatgtctgtcagaacctccatgactccgagcacttaacgtggctcattgtaaatcacattcaagatctgatcagcctttcccacgagcctccagtacaggacttcatcagtgccgttcatcggaactctgctgccagcggcctgttcatccaggcaattcagtctcgttgtgaaaacctttcaactccaaccatgctgaagaaaactcttcagtgcttggaggggatccatctcagccagtcgggagctgtgctcacgctgtatgtggacaggcttctgtgcacccctttccgtgtgctggctcgcatggtcgacatccttgcttgtcgccgggtagaaatgcttctggctgcaaatttacagagcagcatggcccagttgccaatggaagaactcaacagaatccaggaataccttcagagcagcgggctcgctcagagacaccaaaggctctattccctgctggacaggtttcgtctctccaccatgcaagactcacttagtccctctcctccagtctcttcccacccgctggacggggatgggcacgtgtcactggaaacagtgagtccggacaaagactggtacgttcatcttgtcaaatcccagtgttggaccaggtcagattctgcactgctggaaggtgcagagctggtgaatcggattcctgctgaagatatgaatgccttcatgatgaactcggagttcaacctaagcctgctagctccatgcttaagcctagggatgagtgaaatttctggtggccagaagagtgccctttttgaagcagcccgtgaggtgactctggcccgtgtgagcggcaccgtgcagcagctccctgctgtccatcatgtcttccagcccgagctgcctgcagagccggcggcctactggagcaagttgaatgatctgtttggggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgaggccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccgttgcagtgcagcctggagagcagcttcttagtccagaaagaaggacaaataccccaaaagccatcagcgaggaggaggaggaagtagatccaaacacacagaatcctaagtatatcactgcagcctgtgagatggtggcagaaatggtggagtctctgcagtcggtgttggccttgggtcataaaaggaatagcggcgtgccggcgtttctcacgccattgctaaggaacatcatcatcagcctggcccgcctgccccttgtcaacagctacacacgtgtgcccccactggtgtggaagcttggatggtcacccaaaccgggaggggattttggcacagcattccctgagatccccgtggagttcctccaggaaaaggaagtctttaaggagttcatctaccgcatcaacacactaggctggaccagtcgtactcagtttgaagaaacttgggccaccctccttggtgtcctggtgacgcagcccctcgtgatggagcaggaggagagcccaccagaagaagacacagagaggacccagatcaacgtcctggccgtgcaggccatcacctcactggtgctcagtgcaatgactgtgcctgtggccggcaacccagctgtaagctgcttggagcagcagccccggaacaagcctctgaaagctctcgacaccaggtttgggaggaagctgagcattatcagagggattgtggagcaagagattcaagcaatggtttcaaagagagagaatattgccacccatcatttatatcaggcatgggatcctgtcccttctctgtctccggctactacaggtgccctcatcagccacgagaagctgctgctacagatcaaccccgagcgggagctggggagcatgagctacaaactcggccaggtgtccatacactccgtgtggctggggaacagcatcacacccctgagggaggaggaatgggacgaggaagaggaggaggaggccgacgcccctgcaccttcgtcaccacccacgtctccagtcaactccaggaaacaccgggctggagttgacatccactcctgttcgcagtttttgcttgagttgtacagccgctggatcctgccgtccagctcagccaggaggaccccggccatcctgatcagtgaggtggtcagatcccttctagtggtctcagacttgttcaccgagcgcaaccagtttgagctgatgtatgtgacgctgacagaactgcgaagggtgcacccttcagaagacgagatcctcgctcagtacctggtgcctgccacctgcaaggcagctgccgtccttgggatggacaaggccgtggcggagcctgtcagccgcctgctggagagcacgctcaggagcagccacctgcccagcagggttggagccctgcacggcgtcctctatgtgctggagtgcgacctgctggacgacactgccaagcagctcatcccggtcatcagcgactatctcctctccaacctgaaagggatcgcccactgcgtgaacattcacagccagcagcacgtactggtcatgtgtgccactgcgttttacctcattgagaactatcctctggacgtagggccggaattttcagcatcaataatacagatgtgtggggtgatgctgtctggaagtgaggagtccaccccctccatcatttaccactgtgccctcagaggcctggagcgcctcctgctctctgagcagctctcccgcctggatgcagaatcgctggtcaagctgagtgtggacagagtgaacgtgcacagcccgcaccgggccatggcggctctgggcctgatgctcacctgcatgtacacaggaaaggagaaagtcagtccgggtagaacttcagaccctaatcctgcagcccccgacagcgagtcagtgattgttgctatggagcgggtatctgttctttttgataggatcaggaaaggctttccttgtgaagccagagtggtggccaggatcctgccccagtttctagacgacttcttcccaccccaggacatcatgaacaaagtcatcggagagtttctgtccaaccagcagccatacccccagttcatggccaccgtggtgtataaggtgtttcagactctgcacagcaccgggcagtcgtccatggtccgggactgggtcatgctgtccctctccaacttcacgcagagggccccggtcgccatggccacgtggagcctctcctgcttctttgtcagcgcgtccaccagcccgtgggtcgcggcgatcctcccacatgtcatcagcaggatgggcaagctggagcaggtggacgtgaaccttttctgcctggtcgccacagacttctacagacaccagatagaggaggagctcgaccgcagggccttccagtctgtgcttgaggtggttgcagccccaggaagcccatatcaccggctgctgacttgtttacgaaatgtccacaaggtcaccacctgctga

SEQ ID NO: 14 - DNA, 17 bp, synthetic construct
  misc_feature 1..17: note = primer
  source 1..17: mol_type = other DNA
cggctgtggctgaggag

SEQ ID NO: 15 - DNA, 20 bp, synthetic construct
  misc_feature 1..20: note = primer
  source 1..20: mol_type = other DNA
ccaaggtctcctggactgat

SEQ ID NO: 16 - DNA, 21 bp, synthetic construct
  misc_feature 1..21: note = primer
  source 1..21: mol_type = other DNA
ctggatcagcagtgagcatct

SEQ ID NO: 17 - DNA, 20 bp, synthetic construct
  misc_feature 1..20: note = primer
  source 1..20: mol_type = other DNA
ttgaaaggacagggctgcat

SEQ ID NO: 18 - DNA, 24 bp, synthetic construct
  misc_feature 1..24: note = primer
  source 1..24: mol_type = other DNA
tgactctgaatcgagatcggatgt

SEQ ID NO: 19 - DNA, 18 bp, synthetic construct
  misc_feature 1..18: note = primer
  source 1..18: mol_type = other DNA
cagaaggctgcctgcagt

SEQ ID NO: 20 - DNA, 22 bp, synthetic construct
  misc_feature 1..22: note = primer
  source 1..22: mol_type = other DNA
gactctgcacctcttgtccatt

SEQ ID NO: 21 - DNA, 22 bp, Homo sapiens
  source 1..22: mol_type = genomic DNA
ctgttcctcagagtcagcacat

SEQ ID NO: 22 - DNA, 21 bp, Homo sapiens
  source 1..21: mol_type = genomic DNA
ggtgagctttttggaggcaaa

SEQ ID NO: 23 - DNA, 22 bp, synthetic construct
  misc_feature 1..22: note = primer
  source 1..22: mol_type = other DNA
ggtcagaatcattgtggccatc

SEQ ID NO: 24 - DNA, 26 bp, synthetic construct
  misc_feature 1..26: note = primer
  source 1..26: mol_type = other DNA
acctgctgaaggtgattaacatttgt

SEQ ID NO: 25 - DNA, 23 bp, synthetic construct
  misc_feature 1..23: note = primer
  source 1..23: mol_type = other DNA
gggttggaagataagccatcaaa

SEQ ID NO: 26 - DNA, 25 bp, synthetic construct
  misc_feature 1..25: note = primer
  source 1..25: mol_type = other DNA
cagaaagtgtctacccagttgaaga

SEQ ID NO: 27 - DNA, 21 bp, synthetic construct
  misc_feature 1..21: note = primer
  source 1..21: mol_type = other DNA
agacagtcgcttccacttgtc

SEQ ID NO: 28 - DNA, 19 bp, synthetic construct
  misc_feature 1..19: note = primer
  source 1..19: mol_type = other DNA
tccgtccggtagacatgct

SEQ ID NO: 29 - DNA, 24 bp, synthetic construct
  misc_feature 1..24: note = primer
  source 1..24: mol_type = other DNA
aagtcagaatcctcctcttctcca

SEQ ID NO: 30 - DNA, 18 bp, synthetic construct
  misc_feature 1..18: note = primer
  source 1..18: mol_type = other DNA
cagcggcctgttcatcca

SEQ ID NO: 31 - DNA, 27 bp, synthetic construct
  misc_feature 1..27: note = primer
  source 1..27: mol_type = other DNA
cagaaatttcactcatccctaggctta

SEQ ID NO: 32 - DNA, 20 bp, synthetic construct
  misc_feature 1..20: note = primer
  source 1..20: mol_type = other DNA
tgcccagtcatttgcacctt

SEQ ID NO: 33 - DNA, 18 bp, synthetic construct
  misc_feature 1..18: note = primer
  source 1..18: mol_type = other DNA
tctcctcctgctccatca

SEQ ID NO: 34 - DNA, 20 bp, synthetic construct
  misc_feature 1..20: note = primer
  source 1..20: mol_type = other DNA
ccagctgtaagctgcttgga

SEQ ID NO: 35 - DNA, 18 bp, synthetic construct
  misc_feature 1..18: note = primer
  source 1..18: mol_type = other DNA
gtgcacccttcgcagttc

SEQ ID NO: 36 - DNA, 19 bp, synthetic construct
  misc_feature 1..19: note = primer
  source 1..19: mol_type = other DNA
cactgccaagcagctcatc

SEQ ID NO: 37 - DNA, 21 bp, synthetic construct
  misc_feature 1..21: note = primer
  source 1..21: mol_type = other DNA
gttggagagggacagcatgac

SEQ ID NO: 38 - skipped

SEQ ID NO: 39 - DNA, 393 bp, Homo sapiens
  source 1..393: mol_type = genomic DNA
tgtaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgctgcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcccaagatct

SEQ ID NO: 40 - DNA, 146 bp, Homo sapiens
  source 1..146: mol_type = genomic DNA
gggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgag

SEQ ID NO: 41 - protein, 59 aa, Homo sapiens
ALSWHLIHEQIPLSLDLQAGLDCCCLALQLPGLWSVVSSTEFVTHACSLIYCVHFILEA

SEQ ID NO: 42 - DNA, 178 bp, Homo sapiens
  source 1..178: mol_type = genomic DNA
gccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccg

SEQ ID NO: 43 - protein, 107 aa, Homo sapiens
DAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEA

SEQ ID NO: 44 - DNA, 324 bp, Homo sapiens
  source 1..324: mol_type = genomic DNA
gggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgaggccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccg

SEQ ID NO: 45 - DNA, 439 bp, Homo sapiens
  source 1..439: mol_type = genomic DNA
gggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgagaggcaagccctggtgctgtgggagccccaaggaagagcctctggcctggtggccacgtagcccaggagagatttctacaggagcccacagcgctgaaggagagagaggcagcagagccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccg

SEQ ID NO: 46 - DNA, 115 bp, Homo sapiens
  source 1..115: mol_type = genomic DNA
aggcaagccctggtgctgtgggagccccaaggaagagcctctggcctggtggccacgtagcccaggagagatttctacaggagcccacagcgctgaaggagagagaggcagcaga

SEQ ID NO: 47 - DNA, 23 bp, Homo sapiens
  source 1..23: mol_type = genomic DNA
ccaaggcctgctatccctagaac

SEQ ID NO: 48 - DNA, 1248 bp, Homo sapiens
  source 1..1248: mol_type = genomic DNA
gtaagaggcagctcgggagctcagtgttgctgtggggagggggcatggggctgacactgaagagggtaaagcagttttatttgaaaagcaagatctctgaccagtccagtcacttttccatctcagcctggcagtaagtcttgtcaccgtcaagttattgtagccatccttcaccctcacctcgccactcctcatggtggcctgtgaggtcagccaggtccccttctcatctgcacctaccatgttaggtggatcctaattttagagacatgaaaaataatcatctggaagtactttatgtcttaagttggcctggacatgtcagccaaggaatacttacttggtttgtgttagtgcttgtaattcgcccccagaatgtgtacacgttctggatgcattaaagtctggcctgtatccttaaagggccatcgctgtgctgcctgccctcagcaaggacacactttgcagacccacagaggctccgcctccacctcacaccaaagaaagggaggagtccaaagggcatcagtgccattactcacaaaatgataaatacacccttattctgaaccacgtggagtcatatggtttgtgatccctgtccttcaggtttcagcttagtggggaagtgggaaagtcagcgtgtgatcacagcacagggtgattgctgctgattatattatgtgcctgctgtatgcaggatgaaatactttatatgcgtcatcttatttgactctcacaaccccctgtgagataggctctgttactcccatttgacaggtgaggaaagcaaggcttagagaatttcagtgacttgcccaggtcctctgagctaggaagtagccattctggcatttgaacccaaggcctgctatccctagaacccacgctctcaaattcaacctatgacagaggcaagccctggtgctgtgggagccccaaggaagagcctctggcctggtggccacgtagcccaggagagatttctacaggagcccacagcgctgaaggagagagaggcagcagagtaagggggctttgtggcagagaggggactggcactttggggaataggtgggtcaggactgaatgtaatggagccatgtcagagctgtccttctggaagggcaagggcacctggacgcgctgcccctcagtgctttggacggttccacaactgtgattcacacggcttccccaaacgaaggtacacgagtgggcattctgtgactcggtacttccctttag

SEQ ID NO: 49 - DNA, 146 bp, Homo sapiens
  source 1..146: mol_type = genomic DNA
aacccacgctctcaaattcaacctatgacagaggcaagccctggtgctgtgggagccccaaggaagagcctctggcctggtggccacgtagcccaggagagatttctacaggagcccacagcgctgaaggagagagaggcagcaga

SEQ ID NO: 50 - DNA, 10 bp, Homo sapiens
  source 1..10: mol_type = genomic DNA
ttgagaaccc

SEQ ID NO: 51 - DNA, 10 bp, Homo sapiens
  source 1..10: mol_type = genomic DNA
gcagagccct

SEQ ID NO: 52 - DNA, 470 bp, Homo sapiens
  source 1..470: mol_type = genomic DNA
gggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgagaacccacgctctcaaattcaacctatgacagaggcaagccctggtgctgtgggagccccaaggaagagcctctggcctggtggccacgtagcccaggagagatttctacaggagcccacagcgctgaaggagagagaggcagcagagccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccg

SEQ ID NO: 53 - DNA, 13 bp, Homo sapiens
  source 1..13: mol_type = genomic DNA
tgagaggcaagcc

SEQ ID NO: 54 - protein, 102 aa, synthetic construct
  REGION 1..102: note = Truncated HTT polypeptide
DAALYQSLPTLARALAQYLVVVSKLPSHLHLPPEKEKDIVKFVVATLENPRSQIQPMTEASPGAVGAPRKSLWPGGHVAQERFLQEPTALKEREAAEPCPGI

SEQ ID NO: 55 - DNA, 12 bp, Homo sapiens
  source 1..12: mol_type = genomic DNA
gcagagccctgt

SEQ ID NO: 56 - DNA, 20 bp, synthetic construct
  misc_feature 1..20: note = primer
  source 1..20: mol_type = other DNA
taatacgactcactataggg

SEQ ID NO: 57 - protein, 67 aa, Homo sapiens
DAALYQSLPTLARALAQYLVVVSKLPSHLHLPPEKEKDIVKFVVATLERQALVLWEPQGRASGLVAT

SEQ ID NO: 58 - DNA, 39 bp, Homo sapiens
  source 1..39: mol_type = genomic DNA
gctccctcatctactgtgtgcacttcatcctggaggccg

SEQ ID NO: 59 - DNA, 18 bp, synthetic construct
  misc_feature 1..18: note = primer
  source 1..18: mol_type = other DNA
tagaaggcacagtcgagg

SEQ ID NO: 60 - protein, 4 aa, Homo sapiens
ALSW

SEQ ID NO: 61 - protein, 19 aa, Homo sapiens
RQALVLWEPQGRASGLVAT

SEQ ID NO: 62 - protein, 11 aa, synthetic construct
  REGION 1..11: note = polypeptide
PRRDFYRSPQR

SEQ ID NO: 63 - DNA, 393 bp, Homo sapiens; identical to SEQ ID NO: 39
  source 1..393: mol_type = genomic DNA

SEQ ID NO: 64 - RNA, 21 nt, Homo sapiens
  source 1..21: mol_type = other RNA
gagaggcagcagagtaagggg

SEQ ID NO: 65 - RNA, 21 nt, Homo sapiens
  source 1..21: mol_type = other RNA
atacttactctgcaggggaga

SEQ ID NO: 66 - DNA, 602 bp, synthetic construct
  misc_feature 1..602: note = plasmid sequence
  source 1..602: mol_type = other DNA
gacggatcgggagatctgtaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgctgcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcccaagatctcatacttactctgcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccctgactttctggagtttcaaaagtagactgt

SEQ ID NO: 67 - DNA, 1572 bp, synthetic construct
  misc_feature 1..1572: note = Human HTT minigene
  exon 1..146: note = Human HTT EXON 49 (146 nt)
  intron 147..1394: note = Human HTT INTRON 49 (1248 nt)
  exon 1395..1572: note = HUMAN HTT EXON 50 (178 nt)
  source 1..1572: mol_type = other DNA
gggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgaggtaagaggcagctcgggagctcagtgttgctgtggggagggggcatggggctgacactgaagagggtaaagcagttttatttgaaaagcaagatctctgaccagtccagtcacttttccatctcagcctggcagtaagtcttgtcaccgtcaagttattgtagccatccttcaccctcacctcgccactcctcatggtggcctgtgaggtcagccaggtccccttctcatctgcacctaccatgttaggtggatcctaattttagagacatgaaaaataatcatctggaagtactttatgtcttaagttggcctggacatgtcagccaaggaatacttacttggtttgtgttagtgcttgtaattcgcccccagaatgtgtacacgttctggatgcattaaagtctggcctgtatccttaaagggccatcgctgtgctgcctgccctcagcaaggacacactttgcagacccacagaggctccgcctccacctcacaccaaagaaagggaggagtccaaagggcatcagtgccattactcacaaaatgataaatacacccttattctgaaccacgtggagtcatatggtttgtgatccctgtccttcaggtttcagcttagtggggaagtgggaaagtcagcgtgtgatcacagcacagggtgattgctgctgattatattatgtgcctgctgtatgcaggatgaaatactttatatgcgtcatcttatttgactctcacaaccccctgtgagataggctctgttactcccatttgacaggtgaggaaagcaaggcttagagaatttcagtgacttgcccaggtcctctgagctaggaagtagccattctggcatttgaacccaaggcctgctatccctagaacccacgctctcaaattcaacctatgacagaggcaagccctggtgctgtgggagccccaaggaagagcctctggcctggtggccacgtagcccaggagagatttctacaggagcccacagcgctgaaggagagagaggcagcagagtaagggggctttgtggcagagaggggactggcactttggggaataggtgggtcaggactgaatgtaatggagccatgtcagagctgtccttctggaagggcaagggcacctggacgcgctgcccctcagtgctttggacggttccacaactgtgattcacacggcttccccaaacgaaggtacacgagtgggcattctgtgactcggtacttccctttaggccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccg

SEQ ID NO: 68 - DNA, 1558 bp, synthetic construct
  misc_feature 1..1558: note = Mouse HTT minigene
  exon 1..146: note = MOUSE HTT EXON 49 (146 nt)
  source 1..1558: mol_type = other DNA
gtgataccacatcataccagtctctgaccatacttgcccgtgccctggcacagtacctggtggtgctctccaaagtgcctgctcatttgcaccttcctcctgagaaggagggggacacggtgaagtttgtggtaatgacagttgaggtaagagcagctctgaaattatgtgtccctgtgaggacaggatatgtgagtagcactaagatgaaagtccttgaaaaccgacagtgtggagtacaatagtgcacacattagcccagctgccttggaggcagaggcagaattgtgggttcctggtctgtaaggatgtgcctgagtatacagctagaccctatttgaataaacaggaaggcagggaatacctattggcaaagtctgattcacctgatggtacagagtgcctttcaccctcaccactgggaagcaaggaggtctgtaagacatcctgttatccctacactataaacctaatgtgggtcctaaataaaatctagacagtgttacattttaaattgggcagtgaagctggacatttcacccagaaacacttggcccctcaaaatgtatctatacgtgcactatagttttattaccttgccatgggcatgctgggaaagagcctcactgtgccagagctgtgctgccaatcctgaacaagggttgacaccttaccctaagagaagaaagtcagtatcctgagggtgtatggtacaaaggcaccaggtgaaccaggctaagttaggtggtctttgagcttgtcttagcccagtgaagacaggaaagcaaatgtgtgtgtaaagtattgggtggcagctcctagtcatactctgcgctgcacaggccatgccatgacacttgtttcctataaaaactctgtccccatttcacacatggggaaagaagctcagagaggttcggggacttgctagaagtcactagtcataaatcatactccaaaactcagtgttgtgactgagatacaaaacaaaacacattctgtttctttaaaaaaaaaaaaaaaagtccaatgttacaggagccctcaagaccctggctggaggctttgtatatggctcagatcaagtttctatgggcacccatggttctaaaggaaaaagacacctagggtaaggttgggcattctggcagagggaaggctggaacttggggtataggtggatcaggactgaatattaagaaaactaggaatgacaacctagaaatgtgggtcaggggccattcttctgcaaggaggttgtctgatctcctgccctctccatctatccatggtcttccatatcttttattggcactgcttccctagaggtccctgagatagagtcctgggtgagcattctaacacagtgcttccctttaggccctgtcatggcatttgatccatgagcagatcccactgagtctggacctccaagccgggctagactgctgctgcctggcactacaggtgcctggcctctggggggtgctgtcctccccagagtacgtgactcatgcctgctccctcatccattgtgtgcgattcatcctggaagcca

SEQ ID NO: 69 - DNA, 1558 bp, synthetic construct
  misc_feature 1..1558: note = Hybrid HTT minigene
  exon 1..146: note = MOUSE HTT EXON 49 (146 nt)
  intron 147..1380: note = MOUSE HTT INTRON 49 (1234 nt)
  exon 1381..1558: note = MOUSE HTT EXON 50 (178 nt)
  source 1..1558: mol_type = other DNA
gtgataccacatcataccagtctctgaccatacttgcccgtgccctggcacagtacctggtggtgctctccaaagtgcctgctcatttgcaccttcctcctgagaaggagggggacacggtgaagtttgtggtaatgacagttgaggtaagagcagctctgaaattatgtgtccctgtgaggacaggatatgtgagtagcactaagatgaaagtccttgaaaaccgacagtgtggagtacaatagtgcacacattagcccagctgccttggaggcagaggcagaattgtgggttcctggtctgtaaggatgtgcctgagtatacagctagaccctatttgaataaacaggaaggcagggaatacctattggcaaagtctgattcacctgatggtacagagtgcctttcaccctcaccactgggaagcaaggaggtctgtaagacatcctgttatccctacactataaacctaatgtgggtcctaaataaaatctagacagtgttacattttaaattgggcagtgaagctggacatttcacccagaaacacttggcccctcaaaatgtatctatacgtgcactatagttttattaccttgccatgggcatgctgggaaagagcctcactgtgccagagctgtgctgccaatcctgaacaagggttgacaccttaccctaagagaagaaagtcagtatcctgagggtgtatggtacaaaggcaccaggtgaaccaggctaagttaggtggtctttgagcttgtcttagcccagtgaagacaggaaagcaaatgtgtgtgtaaagtattgggtggcagctcctagtcatactctgcgctgcacaggccatgccatgacacttgtttcctataaaaactctgtccccatttcacacatggggaaagaagctcagagaggttcggggacttgctagaagtcactagtcataaatcatactccaaaactccaaggcctgctatccctagaacccacgctctcaaattcaacctatgacagaggcaagccctggtgctgtgggagccccaaggaagagcctctggcctggtggccaatatggctcagatcaagtttctatgggcacccatggttctaaaggaaaaagacacctagggtaaggttgggcattctggcagagggaaggctggaacttggggtataggtggatcaggactgaatattaagaaaactaggaatgacaacctagaaatgtgggtcaggggccattcttctgcaaggaggttgtctgatctcctgccctctccatctatccatggtcttccatatcttttattggcactgcttccctagaggtccctgagatagagtcctgggtgagcattctaacacagtgcttccctttaggccctgtcatggcatttgatccatgagcagatcccactgagtctggacctccaagccgggctagactgctgctgcctggcactacaggtgcctggcctctggggggtgctgtcctccccagagtacgtgactcatgcctgctccctcatccattgtgtgcgattcatcctggaagcca

SEQ ID NO: 70 - DNA, 1558 bp, synthetic construct
  misc_feature 1..1558: note = Minigene construct
  exon 1..146: note = Human HTT EXON 49 (146 nt)
  intron 147..964: note = Mouse HTT intron
  intron 965..1014: note = Human HTT intron
  exon 1015..1129: note = Human HTT PsiEXON 49 (115 nt)
  intron 1130..1179: note = Human HTT intron
  intron 1180..1380: note = Mouse HTT intron
  exon 1381..1558: note = MOUSE HTT EXON 50 (178 nt)
  source 1..1558: mol_type = other DNA
gtgataccacatcataccagtctctgaccatacttgcccgtgccctggcacagtacctggtggtgctctccaaagtgcctgctcatttgcaccttcctcctgagaaggagggggacacggtgaagtttgtggtaatgacagttgaggtaagagcagctctgaaattatgtgtccctgtgaggacaggatatgtgagtagcactaagatgaaagtccttgaaaaccgacagtgtggagtacaatagtgcacacattagcccagctgccttggaggcagaggcagaattgtgggttcctggtctgtaaggatgtgcctgagtatacagctagaccctatttgaataaacaggaaggcagggaatacctattggcaaagtctgattcacctgatggtacagagtgcctttcaccctcaccactgggaagcaaggaggtctgtaagacatcctgttatccctacactataaacctaatgtgggtcctaaataaaatctagacagtgttacattttaaattgggcagtgaagctggacatttcacccagaaacacttggcccctcaaaatgtatctatacgtgcactatagttttattaccttgccatgggcatgctgggaaagagcctcactgtgccagagctgtgctgccaatcctgaacaagggttgacaccttaccctaagagaagaaagtcagtatcctgagggtgtatggtacaaaggcaccaggtgaaccaggctaagttaggtggtctttgagcttgtcttagcccagtgaagacaggaaagcaaatgtgtgtgtaaagtattgggtggcagctcctagtcatactctgcgctgcacaggccatgccatgacacttgtttcctataaaaactctgtccccatttcacacatggggaaagaagctcagagaggttcggggacttgctagaagtcactagtcataaatcatactccaaaactccaaggcctgctatccctagaacccacgctctcaaattcaacctatgacagaggcaagccctggtgctgtgggagccccaaggaagagcctctggcctggtggccacgtagcccaggagagatttctacaggagcccacagcgctgaaggagagagaggcagcagagtaagggggctttgtggcagagaggggactggcactttggggaataggtgggatcaggactgaatattaagaaaactaggaatgacaacctagaaatgtgggtcaggggccattcttctgcaaggaggttgtctgatctcctgccctctccatctatccatggtcttccatatcttttattggcactgcttccctagaggtccctgagatagagtcctgggtgagcattctaacacagtgcttccctttaggccctgtcatggcatttgatccatgagcagatcccactgagtctggacctccaagccgggctagactgctgctgcctggcactacaggtgcctggcctctggggggtgctgtcctccccagagtacgtgactcatgcctgctccctcatccattgtgtgcgattcatcctggaagcca

SEQ ID NO: 71 - DNA, 1558 bp, synthetic construct
  misc_feature 1..1558: note = Minigene construct
  exon 1..146: note = MOUSE HTT EXON 49
  intron 147..1069: note = MOUSE HTT INTRON 49 (923 nt)
  exon 1070..1129: note = Human HTT psiExon (60nt)
  intron 1130..1179: note = Human intron (50 nt)
  intron 1180..1380: note = MOUSE HTT INTRON (201 nt)
  exon 1381..1558: note = MOUSE HTT EXON 50 (178 nt)
  source 1..1558: mol_type = other DNA
gtgataccacatcataccagtctctgaccatacttgcccgtgccctggcacagtacctggtggtgctctccaaagtgcctgctcatttgcaccttcctcctgagaaggagggggacacggtgaagtttgtggtaatgacagttgaggtaagagcagctctgaaattatgtgtccctgtgaggacaggatatgtgagtagcactaagatgaaagtccttgaaaaccgacagtgtggagtacaatagtgcacacattagcccagctgccttggaggcagaggcagaattgtgggttcctggtctgtaaggatgtgcctgagtatacagctagaccctatttgaataaacaggaaggcagggaatacctattggcaaagtctgattcacctgatggtacagagtgcctttcaccctcaccactgggaagcaaggaggtctgtaagacatcctgttatccctacactataaacctaatgtgggtcctaaataaaatctagacagtgttacattttaaattgggcagtgaagctggacatttcacccagaaacacttggcccctcaaaatgtatctatacgtgcactatagttttattaccttgccatgggcatgctgggaaagagcctcactgtgccagagctgtgctgccaatcctgaacaagggttgacaccttaccctaagagaagaaagtcagtatcctgagggtgtatggtacaaaggcaccaggtgaaccaggctaagttaggtggtctttgagcttgtcttagcccagtgaagacaggaaagcaaatgtgtgtgtaaagtattgggtggcagctcctagtcatactctgcgctgcacaggccatgccatgacacttgtttcctataaaaactctgtccccatttcacacatggggaaagaagctcagagaggttcggggacttgctagaagtcactagtcataaatcatactccaaaactcagtgttgtgactgagatacaaaacaaaacacattctgtttctttaaaaaaaaaaaaaaaagtccaatgttacaggagccctcaagaccctggctggaggctttgtcgtagcccaggagagatttctacaggagcccacagcgctgaaggagagagaggcagcagagtaagggggctttgtggcagagaggggactggcactttggggaataggtgggatcaggactgaatattaagaaaactaggaatgacaacctagaaatgtgggtcaggggccattcttctgcaaggaggttgtctgatctcctgccctctccatctatccatggtcttccatatcttttattggcactgcttccctagaggtccctgagatagagtcctgggtgagcattctaacacagtgcttccctttaggccctgtcatggcatttgatccatgagcagatcccactgagtctggacctccaagccgggctagactgctgctgcctggcactacaggtgcctggcctctggggggtgctgtcctccccagagtacgtgactcatgcctgctccctcatccattgtgtgcgattcatcctggaagcca

SEQ ID NO: 72 - DNA, 1558 bp, synthetic construct; identical to SEQ ID NO: 69
  misc_feature 1..1558: note = Minigene construc
  exon 1..146: note = MOUSE HTT EXON 49 (146 nt)
  exon 147..964: note = MOUSE HTT EXON 49 (146 nt)
  exon 965..1014: note = Human HTT EXON 49 (50 nt)
  exon 1015..1069: note = Human HTT PsiEXON 49 (55 nt)
  intron 1070..1380: note = MOUSE HTT INTRON 49 (311 nt)
  exon 1381..1558: note = MOUSE HTT EXON 50 (178 nt)
  source 1..1558: mol_type = other DNA

SEQ ID NO: 73 - DNA, 1547 bp, synthetic construct
  misc_feature 1..1547: note = Minigene construc
  exon 1..146: note = MOUSE HTT EXON 49 (146 nt)
  intron 147..964: note = MOUSE HTT INTRON (818 nt)
  intron 965..1014: note = Human HTT intron-1 US iExon (50 nt)
  intron 1015..1118: note = Human HTT intron-1 US iExon (50 nt)
  intron 1119..1158: note = Human HTT intron-1 DS iExon (50nt)
  intron 1169..1369: note = MOUSE HTT INTRON (201 nt)
  exon 1370..1547: note = MOUSE HTT EXON 50 (178 nt)
  source 1..1547: mol_type = other DNA
gtgataccacatcataccagtctctgaccatacttgcccgtgccctggcacagtacctggtggtgctctccaaagtgcctgctcatttgcaccttcctcctgagaaggagggggacacggtgaagtttgtggtaatgacagttgaggtaagagcagctctgaaattatgtgtccctgtgaggacaggatatgtgagtagcactaagatgaaagtccttgaaaaccgacagtgtggagtacaatagtgcacacattagcccagctgccttggaggcagaggcagaattgtgggttcctggtctgtaaggatgtgcctgagtatacagctagaccctatttgaataaacaggaaggcagggaatacctattggcaaagtctgattcacctgatggtacagagtgcctttcaccctcaccactgggaagcaaggaggtctgtaagacatcctgttatccctacactataaacctaatgtgggtcctaaataaaatctagacagtgttacattttaaattgggcagtgaagctggacatttcacccagaaacacttggcccctcaaaatgtatctatacgtgcactatagttttattaccttgccatgggcatgctgggaaagagcctcactgtgccagagctgtgctgccaatcctgaacaagggttgacaccttaccctaagagaagaaagtcagtatcctgagggtgtatggtacaaaggcaccaggtgaaccaggctaagttaggtggtctttgagcttgtcttagcccagtgaagacaggaaagcaaatgtgtgtgtaaagtattgggtggcagctcctagtcatactctgcgctgcacaggccatgccatgacacttgtttcctataaaaactctgtccccatttcacacatggggaaagaagctcagagaggttcggggacttgctagaagtcactagtcataaatcatactccaaaactccttgactaaaaatacaaaaattagccgggcatggtggtgtgcacctgtagtcccagctacttgggaggctgaggcaagagaactgcttgaacccgaggggcagaggttgcagtgagctgagagtgcgccattgcacttcagcctgtgtgacagagtaagactccatctccaaaaaaaaaaaaccaagatcaattaaaatacagcggatcaggactgaatattaagaaaactaggaatgacaacctagaaatgtgggtcaggggccattcttctgcaaggaggttgtctgatctcctgccctctccatctatccatggtcttccatatcttttattggcactgcttccctagaggtccctgagatagagtcctgggtgagcattctaacacagtgcttccctttaggccctgtcatggcatttgatccatgagcagatcccactgagtctggacctccaagccgggctagactgctgctgcctggcactacaggtgcctggcctctggggggtgctgtcctccccagagtacgtgactcatgcctgctccctcatccattgtgtgcgattcatcctggaagcca

SEQ ID NO: 74 - DNA, 1534 bp, synthetic construct
  misc_feature 1..1534: note = Minigene construc
  exon 1..146: note = MOUSE HTT EXON 49 (146 nt)
  intron 147..964: note = Human intron-8 US iExon (50 nt)
  intron 965..1014: note = Human intron-8 US iExon (50 nt)
  intron 1015..1105: note = Human intron-8 psiExon (91 nt)
  intron 1106..1155: note = Human intron-8 DS iExon (50 nt)
  intron 1156..1356: note = MOUSE HTT INTRON (201 nt)
  exon 1357..1534: note = MOUSE HTT EXON 50 (178 nt)
  source 1..1534: mol_type = other DNA
gtgataccacatcataccagtctctgaccatacttgcccgtgccctggcacagtacctggtggtgctctccaaagtgcctgctcatttgcaccttcctcctgagaaggagggggacacggtgaagtttgtggtaatgacagttgaggtaagagcagctctgaaattatgtgtccctgtgaggacaggatatgtgagtagcactaagatgaaagtccttgaaaaccgacagtgtggagtacaatagtgcacacattagcccagctgccttggaggcagaggcagaattgtgggttcctggtctgtaaggatgtgcctgagtatacagctagaccctatttgaataaacaggaaggcagggaatacctattggcaaagtctgattcacctgatggtacagagtgcctttcaccctcaccactgggaagcaaggaggtctgtaagacatcctgttatccctacactataaacctaatgtgggtcctaaataaaatctagacagtgttacattttaaattgggcagtgaagctggacatttcacccagaaacacttggcccctcaaaatgtatctatacgtgcactatagttttattaccttgccatgggcatgctgggaaagagcctcactgtgccagagctgtgctgccaatcctgaacaagggttgacaccttaccctaagagaagaaagtcagtatcctgagggtgtatggtacaaaggcaccaggtgaaccaggctaagttaggtggtctttgagcttgtcttagcccagtgaagacaggaaagcaaatgtgtgtgtaaagtattgggtggcagctcctagtcatactctgcgctgcacaggccatgccatgacacttgtttcctataaaaactctgtccccatttcacacatggggaaagaagctcagagaggttcggggacttgctagaagtcactagtcataaatcatactccaaaactcatatattatcaacaaggagggagaaaaaggcaattagtaaccatccataggagggtcagcaagatttataaaggaaatttgtgatccaagtatgaagcaaaataaggtgcagaataaattttaagcaagtaatagattagagtaagagaacccatttgaccattaaccttgggacattctctttcaaatgaggatcaggactgaatattaagaaaactaggaatgacaacctagaaatgtgggtcaggggccattcttctgcaaggaggttgtctgatctcctgccctctccatctatccatggtcttccatatcttttattggcactgcttccctagaggtccctgagatagagtcctgggtgagcattctaacacagtgcttccctttaggccctgtcatggcatttgatccatgagcagatcccactgagtctggacctccaagccgggctagactgctgctgcctggcactacaggtgcctggcctctggggggtgctgtcctccccagagtacgtgactcatgcctgctccctcatccattgtgtgcgattcatcctggaagcca

SEQ ID NO: 75 - DNA, 1541 bp, synthetic construct
  misc_feature 1..1541: note = Minigene construc
  exon 1..146: note = MOUSE HTT EXON 49 (146 nt)
  intron 147..964: note = MOUSE HTT INTRON (818 nt)
  intron 965..1014: note = Human intron-40a US iExon (50nt)
  intron 1015..1112: note = Human intron-40a psiExon (98 nt)
  intron 1113..1162: note = Human intron-40a DS iExon (50 nt)
  intron 1163..1363: note = MOUSE HTT INTRON (201 nt)
  exon 1364..1541: note = MOUSE HTT EXON 50 (178 nt)
  source 1..1541: mol_type = other DNA
gtgataccacatcataccagtctctgaccatacttgcccgtgccctggcacagtacctggtggtgctctccaaagtgcctgctcatttgcaccttcctcctgagaaggagggggacacggtgaagtttgtggtaatgacagttgaggtaagagcagctctgaaattatgtgtccctgtgaggacaggatatgtgagtagcactaagatgaaagtccttgaaaaccgacagtgtggagtacaatagtgcacacattagcccagctgccttggaggcagaggcagaattgtgggttcctggtctgtaaggatgtgcctgagtatacagctagaccctatttgaataaacaggaaggcagggaatacctattggcaaagtctgattcacctgatggtacagagtgcctttcaccctcaccactgggaagcaaggaggtctgtaagacatcctgttatccctacactataaacctaatgtgggtcctaaataaaatctagacagtgttacattttaaattgggcagtgaagctggacatttcacccagaaacacttggcccctcaaaatgtatctatacgtgcactatagttttattaccttgccatgggcatgctgggaaagagcctcactgtgccagagctgtgctgccaatcctgaacaagggttgacaccttaccctaagagaagaaagtcagtatcctgagggtgtatggtacaaaggcaccaggtgaaccaggctaagttaggtggtctttgagcttgtcttagcccagtgaagacaggaaagcaaatgtgtgtgtaaagtattgggtggcagctcctagtcatactctgcgctgcacaggccatgccatgacacttgtttcctataaaaactctgtccccatttcacacatggggaaagaagctcagagaggttcggggacttgctagaagtcactagtcataaatcatactccaaaactccatctgtacaaacaaactagccggggatagtggtgtgcatgtggtcccagctactcaggagactgaggctggaggatcgcttgagcccaggaggttaagtctctagtgagatgtgttcatgccactgcactccagcctcggctatagagtaagaccctgcctcaaaaaaacaaaacaaaacaagacaagagccaaaaaggatcaggactgaatattaagaaaactaggaatgacaacctagaaatgtgggtcaggggccattcttctgcaaggaggttgtctgatctcctgccctctccatctatccatggtcttccatatcttttattggcactgcttccctagaggtccctgagatagagtcctgggtgagcattctaacacagtgcttccctttaggccctgtcatggcatttgatccatgagcagatcccactgagtctggacctccaagccgggctagactgctgctgcctggcactacaggtgcctggcctctggggggtgctgtcctccccagagtacgtgactcatgcctgctccctcatccattgtgtgcgattcatcctggaagcca

SEQ ID NO: 76 - DNA, 1609 bp, synthetic construct
  misc_feature 1..1609: note = Minigene construc
  exon 1..146: note = MOUSE HTT EXON 49 (146 nt)
  intron 147..964: note = MOUSE HTT INTRON (818 nt)
  intron 965..1014: note = Human intron-40b US iExon (50nt)
  intron 1015..1180: note = Human intron-40b psiExon (166 nt)
  intron 1181..1230: note = Human intron-40b DS iExon (50nt)
  intron 1231..1431: note = MOUSE HTT INTRON (201 nt)
  exon 1432..1609: note = MOUSE HTT EXON 50 (178 nt)
  source 1..1609: mol_type = other DNA
gtgataccacatcataccagtctctgaccatacttgcccgtgccctggcacagtacctggtggtgctctccaaagtgcctgctcatttgcaccttcctcctgagaaggagggggacacggtgaagtttgtggtaatgacagttgaggtaagagcagctctgaaattatgtgtccctgtgaggacaggatatgtgagtagcactaagatgaaagtccttgaaaaccgacagtgtggagtacaatagtgcacacattagcccagctgccttggaggcagaggcagaattgtgggttcctggtctgtaaggatgtgcctgagtatacagctagaccctatttgaataaacaggaaggcagggaatacctattggcaaagtctgattcacctgatggtacagagtgcctttcaccctcaccactgggaagcaaggaggtctgtaagacatcctgttatccctacactataaacctaatgtgggtcctaaataaaatctagacagtgttacattttaaattgggcagtgaagctggacatttcacccagaaacacttggcccctcaaaatgtatctatacgtgcactatagttttattaccttgccatgggcatgctgggaaagagcctcactgtgccagagctgtgctgccaatcctgaacaagggttgacaccttaccctaagagaagaaagtcagtatcctgagggtgtatggtacaaaggcaccaggtgaaccaggctaagttaggtggtctttgagcttgtcttagcccagtgaagacaggaaagcaaatgtgtgtgtaaagtattgggtggcagctcctagtcatactctgcgctgcacaggccatgccatgacacttgtttcctataaaaactctgtccccatttcacacatggggaaagaagctcagagaggttcggggacttgctagaagtcactagtcataaatcatactccaaaactcaaatatagaggcttcagaggtctccaaatagaactaaacatgtaattcagattgttaggaggtagtataaatgagctaaatctcatctttattacggtagagttaatgggtgatgtctaaagttgtctgaagtctataaatcatgacaaattatgatgtggtgattgtattcaacagtctttcagttgcagggataaaaccccagtttaaactagagtaagagaaagaatgtgttggtttaagctcctggaaagtgcaggcaagggggatcaggactgaatattaagaaaactaggaatgacaacctagaaatgtgggtcaggggccattcttctgcaaggaggttgtctgatctcctgccctctccatctatccatggtcttccatatcttttattggcactgcttccctagaggtccctgagatagagtcctgggtgagcattctaacacagtgcttccctttaggccctgtcatggcatttgatccatgagcagatcccactgagtctggacctccaagccgggctagactgctgctgcctggcactacaggtgcctggcctctggggggtgctgtcctccccagagtacgtgactcatgcctgctccctcatccattgtgtgcgattcatcctggaagcca

SEQ ID NO: 77 - DNA, 1780 bp, Homo sapiens
  source 1..1780: mol_type = genomic DNA
cagattctcagagaattgcttgactgcctttcgaagttgatgcatctgtgctcacgtttgcacccacccacgaggtccttctgtttcaggggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgaggtaagaggcagctcgggagctcagtgttgctgtggggagggggcatggggctgacactgaagagggtaaagcagttttatttgaaaagcaagatctctgaccagtccagtcacttttccatctcagcctggcagtaagtcttgtcaccgtcaagttattgtagccatccttcaccctcacctcgccactcctcatggtggcctgtgaggtcagccaggtccccttctcatctgcacctaccatgttaggtggatcctaattttagagacatgaaaaataatcatctggaagtactttatgtcttaagttggcctggacatgtcagccaaggaatacttacttggtttgtgttagtgcttgtaattcgcccccagaatgtgtacacgttctggatgcattaaagtctggcctgtatccttaaagggccatcgctgtgctgcctgccctcagcaaggacacactttgcagacccacagaggctccgcctccacctcacaccaaagaaagggaggagtccaaagggcatcagtgccattactcacaaaatgataaatacacccttattctgaaccacgtggagtcatatggtttgtgatccctgtccttcaggtttcagcttagtggggaagtgggaaagtcagcgtgtgatcacagcacagggtgattgctgctgattatattatgtgcctgctgtatgcaggatgaaatactttatatgcgtcatcttatttgactctcacaaccccctgtgagataggctctgttactcccatttgacaggtgaggaaagcaaggcttagagaatttcagtgacttgcccaggtcctctgagctaggaagtagccattctggcatttgaacccaaggcctgctatccctagaacccacgctctcaaattcaacctatgacagaggcaagccctggtgctgtgggagccccaaggaagagcctctggcctggtggccacgtagcccaggagagatttctacaggagcccacagcgctgaaggagagagaggcagcagagtaagggggctttgtggcagagaggggactggcactttggggaataggtgggtcaggactgaatgtaatggagccatgtcagagctgtccttctggaagggcaagggcacctggacgcgctgcccctcagtgctttggacggttccacaactgtgattcacacggcttccccaaacgaaggtacacgagtgggcattctgtgactcggtacttccctttaggccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccggtgagtccccgtccatgaacggtgggttcctatcatagttcctgtctgcttcaccatgtttttattttgtgctgcctgtttgccaggtactaagctaggaattggggatggagaggtag

SEQ ID NO: 78 - skipped

SEQ ID NO: 79 - DNA, 35 bp, Homo sapiens
  source 1..35: mol_type = genomic DNA
gagatttctacaggagcccacagcgctgaaggaga

SEQ ID NO: 80 - DNA, 35 bp, Homo sapiens
  source 1..35: mol_type = genomic DNA
gagatttctatttgagcccacagcgctgaaggaga

SEQ ID NO: 81 - DNA, 35 bp, Homo sapiens
  source 1..35: mol_type = genomic DNA
gagatttctattttagcccacagcgctgaaggaga

SEQ ID NO: 82 - DNA, 35 bp, Homo sapiens
  source 1..35: mol_type = genomic DNA
gagatttctatttttgcccacagcgctgaaggaga

SEQ ID NO: 83 - DNA, 35 bp, Homo sapiens
  source 1..35: mol_type = genomic DNA
gagatttctttttgagcccacagcgctgaaggaga

SEQ ID NO: 84 - DNA, 31 bp, Homo sapiens
  source 1..31: mol_type = genomic DNA
gagatttctacaggagcagcgctgaaggaga

SEQ ID NO: 85 - DNA, 20 bp, Homo sapiens
  source 1..20: mol_type = genomic DNA
caggagcccacagcgctgaa

SEQ ID NO: 86 - DNA, 18 bp, Homo sapiens
  source 1..18: mol_type = genomic DNA
ggagcccacagcgctgaa

SEQ ID NO: 87 - DNA, 16 bp, Homo sapiens
  source 1..16: mol_type = genomic DNA
agcccacagcgctgaa

SEQ ID NO: 88 - DNA, 14 bp, Homo sapiens
  source 1..14: mol_type = genomic DNA
cccacagcgctgaa

SEQ ID NO: 89 - DNA, 12 bp, Homo sapiens
  source 1..12: mol_type = genomic DNA
cacagcgctgaa

SEQ ID NO: 90 - DNA, 10 bp, Homo sapiens
  source 1..10: mol_type = genomic DNA
cagcgctgaa

SEQ ID NO: 91 - DNA, 18 bp, Homo sapiens
  source 1..18: mol_type = genomic DNA
caggagcccacagcgctg

SEQ ID NO: 92 - DNA, 16 bp, Homo sapiens
  source 1..16: mol_type = genomic DNA
caggagcccacagcgc

SEQ ID NO: 93 - DNA, 14 bp, Homo sapiens
  source 1..14: mol_type = genomic DNA
caggagcccacagc

SEQ ID NO: 94 - DNA, 13 bp, Homo sapiens
  source 1..13: mol_type = genomic DNA
acaggagcccaca

SEQ ID NO: 95 - DNA, 10 bp, Homo sapiens
  source 1..10: mol_type = genomic DNA
caggagccca

SEQ ID NO: 96 - DNA, 215 bp, Homo sapiens
  source 1..215: mol_type = genomic DNA
caaggcctgctatccctagaacccacgctctcaaattcaacctatgacagaggcaagccctggtgctgtgggagccccaaggaagagcctctggcctggtggccacgtagcccaggagagatttctacaggagcccacagcgctgaaggagagagaggcagcagagtaagggggctttgtggcagagaggggactggcactttggggaataggtg

SEQ ID NO: 97 - protein, 48 aa, Homo sapiens
DAALYQSLPTLARALAQYLVVVSKLPSHLHLPPEKEKDIVKFVVATLE

SEQ ID NO: 98 - DNA, 10 bp, Homo sapiens
  source 1..10: mol_type = genomic DNA
cagagtaaga

SEQ ID NO: 99 - DNA, 10 bp, Homo sapiens
  source 1..10: mol_type = genomic DNA
tagagtaaga

SEQ ID NO: 100 - DNA, 10 bp, Homo sapiens
  source 1..10: mol_type = genomic DNA
tagagtaaga

SEQ ID NO: 101 - DNA, 10 bp, Homo sapiens
  source 1..10: mol_type = genomic DNA
tagagtaaga

SEQ ID NO: 102 - DNA, 1538 bp, synthetic construct
  misc_feature 1..1538: note = minigene construct
  exon 1..146: note = MOUSE HTT EXON 49 (146 nt)
  intron 147..964: note = MOUSE HTT INTRON (818 nt)
  intron 965..1014: note = Human HTT intron (50 nt)
  exon 1015..1109: note = Human HTT PSIEXON 49 20 nt deletion (95 nt; 20 nt deletion mutant)
  intron 1110..1159: note = Human HTT intron (50 nt)
  intron 1160..1360: note = MOUSE HTT INTRON (201 nt)
  exon 1361..1538: note = MOUSE HTT EXON 50 (178 nt)
  source 1..1538: mol_type = other DNA
gtgataccacatcataccagtctctgaccatacttgcccgtgccctggcacagtacctggtggtgctctccaaagtgcctgctcatttgcaccttcctcctgagaaggagggggacacggtgaagtttgtggtaatgacagttgaggtaagagcagctctgaaattatgtgtccctgtgaggacaggatatgtgagtagcactaagatgaaagtccttgaaaaccgacagtgtggagtacaatagtgcacacattagcccagctgccttggaggcagaggcagaattgtgggttcctggtctgtaaggatgtgcctgagtatacagctagaccctatttgaataaacaggaaggcagggaatacctattggcaaagtctgattcacctgatggtacagagtgcctttcaccctcaccactgggaagcaaggaggtctgtaagacatcctgttatccctacactataaacctaatgtgggtcctaaataaaatctagacagtgttacattttaaattgggcagtgaagctggacatttcacccagaaacacttggcccctcaaaatgtatctatacgtgcactatagttttattaccttgccatgggcatgctgggaaagagcctcactgtgccagagctgtgctgccaatcctgaacaagggttgacaccttaccctaagagaagaaagtcagtatcctgagggtgtatggtacaaaggcaccaggtgaaccaggctaagttaggtggtctttgagcttgtcttagcccagtgaagacaggaaagcaaatgtgtgtgtaaagtattgggtggcagctcctagtcatactctgcgctgcacaggccatgccatgacacttgtttcctataaaaactctgtccccatttcacacatggggaaagaagctcagagaggttcggggacttgctagaagtcactagtcataaatcatactccaaaactccaaggcctgctatccctagaacccacgctctcaaattcaacctatgacagaggcaagccctggtgctgtgggagccccaaggaagagcctctggcctggtggccacgtagcccaggagagatttctaggagagagaggcagcagagtaagggggctttgtggcagagaggggactggcactttggggaataggtgggatcaggactgaatattaagaaaactaggaatgacaacctagaaatgtgggtcaggggccattcttctgcaaggaggttgtctgatctcctgccctctccatctatccatggtcttccatatcttttattggcactgcttccctagaggtccctgagatagagtcctgggtgagcattctaacacagtgcttccctttaggccctgtcatggcatttgatccatgagcagatcccactgagtctggacctccaagccgggctagactgctgctgcctggcactacaggtgcctggcctctggggggtgctgtcctccccagagtacgtgactcatgcctgctccctcatccattgtgtgcgattcatcctggaagcca

SEQ ID NO: 103 - DNA, 104 bp, Homo sapiens
  source 1..104: mol_type = genomic DNA
tcccagctacttgggaggctgaggcaagagaactgcttgaacccgaggggcagaggttgcagtgagctgagagtgcgccattgcacttcagcctgtgtgacaga

SEQ ID NO: 104 - DNA, 91 bp, Homo sapiens
  source 1..91: mol_type = genomic DNA
gagggtcagcaagatttataaaggaaatttgtgatccaagtatgaagcaaaataaggtgcagaataaattttaagcaagtaatagattaga

SEQ ID NO: 105 - DNA, 98 bp, Homo sapiens
  source 1..98: mol_type = genomic DNA
ctactcaggagactgaggctggaggatcgcttgagcccaggaggttaagtctctagtgagatgtgttcatgccactgcactccagcctcggctataga

SEQ ID NO: 106 - DNA, 166 bp, Homo sapiens
  source 1..166: mol_type = genomic DNA
attgttaggaggtagtataaatgagctaaatctcatctttattacggtagagttaatgggtgatgtctaaagttgtctgaagtctataaatcatgacaaattatgatgtggtgattgtattcaacagtctttcagttgcagggataaaaccccagtttaaactaga

SEQ ID NO: 107 - DNA, 881 bp, Homo sapiens
  source 1..881: mol_type = genomic DNA
gtaagaggcagctcgggagctcagtgttgctgtggggagggggcatggggctgacactgaagagggtaaagcagttttatttgaaaagcaagatctctgaccagtccagtcacttttccatctcagcctggcagtaagtcttgtcaccgtcaagttattgtagccatccttcaccctcacctcgccactcctcatggtggcctgtgaggtcagccaggtccccttctcatctgcacctaccatgttaggtggatcctaattttagagacatgaaaaataatcatctggaagtactttatgtcttaagttggcctggacatgtcagccaaggaatacttacttggtttgtgttagtgcttgtaattcgcccccagaatgtgtacacgttctggatgcattaaagtctggcctgtatccttaaagggccatcgctgtgctgcctgccctcagcaaggacacactttgcagacccacagaggctccgcctccacctcacaccaaagaaagggaggagtccaaagggcatcagtgccattactcacaaaatgataaatacacccttattctgaaccacgtggagtcatatggtttgtgatccctgtccttcaggtttcagcttagtggggaagtgggaaagtcagcgtgtgatcacagcacagggtgattgctgctgattatattatgtgcctgctgtatgcaggatgaaatactttatatgcgtcatcttatttgactctcacaaccccctgtgagataggctctgttactcccatttgacaggtgaggaaagcaaggcttagagaatttcagtgacttgcccaggtcctctgagctaggaagtagccattctggcatttgaacccaaggcctgctatccctag

SEQ ID NO: 108 - DNA, 221 bp, Homo sapiens
  source 1..221: mol_type = genomic DNA
gtaagggggctttgtggcagagaggggactggcactttggggaataggtgggtcaggactgaatgtaatggagccatgtcagagctgtccttctggaagggcaagggcacctggacgcgctgcccctcagtgctttggacggttccacaactgtgattcacacggcttccccaaacgaaggtacacgagtgggcattctgtgactcggtacttccctttag

SEQ ID NO: 109-155 - skipped (47 entries)

SEQ ID NO: 156 - DNA, 14 bp, Homo sapiens
  source 1..14: mol_type = genomic DNA
gtttcaaaagtaga

SEQ ID NO: 157 - skipped

SEQ ID NO: 158 - DNA, 164 bp, Homo sapiens
  source 1..164: mol_type = genomic DNA
atacttactctgcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccctg

SEQ ID NO: 159-160 - skipped (2 entries)

SEQ ID NO: 161 - DNA, 32 bp, Homo sapiens
  source 1..32: mol_type = genomic DNA
aacccacgctctcaaattcaacctatgacaga

(19) United States
(12) Patent Application Publication
(10) Pub. No.: US 2024/0409928 A1
(43) Pub. Date: Dec. 12, 2024

(54) NOVEL RNA TRANSCRIPT

(71) Applicant: PTC THERAPEUTICS, INC., South Plainfield, NJ (US)

(72) Inventors: Anuradha BHATTACHARYYA, Edison, NJ (US); Yaofeng CHENG, Basking Ridge, NJ (US); Kerstin Annemarie EFFENBERGER, Metuchen, NJ (US); Wencheng LI, Martinsville, NJ (US); Jana NARASIMHAN, Scotch Plains, NJ (US); Christopher Robert TROTTA, Somerset, NJ (US); Marla L. WEETALL, Morristown, NJ (US); Matthew G. WOLL, Dunellen, NJ (US)

(21) Appl. No.: 18/586,114

(22) Filed: Feb. 23, 2024

Related U.S. Application Data
(63) Continuation of application No. 17/524,586, filed on Nov. 11, 2021, now abandoned.
(60) Provisional application No. 63/113,182, filed on Nov. 12, 2020.

(51) Int. Cl. C12N 15/113

(52) U.S. Cl. CPC C12N 15/113; C12N 2310/11; C12N 2320/33

(57) ABSTRACT

An alternatively spliced intronic sequence is disclosed, the splicing of which can be induced in the presence of a small molecule, e.g., Compound (I), as described herein.

### CROSS-REFERENCE TO RELATED APPLICATIONS

This application is a continuation application of and claims priority to U.S. application Ser. No. 17/524,586, filed Nov. 11, 2021, which claims the benefit of, and priority to, U.S. Provisional Patent Application Ser. No. 63/113,182 filed Nov. 12, 2020, U.S. Provisional Patent Application Ser. No. 63/113,826 filed Nov. 13, 2020, U.S. Provisional Patent Application Ser. No. 63/192,203 filed May 24, 2021, U.S. Provisional Patent Application Ser. No. 63/245,927 filed Sep. 19, 2021, U.S. Provisional Patent Application Ser. No. 63/261,467 filed Sep. 21, 2021, U.S. Provisional Patent Application Ser. No. 63/261,495 filed Sep. 22, 2021, and U.S. Provisional Patent Application Ser. No. 63/255,745 filed Oct. 14, 2021, the contents of which are hereby expressly incorporated by reference into the present application in their entireties.

### FIELD OF THE DISCLOSURE

The disclosure generally relates to the treatment of Huntington's Disease and the identification of to Huntingtin pre-mRNA sequences required for the production of a small molecule-induced alternatively spliced transcript.

### SEQUENCE LISTING

The instant application contains a Sequence Listing conforming to the rules of WIPO Standard ST.26 and filed as an electronic document via PatentCenter encoded as XML in UTF-8 text, which is hereby incorporated by reference in its entirety. The electronic document, created on Aug. 20, 2024, is entitled “1368-126 CON_ST26.xml”, and is 181,950 bytes in size.

### BACKGROUND

Huntington's disease (HD) is an autosomal dominant, progressive, neurodegenerative disorder. HD is characterized by motor, cognitive, and psychiatric symptoms due to progressive atrophy of the striatum as well as of cortical and other extra-striatal areas of the brain. In advanced cases, there is also loss of neurons in the thalamus, substantia nigra pars reticulata and in the subthalamic nucleus. The huntingtin gene is widely expressed and is required for normal development. HD pathology is thought to be caused by an expanded, unstable trinucleotide repeat in the huntingtin gene leading to the production of a mutant HTT protein (mHTT) having an extended polyglutamine repeat. A range of 10-35 trinucleotide repeats is found in wild type HTT protein but repeat numbers greater than 36 are generally pathogenic by a dominant toxic gain of function mechanism.

Several small molecules compounds are being evaluated for use in the treatment of Huntington's Disease. These compounds have been disclosed in International Application Number PCT/US2016/066042 filed Dec. 11, 2016 and published as International Publication Number WO2017/100726 on Jun. 15, 2017; International Application Number PCT/US2018/035954 filed Jun. 5, 2018 and published as International Publication Number WO2018/226622 on Dec. 13, 2018; International Application Number PCT/US2018/039775 filed Jun. 27, 2018 and published as International Publication Number WO2019/005980 on Jan. 3, 2019; International Application Number PCT/US2018/039794 filed Jun. 27, 2018 and published as International Publication Number WO2019/005993 on Jan. 3, 2019; and, International Application Number PCT/US2019/038889 filed Jun. 25, 2019 and published as International Publication Number WO2020/005873 on Jan. 2, 2020, each of which are incorporated by reference herein in their entirety as if fully set forth herein.

Nevertheless, currently there are no FDA approved disease-modifying medications for HD. Accordingly, there is an urgent need in the art for systemically administered therapeutics that can traverse the blood brain barrier to treat HD.

### SUMMARY

This disclosure describes the discovery of pre-mRNA sequences required for alternative splicing of an intronic sequence that is contingent on the presence of a small molecule, e.g., Compound (I), as described herein. Thus, in the presence of Compound (I), the intronic sequence is converted into an “intron-derived exon” that can be spliced into the mature spliced mRNA, an event leading to a frameshift in the mRNA's open reading frame and the production of premature stop codons. The ensuing premature termination of translation results in nonsense mediated decay of the mRNA and a concomitant reduction in the amount of protein encoded by the mRNA. Conversely, in the absence of Compound (I), the intronic sequence remains dormant and is spliced out of the pre-mRNA without causing a change to the mRNA's reading frame.

In one aspect, a small molecule-inducible intronic sequence is disclosed, the splicing of which is inducible only in the presence of a small molecule composition, wherein the intronic sequence comprises a noncanonical 5′ splice site and a 3′ splice site, wherein the intronic sequence is not inducible in the absence of a pseudo-exonic splicing enhancer (pseudo-ESE).

In one aspect, the pseudo-ESE is proximal to the 5′ splice site, for example, within 6-200 nucleotides upstream of the 5′ splice site.

In one aspect, the pseudo-ESE is proximal to the 5′ splice site, for example, within 100 nucleotides upstream of the 5′ splice site.

In one aspect, the 5′ splice site is a noncanonical 5′ splice site.

In one aspect, the noncanonical 5′ splice site comprises an RNA sequence of 5′-NNGAguragu-3′ (SEQ ID NO: 109), where N is A, G, C, or U and r is A or G.

In one aspect, the noncanonical 5′ splice site comprises an RNA sequence of 5′-CAGAgtaag 3′ (SEQ ID NO: 98).

In one aspect, the noncanonical 5′ splice site comprises a nucleotide sequence of SEQ ID NO: 5.

In one aspect, the intronic sequence without the pseudo-ESE is not inducible in the presence of a variant U1 snRNA comprising the nucleotide sequence of SEQ ID NO: 65.

In one aspect, the 3′ splice site comprises a nucleotide sequence of SEQ ID NO: 47.

In one aspect, the 3′ splice site comprises a nucleotide sequence of SEQ ID NO: 4.

In one aspect, the pseudo-ESE comprises at least 10 nucleotides of the nucleotide sequence of SEQ ID NO: 85.

In one aspect, the intronic sequence has the nucleotide sequence of SEQ ID NO: 46 or 49.

In a second aspect, a small molecule-inducible intronic sequence is disclosed, the splicing of which is inducible only in the presence of a small molecule composition, said intronic sequence comprising in 5′ to 3′ order:
a 5′ exonic splice site,
a first intronic branch point,
an intronic 3′ splice site,
a pseudo-ESE (Exonic Splice Enhancer),
a noncanonical 5′ exonic splice site,
a second intronic branch point, and
a 3′ exonic splice site.

In one aspect, the pseudo-ESE comprises at least 10 nucleotides of the nucleotide sequence of SEQ ID NO: 85; the 5′ splice site comprises a nucleotide sequence of SEQ ID NO: 5, and the 3′ splice site comprises a nucleotide sequence of SEQ ID NO: 4 or 47.

In another aspect, the intronic sequence between the intronic 3′ splice site and the 5′ exonic splice site comprises at least 100 nucleotides of the nucleotide sequence of SEQ ID NO: 46 or 49.

In a third aspect, an mRNA is disclosed comprising the intronic sequence, the splicing of which is inducible only in the presence of a small molecule composition, wherein the intronic sequence comprises a noncanonical 5′ splice site and a 3′ splice site, wherein the intronic sequence is not inducible in the absence of a pseudo-exonic splicing enhancer (pseudo-ESE).

In one aspect, the small molecule composition comprises an effective amount of a compound selected from the group consisting of:

[symbol]

or a pharmaceutically acceptable salt thereof,
effective at inducing the splicing of the intronic sequence.

In another aspect, splicing of the intronic sequence induced by an effective amount of any one of the compounds HTT-C1, HTT-C3, HT-D1, HTT-D2, HTT-D3 and HTT-D4 can also be induced by an effective amount of the compound having the structure of

[symbol]

or a pharmaceutically acceptable salt thereof.

In another aspect, splicing of the intronic sequence not induced by an effective amount of any one of the compounds HTT-C1, HTf-C3, HTTD1, HTT-D2, HTT-D3 and HTT-D4 can be induced by an effective amount of the compound having the structure of

[symbol]

or a pharmaceutically acceptable salt thereof,

In another aspect, splicing of the intronic sequence induced by an effective amount of the compound having the structure of

[symbol]

or a pharmaceutically acceptable salt thereof,
can also be induced by an effective amount of any one of the compounds HTT-C1, HTT-C3, HTT-D1, HTT-D2, HTT-D3 and HTT-D4.

In another aspect, splicing of the intronic sequence not induced by an effective amount of the compound having the structure of

[symbol]

or a pharmaceutically acceptable salt thereof,
can be induced by an effective amount of any one of the compounds HTT-C1, HTT-C3, HTTD1, HT-D2, HTT-D3 and HTT-D4.

In another aspect, the small molecule composition comprises an effective amount of the compound having the structure of

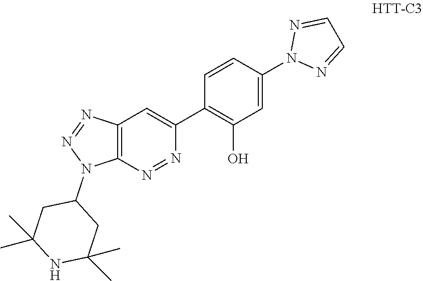 [SMILES: CC1(C)CC(n2nnc3cc(-c4ccc(-n5nccn5)cc4O)nnc32)CC(C)(C)N1]

or a pharmaceutically acceptable salt thereof,
effective at inducing the splicing of the intronic sequence.

In another aspect, the mRNA is huntingtin (HTT) mRNA.

In another aspect, the HTT mRNA comprises a CAG repeat mutant HTT mRNA.

In another aspect, the HTT mRNA comprises a wild-type huntingtin mRNA.

In another aspect, the mRNA comprises an RNA sequence selected from the group consisting of SEQ ID NO: 4 and 5.

In another aspect, the huntingtin mRNA does not comprise any 25 nucleotide fragments of SEQ ID NO: 107 or SEQ ID NO: 108.

In a fourth aspect, a method for reducing the expression of a gene in a cell is disclosed comprising contacting the cell with a therapeutically effective amount of a small molecule composition comprising a compound having the structure of

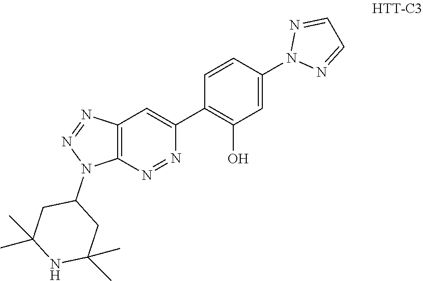 [SMILES: CC1(C)CC(n2nnc3cc(-c4ccc(-n5nccn5)cc4O)nnc32)CC(C)(C)N1]

or a pharmaceutically acceptable salt thereof,
wherein the gene comprises a small molecule-inducible intronic sequence, the splicing of which is inducible only in the presence of the small molecule composition, wherein the intronic sequence comprises a noncanonical 5′ splice site and a 3′ splice site, wherein the intronic sequence is not inducible in the absence of a pseudo-exonic splicing enhancer (pseudo-ESE).

In a fifth aspect, a method for reducing the expression of a gene in a subject is disclosed comprising administering a therapeutically effective amount of a small molecule composition comprising a compound having the structure of

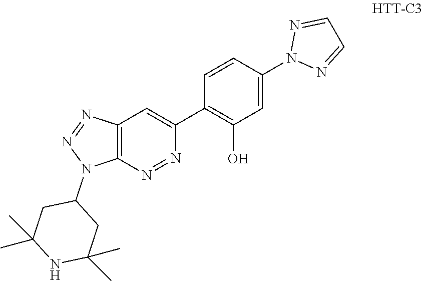 [SMILES: CC1(C)CC(n2nnc3cc(-c4ccc(-n5nccn5)cc4O)nnc32)CC(C)(C)N1]

or a pharmaceutically acceptable salt thereof,
to said subject,
wherein the gene comprises a small molecule-inducible intronic sequence, the splicing of which is inducible only in the presence of the small molecule composition, wherein the intronic sequence comprises a noncanonical 5′ splice site and a 3′ splice site, wherein the intronic sequence is not inducible in the absence of a pseudo-exonic splicing enhancer (pseudo-ESE).

In one aspect, the subject has Huntington's disease.

In one aspect, the amount of the small molecule composition is therapeutically effective if it decreases huntingtin protein expression by about 30 to about 50% relative to a control.

In a sixth aspect, a method for determining a therapeutic amount of a small molecule composition effective at reducing the amount of protein in a subject is disclosed comprising measuring the amount of mRNA encoding the protein containing an intronic sequence in a sample taken from the subject before and after administration of the small molecule composition, wherein splicing of the intronic sequence is inducible only in the presence of the small molecule composition, wherein the intronic sequence comprises a noncanonical 5′ splice site and a 3′ splice site, and the intronic sequence is not inducible in the absence of a pseudo-exonic splicing enhancer (pseudo-ESE).

In another aspect, the small molecule composition has the structure of

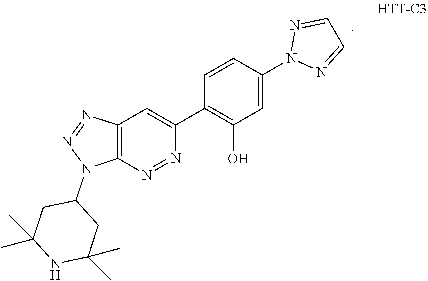 [SMILES: CC1(C)CC(n2nnc3cc(-c4ccc(-n5nccn5)cc4O)nnc32)CC(C)(C)N1]

In one aspect, the mRNA encodes a CAG repeat mutant HTT protein.

In one aspect, the subject has Huntington's disease.

In one aspect, the sample comprises blood cells.

In one aspect, wherein the percent reduction in the amount of protein in the blood cells indicates the percent reduction in the subject's central nervous system.

### BRIEF DESCRIPTION OF THE DRAWINGS

FIG. 1A shows chemical structures of exemplary compounds HTT-C1 and HTT-D1 having HTT-lowering activity on HTT mRNA and HTT protein expression. (IC₅₀): compound's concentration required for the reduction of HTT protein by 50%; (CC₅₀): compound's concentration required for the reduction of cell viability by 50%.

FIG. 1B depicts an exemplary RT-qPCR analysis of HTT mRNA in HD patient fibroblasts (Coriell Cell Repositories) after 24 hours of treatment with HTT-C1 and HTT-D1 (0.01-1.0 μM). Representative graphs show percent of HTT mRNA remaining relative to DMSO control; normalized to the expression of the housekeeping gene, TATA-Box Binding Protein (TBP).

FIG. 1C shows an exemplary RT-qPCR analysis of HTT mRNA in B-lymphocytes from the same patient (Coriell Cell Repositories) after 24 hours of treatment with HTT-C1 and HTT-D1 (0.25 μM). Representative graphs show percent remaining relative to DMSO control; normalized to the expression of the housekeeping gene, glyceraldehyde-3-phosphate dehydrogenase (GAPDH).

FIG. 1D shows an exemplary electrochemiluminescence (ECL) analysis of total HTT protein in fibroblasts derived from a patient with HD (Coriell Cell Repositories) after 96 hours of continuous treatment with HTT-C1 and HTT-D1 (0.01-1.0 μM). Representative graphs show percent HTT protein remaining relative to the DMSO control. Cell viability assays were performed in parallel.

FIG. 1E shows an exemplary Western Blot of HTT protein and housekeeping proteins, β-actin, α-serine/threonine-protein kinase (AKT), prolyl-4-hydroxylase inhibitors (PDI) and glyceraldehyde-3-phosphate dehydrogenase (GAPDH) in HD patient fibroblasts (Coriell Cell Repositories) after 96 hours of continuous treatment with HIT-C1 (0.015-1.0 μM). Utrophin (UTRN) was used as a loading control.

FIG. 1F shows an exemplary MSD-ECL (Meso Scale Discovery®ECL) analysis of HTT protein after 96-hour treatment with HTT-C1 in fibroblasts derived from a HD patient and an unaffected individual (Coriell Cell Repositories). Compound treatment resulted in a concentration-dependent decrease in both wild type and mutant HTT protein levels. Percent HTT remaining was calculated relative to the DMSO (no compound) control. mHTT: mutant Huntington protein; WT HTT: wild type Huntington protein.

FIG. 1G shows the chemical structures of exemplary HTT-A and HTT-B compounds identified through a the library screen.

FIG. 1H shows an exemplary ECL analysis of total HTT protein from fibroblasts derived from a patient with HD (Coriell Cell Repositories) after treatment with HTT-A and HTT-B (0.01-10.0 μM). Representative graphs show percent HTT protein remaining relative to the DMSO control. Cell viability assays were performed in parallel.

FIG. 1I shows an exemplary Western Blot of HTT protein in HD patient fibroblasts (Coriell Cell Repositories) after treatment with HTT-A and HTT-B (0.015-10.0 μM). Utrophin (UTRN) was used as a loading control.

FIG. 2A depicts an exemplary quantitative RT-PCR analysis of HTT mRNA after 24-hour treatment with HTT-C1 in B-lymphocytes derived from an HD patient (Coriell Cell Repositories). The graph confirms HTT-C1 induced HTT mRNA decay that lowered the amount of HTT mRNA by about 85% as compared to the DMSO control. Aliquots from these samples were used for primer walking and Ampliseq experiments.

FIG. 2B shows an exemplary primer walking analysis of the HTT mRNA sample of FIG. 2A using twelve distinct primer pairs (see TABLE V) encompassing all 67 HTT exons to identify a modification in HTT mRNA splicing.

FIG. 3A shows an exemplary schematic diagram illustration of an Ampliseq workflow.

FIG. 3B shows an exemplary method of calculating the Junction Expression Index (JEI).

FIG. 3Ci shows an exemplary count and JEI calculation for HTT introns 1-32 using the Ampliseq data normalized relative to +DMSO samples.

FIG. 3Cii shows an exemplary count and JEI calculation for HTT introns 33-66 using the Ampliseq data normalized relative to +DMSO samples. Each row represents an intron of HTT gene. “num_”, total read counts for all junction reads using either the 5′ splice site or the 3′ splice site of the intron. “JEI_”, the Junction Expression Index for a particular intron. “JEI average”, the average JEI for the three replicates of a treatment. “JEI_sd”, the standard deviation of JEI of the three replicates of a treatment. “delta-JEI_(Cpd vs. Ctl)”, the change of JEI between compound-treated samples and control (DMSO). “P value (T-test)”, the P-value using the Student's t-test. Cpd is 125 nM of HTT-C1.

FIG. 3Di shows an exemplary bar graph representation of the % Junction Expression Index (JEI) of 66 introns of the human HTT gene as calculated in FIGS. 3Ci and 3Cii. Error bar represents standard deviation. Data were based on three biological replicates of next generation sequencing data.

FIG. 3Dii shows an exemplary bar graph representation of the % Junction Expression Index (JEI) of 66 introns of the human HTT gene normalized to DMSO as calculated in FIG. 3Ci and 3Cii. Error bar represents standard deviation. Data were based on three biological replicates of next generation sequencing data.

FIG. 3E shows exemplary features of the pseudoexon(s) in intron 49 of HTT gene as identified from Ampliseq data. The 5′ and 3′ splice site MAXENT scores were calculated using MaxEntScan representing the strength of splice sites. The sequences and scores of the splice sites of the pseudoexon are shown. The sizes of the pseudoexon were also indicated in the “Ampliseq reads” track. More reads support the splicing that generates the 115 bp pseudoexon compared to the 146 bp pseudoexon in a compound-treated sample. Cpd is 125 nM of HTT-C1.

FIG. 3F shows an exemplary Integrated Genome Viewer (IGV) plot of Ampliseq and RNAseq reads supporting inclusion of pseudoexon 49a in cells treated with a compound (HTT-C1, HTT-C2 or HTT-C3). The sequencing protocol (Ampliseq or RNAseq), cell type, compound name and concentration are indicated. Only one biological replicate for each treatment condition is shown. The positions and sequences of the 5′ and 3′ splice sites (ss) of the pseudoexon 49a are indicated. In the three read tracks, each read is visualized as a bar. A thin line between the bars indicates the splicing/removal of an intron as sequenced as a single read. Each read is visualized as a bar. Refseq transcript annotated exon 49 and 50 of HTT gene are indicated on the bottom of the plot.

FIG. 3G shows an exemplary Integrated Genome Viewer (IGV) plot of RNAseq reads supporting inclusion of pseudoexon 49a in cells treated with DMSO, 10 nM or 300 nM HTT-D3 in MRC5 cells. Only one biological replicate for each treatment condition is shown. The positions and sequences of the 5′ and 3′ splice sites (ss) of the pseudoexon 49a are indicated. In the three read tracks, each read is visualized as a bar. A thin line between the bars indicates the splicing/removal of an intron as sequenced as a single read. Refseq transcript annotated exon 49 and 50 of the HTT gene are indicated on the bottom of the plot.

FIG. 3H shows an Integrated Genome Viewer (IGV) plot of RNAseq reads supporting inclusion of pseudoexon 49a-1 in cells treated with DMSO, 30 nM or 1 μM risdiplam in human dermal fibroblasts or type 1 SMA patient fibroblasts. Only one biological replicate for each treatment condition is shown. The positions and sequences of the 5′ and 3′ splice sites (ss) of the pseudoexon 49a are indicated. In read tracks, each read is visualized as a bar. A thin line between the bars indicates the splicing/removal of an intron as sequenced as a single read. Refseq transcript annotated exon 49 and 50 of HTT gene are indicated on the bottom of the plot.

FIG. 3I depicts an exemplary Sashimi plot of alternative splicing in intron 49 of HTT gene using Ampliseq data. Exons 49 and 50 are indicated as E49 and E50 respectively. A threshold of minimum 5 reads were used to visualize the Integrated Genome Viewer (IGV) plot of RNAseq reads. Cpd is 125 nM of HTT-C1.

FIG. 3J shows HTT gene expression as quantified using RNAseq in SHSY5Y cells treated with compound 100 nM HTT-C2 and in TK6 cells treated with compound 100 nM HTT-C3. The cell type (SHSY5Y or TK6 cells), compound name and concentration are indicated. Y-axis shows the normalized gene expression values as Fragment Per Kb per Million total reads (FPKM). P-values are based on two tailed Student's t-test.

FIGS. 4Ai-iv depicts the HTT pre-mRNA nucleotide sequences between exons 49 and 50 before the initiation of splicing. Sequence elements depicted include Exon 49 (SEQ ID NO: 40), Intron 49 (SEQ ID NO: 48), pseudoexon 49a-1 (SEQ ID NO: 46), pseudoexon 49a-2 (SEQ ID NO: 49), Exon 50 (SEQ ID NO: 42). Exemplary splice site sequences include sequences identified by rectangular boxes and comprise pseudoexon 49a 3′ splice site-1 (SEQ ID NO: 4), pseudoexon 49a 3′ splice site-2 (SEQ ID NO: 47) and a pseudoexon 49a 5′ splice site (SEQ ID NO: 5). “ss:” splice site.

FIG. 4Bi-ii depicts an exemplary small molecule-induced spliced HTT mRNA containing a 115 nucleotide pseudoexon 49a-1. Exemplary nucleotide sequences of the Exon 49-pseudoexon 49a-1 splice junction (SEQ ID NO: 53) and pseudoexon 49a-1-Exon 50 splice junction (SEQ ID NO: 55) are highlighted with a diagonal striped bar. The small molecule-induced splicing event results in a frameshift mutation and a truncated HTT protein (SEQ ID NO: 57). The shift in the reading frame produces three premature STOP codons immediately downstream of Exon 49, two within the spliced pseudoexon 49a-1 nucleotide sequence and one within the Exon 50 nucleotide sequence.

FIG. 4C shows the inclusion by splicing of the pseudoexon 49a-1 (115 nt.; SEQ ID NO: 49) between the C-terminal nucleotide sequence of Exons 49 (SEQ ID NO: 8) and the N-terminal nucleotide sequence of Exon 50 (SEQ ID NO: 9). The location of two premature stop codons within pseudoexon 49a-1 are indicated with arrows. The premature STOP codon most proximal to Exon 49 is predicted to result in a truncation of the HTT polypeptide.

FIG. 4D shows the predicted location of the branchpoint (BP) upstream of pseudoexon 49a-1 (115 nt) and exon 50 of human HTT gene. The branchpoint of pseudoexon 49a-1 (115 nt) was predicted based on the consensus sequence motif described in Mercer et al. (2015) Genome research 25, 290-303 (the content of which is incorporated by reference herein in its entirety).

FIG. 4Ei-ii depicts an exemplary small molecule-induced spliced HTT mRNA containing a 146 nucleotide pseudoexon 49a-2 (SEQ ID NO: 49). The exemplary nucleotide sequences of the Exon 49-pseudoexon 49a-2 splice junction (SEQ ID NO: 50) and pseudoexon 49a-2—Exon 50 splice junction (SEQ ID NO: 51) are highlighted with a diagonal striped bar. The small molecule-induced splicing event results in a frameshift mutation and a truncated HTT protein (SEQ ID NO: 54). The location of the nucleotide sequence of SEQ ID NO: 161 is depicted with a white triangle. The shift in the reading frame produced a premature STOP codon within the Exon 50 nucleotide sequence which is predicted to result in a truncation of the HTT polypeptide.

FIG. 5Ai-ii shows a volcano plot of RNA-seq analysis comparing gene expression in SHSY5Y cells treated with either 24 nM or 100 nM of HTT-C2 with DMSO treated SHSY5Y cells. Genes (>1.5 fold, False Discovery Rate (FDR)<5%) are shown as down-regulated and up-regulated, respectively. HTT is one of the most downregulated genes in HTT-C2 treated SHSY5Y cells.

FIG. 5Bi shows a schematic diagram of alternative splicing (AS) events. CE, cassette exon; A3SS, alternative 3′ splice site (ss); A5SS, alternative 5′ splice site.

FIG. 5Bii shows the number of regulated AS events in SHSY5Y RNA-seq data following treatment with 24 nM and 100 nM HTT-C2.

FIG. 5Biii shows the number of CEs included (UP) or excluded (DN) after treatment with 24 nM and 100 nM HTT-C2; ratio of UP/DN are shown in text.

FIG. 5Biv shows the percentage of exons with 3′ and 5′ splice sites annotated by public databases (Refseq, Ensembl or UCSC Known Genes) for NC (exons changed in neither condition) or UP exons following treatment with 24 nM and 100 nM HTT-C2.

FIG. 5Ci-ii shows an RT-PCR analysis of 16 HTT-C2 induced splicing isoforms incorporating a pseudoexon (shown as open triangles). Back filled triangle denote wild type splicing isoforms.

FIG. 5D shows Cumulative Distribution Function (CDF) curves of basal percent spliced in index (PSI; average PSI in DMSO samples). Graph shows data for exons separated into three groups; UP is based on ΔPSI>20% and Fisher's Exact test P<0.001 in any one of the two conditions (24 nM or 100 nM HTT-C2 vs. DMSO). Median values are shown as dashed vertical lines for each group. “None change” (NC) are exons not changed in all three conditions. “Annotated” and “psiExons” are the “Both” and “None” group respectively. Median values are shown as dashed vertical lines for each group.

FIG. 5E shows sequence conservation of 3′ and 5′ splice site region. Conservation is based on PhastCons score for 46 way placental mammals. Mean (standard error of mean [SEM]) conservation scores are shown.

FIG. 5Fi shows Cumulative Distribution Function (CDF) curves of splice site scores for cryptic (unannotated) exons up-regulated (PSI increase by >20% and Fisher's Exact Test P<0.001) in compound-treated cells (black solid line) compared to up-regulated annotated exons (black dotted line) and Refseq-annotated exons with no significant change (gray dashed line). Four types of splice site were examined: 3′ splice site and 5′ splice site of the pseudoexon, upstream (U-) 5′ splice site and downstream (D-) 3′ splice site. P-values are based on Wilcoxon Rank-Sum test. Vertical lines indicate median values in different ss groups.

FIG. 5Fii shows Cumulative Distribution Function (CDF) curves of intron and exon sizes for cryptic (unannotated) exons up-regulated (PSI increase by >20% and Fisher's Exact Test P<0.001) in compound-treated cells (black solid line) compared to up-regulated annotated exons (black dotted line) and Refseq-annotated exons with no significant change (gray dashed line). P-values are based on Wilcoxon Rank-Sum test. Vertical lines indicate median values in different groups.

FIG. 5G shows cryptic exon activation is related to a decrease in gene abundance. Cumulative Distribution Function (CDF) curves of RNA-seq gene abundance change for genes with predicted NMD-psiExons. NMD-psiExons are psiExons with predicted premature termination codon or causing frame-shift of the host gene or both and are included (UP) following HTT-C2 treatment. Number of genes (n) and P-value are indicated. P-value is based on comparison with “all other genes” group using Wilcoxon Rank-Sum Test.

FIG. 5H shows that nonsense-containing transcripts induced by HTT-C1 are stabilized by treatment with cycloheximide (CHX), a potent inhibitor of protein translation. Nonsense-containing transcripts are therefore degraded by nonsense-mediated decay (NMD). Lymphocytes derived from HD patients were treated with DMSO (control) or HTT-C1 (250 nM) in the absence or presence of cycloheximide (CHX) for 0, 2 h, 4 h or 8 h at which time total RNA was isolated and probed by RT-PCR using primers that anneal within Exons 49 and 51. PCR products were then visualized by gel electrophoresis according to standard procedures. PsiExon: pseudoexon; CPD: HTT-C1; DMSO: dimethyl sulfoxide.

FIG. 6Ai shows 5′splice site (ss) sequence having two regions (−4 to −4 and +1 to +6).

FIG. 6Aii shows enriched or depleted exons up-regulated (defined by PSI increase by >20% and Fisher's Exact Test P<0.001) compared to no change exons in SHSY5Y cells treated with either 24 nM or 100 nM HTT-C2 for 24 hours. Values in the figure are significance scores SS=−S×log 10 P-value, in which S=1 for enrichment and −1 for depletion. P-value is based on Fisher's Exact Test comparing k-mer frequencies of up-regulated and no change exons. Sequences in dark gray are those which are different compared to 5′ splice site (ss) sequences in no change exons.

FIG. 6Bi-ii shows the nucleotide sequence (SEQ ID NO: 66) of the human U1 snRNA promoter and the U1-GA snRNA sequence found within a U1-GA snRNA expression vector. Diagonal striped rectangle indicates region annealing to pre-mRNA (SEQ ID NO: 38).

FIG. 6Ci shows the 5′ end of the U1-GA snRNA annealing to the HTT pseudoexon 49a-1 noncanonical 5′ splice site. FIG. 6Cii shows the sequence 5′-CAGguaag-3′ at the 5′ end of U1 snRNA annealing with a canonical 5′ splice site.

FIG. 6D shows a Venn diagram of pseudoexons identified from three datasets. Sequence logo of a 5′ splice site (ss) in different gene groups. The compound-activated (100 nM HTT-C2) 5′ splice site is defined by exon PSI increase by >20% and Fisher's Exact Test P<0.001. psiExons have a strong preference for GA at −2 to −1 position of 5′ splice site, but do not show any preference for A at the −3 or +3 position). Both HTT-C2 and variant U1-GA can enhance U1 recruitment to the 5′ splice site with GA at −2 to −1 position and demonstrate the specificity of HTT-C2 for sequences with a −3 A sequence.

FIG. 7A shows the design of hybrid mouse/human HTT minigene constructs.

FIG. 7Bi shows a schematic diagram of a [human HTT Exon 49]—[human HTintron 49]—[human HTT Exon 50] minigene construct (SEQ ID NO: 67) together with a PCR analysis of RNA extracts from HEK293 transfected with the HTT minigene and treated with either DMSO or HTT-C2 (0.010-1 μM).

FIG. 7Bii shows the nucleotide sequence of the [human HTT Exon 49]—[human HTT intron 49]—[human HTT Exon 50] minigene construct (SEQ ID NO: 67).

FIG. 7Biii shows a schematic diagram of a [mouse Htt Exon 49]—[mouse Htt intron 49]-[mouse Htt Exon 50] construct (SEQ ID NO: 68) together with a PCR analysis of RNA extracts from HEK293 transfected with the Htt minigene and treated with either DMSO or HTT-C2 (0.010-1 μM).

FIG. 7Biv shows the nucleotide sequence of the [mouse Htt Exon 49]—[mouse Htt intron 49]—[mouse Htt Exon 50] minigene construct (SEQ ID NO: 68).

FIG. 7Bv shows a schematic diagram of a hybrid [mouse Htt Exon 49] [human HTT intron 49]—[mouse Htt Exon 50] minigene construct (SEQ ID NO: 69) together with a PCR analysis of RNA extracts from the HEK293 transfected with the hybrid HTT minigene and treated with either DMSO or HTT-C2 (0.010-1 μM).

FIG. 7Bvi shows the nucleotide sequence of the hybrid [mouse Htt Exon 49]—[human HTT intron 49]—[mouse Htt Exon 50] minigene construct (SEQ ID NO: 69).

FIG. 7Bvii shows a schematic diagram of a hybrid [mouse Hit Exon 49—mouse Htt intron 49]—[human HTT intron 49 (50 nt)]—[human HTT psiExon 49a (115 nt)]—[human HTT intron 49 (50 nt)]—[mouse Hit intron 49]—[human Exon 50] (SEQ ID NO: 70) together with a PCR analysis of RNA extracts from HEK293 transfected with the hybrid HTT minigene and treated with either DMSO or HTT-C2 (0.010-1 μM).

FIG. 7Bviii shows the nucleotide sequence of the hybrid [mouse Hit Exon 49—mouse Hit intron 49]—[human HT intron 49 (50 nt)]—[human=T7 psiExon 49a (115 nt)]—[human HTT intron 49 (50 nt)]—[mouse Hit intron 49]—[human Exon 50] minigene construct (SEQ ID NO: 70).

FIG. 7Bix shows a schematic diagram of a hybrid [mouse Hit Exon 49]—[mouse Hit intron 49]—[human HTT psiExon 49a (115 nt)]—[human HTT intron 49 (50 nt)]—[mouse Hit intron 49]—[human HTT Exon 50] (SEQ ID NO: 71) together with a PCR analysis of RNA extracts from HEK293 transfected with the hybrid HTT minigene and treated with either DMSO or HTT-C2 (0.010-1 μM).

FIG. 7Bx shows the nucleotide sequence of the hybrid [mouse Htt Exon 49]—[mouse Hit intron 49]—[human HTT psiExon 49a (115 nt)]—[human HTT intron 49 (50 nt)]—[mouse Hit intron 49] [human HTT Exon 50] minigene construct (SEQ ID NO: 71).

FIG. 7Bxi shows a schematic diagram of a hybrid [mouse Hit Exon 49-mouse Htt intron 49]—[human HTT intron 49 (50 nt)]—[human HTT psi Exon 49a (115 nt)]—[mouse Htt intron 49]—[human HTT Exon 50] (SEQ ID NO: 72) together with a PCR analysis of RNA extracts from HEK293 transfected with the hybrid HTT minigene and treated with either DMSO or HTT-C2 (0.010-1 μM).

FIG. 7Bxii shows the nucleotide sequence of the hybrid [mouse Htt Exon 49]—[mouse Htt intron 49]—[human HTT intron 49 (50 nt)]—[human HTT psiExon 49a (115 nt)]—[mouse Htt intron 49]—[human HTT Exon 50] minigene cassette (SEQ ID NO: 72).

FIG. 7Ci shows a schematic diagram of the numbering of nucleotides (from −4 to +6; SEQ ID NO: 78) within the HTT pseudoexon-49a 5′ splice site.

FIG. 7Cii shows a PCR analysis of RNA extracts from HEK293 transfected with mouse-human hybrid HTT minigenes of SEQ ID NO: 70 with either no mutations (wt) or a single mutation within the 5′ splice site of the human pseudoexon 49a and treated with either DMSO or HTT-C2 (0.010-1 μM).

FIG. 7Di shows a PCR analysis of RNA extracts from HEK293 transfected with a hybrid HTT psiExon 49a minigene and a hybrid HTT psiExon 1 minigene and treated with either DMSO or HTT-C2 (0.010-1 μM). DS: downstream; US: Upstream.

FIG. 7Dii shows a schematic diagram and the nucleotide sequence of a hybrid [mouse Hit Exon 49]—[mouse Htt intron 49]—[human HTT intron 49 (50 nt)]—[human HTT intron 1 US]-[human HT intron psiExon-1]—[human HTT intron 1 DS]—[mouse Htt intron 49]—[mouse Htt Exon 50] minigene construct (SEQ ID NO: 73).

FIG. 7Diii shows a PCR analysis of RNA extracts from HEK293 transfected with a hybrid human HTT psiExon 49a HTT minigenes or a hybrid human HTT psiExon-8 minigene and treated with either DMSO or HTT-C2 (0.010-1 μM).

FIG. 7Div shows a schematic diagram and the nucleotide sequence of the hybrid [mouse Htt Exon 49]—[mouse Hit intron 49]—[human HTT intron-8 US (50 nt)]—[human HTT intron-8 psiExon]—[human HTT intron-8 DS]—[mouse Htt intron 49]—Mouse Htt Exon 50] (SEQ ID NO: 74). DS: downstream; US: Upstream.

FIG. 7Dv shows a PCR analysis of RNA extracts from HEK293 transfected with a hybrid human HTT psiExon 49a minigene or a hybrid human HTT psiExon-40a minigene and treated with either DMSO or HTT-C2 (0.010-1 μM).

FIG. 7vi shows a schematic diagram and the nucleotide sequence of the hybrid [mouse Htt Exon 49]—[mouse Htt intron 49]—[human HTT intron-40a US iExon (50 nt)]—[human HTT intron-40a psiExon]—[human HTT intron-40a DS iExon]—[mouse Htt intron 49]—mouse Hit Exon 50](SEQ ID NO: 75). DS: downstream; US: Upstream.

FIG. 7Dvii shows a PCR analysis of RNA extracts from HEK293 transfected with the hybrid human HTT psiExon-49a minigene or the hybrid human HTT psiExon-40b minigene and treated with either DMSO or HTT-C2 (0.010-1 μM).

FIG. 7Dviii shows a schematic diagram and the nucleotide sequence of the hybrid [mouse Htt Exon 49]—[mouse Htt intron 49]—[human HTT intron-40b US iExon (50 nt)]—[human HTT intron-40b psiExon]—[human HTT intron-40b DS iExon]—[mouse Htt intron 49]—mouse Htt Exon 50] (SEQ ID NO: 76). DS: downstream; US: Upstream.

FIG. 7Dix shows, for each potential HTT iExon (49, 1, 8, 40a and 40b corresponding to SEQ ID Nos: 46, 103, 104, 105 and 106 respectively), the sequence of the crypic 5′ splice site (SEQ ID Nos: 5, 98, 99, 100 and 101 respectively), the length of the iExon, its location within the HTT gene and its splicing activity in the presence of the HTT-C2 (0.010-1 JIM).

FIG. 7Dx shows a schematic diagram of a generic hybrid iEx pseudoexon containing minigene together with a PCR analysis of RNA extracts from HEK293 transfected with a hybrid HTT iExon49, iExon1, iExon8, iExon40a or iExon40b minigene comprising either GAgt or AGgt 5′ splice site and treated with either DMSO or HTT-C2 (0.010-1 μM).

FIG. 7Ei shows a PCR analysis of RNA extracts from HEK293 transfected with a hybrid human HTT psiExon-49a HTT minigenes or a hybrid human HTT psiExon-49a HTT minigene having a 20 nucleotide deletion within the pseudoexon and treated with either DMSO or HTT-C2 (0.010-1 μM).

FIG. 7Eii shows a schematic diagram and the nucleotide sequence of a hybrid [mouse Htt Exon 49]—[mouse Htt intron 49]—[human HTT intron 49 (50 nt)]—[Human HTT psiExon 49a (9445 nt) with a 20 nt deletion (from −38 to −19)]—[human HTT intron 49 (50 nt)]—[mouse Htt intron 49]—[mouse Htt Exon 50] (SEQ ID NO: 102).

FIG. 7Eiii shows the −38 to −19 wt nucleotide sequence of human HTT psiExon 49a (SEQ ID No: 85) and deletion mutants A-K (SEQ ID Nos: 86-95).

FIG. 7Eiv shows a PCR analysis of RNA extracts from HEK293 transfected with the mutant or non-mutant hybrid HTT minigenes described in FIG. 7Eiii and treated with either DMSO or HTT-C2 (0.010-1 μM).

FIG. 7Ev shows the location of mutations within the sequence from −39 to −4 of the human HTT pseudoexon-49a upstream of the 5′ splice site.

FIG. 7Evi shows a PCR analysis of RNA extracts from HEK293 transfected with the mouse-human hybrid HTT minigenes of SEQ ID NO: 70 with the aforementioned mutations in FIG. 7Ev within the pseudoexon or no mutation (wt hybrid minigene) and treated with either DMSO or HTT-C2 (0.010-1 μM).

FIG. 7Fi shows a bioinformatic analysis of HTT psiExon 49 (grey rectangle) that identifies the location of potential sites of SR protein binding and splicing enhancers. The rectangles with diagonal stripes denote the location of exonic splicing enhancer (RESCUE-ESEs, see legend) sequences upstream of HTT psiExon 49 noncanonical 5′ splice site.

FIG. 7Fii shows an exemplary depiction of the nucleotide sequence of the HTT pseudoexon 49a-1 together with the location of the 3′ splice site, 5′ splice site and pseudo-exonic splicing enhancer (ESE) sequence (SEQ ID No.: 85).

FIG. 8Ai shows % mouse and human HTT protein remaining in plasma as a function of the concentration of administered HTT-C1 compound (0.01-1 μM).

FIG. 8Aii shows the plasma concentration in wild type mice after systemic administration of 10 mg/kg of HT-C1, HIT-D1 and HTT-C2 over 24 hours.

FIG. 8B shows a western blot analysis of human HTT protein within the brain tissue of BACHD mice treated with HTT-C2 (3 mg/kg or 10 mg/kg); Graph shows percent lowering relative to vehicle control and normalised to mouse Htt protein.

FIG. 8C shows western blot analysis of 10 mg/kg HTT-C2 induced lowering of human HTT protein within brains of BACHD mice over time (4-63 days). Graph shows percent lowering relative to vehicle control and normalised to mouse Htt protein. Example western blot shown below graph with mouse Htt as a loading control.

FIG. 8D shows a western blot analysis of human HTT protein expression levels in brain tissue over time following cessation of 10 mg/kg HTT-C2 treatment in BACHD mice. Graph shows percent lowering of human HTT protein relative to vehicle control and normalised to mouse Hit protein.

FIG. 8Ei shows an ECL analysis of human HTT protein expression levels within different parts of the brain from BACHD mice treated with 10 mg/kg HTT-C2. Graphs show percent HTT remaining relative to vehicle control and normalised to utrophin (UTRN).

FIG. 8Eii shows an ECL analysis of human HTT protein expression levels within different tissues (brain, muscle, heart, white blood cells (WBC), liver and kidney) from BACHD mice treated with 10 mg/kg HTT-C2. Graphs show percent HTT remaining relative to vehicle control and normalised to utrophin (UTRN).

FIG. 8F shows an ECL analysis of human HT protein expression levels within different tissues from Hu97/18 mice (top bottom graph) and BACHD mice (bottom top graph) treated with HTT-D3. Graphs show percent remaining relative to vehicle control and normalised to Kirsten rat sarcoma viral oncogene homolog (KRAS).

FIG. 8G shows an ECL analysis of human HTT protein expression levels within striatum and cortex of the brain from Hu97/18 mice treated with different doses of HTT-D3 (2 mg/kg/6 mg/kg/12 mg/kg). Graphs show percent remaining relative to vehicle control and normalised to Kirsten rat sarcoma viral oncogene homolog (KRAS).

FIG. 8H shows HTT protein in CSF or plasma is responsive to lowering in brain HTT protein in Hu97/Hu18 mice. The graphs show a correlation between different parts of the brain and CSF HTT levels, as well as between plasma and CSF HTT levels in HTT-D3 treated Hu97/18 mice.

FIG. 9A shows graphical representation of percent spliced in (PSI) HTT transcripts effected by HTT-C2 versus HTT-C3.

FIG. 9B shows graphical representation of percent spliced in (PSI) TNRC6A transcripts effected by HTT-C2 versus HTT-C3.

FIG. 9C shows graphical representation of percent spliced in (PSI) SF3B3 transcripts effected by HTT-C2 versus HTT-C3.

FIG. 9D shows graphical representation of percent spliced in (PSI) NUPL1 transcripts effected by HTT-C2 versus HTT-C3.

FIG. 9E shows graphical representation of percent spliced in (PSI) ZNF680 transcripts effected by HTT-C2 versus HTT-C3.

FIG. 9F shows graphical representation of percent spliced in (PSI) DENND4A transcripts effected by HTT-C2 versus HTT-C3.

FIG. 9G shows graphical representation of percent spliced in (PSI) FOXM1 transcripts effected by HTT-C2 versus HTT-C3.

FIG. 9H shows graphical representation of percent spliced in (PSI) GXYLT1 transcripts effected by HTT-C2 versus HT-C3.

FIG. 9I shows graphical representation of percent spliced in (PSI) IVD transcripts effected by HTT-C2 versus HTT-C3.

FIG. 9J shows graphical representation of percent spliced in (PSI) HTT transcripts effected by HTT-C2 versus HTT-C3.

FIG. 9K shows graphical representation of percent spliced in (PSI) POMT2 transcripts effected by HTT-C2 versus HTT-C3.

FIG. 9L shows graphical representation of percent spliced in (PSI) PDXDC1 transcripts effected by HTT-C2 versus HTT-C3.

FIG. 9M shows graphical representation of percent spliced in (PSI) ARL15 transcripts effected by HTT-C2 versus HTT-C3.

FIG. 9N shows graphical representation of percent spliced in (PSI) c12orf4 transcripts effected by HTT-C2 versus HTT-C3.

FIG. 9O shows graphical representation of percent spliced in (PSI) PMS1 transcripts effected by HTT-C2 versus HTT-C3.

FIG. 9P shows graphical representation of percent spliced in (PSI) PPIP5K2 transcripts effected by HTT-C2 versus HTT-C3.

FIG. 9Q shows graphical representation of percent spliced in (PSI) RAPGEF1 transcripts effected by HTT-C2 versus HTT-C3.

FIG. 9R shows graphical representation of percent spliced in (PSI) XRN2 transcripts effected by HTT-C2 versus HTT-C3.

FIG. 9S shows graphical representation of percent spliced in (PSI) SAMD4A transcripts effected by HTT-C2 versus HTT-C3.

All the curves are fit to a dose response for each compound and dashed lines represent the EC50 from maximum response. In addition to HTT, GXYLT1, POMT2, PDXDC1, ARL15 and c12orf4 are affected at the EC50.

FIG. 10 is a plot of individual plasma concentrations of Compound 1 over time after oral administration of a Compound 1 suspension formulation (Batch 21) in 0.5% hydroxypropyl methyl cellulose (HPMC) in water at 30 mg in Male Cynomolgus Monkeys (Leg 1)

FIG. 11 is a plot of mean plasma concentrations of Compound 1 over time after oral administration of a Compound 1 suspension (Batch 21) in 0.5% HPMC in water at 30 mg in Male Cynomolgus Monkeys (Leg 1).

FIG. 12 is a plot of individual plasma concentrations of Compound 1 over time after oral administration of Tablet Formulation A (wet granulation Batch 15) at 30 mg in Male Cynomolgus Monkeys (Leg 2).

FIG. 13 is a plot of mean plasma concentrations of Compound 1 over time after oral administration of Tablet Formulation A (wet granulation Batch 15) at 30 mg in Male Cynomolgus Monkeys (Leg 2).

FIG. 14 is a plot of individual plasma concentrations of Compound 1 over time after oral administration of Tablet Formulation B (dry granulation Batch 20) at 30 mg in Male Cynomolgus Monkeys (Leg 3).

FIG. 15 is a plot of individual plasma concentrations of Compound 1 over time after oral administration of Tablet Formulation B (dry granulation Batch 20) at 30 mg in Male Cynomolgus Monkeys (Leg 3).

FIG. 16 is dissolution profiles (% dissolved Compound 1 over time) of 5 mg tablets produced from Batch 23 before and after storage at 2 weeks at 50° C. or 1 month at 40° C./75% relative humidity.

FIG. 17 is dissolution profiles (% dissolved Compound 1 over time) of 50 mg tablets produced from Batch 23 before and after storage at 2 weeks at 50° C. or 1 month at 40° C./75% relative humidity.

FIGS. 18A and 18B shows a dose-dependent reduction in HTT mRNA in whole blood taken from healthy volunteers participating in a Single Ascending Dose (SAD; FIG. 18A) and Multiple Ascending Dose (MAD; FIG. 18B) study of a Phase I clinical trial.

FIG. 18A shows a lowering of HTT mRNA in whole blood taken from healthy volunteers in the SAD cohort where splicing was evaluated 24 hours after they were administered a one day, single dose of either placebo, 5 mg, 15 mg, 45 mg, 90 mg, or 135 mg of Compound 1.

FIG. 18B shows the lowering of HTT mRNA in whole blood taken from healthy volunteers in the MAD cohort dosed daily with either placebo, 15 mg or 30 mg of Compound 1 for 14 days. HTT splicing was then evaluated by RT-PCR 6 hours after administration of Compound 1 on day 14.

FIG. 19 shows how decay rates can be modeled to predict drug-dependent decrease in mRNA and protein Concentration over time.

FIG. 20 shows graphs that model the rate of HTT mRNA (FIG. 20A) and HTT protein (FIG. 20B) decay based on their half-lives and then predicted the time to reach steady state after Compound 1 treatment at 30 mg daily dose. For HTT mRNA, the half-life is estimated to be about 24 hours. HTT mRNA in FIG. 20A reaches steady state after approximately 5 days. For HTT protein, the half-life is estimated to be 5-7 days and consequently HTT protein steady state levels should take about 6 weeks from the beginning of treatment.

FIG. 21 compares the trajectory of HTT mRNA (FIG. 21A) and protein (FIG. 21B) lowering seen in Multiple Ascending Dose Study with those values predicted from the half-life of HT mRNA and protein as shown in FIG. 20.

FIG. 22 shows that Compound 1 crosses the Blood Brain Barrier in humans (FIG. 22A) and in non-human primates (FIG. 22B).

FIG. 23 is a plot of % of baseline of HTT RNA measured over time in whole blood of human subjects administered a placebo or a single dose of 90 mg of Compound 1, as described in the Single Ascending Dose (SAD) study in Part 1 of Example 10. The results show that the HTT splicing effect of Compound 1 is reversible and persists for 72 hours post cessation of treatment.

FIG. 24 is a plot of % baseline of HTT RNA measured over time in the whole blood of human subject administered a placebo or 15 or 30 mg of Compound 1, as described in the Multiple Ascending Dose (MAD) study described in Part 2 of Example 10. HTT splicing was monitored after the final dose at day 14, calculated as % HTT remaining from baseline (pre-dose day 0).

FIG. 25 is a bar graph showing the huntingtin mRNA and protein levels in whole blood from MAD cohort 2.3 (30 mg administered for 21 days with 100 mg loading dose (LD) for 2 days), as described in Example VIII, as a percent of baseline, after administration of vehicle or compound 1 to a human, 24 hours after the last dose. The results show HTT mRNA reduction reached steady state. Longer dosing was required for HTT protein levels to reach maximal steady state reduction.

### DETAILED DESCRIPTION OF THE DISCLOSURE

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, the materials, methods, and examples are illustrative only and not intended to be limiting. Other features of the disclosure are apparent from the following detailed description and the claims.

Titles or subtitles may be used in the specification for the sole convenience of the reader but are not intended to influence the scope of the present disclosure or to limit any aspect of the disclosure to any subsection, subtitle, or paragraph.

### I. Definitions

As used herein, the singular forms “a,” “an,” and “the,” are intended to include the plural forms as well, unless the context clearly indicates otherwise.

The phrase “and/or,” as used herein and in the claims, is understood to mean “either or both” of the elements so conjoined, i.e., elements that are conjunctively present in some cases and disjunctively present in other cases. Thus, as a non-limiting example, a reference to “A and/or B,” when used in conjunction with open-ended language such as “comprising” can refer, in one aspect, to A only (optionally including elements other than B); in another aspect, to B only (optionally including elements other than A); in yet another aspect, to both A and B (optionally including other elements); etc

As used herein and in the claims, the phrase “at least one,” in reference to a list of one or more elements, should be understood to mean at least one element selected from any one or more of the elements in the list of elements, but not necessarily including at least one of each and every element specifically listed within the list of elements, and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one aspect, to at least one, optionally including more than one, A, with no B present (and optionally including elements other than B); in another aspect, to at least one, optionally including more than one, B, with no A present (and optionally including elements other than A); in yet another aspect, to at least one, optionally including more than one, A, and at least one, optionally including more than one, B (and optionally including other elements); etc.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain aspects, the term “about” is used to modify a numerical value above and below the stated value by a variance of 10%. In certain aspects, the term “about” is used to modify a numerical value above and below the stated value by a variance of 5%. In certain aspects, the term “about” is used to modify a numerical value above and below the stated value by a variance of 1%.

As used herein, the term “substantial change” in the context of the amount of one or more RNA transcripts, an alternative splice variant thereof or an isoform thereof, or one or more proteins thereof, each expressed as the product of one or more of genes, means that the amount of such products changes by a statistically significant amount such as, in a nonlimiting example, a p value less than a value selected from 0.1, 0.01, 0.001, or 0.0001.

As used herein, the terms “subject” and “patient” are used interchangeably to refer to an animal or any living organism having sensation and the power of voluntary movement, and which requires for its existence oxygen and organic food. Non-limiting examples include members of the human, equine, porcine, bovine, rattus, murine, canine and feline species. In some aspects, the subject is a mammal or a warm-blooded vertebrate animal. In certain aspects, the subject is a non-human animal. In specific aspects, the subject is a human.

When a range of values is listed herein, it is intended to encompass each value and sub-range within that range. For example, “1-5 ng” or a range of “1 ng to 5 ng” is intended to encompass 1 ng, 2 ng, 3 ng, 4 ng, 5 ng, 1-2 ng, 1-3 ng, 1-4 ng, 1-5 ng, 2-3 ng, 2-4 ng, 2-5 ng, 3-4 ng, 3-5 ng, and 4-5 ng.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

As used herein, the terms “treat,” “treatment,” “treating” refer to therapeutic treatments, wherein the object is to reverse, alleviate, ameliorate, inhibit, slow down or stop the progression or severity of a disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disorder is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

The term “sample,” as used herein, generally refers to a biological sample. A sample may be a fluid or tissue sample. The sample may include proteins and nucleic acid molecules, such as deoxyribonucleic acid (DNA) molecules, ribonucleic acid (RNA) molecules, or both. The RNA molecules may be messenger RNA (mRNA) molecules. The sample may be a tissue sample. The sample may be a cellular sample, such as a sample comprising one or more cells. The sample may be plasma, serum or blood (e.g., whole blood sample). The sample may be a cell-free sample (e.g., cell-free DNA, or cfDNA).

As used herein, the term “tissue” refers to an aggregation of morphologically similar cells and associated intercellular matter, i.e., extracellular matrix, acting together to perform one or more specific functions in the body. In some embodiments, tissues fall into one of four basic types: muscle, nerve, epidermal, and connective. In some embodiments, a tissue is substantially solid, e.g., cells within the tissue are strongly associated with one another to form a multicellular solid tissue. In some embodiments, a tissue is substantially non-solid, e.g, cells within the tissue are loosely associated with one another, or not at all physically associated with one another, but may be found in the same space, bodily fluid, etc. For example, blood cells are considered a tissue in non-solid form.

As used herein, the term “RNA” means a molecule comprising at least one ribonucleotide residue. By “ribonucleotide” is meant a nucleotide with a hydroxyl group at the 2′ position of a beta-D-ribo-furanose moiety. The terms include double stranded RNA, single stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. RNAs can be synthesized in a cell by RNA polymerase I, II or III.

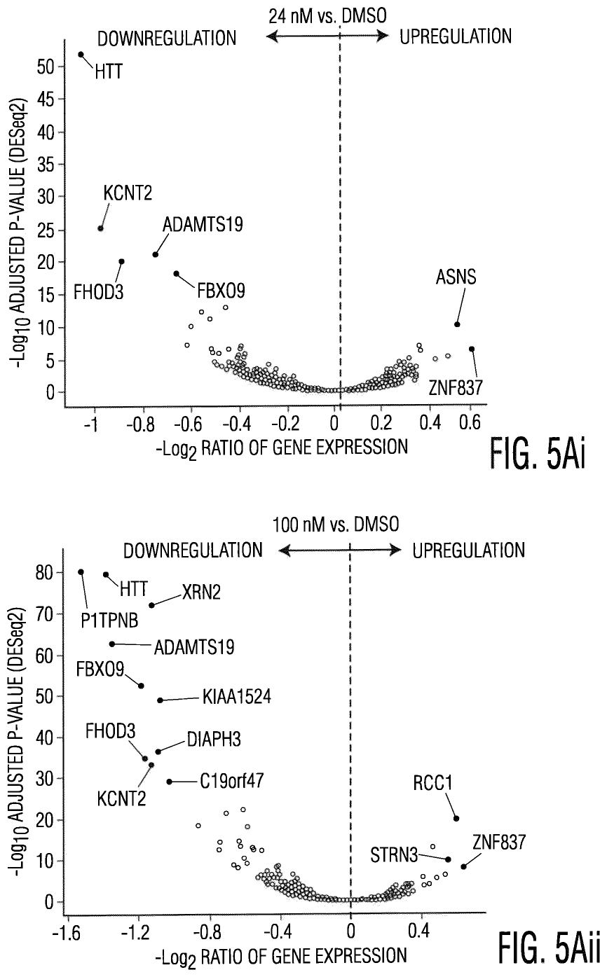

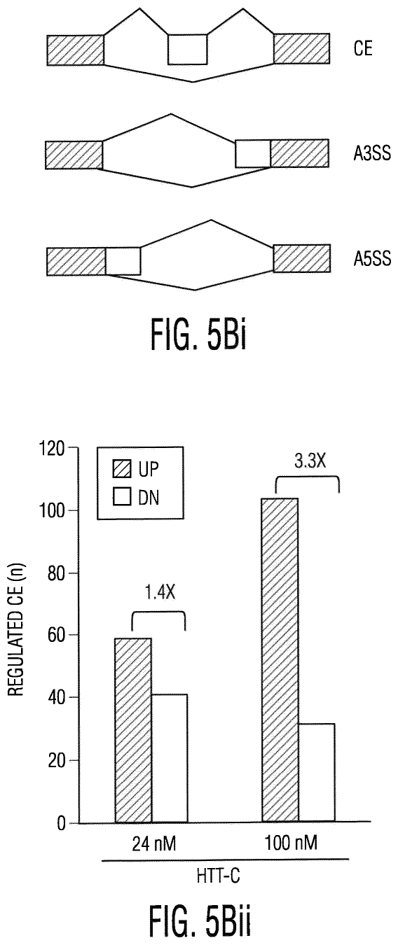

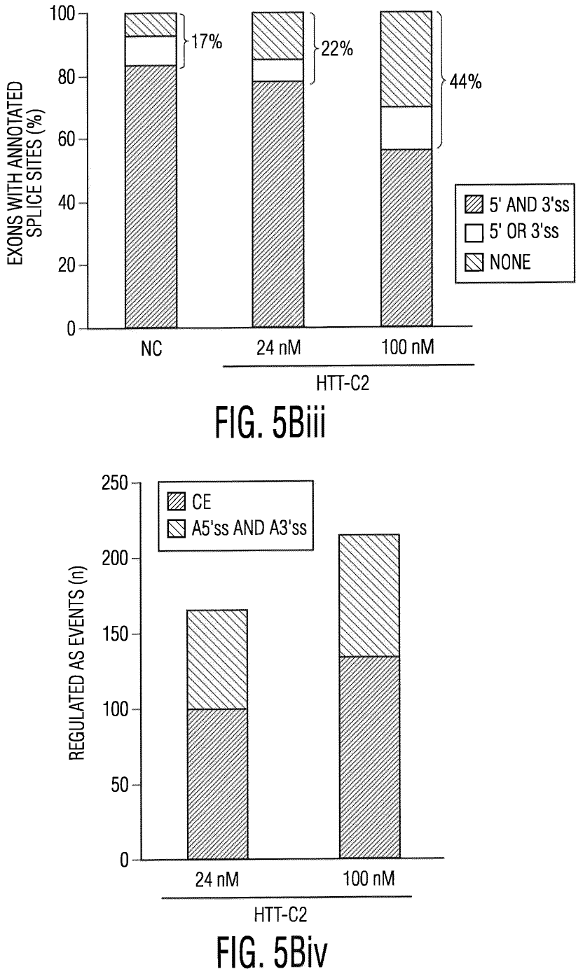

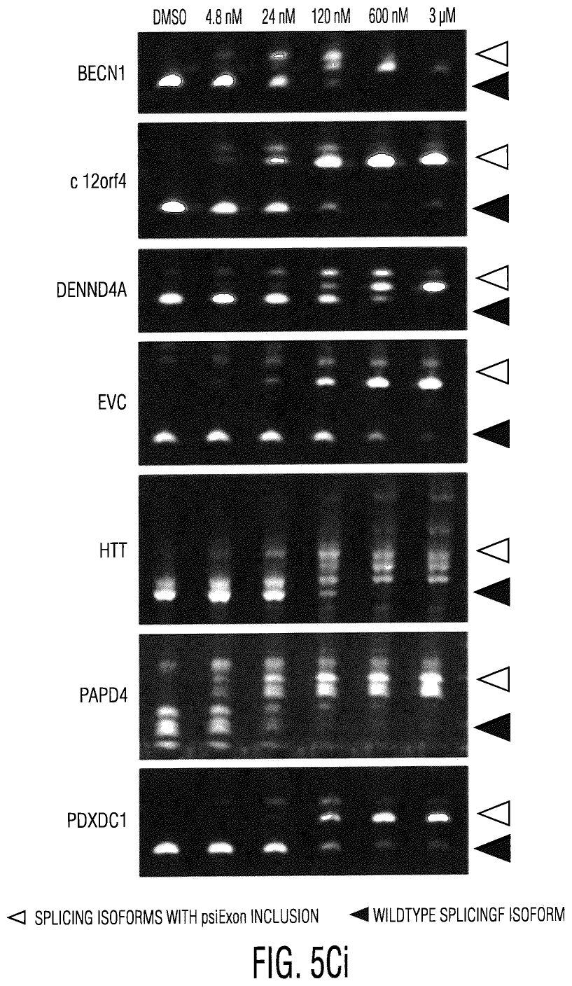

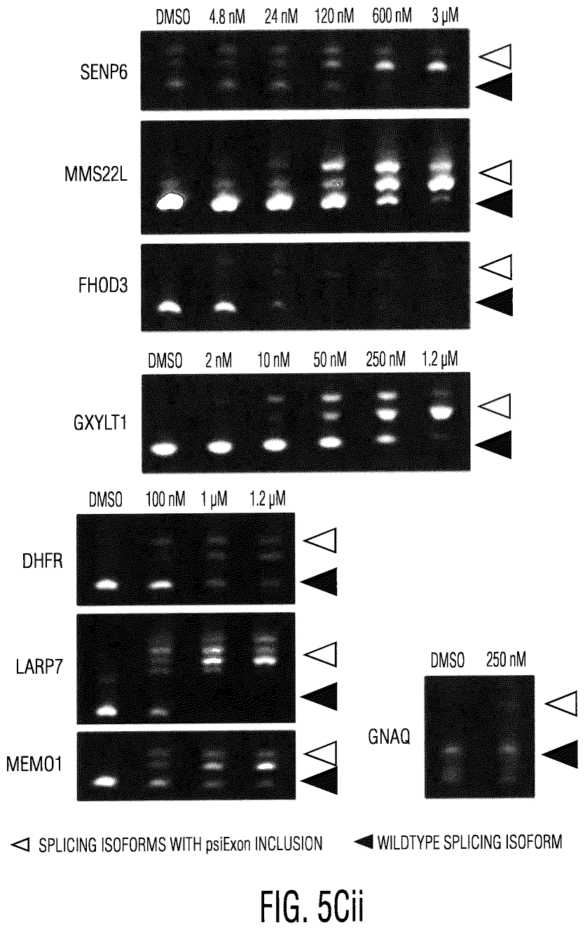

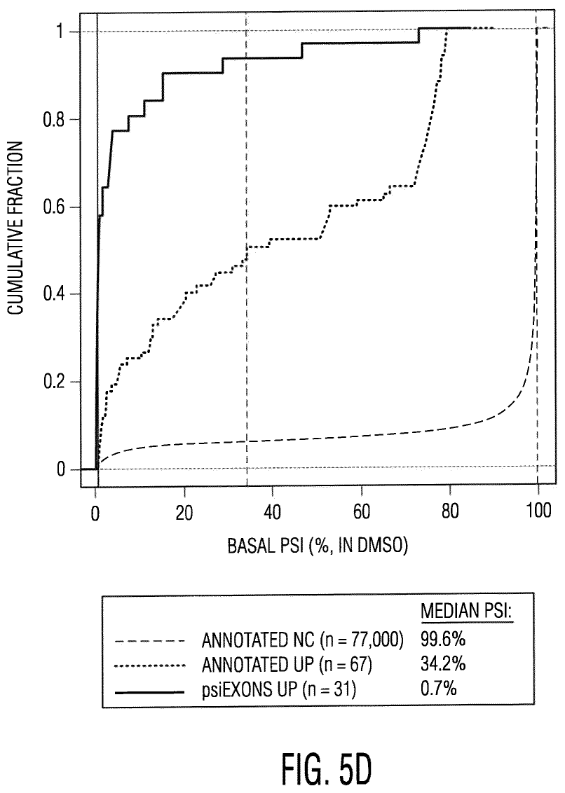

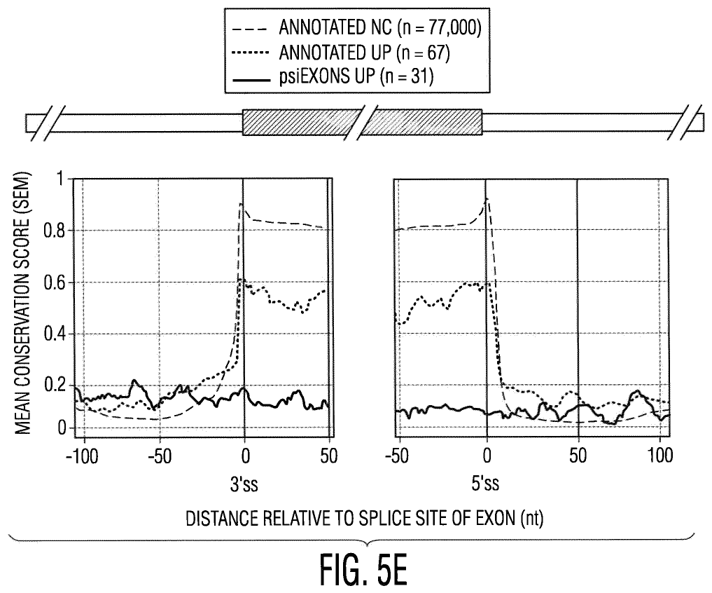

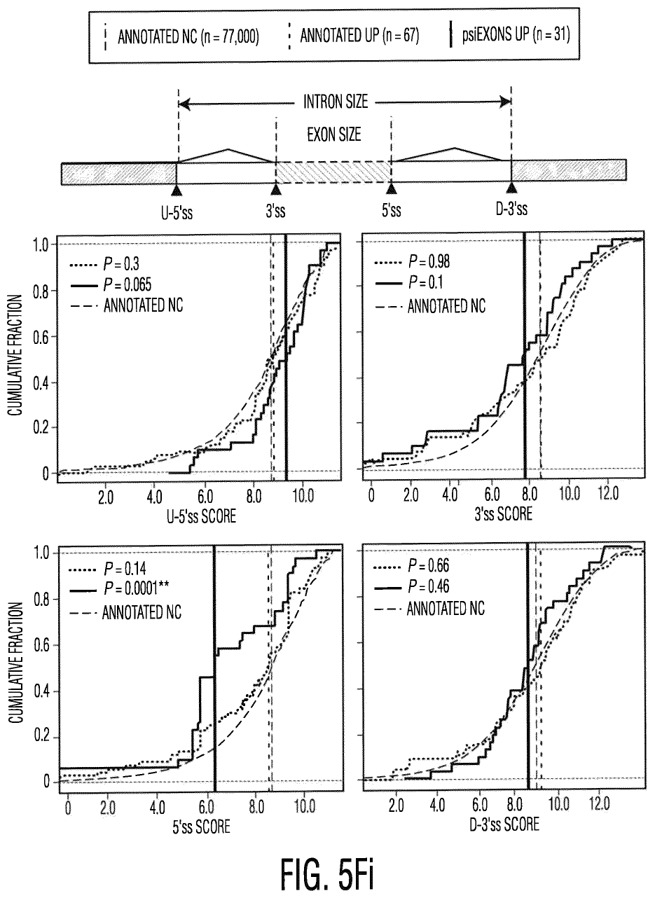

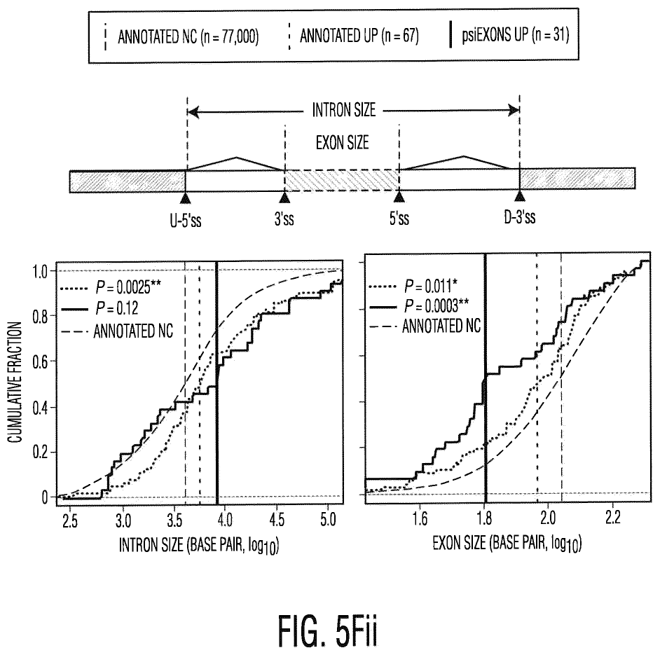

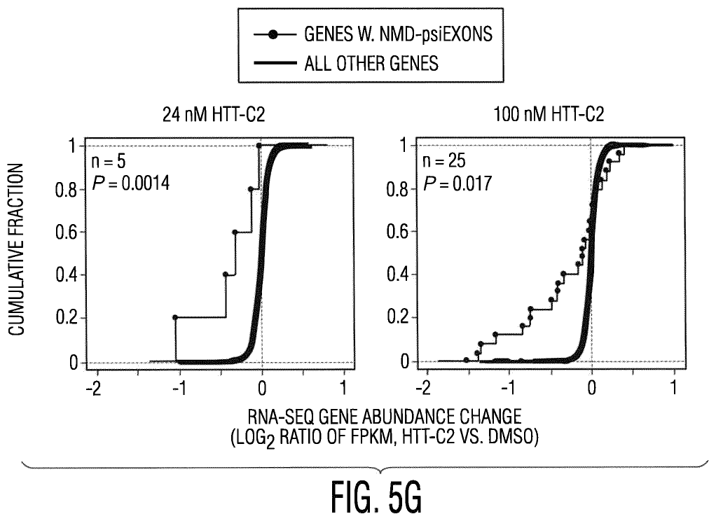

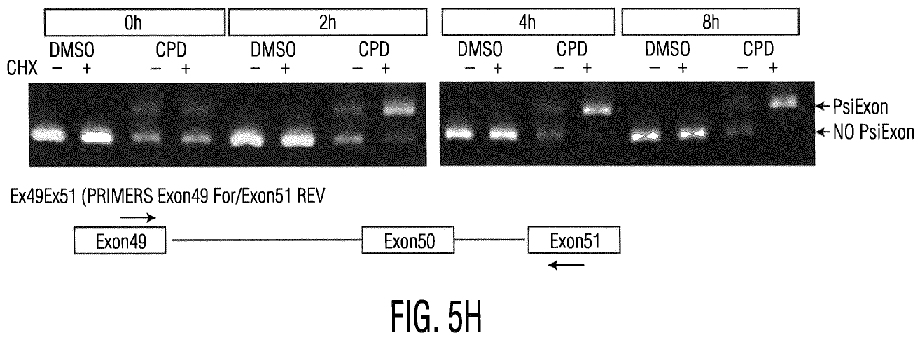

The term “mRNA” refers to any RNA that is produced in a cell by RNA polymerase II transcription of a gene. In one aspect, the mRNA of the disclosure is capped and polyadenylated. In one aspect, an mRNA of the disclosure encodes one or more proteins. In one aspect, the mRNA does not encode a protein. In another aspect, mRNA can refer to processed or unprocessed pre-mRNA. In another aspect, the mRNA of this disclosure includes, but is not limited to, pre-mRNA, spliced mRNA, partially spliced mRNA and alternatively spliced mRNA. In one aspect, the mRNA of the disclosure is a transcript that undergoes nonsense-mediated decay (NMD) in the presence of a compound as described herein (e.g., the compounds of TABLE IV). In other aspects, the mRNA of the disclosure is transcribed from the ITT gene. In yet another aspect, the mRNA of the disclosure is transcribed from any one of the genes listed in FIG. 5Ai or FIGS. 5Ci and 5Cii.

Splicing is a natural biological mechanism that may occur within human cells. Splicing processes primary messenger ribonucleic acid (mRNA) that has been transcribed from deoxyribonucleic acid (DNA) before the mRNA is translated into a protein. Splicing involves removing one or more contiguous segments of mRNA and is directed, in part, by a spliceosome. The segments that are removed are often referred to as introns, but the spliceosome may remove segments that contain both introns and exons.

An “exon” can be any part of a gene that is a part of the final mature RNA produced by that gene after introns have been removed by RNA splicing. The term “exon” refers to both the DNA sequence within a gene and to the corresponding sequence in RNA transcripts.

The term “intron” refers to both the DNA sequence within a gene and the corresponding sequence in the unprocessed RNA transcript. As part of the RNA processing pathway, introns can be removed by RNA splicing either shortly after or concurrent with transcription. They can be found in a wide range of genes, including those that generate proteins, ribosomal RNA (rRNA), and transfer RNA (tRNA).

As used herein, the term “isolated” means the physical state of Compound (I) after being isolated and/or purified from a synthetic process (e.g., from a reaction mixture) or natural source or combination thereof according to an isolation or purification process or processes described herein or which are well known to the skilled artisan (e.g., chromatography, recrystallization and the like) in sufficient purity to be characterized by standard analytical techniques described herein or well known to the skilled artisan.

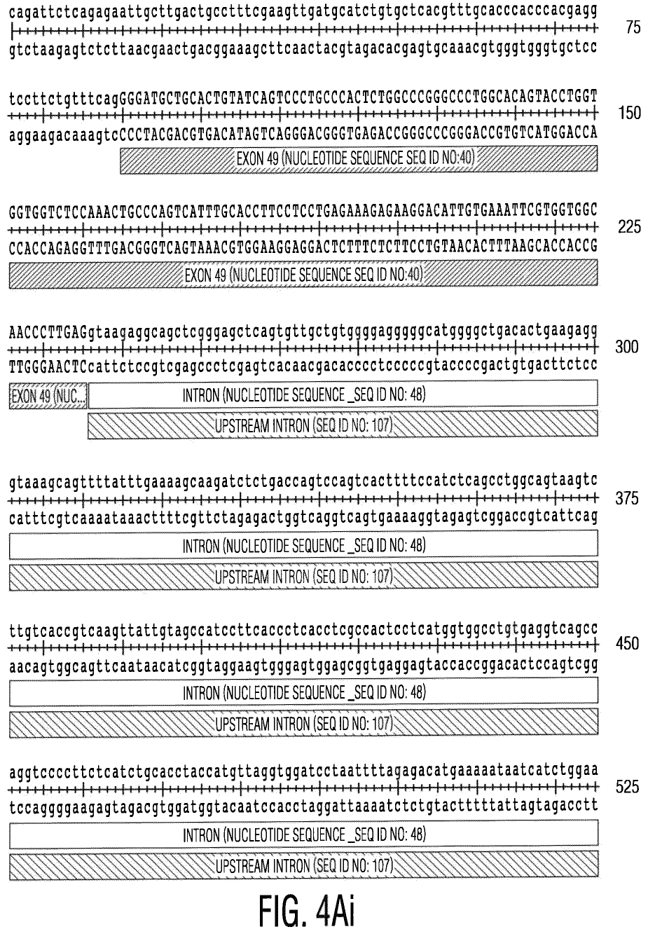

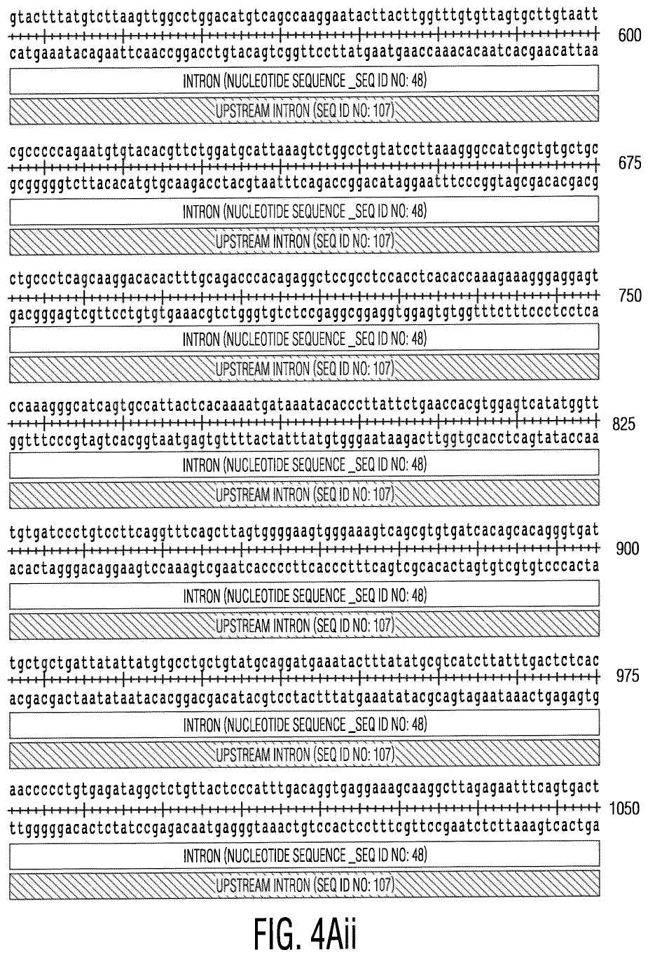

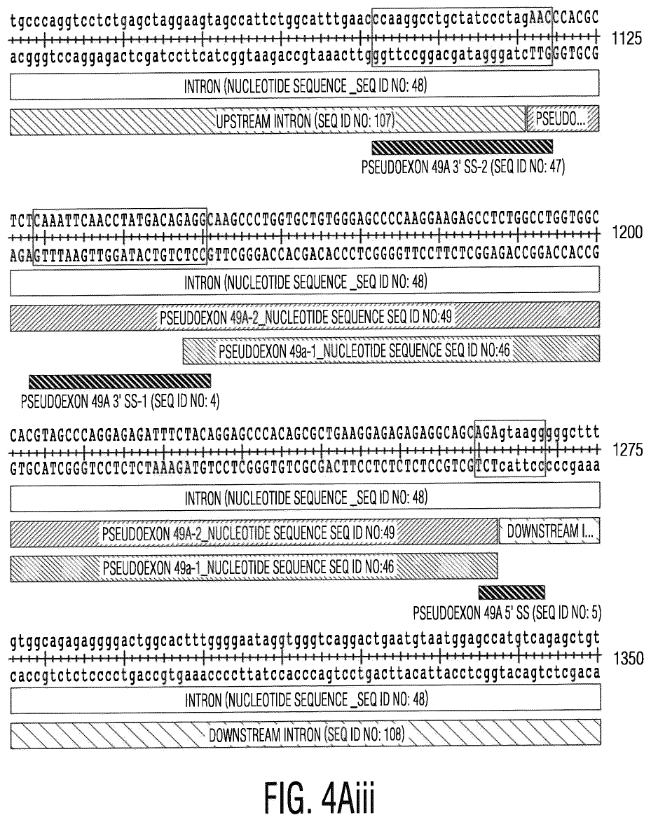

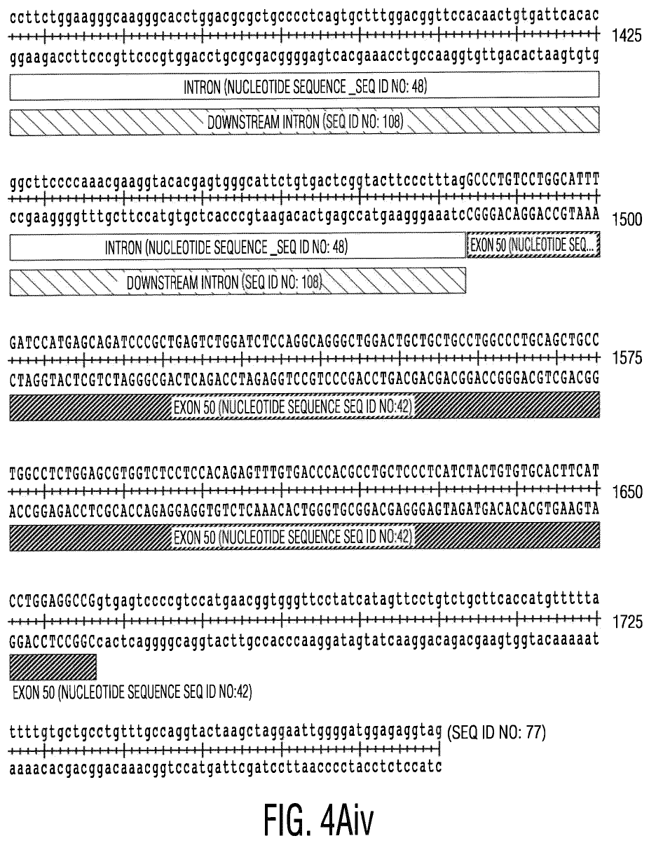

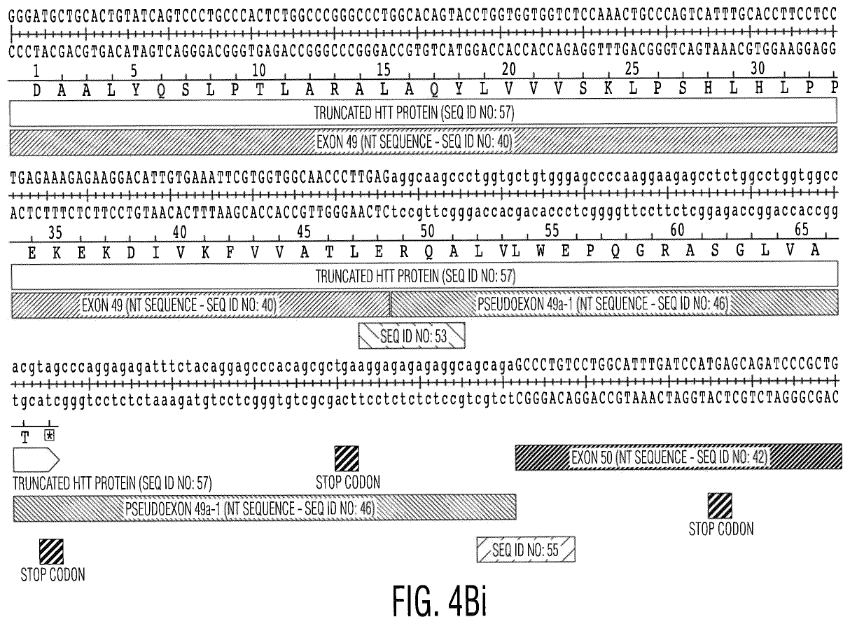

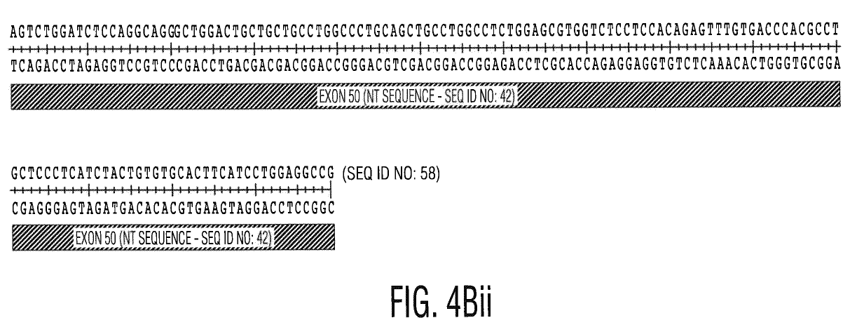

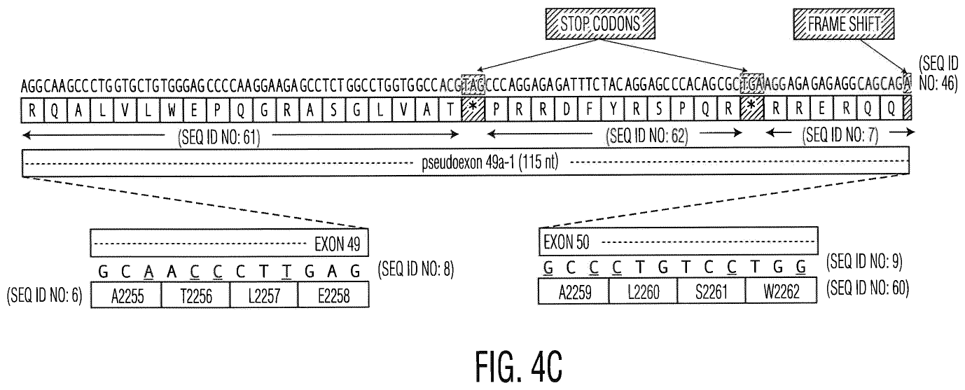

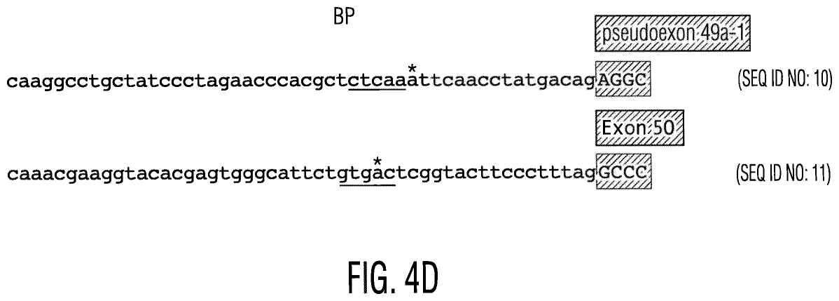

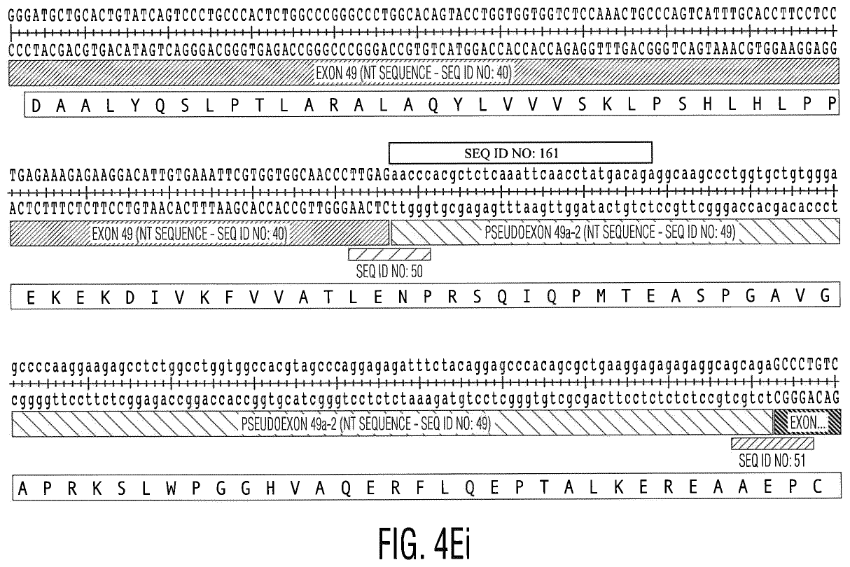

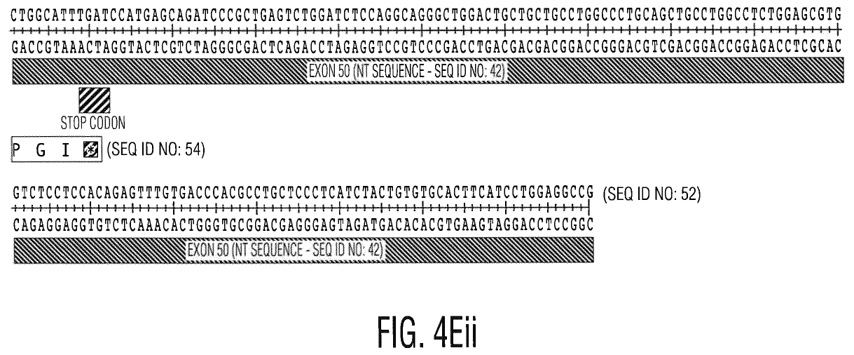

As used herein, the terms pseudoexon, psiExon, iExon, are used interchangeably throughout this disclosure to refer to a small molecule-inducible intronic sequence that can be converted, by small molecule-induced alternative splicing, into an “intron-derived exon.” For example, an “intron-derived exon” in HTT pre-mRNA is depicted in FIGS. 4Ai-iii, 4-C, and 4Ei-ii.

The terms “manifest HD” or “manifest Huntington's disease”, as used herein, refer to having diagnosis of HD as clinically established (e.g., on the basis of: confirmed family history or positive genetic test (confirmation of CAG repeat expansion ≥36); and onset of motor disturbances [diagnostic confidence score (DCS) of 4, as defined by the Unified Huntington Rating Scale (UHDRS) total motor score (TMS)]. In one aspect, the term “manifest HD” or “manifest Huntington's disease”, as used herein, refers to a patient having diagnosis of HD as clinically established [e.g., on the basis of confirmed family history or positive genetic test (confirmation of CAG repeat expansion ≥36)]; and onset of motor disturbances [e.g., on the basis of diagnostic confidence score (DCS) of 4, as defined by the Unified Huntington Rating Scale (UHDRS) total motor score (TMS)].

The terms “pre-manifest HD” or “pre-manifest Huntington's disease”, as used herein, refer to having genetic diagnosis of HD [e.g. on the basis of: positive genetic test (confirmation of CAG repeat expansion ≥40) without onset of motor disturbances as clinically stablished, for example, as assessed according to standard scales, such as, clinical scales [e.g. on the basis of a diagnostic confidence score (DCS) of <4, as defined by the Unified Huntington Rating Scale (UHDRS) total motor score (TMS)]. In one aspect, term “pre-manifest HD” or “pre-manifest Huntington's disease”, as used herein, refers to a patient having genetic diagnosis of HD [e.g. on the basis of: positive genetic test (confirmation of CAG repeat expansion ≥40)] without onset of motor disturbances as clinically stablished, for example, as assessed according to standard scales, such as, clinical scales [e.g. on the basis of a diagnostic confidence score (DCS) of <4, as defined by the Unified Huntington Rating Scale (UHDRS) total motor score (TMS)].

The terms “HD patient”, “Huntington's disease patient” or “patient with HD” refer to a “patient with Huntington's disease”, as defined herein.

As used herein, “huntingtin” refers to the huntingtin (HTT) gene, or any fragment thereof. The huntingtin gene is also known as the IT15, the Huntington Disease gene, HD gene, LOMARS gene or the HTT gene. Located on chromosome 4 at 4p16.3 in humans, the huntingtin gene (HGNC: 4851; Entrez Gene: 3064; Ensembl: ENSG00000197386; OMIM: 613004) is approximately 180 kb in length and consists of 67 exons that encode a 347 kD huntingtin protein (UniProtKB: P42858). The huntingtin gene is expressed as 2 alternatively polyadenylated forms displaying different relative abundance in various fetal and adult tissues. The larger transcript is approximately 13.7 kb and is expressed predominantly in adult and fetal brain whereas the smaller transcript of approximately 10.3 kb is ubiquitously expressed. Diseases associated with HTT include Huntington Disease and Lopes-Maciel-Rodan Syndrome.

Huntington Disease is a neurodegenerative disorder characterized by involuntary movements (chorea), general motor impairment, psychiatric disorders and dementia. Onset of the disease occurs usually in the third or fourth decade of life. Onset and clinical course depend on the degree of poly-Gin repeat expansion, longer expansions resulting in earlier onset and more severe clinical manifestations. Neuropathology of Huntington disease displays a distinctive pattern with loss of neurons, especially in the caudate and putamen. Huntington disease affects an estimated 3 to 7 per 100,000 people of European ancestry. The disorder appears to be less common in some other populations, including people of Japanese, Chinese, and African descent.

Lopes-Maciel-Rodan syndrome (LOMARS) is a rare autosomal recessive neurodevelopmental disorder characterized by developmental regression in infancy, delayed psychomotor development, severe intellectual disability, and cerebral and cerebellar atrophy. Additional features include swallowing problems, dystonia, bradykinesia, and continuous manual stereotypies without chorea. Some patients manifest seizures.

An exemplary Homo sapiens huntingtin cDNA transcript variant 2 (NCBI Reference Sequence: NM_002111.8) has a nucleotide sequence of SEQ ID NO: 13 (see TABLE I below).

In certain aspects, an exemplary Homo sapiens huntingtin can refer to a polypeptide having the amino acid sequence of SEQ ID NO: 12 (NCBI Reference Sequence: NP_002102.4) or fragment thereof (see TABLE 1).

In one aspect, a Homo sapiens huntingtin cDNA comprises at least 10, 20, 30, 40, 50 or 100 nucleotides of the sequence of SEQ ID NO: 13.

In another aspect, a Homo sapiens huntingtin protein comprises at least 10, 20, 30, 40, 50 or 100 amino acids of the polypeptide sequence of SEQ ID NO: 12.

[symbol]

TABLE I
HUMAN HTT NUCLEOTIDE AND AMINO ACID SEQUENCES

| | HUMAN HUNTINGTIN mRNA SEQUENCE | |
|---|---|---|
| | AMINO ACID SEQUENCE (SEQ ID NO: 12) | |
| | cDNA SEQUENCE (SEQ ID NO: 13) | |
| 1 | M A T L E K L K A F E S S L K S F Q Q Q | 20 |
| 1 | ATGGCGACCCTGGAAAAGCTGATGAAGGCCTTCGAGTCCCTCAAGTCCTTCCAGCAGCAG | 60 |
| 21 | Q Q Q Q Q Q Q Q Q Q Q Q Q Q Q Q Q Q Q Q | 40 |
| 61 | CAGCAGCAGCAGCAGCAGCAGCAGCAGCAGCAGCAGCAGCAGCAGCAGCAGCAGCAACAG | 120 |
| 41 | P P P P P P P P P P P Q L P Q P P P Q A | 60 |
| 121 | CCGCCACCGCCGCCGCCGCCGCCGCCGCCTCCTCAGCTTCCTCAGCCGCCGCCGCAGGCA | 180 |
| 61 | Q P L L P Q P Q P P P P P P P P P P G P | 80 |
| 181 | CAGCCGCTGCTGCCTCAGCCGCAGCCGCCCCCGCCGCCGCCCCCGCCGCCACCCGGCCCG | 240 |
| 81 | A V A E E P L H R P K K E L S A T K K D | 100 |
| 241 | GCTGTGGCTGAGGAGCCGCTGCACCGACCAAAGAAAGAACTTTCAGCTACCAAGAAAGAC | 300 |
| 101 | R V N H C L T I C E N I V A Q S V R N S | 120 |
| 301 | CGTGTGAATCATTGTCTGACAATATGTGAAAACATAGTGGCACAGTCTGTCAGAAATTCT | 360 |
| 121 | P E F Q K L L G I A M E L F L L C S D D | 140 |
| 361 | CCAGAATTTCAGAAACTTCTGGGCATCGCTATGGAACTTTTTCTGCTGTGCAGTGATGAC | 420 |
| 141 | A E S D V R M V A D E C L N K V I K A L | 160 |
| 421 | GCAGAGTCAGATGTCAGGATGGTGGCTGACGAATGCCTCAACAAAGTTATCAAAGCTTTG | 480 |
| 161 | M D S N L P R L Q L E L Y K E I K K N G | 180 |
| 481 | ATGGATTCTAATCTTCCAAGGTTACAGCTCGAGCTCTATAAGGAAATTAAAAAGAATGGT | 540 |
| 181 | A P R S L R A A L W R F A E L A H L V R | 200 |
| 541 | GCCCCTCGGAGTTTGCGTGCTGCCCTGTGGAGGTTTGCTGAGCTGGCTCACCTGGTTCGG | 600 |
| 201 | P Q K C R P Y L V N L L P C L T R T S K | 220 |
| 601 | CCTCAGAAATGCAGGCCTTACCTGGTGAACCTTCTGCCGTGCCTGACTCGAACAAGCAAG | 660 |
| 221 | R P E E S V Q E T L A A A V P K I M A S | 240 |
| 661 | AGACCCGAAGAATCAGTCCAGGAGACCTTGGCTGCAGCTGTTCCCAAAATTATGGCTTCT | 720 |
| 241 | F G N F A N D N E I K V L L K A F I A N | 260 |
| 721 | TTTGGCAATTTTGCAAATGACAATGAAATTAAGGTTTTGTTAAAGGCCTTCATAGCGAAC | 780 |
| 261 | L K S S S P T I R R T A A G S A V S I C | 280 |
| 781 | CTGAAGTCAAGCTCCCCCACCATTCGGCGGACAGCGGCTGGATCAGCAGTGAGCATCTGC | 840 |
| 281 | Q H S R R T Q Y F Y S W L L N V L L G L | 300 |
| 841 | CAGCACTCAAGAAGGACACAATATTTCTATAGTTGGCTACTAAATGTGCTCTTAGGCTTA | 900 |
| 301 | L V P V E D E H S T L L I L G V L L T L | 320 |
| 901 | CTCGTTCCTGTCGAGGATGAACACTCCACTCTGCTGATTCTTGGCGTGCTGCTCACCCTG | 960 |
| 321 | R Y L V P L L Q Q Q V K D T S L K G S F | 340 |
| 961 | AGGTATTTGGTGCCCTTGCTGCAGCAGCAGGTCAAGGACACAAGCCTGAAAGGCAGCTTC | 1020 |
| 341 | G V T R K E M E V S P S A E Q L V Q V Y | 360 |
| 1021 | GGAGTGACAAGGAAAGAAATGGAAGTCTCTCCTTCTGCAGAGCAGCTTGTCCAGGTTTAT | 1080 |
| 361 | E L T L H H T Q H Q D H N V V T G A L E | 380 |
| 1081 | GAACTGACGTTACATCATACACAGCACCAAGACCACAATGTTGTGACCGGAGCCCTGGAG | 1140 |
| 381 | L L Q Q L F R T P P P E L L Q T L T A V | 400 |
| 1141 | CTGTTGCAGCAGCTCTTCAGAACGCCTCCACCCGAGCTTCTGCAAACCCTGACCGCAGTC | 1200 |
| 401 | G G I G Q L T A A K E E S G G R S R S G | 420 |
| 1201 | GGGGGCATTGGGCAGCTCACCGCTGCTAAGGAGGAGTCTGGTGGCCGAAGCCGTAGTGGG | 1260 |
| 421 | S I V E L I A G G G S S C S P V L S R K | 440 |
| 1261 | AGTATTGTGGAACTTATAGCTGGAGGGGGTTCCTCATGCAGCCCTGTCCTTTCAAGAAAA | 1320 |
| 441 | Q K G K V L L G E E E A L E D D S E S R | 460 |
| 1321 | CAAAAAGGCAAAGTGCTCTTAGGAGAAGAAGAAGCCTTGGAGGATGACTCTGAATCGAGA | 1380 |
| 461 | S D V S S S A L T A S V K D E I S G E L | 480 |
| 1381 | TCGGATGTCAGCAGCTCTGCCTTAACAGCCTCAGTGAAGGATGAGATCAGTGGAGAGCTG | 1440 |
| 481 | A A S S G V S T P G S A G H D I I T E Q | 500 |
| 1441 | GCTGCTTCTTCAGGGGTTTCCACTCCAGGGTCAGCAGGTCATGACATCATCACAGAACAG | 1500 |
| 501 | P R S Q H T L Q A D S V D L A S C D L T | 520 |
| 1501 | CCACGGTCACAGCACACACTGCAGGCGGACTCAGTGGATCTGGCCAGCTGTGACTTGACA | 1560 |
| 521 | S S A T D G D E E D I L S H S S S Q V S | 540 |
| 1561 | ACCTCTGCCACTGATGGGGATGAGGAGGATATCTTGAGCCACAGCTCCAGCCAGGTCAGC | 1620 |
| 541 | A V P S D P A M D L N D G T Q A S S P I | 560 |
| 1621 | GCCGTCCCATCTGACCCTGCCATGGACCTGAATGATGGGACCCAGGCCTCGTCGCCCATC | 1680 |
| 561 | S D S S Q T T T E G P D S A V T P S D S | 580 |
| 1681 | AGCGACAGCTCCCAGACCACCACCGAAGGGCCTGATTCAGCTGTTACCCCTTCAGACAGT | 1740 |
| 581 | S E I V L D G T D N Q Y L G L Q I G Q P | 600 |
| 1741 | TCTGAAATTGTGTTAGACGGTACCGACAACCAGTATTTGGGCCTGCAGATTGGACAGCCC | 1800 |
| 601 | Q D E D E E A T G I L P D E A S E A F R | 620 |
| 1801 | CAGGATGAAGATGAGGAAGCCACAGGTATTCTTCCTGATGAAGCCTCGGAGGCCTTCAGG | 1860 |
| 621 | N S S M A L Q Q A H L L K N M S H C R Q | 640 |
| 1861 | AACTCTTCCATGGCCCTTCAACAGGCACATTTATTGAAAAACATGAGTCACTGCAGGCAG | 1920 |
| 641 | P S D S S V D K F V L R D E A T E P G D | 660 |
| 1921 | CCTTCTGACAGCAGTGTTGATAAATTTGTGTTGAGAGATGAAGCTACTGAACCGGGTGAT | 1980 |
| 661 | Q E N K P C R I K G D I G Q S T D D D S | 680 |
| 1981 | CAAGAAAACAAGCCTTGCCGCATCAAAGGTGACATTGGACAGTCCACTGATGATGACTCT | 2040 |
| 681 | A P L V H C V R L L S A S F L L T G G K | 700 |
| 2041 | GCACCTCTTGTCCATTGTGTCCGCCTTTTATCTGCTTCGTTTTTGCTAACAGGGGGAAAA | 2100 |
| 701 | N V L V P D R D V R V S V K A L A L S C | 720 |
| 2101 | AATGTGCTGGTTCCGGACAGGGATGTGAGGGTCAGCGTGAAGGCCCTGGCCCTCAGCTGT | 2160 |
| 721 | V G A A V A L H P E S F F S K L Y K V P | 740 |
| 2161 | GTGGGAGCAGCTGTGGCCCTCCACCCGGAATCTTTCTTCAGCAAACTCTATAAAGTTCCT | 2220 |
| 741 | L D T T E Y P E E Q Y V S D I L N Y I D | 760 |
| 2221 | CTTGACACCACGGAATACCCTGAGGAACAGTATGTCTCAGACATCTTGAACTACATCGAT | 2280 |
| 761 | H G D P Q V R G A T A I L C G T L I C S | 780 |
| 2281 | CATGGAGACCCACAGGTTCGAGGAGCCACTGCCATTCTCTGTGGGACCCTCATCTGCTCC | 2340 |
| 781 | I L S R S R F H V G D W M G T I R T L T | 800 |
| 2341 | ATCCTCAGCAGGTCCCGCTTCCACGTGGGAGATTGGATGGGCACCATTAGAACCCTCACA | 2400 |
| 801 | G N T F S L A D C I P L L R K T L K D E | 820 |
| 2401 | GGAAATACATTTTCTTTGGCGGATTGCATTCCTTTGCTGCGGAAAACACTGAAGGATGAG | 2460 |
| 821 | S S V T C K L A C T A V R N C V M S L C | 840 |
| 2461 | TCTTCTGTTACTTGCAAGTTAGCTTGTACAGCTGTGAGGAACTGTGTCATGAGTCTCTGC | 2520 |
| 841 | S S S Y S E L G L Q L I I D V L T L R N | 860 |
| 2521 | AGCAGCAGCTACAGTGAGTTAGGACTGCAGCTGATCATCGATGTGCTGACTCTGAGGAAC | 2580 |
| 861 | S S Y W L V R T E L L E T L A E I D F R | 880 |
| 2581 | AGTTCCTATTGGCTGGTGAGGACAGAGCTTCTGGAAACCCTTGCAGAGATTGACTTCAGG | 2640 |
| 881 | L V S F L E A K A E N L H R G A H H Y T | 900 |
| 2641 | CTGGTGAGCTTTTTGGAGGCAAAAGCAGAAAACTTACACAGAGGGGCTCATCATTATACA | 2700 |
| 901 | G L L K L Q E R V L N N V V I H L L G D | 920 |
| 2701 | GGGCTTTTAAAACTGCAAGAACGAGTGCTCAATAATGTTGTCATCCATTTGCTTGGAGAT | 2760 |
| 921 | E D P R V R H V A A A S L I R L V P K L | 940 |
| 2761 | GAAGACCCCAGGGTGCGACATGTTGCCGCAGCATCACTAATTAGGCTTGTCCCAAAGCTG | 2820 |
| 941 | F Y K C D Q G Q A D P V V A V A R D Q S | 960 |
| 2821 | TTTTATAAATGTGACCAAGGACAAGCTGATCCAGTAGTGGCCGTGGCAAGAGATCAAAGC | 2880 |
| 961 | S V Y L K L L M H E T Q P P S H F S V S | 980 |
| 2881 | AGTGTTTACCTGAAACTTCTCATGCATGAGACGCAGCCTCCATCTCATTTCTCCGTCAGC | 2940 |
| 981 | T I T R I Y R G Y N L L P S I T D V T M | 1000 |
| 2941 | ACAATAACCAGAATATATAGAGGCTATAACCTACTACCAAGCATAACAGACGTCACTATG | 3000 |
| 1001 | E N N L S R V I A A V S H E L I T S T T | 1020 |
| 3001 | GAAAATAACCTTTCAAGAGTTATTGCAGCAGTTTCTCATGAACTAATCACATCAACCACC | 3060 |
| 1021 | R A L T F G C C E A L C L L S T A F P V | 1040 |
| 3061 | AGAGCACTCACATTTGGATGCTGTGAAGCTTTGTGTCTTCTTTCCACTGCCTTCCCAGTT | 3120 |
| 1041 | C I W S L G W H C G V P P L S A S D E S | 1060 |
| 3121 | TGCATTTGGAGTTTAGGTTGGCACTGTGGAGTGCCTCCACTGAGTGCCTCAGATGAGTCT | 3180 |
| 1061 | R K S C T V G M A T M I L T L L S S A W | 1080 |
| 3181 | AGGAAGAGCTGTACCGTTGGGATGGCCACAATGATTCTGACCCTGCTCTCGTCAGCTTGG | 3240 |
| 1081 | F P L D L S A H Q D A L I L A G N L L A | 1100 |
| 3241 | TTCCCATTGGATCTCTCAGCCCATCAAGATGCTTTGATTTTGGCCGGAAACTTGCTTGCA | 3300 |
| 1101 | A S A P K S L R S S W A S E E E A N P A | 1120 |
| 3301 | GCCAGTGCTCCCAAATCTCTGAGAAGTTCATGGGCCTCTGAAGAAGAAGCCAACCCAGCA | 3360 |
| 1121 | A T K Q E E V W P A L G D R A L V P M V | 1140 |
| 3361 | GCCACCAAGCAAGAGGAGGTCTGGCCAGCCCTGGGGGACCGGGCCCTGGTGCCCATGGTG | 3420 |
| 1141 | E Q L F S H L L K V I N I C A H V L D D | 1160 |
| 3421 | GAGCAGCTCTTCTCTCACCTGCTGAAGGTGATTAACATTTGTGCCCACGTCCTGGATGAC | 3480 |
| 1161 | V A P G P A I K A A L P S L T N P P S L | 1180 |
| 3481 | GTGGCTCCTGGACCCGCAATAAAGGCAGCCTTGCCTTCTCTAACAAACCCCCCTTCTCTA | 3540 |
| 1181 | S P I R R K G K E K E P G E Q A S V P L | 1200 |
| 3541 | AGTCCCATCCGACGAAAGGGGAAGGAGAAAGAACCAGGAGAACAAGCATCTGTACCGTTG | 3600 |
| 1201 | S P K K G S E A S A A S R Q S D T S G P | 1220 |
| 3601 | AGTCCCAAGAAAGGCAGTGAGGCCAGTGCAGCTTCTAGACAATCTGATACCTCAGGTCCT | 3660 |
| 1221 | V T T S K S S S L G S F Y H L P S Y L K | 1240 |
| 3661 | GTTACAACAAGTAAATCCTCATCACTGGGGAGTTTCTATCATCTTCCTTCATACCTCAAA | 3720 |
| 1241 | L H D V L K A T H A N Y K V T L D L Q N | 1260 |
| 3721 | CTGCATGATGTCCTGAAAGCTACACACGCTAACTACAAGGTCACGCTGGATCTTCAGAAC | 3780 |
| 1261 | S T E K F G G F L R S A L D V L S Q I L | 1280 |
| 3781 | AGCACGGAAAAGTTTGGAGGGTTTCTCCGCTCAGCCTTGGATGTTCTTTCTCAGATACTA | 3840 |
| 1281 | E L A T L Q D I G K C V E E I L G Y L K | 1300 |
| 3841 | GAGCTGGCCACACTGCAGGACATTGGGAAGTGTGTTGAAGAGATCCTAGGATACCTGAAA | 3900 |
| 1301 | S C F S R E P M M A T V C V Q Q L L K T | 1320 |
| 3901 | TCCTGCTTTAGTCGAGAACCAATGATGGCAACTGTTTGTGTTCAACAATTGTTGAAGACT | 3960 |
| 1321 | L F G T N L A S Q F D G L S S N P S K S | 1340 |
| 3961 | CTCTTTGGCACAAACTTGGCCTCCCAGTTTGATGGCTTATCTTCCAACCCCAGCAAGTCA | 4020 |
| 1341 | Q G R A Q R L G S S S V R P G L Y H Y C | 1360 |
| 4021 | CAAGGCCGAGCACAGCGCCTTGGCTCCTCCAGTGTGAGGCCAGGCTTGTACCACTACTGC | 4080 |
| 1361 | F M A P Y T H F T Q A L A D A S L R N M | 1380 |
| 4081 | TTCATGGCCCCGTACACCCACTTCACCCAGGCCCTCGCTGACGCCAGCCTGAGGAACATG | 4140 |
| 1381 | V Q A E Q E N D T S G W F D V L Q K V S | 1400 |
| 4141 | GTGCAGGCGGAGCAGGAGAACGACACCTCGGGATGGTTTGATGTCCTCCAGAAAGTGTCT | 4200 |
| 1401 | T Q L K T N L T S V T K N R A D K N A I | 1420 |
| 4201 | ACCCAGTTGAAGACAAACCTCACGAGTGTCACAAAGAACCGTGCAGATAAGAATGCTATT | 4260 |
| 1421 | H N H I R L F E P L V I K A L K Q Y T T | 1440 |
| 4261 | CATAATCACATTCGTTTGTTTGAACCTCTTGTTATAAAAGCTTTAAAACAGTACACGACT | 4320 |
| 1441 | T T C V Q L Q K Q V L D L L A Q L V Q L | 1460 |
| 4321 | ACAACATGTGTGCAGTTACAGAAGCAGGTTTTAGATTTGCTGGCGCAGCTGGTTCAGTTA | 4380 |
| 1461 | R V N Y C L L D S D Q V F I G F V L K Q | 1480 |
| 4381 | CGGGTTAATTACTGTCTTCTGGATTCAGATCAGGTGTTTATTGGCTTTGTATTGAAACAG | 4440 |
| 1481 | F E Y I E V G Q F R E S E A I I P N I F | 1500 |
| 4441 | TTTGAATACATTGAAGTGGGCCAGTTCAGGGAATCAGAGGCAATCATTCCAAACATCTTT | 4500 |
| 1501 | F F L V L L S Y E R Y H S K Q I I G I P | 1520 |
| 4501 | TTCTTCTTGGTATTACTATCTTATGAACGCTATCATTCAAAACAGATCATTGGAATTCCT | 4560 |
| 1521 | K I I Q L C D G I M A S G R K A V T H A | 1540 |
| 4561 | AAAATCATTCAGCTCTGTGATGGCATCATGGCCAGTGGAAGGAAGGCTGTGACACATGCC | 4620 |
| 1541 | I P A L Q P I V H D L F V L R G T N K A | 1560 |
| 4621 | ATACCGGCTCTGCAGCCCATAGTCCACGACCTCTTTGTATTAAGAGGAACAAATAAAGCT | 4680 |
| 1561 | D A G K E L E T Q K E V V V S M L L R L | 1580 |
| 4681 | GATGCAGGAAAAGAGCTTGAAACCCAAAAAGAGGTGGTGGTGTCAATGTTACTGAGACTC | 4740 |
| 1581 | I Q Y H Q V L E M F I L V L Q Q C H K E | 1600 |
| 4741 | ATCCAGTACCATCAGGTGTTGGAGATGTTCATTCTTGTCCTGCAGCAGTGCCACAAGGAG | 4800 |
| 1601 | N E D K W K R L S R Q I A D I I L P M L | 1620 |
| 4801 | AATGAAGACAAGTGGAAGCGACTGTCTCGACAGATAGCTGACATCATCCTCCCAATGTTA | 4860 |
| 1621 | A K Q Q M H I D S H E A L G V L N T L F | 1640 |
| 4861 | GCCAAACAGCAGATGCACATTGACTCTCATGAAGCCCTTGGAGTGTTAAATACATTATTT | 4920 |
| 1641 | E I L A P S S L R P V D M L L R S M F V | 1660 |
| 4921 | GAGATTTTGGCCCCTTCCTCCCTCCGTCCGGTAGACATGCTTTTACGGAGTATGTTCGTC | 4980 |
| 1661 | T P N T M A S V S T V Q L W I S G I L A | 1680 |
| 4981 | ACTCCAAACACAATGGCGTCCGTGAGCACTGTTCAACTGTGGATATCGGGAATTCTGGCC | 5040 |
| 1681 | I L R V L I S Q S T E D I V L S R I Q E | 1700 |
| 5041 | ATTTTGAGGGTTCTGATTTCCCAGTCAACTGAAGATATTGTTCTTTCTCGTATTCAGGAG | 5100 |
| 1701 | L S F S P Y L I S C T V I N R L R D G D | 1720 |
| 5101 | CTCTCCTTCTCTCCGTATTTAATCTCCTGTACAGTAATTAATAGGTTAAGAGATGGGGAC | 5160 |
| 1721 | S T S T L E E H S E D K Q I K N L P E E | 1740 |
| 5161 | AGTACTTCAACGCTAGAAGAACACAGTGAAGGGAAACAAATAAAGAATTTGCCAGAAGAA | 5220 |
| 1741 | T F S R F L L Q L V G I L L E D I V T K | 1760 |
| 5221 | ACATTTTCAAGGTTTCTATTACAACTGGTTGGTATTCTTTTAGAAGACATTGTTACAAAA | 5280 |
| 1761 | Q L K V E M S E Q Q H T F Y C Q E L G T | 1780 |
| 5281 | CAGCTGAAGGTGGAAATGAGTGAGCAGCAACATACTTTCTATTGCCAGGAACTAGGCACA | 5340 |
| 1781 | L L M C L I H I F K S G M F R R I T A A | 1800 |
| 5341 | CTGCTAATGTGTCTGATCCACATCTTCAAGTCTGGAATGTTCCGGAGAATCACAGCAGCT | 5400 |
| 1801 | A T R L F R S D G C G G S F Y T L D S L | 1820 |
| 5401 | GCCACTAGGCTGTTCCGCAGTGATGGCTGTGGCGGCAGTTTCTACACCCTGGACAGCTTG | 5460 |
| 1821 | N L R A R S M I T T H P A L V L L W C Q | 1840 |
| 5461 | AACTTGCGGGCTCGTTCCATGATCACCACCCACCCGGCCCTGGTGCTGCTCTGGTGTCAG | 5520 |
| 1841 | I L L L V N H T D Y R W W A E V Q Q T P | 1860 |
| 5521 | ATACTGCTGCTTGTCAACCACACCGACTACCGCTGGTGGGCAGAAGTGCAGCAGACCCCG | 5580 |
| 1861 | K R H S L S S T K L L S P Q M S G E E E | 1880 |
| 5581 | AAAAGACACAGTCTGTCCAGCACAAAGTTACTTAGTCCCCAGATGTCTGGAGAAGAGGAG | 5640 |
| 1881 | D S D L A A K L G M C N R E I V R R G A | 1900 |
| 5641 | GATTCTGACTTGGCAGCCAAACTTGGAATGTGCAATAGAGAAATAGTACGAAGAGGGGCT | 5700 |
| 1901 | L I L F C D Y V C Q N L H D S E H L T W | 1920 |
| 5701 | CTCATTCTCTTCTGTGATTATGTCTGTCAGAACCTCCATGACTCCGAGCACTTAACGTGG | 5760 |
| 1921 | L I V N H I Q D L I S L S H E P P V Q D | 1940 |
| 5761 | CTCATTGTAAATCACATTCAAGATCTGATCAGCCTTTCCCACGAGCCTCCAGTACAGGAC | 5820 |
| 1941 | F I S A V H R N S A A S G L F I Q A I Q | 1960 |
| 5821 | TTCATCAGTGCCGTTCATCGGAACTCTGCTGCCAGCGGCCTGTTCATCCAGGCAATTCAG | 5880 |
| 1961 | S R C E N L S T P T M L K K T L Q C L E | 1980 |
| 5881 | TCTCGTTGTGAAAACCTTTCAACTCCAACCATGCTGAAGAAAACTCTTCAGTGCTTGGAG | 5940 |
| 1981 | G I H L S Q S G A V L T L Y V D R L L C | 2000 |
| 5941 | GGGATCCATCTCAGCCAGTCGGGAGCTGTGCTCACGCTGTATGTGGACAGGCTTCTGTGC | 6000 |
| 2001 | T P F R V L A R M V D I L A C R R V E M | 2020 |
| 6001 | ACCCCTTTCCGTGTGCTGGCTCGCATGGTCGACATCCTTGCTTGTCGCCGGGTAGAAATG | 6060 |
| 2021 | L L A A N L Q S S M A Q L P M E E L N R | 2040 |
| 6061 | CTTCTGGCTGCAAATTTACAGAGCAGCATGGCCCAGTTGCCAATGGAAGAACTCAACAGA | 6120 |
| 2041 | I Q E Y L Q S S G L A Q R H Q R L Y S L | 2060 |
| 6121 | ATCCAGGAATACCTTCAGAGCAGCGGGCTCGCTCAGAGACACCAAAGGCTCTATTCCCTG | 6180 |
| 2061 | L D R F R L S T M Q D S L S P S P P V S | 2080 |
| 6181 | CTGGACAGGTTTCGTCTCTCCACCATGCAAGACTCACTTAGTCCCTCTCCTCCAGTCTCT | 6240 |
| 2081 | S H P L D G D G H V S L E T V S P D K D | 2100 |
| 6241 | TCCCACCCGCTGGACGGGGATGGGCACGTGTCACTGGAAACAGTGAGTCCGGACAAAGAC | 6300 |
| 2101 | W Y V H L V K S Q C W T R S D S A L L E | 2120 |
| 6301 | TGGTACGTTCATCTTGTCAAATCCCAGTGTTGGACCAGGTCAGATTCTGCACTGCTGGAA | 6360 |
| 2121 | G A E L V N R I P A E D M N A F M M N S | 2140 |
| 6361 | GGTGCAGAGCTGGTGAATCGGATTCCTGCTGAAGATATGAATGCCTTCATGATGAACTCG | 6420 |
| 2141 | E F N L S L L A P C L S L G M S E I S G | 2160 |
| 6421 | GAGTTCAACCTAAGCCTGCTAGCTCCATGCTTAAGCCTAGGGATGAGTGAAATTTCTGGT | 6480 |
| 2161 | G Q K S A L F E A A R E V T L A R V S G | 2180 |
| 6481 | GGCCAGAAGAGTGCCCTTTTTGAAGCAGCCCGTGAGGTGACTCTGGCCCGTGTGAGCGGC | 6540 |
| 2181 | T V Q Q L P A V H H V F Q P E L P A E P | 2200 |
| 6541 | ACCGTGCAGCAGCTCCCTGCTGTCCATCATGTCTTCCAGCCCGAGCTGCCTGCAGAGCCG | 6600 |
| 2201 | A A Y W S K L N D L F G D A A L Y Q S L | 2220 |
| 6601 | 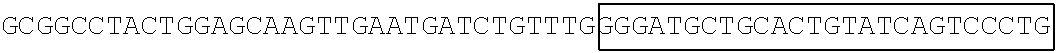 | 6660 |
| 2221 | P T L A R A L A Q Y L V V V S K L P S H | 2240 |
| 6661 | 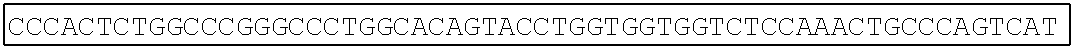 | 6720 |
| 2241 | L H L P P E K H K D I V K F V V A T L E | 2260 |
| 6721 | 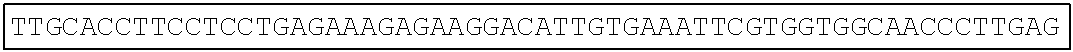 | 6780 |
| 2261 | A L S W H L I H E Q I P L S L D L Q A G | 2280 |
| 6781 | 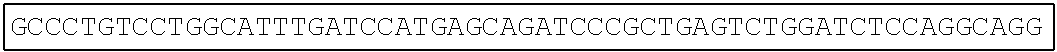 | 6840 |
| 2281 | L D C C C L A L Q L P G L W S V V S S T | 2300 |
| 6841 | 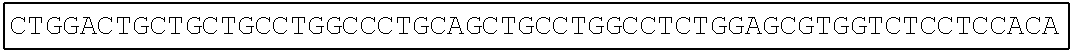 | 6900 |
| 2301 | | 2320 |
| 6901 | | 6960 |
| 2321 | E F V T H A C S L I Y C V H F I L E A V | 2340 |
| 6961 | 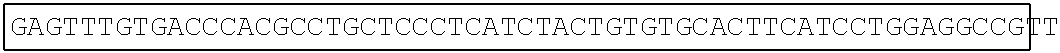 | 7020 |
| 2341 | A V Q P G E Q L L S P E R R T N T P K A | 2360 |
| 7021 | GCAGTGCAGCCTGGAGAGCAGCTTCTTAGTCCAGAAAGAAGGACAAATACCCCAAAAGCC | 7080 |
| 2361 | I S E E E E E V D P N T Q N P K Y I T A | 2380 |
| 7081 | ATCAGCGAGGAGGAGGAGGAAGTAGATCCAAACACACAGAATCCTAAGTATATCACTGCA | 7140 |
| 2381 | A C E M V A E M V E S L Q S V L A L G H | 2400 |
| 7141 | GCCTGTGAGATGGTGGCAGAAATGGTGGAGTCTCTGCAGTCGGTGTTGGCCTTGGGTCAT | 7200 |
| 2401 | K R N S G V P A F L T P L L R N I I I S | 2420 |
| 7201 | AAAAGGAATAGCGGCGTGCCGGCGTTTCTCACGCCATTGCTAAGGAACATCATCATCAGC | 7260 |
| 2421 | L A R L P L V N S Y T R V P P L V W K L | 2440 |
| 7261 | CTGGCCCGCCTGCCCCTTGTCAACAGCTACACACGTGTGCCCCCACTGGTGTGGAAGCTT | 7320 |
| 2441 | G W S P K P G G D F G T A F P E I P V E | 2460 |
| 7321 | GGATGGTCACCCAAACCGGGAGGGGATTTTGGCACAGCATTCCCTGAGATCCCCGTGGAG | 7380 |
| 2461 | F L Q E K E V F K E F I Y R I N T L G W | 2480 |
| 7381 | TTCCTCCAGGAAAAGGAAGTCTTTAAGGAGTTCATCTACCGCATCAACACACTAGGCTGG | 7440 |
| 2481 | T S R T Q F E E T W A T L L G V L V T Q | 2500 |
| 7441 | ACCAGTCGTACTCAGTTTGAAGAAACTTGGGCCACCCTCCTTGGTGTCCTGGTGACGCAG | 7500 |
| 2501 | P L V M E Q E E S P P E E D T E R T Q I | 2520 |
| 7501 | CCCCTCGTGATGGAGCAGGAGGAGAGCCCACCAGAAGAAGACACAGAGAGGACCCAGATC | 7560 |
| 2521 | N V L A V Q A I T S L V L S A M T V P V | 2540 |
| 7561 | AACGTCCTGGCCGTGCAGGCCATCACCTCACTGGTGCTCAGTGCAATGACTGTGCCTGTG | 7620 |
| 2541 | A G N P A V S C L E Q Q P R N K P L K A | 2560 |
| 7621 | GCCGGCAACCCAGCTGTAAGCTGCTTGGAGCAGCAGCCCCGGAACAAGCCTCTGAAAGCT | 7680 |
| 2561 | L D T R F G R K L S I I R G I V E Q E I | 2580 |
| 7681 | | 7740 |
| 2581 | CTCGACACCAGGTTTGGGAGGAAGCTGAGCATTATCAGAGGGATTGTGGAGCAAGAGATT | 2600 |
| 7741 | Q A M V S K R E N I A T H H L Y Q A W D | 7800 |
| 2601 | CAAGCAATGGTTTCAAAGAGAGAGAATATTGCCACCCATCATTTATATCAGGCATGGGAT | 2620 |
| 7801 | P V P S L S P A T T G A L I S H E K L L | 7860 |
| 2621 | CCTGTCCCTTCTCTGTCTCCGGCTACTACAGGTGCCCTCATCAGCCACGAGAAGCTGCTG | 2640 |
| 7861 | L Q I N P E R E L G S M S Y K L G Q V S | 7920 |
| 2641 | CTACAGATCAACCCCGAGCGGGAGCTGGGGAGCATGAGCTACAAACTCGGCCAGGTGTCC | 2660 |
| 7921 | I H S V W L G N S I T P L R E E E W D E | 7980 |
| 2661 | ATACACTCCGTGTGGCTGGGGAACAGCATCACACCCCTGAGGGAGGAGGAATGGGACGAG | 2680 |
| 7981 | E E E E E A D A P A P S S P P T S P V N | 8040 |
| 2681 | GAAGAGGAGGAGGAGGCCGACGCCCCTGCACCTTCGTCACCACCCACGTCTCCAGTCAAC | 2700 |
| 8041 | S R K H R A G V D I H S C S Q F L L E L | 8100 |
| 2701 | TCCAGGAAACACCGGGCTGGAGTTGACATCCACTCCTGTTCGCAGTTTTTGCTTGAGTTG | 2720 |
| 8101 | Y S R W I L P S S S A R R T P A I L I S | 8160 |
| 2721 | TACAGCCGCTGGATCCTGCCGTCCAGCTCAGCCAGGAGGACCCCGGCCATCCTGATCAGT | 2740 |
| 8161 | E V V R S L L V V S D L F T E R N Q F E | 8220 |
| 2741 | GAGGTGGTCAGATCCCTTCTAGTGGTCTCAGACTTGTTCACCGAGCGCAACCAGTTTGAG | 2760 |
| 8221 | L M Y V T L T E L R R V H P S E D E I L | 8280 |
| 2761 | CTGATGTATGTGACGCTGACAGAACTGCGAAGGGTGCACCCTTCAGAAGACGAGATCCTC | 2780 |
| 8281 | A Q Y L V P A T C K A A A V L G M D K A | 8340 |
| 2781 | GCTCAGTACCTGGTGCCTGCCACCTGCAAGGCAGCTGCCGTCCTTGGGATGGACAAGGCC | 2800 |
| 8341 | V A E P V S R L L E S T L R S S H L P S | 8400 |
| 2801 | GTGGCGGAGCCTGTCAGCCGCCTGCTGGAGAGCACGCTCAGGAGCAGCCACCTGCCCAGC | 2820 |
| 8401 | R V G A L H G V L Y V L E C D L L D D T | 8460 |
| 2821 | AGGGTTGGAGCCCTGCACGGCGTCCTCTATGTGCTGGAGTGCGACCTGCTGGACGACACT | 2840 |
| 8461 | A K Q L I P V I S D Y L L S N L K G I A | 8520 |
| 2841 | GCCAAGCAGCTCATCCCGGTCATCAGCGACTATCTCCTCTCCAACCTGAAAGGGATCGCC | 2860 |
| 8521 | H C V N I H S Q Q H V L V M C A T A F Y | 8580 |
| 2861 | CACTGCGTGAACATTCACAGCCAGCAGCACGTACTGGTCATGTGTGCCACTGCGTTTTAC | 2880 |
| 8581 | L I E N Y P L D V G P E F S A S I I Q M | 8640 |
| 2881 | CTCATTGAGAACTATCCTCTGGACGTAGGGCCGGAATTTTCAGCATCAATAATACAGATG | 2900 |
| 8641 | C G V M L S G S E E S T P S I I Y H C A | 8700 |
| 2901 | TGTGGGGTGATGCTGTCTGGAAGTGAGGAGTCCACCCCCTCCATCATTTACCACTGTGCC | 2920 |
| 8701 | L R G L E R L L L S E Q L S R L D A E S | 8760 |
| 2921 | CTCAGAGGCCTGGAGCGCCTCCTGCTCTCTGAGCAGCTCTCCCGCCTGGATGCAGAATCG | 2940 |
| 8761 | L V K L S V D R V N V H S P H R A M A A | 8820 |
| 2941 | CTGGTCAAGCTGAGTGTGGACAGAGTGAACGTGCACAGCCCGCACCGGGCCATGGCGGCT | 2960 |
| 8821 | L G L M L T C M Y T G K E K V S P G R T | 8880 |
| 2961 | CTGGGCCTGATGCTCACCTGCATGTACACAGGAAAGGAGAAAGTCAGTCCGGGTAGAACT | 2980 |
| 8881 | S D P N P A A P D S E S V I V A M E R V | 8940 |
| 2981 | TCAGACCCTAATCCTGCAGCCCCCGACAGCGAGTCAGTGATTGTTGCTATGGAGCGGGTA | 3000 |
| 8941 | S V L F D R I R K G F P C E A R V V A R | 9000 |
| 3001 | TCTGTTCTTTTTGATAGGATCAGGAAAGGCTTTCCTTGTGAAGCCAGAGTGGTGGCCAGG | 3020 |
| 9001 | I L P Q F L D D F F P P Q D I M N K V I | 9060 |
| 3021 | ATCCTGCCCCAGTTTCTAGACGACTTCTTCCCACCCCAGGACATCATGAACAAAGTCATC | 3040 |
| 9061 | G E F L S N Q Q P Y P Q F M A T V V Y K | 9120 |
| 3041 | GGAGAGTTTCTGTCCAACCAGCAGCCATACCCCCAGTTCATGGCCACCGTGGTGTATAAG | 3060 |
| 9121 | V F Q T L H S T G Q S S M V R D W V M L | 9180 |
| 3061 | GTGTTTCAGACTCTGCACAGCACCGGGCAGTCGTCCATGGTCCGGGACTGGGTCATGCTG | 3080 |
| 9181 | S L S N F T Q R A P V A M A T W S L S C | 9240 |
| 3081 | TCCCTCTCCAACTTCACGCAGAGGGCCCCGGTCGCCATGGCCACGTGGAGCCTCTCCTGC | 3100 |
| 9241 | F F V S A S T S P W V A A I L P H V I S | 9300 |
| 3101 | TTCTTTGTCAGCGCGTCCACCAGCCCGTGGGTCGCGGCGATCCTCCCACATGTCATCAGC | 3120 |
| 9301 | R M G K L E Q V D V N L F C L V A T D F | 9360 |
| 3121 | AGGATGGGCAAGCTGGAGCAGGTGGACGTGAACCTTTTCTGCCTGGTCGCCACAGACTTC | 3140 |
| 9361 | Y R H Q I E E E L D R R A F Q S V L E V | 9420 |
| 3141 | TACAGACACCAGATAGAGGAGGAGCTCGACCGCAGGGCCTTCCAGTCTGTGCTTGAGGTG | 3145 |
| 9421 | V A A P G S P Y H R L L T C L R N V H K | 9435 |
| | GTTGCAGCCCCAGGAAGCCCATATCACCGGCTGCTGACTTGTTTACGAAATGTCCACAAG | |
| | V T T C * | |
| | GTCACCACCTGCTGA | |

TABLE II
SEQUENCE OF SELECTED HUMAN HUNTINGTIN REGIONS
HUMAN HUNTINGTIN SEQUENCES

| A | HUMAN HUNTINGTIN EXON 49 cDNA SEQUENCE: |
|---|---|
| | AMINO ACID SEQUENCE (SEQ ID NO: 39) |
| | cDNA SEQUENCE (SEQ ID NO: 40) |
| | D A A L Y Q S L P T L A R A L A Q Y L V |
| | GGGATGCTGCACTGTATCAGTCCCTGCCCACTCTGGCCCGGGCCCTGGCACAGTACCTGGTG |
| | V V S K L P S H L H L P P E K E K D I V |
| | GTGGTCTCCAAACTGCCCAGTCATTTGCACCTTCCTCCTGAGAAAGAGAAGGACATTGTG |
| | K F V V A T L E |
| | AAATTCGTGGTGGCAACCCTTGAG |
| B | HUMAN HUNTINGTIN EXON 50 cDNA SEQUENCE: |
| | AMINO ACID SEQUENCE (SEQ ID NO: 41) |
| | cDNA SEQUENCE (SEQ ID NO: 42) |
| | A L S W H L I H E Q I P L S L D L Q A G |
| | GCCCTGTCCTGGCATTTGATCCATGAGCAGATCCCGCTGAGTCTGGATCTCCAGGCAGGG |
| | L D C C C L A L Q L P G L W S V V S S T |
| | CTGGACTGCTGCTGCCTGGCCCTGCAGCTGCCTGGCCTCTGGAGCGTGGTCTCCTCCACA |
| | E F V T H A C S L I Y C V H F I L E A |
| | GAGTTTGTGACCCACGCCTGCTCCCTCATCTACTGTGTGCACTTCATCCTGGAGGCCG |
| C | HUMAN HUNTINGTIN EXON 49-EXON 50 (WT SPLICING) cDNA SEQUENCE: |
| | AMINO ACID SEQUENCE (SEQ ID NO: 43) |
| | cDNA SEQUENCE (SEQ ID NO: 44) |
| | D A A L Y Q S L |
| | GGGATGCTGCACTGTATCAGTCCCTG |
| | P T L A R A L A Q Y L V V V S K L P S H |
| | CCCACTCTGGCCCGGGCCCTGGCACAGTACCTGGTGGTGGTCTCCAAACTGCCCAGTCAT |
| | L H L P P E K E K D I V K F V V A T L E |
| | TTGCACCTTCCTCCTGAGAAAGAGAAGGACATTGTGAAATTCGTGGTGGCAACCCTTGAG↑ Splice junction |
| | A L S W H L I H E Q I P L S L D L Q A G ↓ |
| | GCCCTGTCCTGGCATTTGATCCATGAGCAGATCCCGCTGAGTCTGGATCTCCAGGCAGGG |
| | L D C C C L A L Q L P G L W S V V S S T |
| | CTGGACTGCTGCTGCCTGGCCCTGCAGCTGCCTGGCCTCTGGAGCGTGGTCTCCTCCACA |
| | E F V T H A C S L I Y C V H F I L E A |
| | GAGTTTGTGACCCACGCCTGCTCCCTCATCTACTGTGTGCACTTCATCCTGGAGGCCG |
| D | HUMAN HUNTINGTIN EXON 49-PSEUDOEXON 49a-EXON 50 cDNA SEQUENCE (SMALL |
| | MOLECULE-INDUCED SPLICING) |
| | GGGATGCTGCACTGTATCAGTCCCTGCCCACTCTGGCCCGGGCCCTGGCACAGTACCTGGTGGTGGTCTCCAAA |
| | 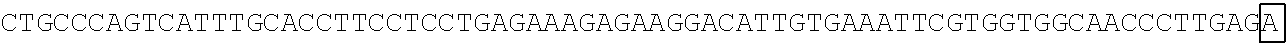 |
| | 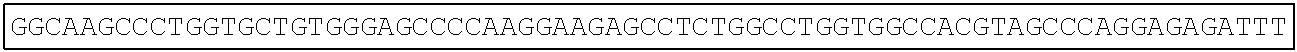 |
| | 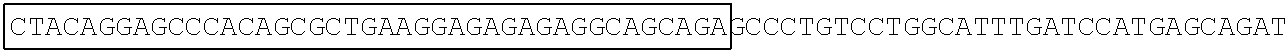 |
| | CCCGCTGAGTCTGGATCTCCAGGCAGGGCTGGACTGCTGCTGCCTGGCCCTGCAGCTGCCTGGCCTCTGGAGCG |
| | TGGTCTCCTCCACAGAGTTTGTGACCCACGCCTGCTCCCTCATCTACTGTGTGCACTTCATCCTGGAGGCCG |
| | (SEQ ID NO: 45) |
| | Sequence highlighted in rectagular box: 115 nt human HTT pseudoexon 49a |
| | (SEQ ID NO: 46) |
| E | HUMAN HUNTINGTIN PSEUDOEXON 49a-1 (115 nt): |
| | AGGCAAGCCCTGGTGCTGTGGGAGCCCCAAGGAAGAGCCTCTGGCCTGGTGGCCACGTAGCCCAGGAGAGATTT |
| | CTACAGGAGCCCACAGCGCTGAAGGAGAGAGAGGCAGCAGA (SEQ ID NO: 46) |
| F | HUMAN HUNTINGTIN PSEUDOEXON 49a-2 (146 nt) |
| | AACCCACGCTCTCAAATTCAACCTATGACAGAGGCAAGCCCTGGTGCTGTGGGAGCCCCAAGGAAGAGCCTCTG |
| | GCCTGGTGGCCACGTAGCCCAGGAGAGATTTCTACAGGAGCCCACAGCGCTGAAGGAGAGAGAGGCAGCAGA |
| | (SEQ ID NO: 49) |
| G | AGAgtaagg (PSEUDOEXON 49A 5′ SS; SEQ ID NO: 5) |
| | ccaaggcctgctatccctagAAC (PSEUDOEXON 49A 3′SS-2; SEQ ID NO: 47) |
| | caaattcaacctatgacagAGG (PSEUDOEXON 49A 3′ SS-1; SEQ ID NO: 4) |
| H | gtaagaggcagctcgggagctcagtgttgctgtggggagggggcatggggctgacactgaagagggtaaagcag |
| | ttttatttgaaaagcaagatctctgaccagtccagtcacttttccatctcagcctggcagtaagtcttgtcacc |
| | gtcaagttattgtagccatccttcaccctcacctcgccactcctcatggtggcctgtgaggtcagccaggtccc |
| | cttctcatctgcacctaccatgttaggtggatcctaattttagagacatgaaaaataatcatctggaagtactt |
| | tatgtcttaagttggcctggacatgtcagccaaggaatacttacttggtttgtgttagtgcttgtaattcgccc |
| | ccagaatgtgtacacgttctggatgcattaaagtctggcctgtatccttaaagggccatcgctgtgctgoctgc |
| | cctcagcaaggacacactttgcagacccacagaggctccgcctccacctcacaccaaagaaagggaggagtcca |
| | aagggcatcagtgccattactcacaaaatgataaatacacccttattctgaaccacgtggagtcatatggtttg |
| | tgatccctgtccttcaggtttcagcttagtggggaagtgggaaagtcagcgtgtgatcacagcacagggtgatt |
| | gctgctgattatattatgtgcctgctgtatgcaggatgaaatactttatatgcgtcatcttatttgactctcac |
| | aaccccctgtgagataggctctgttactcccatttgacaggtgaggaaagcaaggcttagagaatttcagtgac |
| | ttgcccaggtcctctgagctaggaagtagccattctggcatttgaacccaaggoctgctatccctagAACccac |
| | gctctcaaattcaacctatgacagAGGcaagccctggtgctgtgggagccccaaggaagagcctctggectggt |
| | ggccacgtagcccaggagagatttctacaggagcccacagcgctgaaggagagagaggcagcagagtaaggggg |
| | ctttgtggcagagaggggactggcactttggggaataggtgggtcaggactgaatgtaatggagccatgtcaga |
| | gctgtccttctggaagggcaagggcacctggacgcgctgcccctcagtgctttggacggttccacaactgtgat |
| | tcacacggcttccccaaacgaaggtacacgagtgggcattctgtgactcggtacttccctttag |
| | (HUMAN HUNTINGTON INTRON-49-SEQ ID NO: 48) |

### II. Compound (I)

Compound (I) refers to a small molecule that induces nonsense mediated decay of an mRNA thereby lowering the amount of protein(s) encoded by the mRNA.

In one aspect, the Compound (I) of the disclosure can be referred to as a “small molecule” or simply “compound” or “small molecule splicing modifier” (SMSM).

In one aspect, a small molecule of the disclosure having mRNA and protein lowering activity on the mRNA or protein expression of a gene can also be referred to as a small molecule splicing modifier (SMSM).

In one aspect, Compound (I) of the disclosure can refer to any one of the following small molecules:

| COMPOUND | STRUCTURE |
|---|---|
| HTT-A | 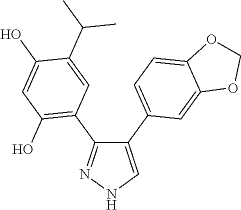 [SMILES: CC(C)c1cc(-c2n[nH]cc2-c2ccc3c(c2)OCO3)c(O)cc1O] |
| HTT-B | 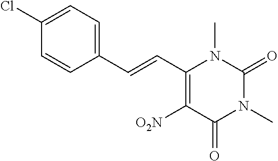 [SMILES: Cn1c(/C=C/c2ccc(Cl)cc2)c([N+](=O)[O-])c(=O)n(C)c1=O] |
| HTT-C1 | 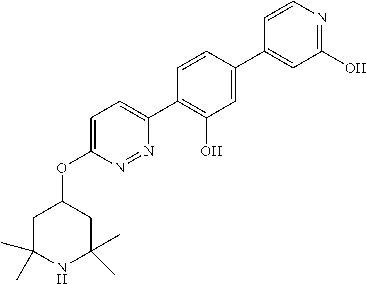 [SMILES: CC1(C)CC(Oc2ccc(-c3ccc(-c4ccnc(O)c4)cc3O)nn2)CC(C)(C)N1] |
| HTT-C2 | 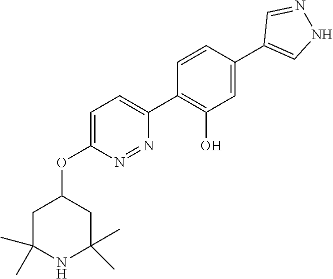 [SMILES: CC1(C)CC(Oc2ccc(-c3ccc(-c4cn[nH]c4)cc3O)nn2)CC(C)(C)N1] |
| HTT-C3 | 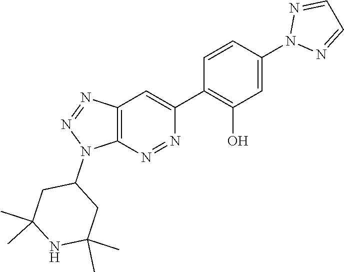 [SMILES: CC1(C)CC(n2nnc3cc(-c4ccc(-n5nccn5)cc4O)nnc32)CC(C)(C)N1] |
| HTT-D1 | 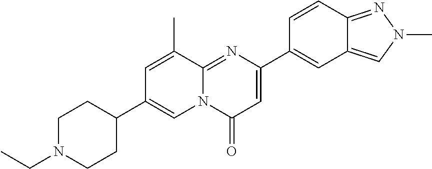 [SMILES: CCN1CCC(c2cc(C)c3nc(-c4ccc5nn(C)cc5c4)cc(=O)n3c2)CC1] |
| HTT-D2 | 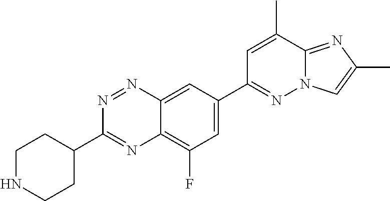 [SMILES: Cc1cn2nc(-c3cc(F)c4nc(C5CCNCC5)nnc4c3)cc(C)c2n1] |
| HTT-D3 | 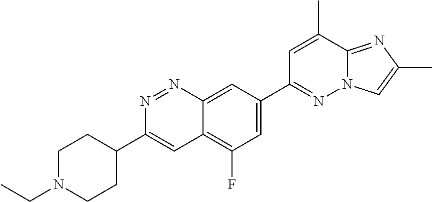 [SMILES: CCN1CCC(c2cc3c(F)cc(-c4cc(C)c5nc(C)cn5n4)cc3nn2)CC1] |
| RG7916 | 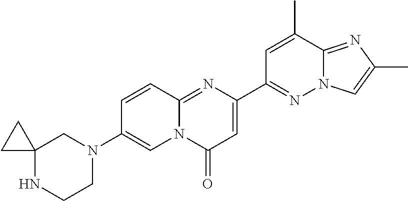 |

In one aspect, Compound (I) of the disclosure induces the inclusion of an intron-derived exon into the coding region of an mRNA thereby introducing a frameshift mutation within an mRNA.

In one aspect, Compound (I) of the disclosure can refer to a small molecule having lower activity on HTT mRNA and protein expression.

In one aspect, Compound (I) of the disclosure induces nonsense mediated decay of an mRNA, e.g., HTT mRNA.

In one aspect, described herein Compound (I) or a pharmaceutically acceptable salt thereof may be prepared by those skilled in the art, such as, by the synthetic methods set forth in International Application Number PCT/US2016/066042, filed Dec. 11, 2016, and published as International Publication Number WO2017/100726 on Jun. 15, 2017; International Application Number PCT/US2018/035954 filed Jun. 5, 2018 and published as International Publication Number WO2018/226622 on Dec. 13, 2018; International Application Number PCT/US2018/039775 filed Jun. 27, 2018 and published as International Publication Number WO2019/005980 on Jan. 3, 2019; International Application Number PCT/US2018/039794 filed Jun. 27, 2018 and published as International Publication Number WO2019/005993 on Jan. 3, 2019; International Application Number PCT/US2019/038889 filed Jun. 25, 2019 and published as International Publication Number WO2020/005873 on Jan. 2, 2020, the contents which are is incorporated by reference herein in their entireties as if fully set forth herein.

### III. Compound Forms

As used herein, Compound (I) may have a form selected from the group consisting of a free acid, free base, prodrug, salt, hydrate, solvate, clathrate, isotopologue, racemate, enantiomer, diastereomer, stereoisomer, polymorph and tautomer form thereof.

In certain aspects described herein, the form of Compound (I) is a free acid, free base or salt form thereof.

In certain aspects described herein, Compound (I) is a salt form.

In certain aspects described herein, the salt form of Compound (I) is a pharmaceutically acceptable salt.

In certain aspects described herein, Compound (I) is isolated for use.

The term “pharmaceutically acceptable salt(s)”, as used herein, means a salt of Compound (I) that is safe and effective (i.e., non-toxic, physiologically acceptable) for use in mammals and possesses biological activity, although other salts may be found useful. A salt of Compound (I) may be formed, for example, by reacting Compound (I) with an amount of acid or base, such as an equivalent amount, in a medium such as one in which the salt precipitates or in an aqueous medium followed by lyophilization.

Pharmaceutically acceptable salts include one or more salts of acidic or basic groups present in compounds described herein. In certain aspects, acid addition salts may include, and are not limited to, acetate, ascorbate, benzoate, benzenesulfonate, bisulfate, bitartrate, borate, bromide, butyrate, chloride, citrate, camphorate, camphorsulfonate, ethanesulfonate, formate, fumarate, gentisinate, gluconate, glucaronate, glutamate, hydrochloride, iodide, isonicotinate, lactate, maleate, methanesulfonate, naphthalenesulfonate, nitrate, oxalate, pamoate, pantothenate, phosphate, propionate, saccharate, salicylate, succinate, sulfate, tartrate, thiocyanate, toluenesulfonate (also known as tosylate), trifluoroacetate and the like. Certain aspects of acid addition salts may further include acetate, bromide, chloride, dichloride, trichloride, hydrochloride, dihydrochloride, formate or trifluoroacetate salts.

All such acid salts and base salts are intended to be included within the scope of pharmaceutically acceptable salts as described herein. In addition, all such acid and base salts are considered equivalent to the free forms of Compound (I).

The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or isotopologues of the instant compounds.

Another aspect, described herein includes Compound (I) selected from a polymorphic crystalline and amorphous form of Compound (I) and a salt, solvate, hydrate or ester of Compound (I).

Nomenclature for Compound (I) may differ slightly from other chemical names known to those skilled in the art; however, such differences will be recognized by one skilled in the art as equivalents for the structure of Compound (I) provided herein.

### IV. Compound (I) Modulation of Gene Expression

In another aspect, provided herein is a method for determining whether Compound (I) modulates the splicing of an RNA transcript (e.g., an mRNA transcript), comprising: (a) culturing a cell(s) in the presence of Compound (I); (b) isolating two or more RNA transcript splice variants from the cell(s) after a certain period of time; and (c) determining the amount of the two or more RNA transcript splice variants produced by the cell(s), wherein modulation in the amount of the two or more RNA transcript in the presence of Compound (I) relative to the amount of the two or more RNA transcript splice variants in the absence of Compound (I) or the presence of a negative control (e.g., a vehicle control such as PBS or DMSO) indicates that Compound (I) modulates the splicing of the RNA transcript.

In another aspect, provided herein is a method for determining whether Compound (I) modulates the splicing of an RNA transcript (e.g., an mRNA transcript), comprising (a) culturing a first cell(s) in the presence of Compound (I); (b) culturing a second cell(s) in the presence of a negative control (e.g., a vehicle control, such as PBS or DMSO); (c) isolating two or more RNA transcript splice variants produced by the first cell(s) and isolating two or more RNA transcript splice variants produced by the second cell(s); (d) determining the amount of the two or more RNA transcript splice variants produced by the first cell(s) and the second cell(s); and (e) comparing the amount of the two or more RNA transcript splice variants produced by the first cell(s) to the amount of the two or more RNA transcript splice variants produced by the second cell(s), wherein modulation in the amount of the two or more RNA transcript splice variants produced by the first cell(s) relative to the amount of the two or more RNA transcript splice variants produced by the second cell(s) indicates that Compound (I) modulates the aplicing of the RNA transcript.

In another aspect, provided herein is a method for determining whether Compound (I) modulates the amount of an RNA transcript (e.g., an mRNA transcript), comprising: (a) contacting a cell-free system with Compound (I), and (b) determining the amount of the RNA transcript produced by the cell-free system, wherein modulation in the amount of the RNA transcript in the presence of Compound (I) relative to the amount of the RNA transcript in the absence of Compound (I) or the presence of a negative control (e.g., a vehicle control such as PBS or DMSO) indicates that Compound (I) modulates the amount of the RNA transcript.

In another aspect, provided herein is a method for determining whether Compound (I) modulates the amount of an RNA transcript (e.g., an mRNA transcript), comprising: (a) contacting a first cell-free system with Compound (I), (b) contacting a second cell-free system with a negative control (e.g., a vehicle control, such as PBS or DMSO); and (c) determining the amount of the RNA transcript produced by the first cell-free system and the second cell-free system; and (d) comparing the amount of the RNA transcript produced by the first cell-free system to the amount of the RNA transcript expressed by the second cell-free system, wherein modulation in the amount of the RNA transcript produced by the first cell-free system relative to the amount of the RNA transcript produced by the second cell-free system indicates that Compound (I) modulates the amount of the RNA transcript. In certain aspects, the cell-free system comprises purely synthetic RNA, synthetic or recombinant (purified) enzymes, and protein factors. In other aspects, the cell-free system comprises RNA transcribed from a synthetic DNA template, synthetic or recombinant (purified) enzymes, and protein factors. In other aspects, the cell-free system comprises purely synthetic RNA and nuclear extract. In other aspects, the cell-free system comprises RNA transcribed from a synthetic DNA template and nuclear extract. In other aspects, the cell-free system comprises purely synthetic RNA and whole cell extract. In other aspects, the cell-free system comprises RNA transcribed from a synthetic DNA template and whole cell extract. In certain aspects, the cell-free system additionally comprises regulatory non-coding RNAs (e.g., microRNAs).

In another aspect, provided herein is a method for determining whether Compound (I) modulates the splicing of an RNA transcript (e.g., an mRNA transcript), comprising: (a) contacting a cell-free system with Compound (I); and (b) determining the amount of two or more RNA transcript splice variants produced by the cell-free system, wherein modulation in the amount of the two or more RNA transcript splice variants in the presence of Compound (I) relative to the amount of the two or more RNA transcript splice variants in the absence of Compound (I) or the presence of a negative control (e.g., a vehicle control such as PBS or DMSO) indicates that Compound (I) modulates the splicing of the RNA transcript.

In another aspect, provided herein is a method for determining whether Compound (I) modulates the splicing of an RNA transcript (e.g., an mRNA transcript), comprising: (a) contacting a first cell-free system with Compound (I); (b) contacting a second cell-free system with a negative control (e.g., a vehicle control, such as PBS or DMSO); and (c) determining the amount of two or more RNA transcript splice variants produced by the first cell-free system and the second cell-free system; and (d) comparing the amount of the two or more RNA transcript splice variants produced by the first cell-free system to the amount of the RNA transcript expressed by the second cell-free system, wherein modulation in the amount of the two or more RNA transcript splice variants produced by the first cell-free system relative to the amount of the two or more RNA transcript splice variants produced by the second cell-free system indicates that Compound (I) modulates the splicing of the RNA transcript. In certain aspects, the cell-free system comprises purely synthetic RNA, synthetic or recombinant (purified) enzymes, and protein factors. In other aspects, the cell-free system comprises RNA transcribed from a synthetic DNA template, synthetic or recombinant (purified) enzymes, and protein factors. In other aspects, the cell-free system comprises purely synthetic RNA and nuclear extract. In other aspects, the cell-free system comprises RNA transcribed from a synthetic DNA template and nuclear extract. In other aspects, the cell-free system comprises purely synthetic RNA and whole cell extract. In other aspects, the cell-free system comprises RNA transcribed from a synthetic DNA template and whole cell extract. In certain aspects, the cell-free system additionally comprises regulatory RNAs (e.g., microRNAs).

In another aspect, provided herein is a method for determining whether Compound (I) modulates the amount of an RNA transcript (e.g., an mRNA transcript), comprising: (a) culturing a cell(s) in the presence of Compound (I), (b) isolating the RNA transcript from the cell(s) after a certain period of time; and (c) determining the amount of the RNA transcript produced by the cell(s), wherein modulation in the amount of the RNA transcript in the presence of Compound (I) relative to the amount of the RNA transcript in the absence of Compound (I) or the presence of a negative control (e.g., a vehicle control such as PBS or DMSO) indicates that Compound (I) modulates the amount of the RNA transcript.

In another aspect, provided herein is a method for determining whether Compound (I) modulates the amount of an RNA transcript (e.g., an mRNA transcript), comprising (a) culturing a first cell(s) in the presence of Compound (I), (b) culturing a second cell(s) in the presence of a negative control (e.g., a vehicle control, such as PBS or DMSO); (c) isolating the RNA transcript produced by the first cell(s) and isolating the RNA transcript produced by the second cell(s); (d) determining the amount of the RNA transcript produced by the first cell(s) and the second cell(s); and (e) comparing the amount of the RNA transcript produced by the first cell(s) to the amount of the RNA transcript produced by the second cell(s), wherein modulation in the amount of the RNA transcript produced by the first cell(s) relative to the amount of the RNA transcript produced by the second cell(s) indicates that Compound (I) modulates the amount of the RNA transcript.

In certain aspects, the cell(s) contacted or cultured with Compound (I) is a primary cell(s) from a subject. In some aspects, the cell(s) contacted or cultured with Compound (I) is a primary cell(s) from a subject with HD disease. In specific aspects, the cell(s) contacted or cultured with Compound (I) is a primary cell(s) from a subject with HD disease associated with an aberrant amount of an RNA transcript(s) for a particular gene(s). In some specific aspects, the cell(s) contacted or cultured with Compound (I) is a primary cell(s) from a subject with HD disease associated with an aberrant amount of an isoform(s) of a particular gene(s). In some aspects, the cell(s) contacted or cultured with Compound (I) is a fibroblast, an immune cell (e.g., a T cell, B cell, natural killer cell, macrophage), a blood cell or a muscle cell. In certain aspects, the cell(s) contacted or cultured with Compound (I) is an immortalized cell. In certain aspects, the cell(s) contacted or cultured with Compound (I) is a cancer cell. In certain aspects, the cell(s) contacted or cultured with Compound (I) is from a cell line. In certain aspects, the cell contacted or cultured with Compound (I) is a cell differentiated from a stem cell, e.g., a human embryonic stem cell(s) or induced pluripotent stem cell(s) (IPSC) or a cell differentiated from induced pluripotent stem cell(s) derived from a patient with HD disease known to have aberrant RNA transcript levels for a particular gene(s). In some aspects, the cell(s) contacted or cultured with Compound (I) is a cell line derived from a subject with HD disease. In certain aspects, the cell(s) contacted or cultured with Compound (I) is from a cell line known to have aberrant RNA transcript levels for a particular gene(s). In specific aspects, the cell(s) contacted or cultured with Compound (I) is from a cell line derived from a subject with HD disease known to have aberrant RNA transcript levels for a particular gene(s.

In another aspect, provided herein is a method for determining whether Compound (I) modulates the amount of an RNA transcript (e.g., an mRNA transcript), comprising: (a) contacting a tissue sample with Compound (I); and (b) determining the amount of the RNA transcript produced by the tissue sample, wherein modulation in the amount of the RNA transcript in the presence of Compound (I) relative to the amount of the RNA transcript in the absence of Compound (I) or the presence of a negative control (e.g., a vehicle control such as PBS or DMSO) indicates that Compound (I) modulates the amount of the RNA transcript.

In another aspect, provided herein is a method for determining whether Compound (I) modulates the amount of an RNA transcript (e.g., an mRNA transcript), comprising: (a) contacting a first tissue sample with Compound (I), (b) contacting a second tissue sample with a negative control (e.g., a vehicle control, such as PBS or DMSO); and (c) determining the amount of the RNA transcript produced by the first tissue sample and the second tissue sample; and (d) comparing the amount of the RNA transcript produced by the first tissue sample to the amount of the RNA transcript produced by the second tissue sample, wherein modulation in the amount of the RNA transcript produced by the first tissue sample relative to the amount of the RNA transcript produced by the second tissue sample indicates that Compound (I) modulates the amount of the RNA transcript.

Any tissue sample containing cells may be used in the accordance with these methods. In certain aspects, the tissue sample is a blood sample, a skin sample, a muscle sample, or a tumor sample. Techniques known to one skilled in the art may be used to obtain a tissue sample from a subject.

In some aspects, a dose-response assay is performed.

In one aspect, the dose response assay comprises: (a) contacting a cell(s) with a concentration of Compound (I); (b) determining the amount of the RNA transcript produced by the cell(s), wherein modulation in the amount of the RNA transcript in the presence of Compound (I) relative to the amount of the RNA transcript in the absence of Compound (I) or the presence of a negative control (e.g., a vehicle control such as PBS or DMSO) indicates that Compound (I) modulates the amount of the RNA transcript; (c) repeating steps (a) and (b), wherein the only experimental variable changed is the concentration of Compound (I) or a form thereof; and (d) comparing the amount of the RNA transcript produced at the different concentrations of Compound (I) or a form thereof.

In another aspect, the dose response assay comprises: (a) culturing a cell(s) in the presence of Compound (I); (b) isolating the RNA transcript from the cell(s) after a certain period; (c) determining the amount of the RNA transcript produced by the cell(s), wherein modulation in the amount of the RNA transcript in the presence of Compound (I) relative to the amount of the RNA transcript in the absence of Compound (I) or the presence of a negative control (e.g., a vehicle control such as PBS or DMSO) indicates that Compound (I) modulates the amount of the RNA transcript; (d) repeating steps (a), (b), and (c), wherein the only experimental variable changed is the concentration of Compound (I) or a form thereof; and (e) comparing the amount of the RNA transcript produced at the different concentrations of Compound (I) or a form thereof. In another aspect, the dose-response assay comprises: (a) contacting each well of a microtiter plate containing cells with a different concentration of Compound (I); (b) determining the amount of an RNA transcript produced by cells in each well; and (c) assessing the change of the amount of the RNA transcript at the different concentrations of Compound (I) or form thereof.

In one aspect, the dose response assay comprises: (a) contacting a cell(s) with a concentration of Compound (I), wherein the cells are within the wells of a cell culture container (e.g., a 96-well plate) at about the same density within each well, and wherein the cells are contacted with different concentrations of Compound (I) in different wells; (b) isolating the RNA from said cells in each well; (c) determining the amount of the RNA transcript produced by the cell(s) in each well; and (d) assessing change in the amount of the RNA transcript in the presence of one or more concentrations of Compound (I) relative to the amount of the RNA transcript in the presence of a different concentration of Compound (I) or the absence of Compound (I) or the presence of a negative control (e.g., a vehicle control such as PBS or DMSO).

In certain aspects, the contacting of the cell(s) with Compound (I) occurs in cell culture. In other aspects, the contacting of the cell(s) with Compound (I) occurs in a subject, such as a non-human subject.

In certain aspects described herein, the cell(s) is contacted or cultured with Compound (I), or a tissue sample is contacted with Compound (I), or a negative control for a period of 15 minutes, 30 minutes, 45 minutes, 1 hour, 2 hours, 3 hours, 4 hours, 5 hours, 6 hours, 8 hours, 12 hours, 18 hours, 24 hours, 48 hours, 72 hours or longer. In other aspects described herein, the cell(s) is contacted or cultured with Compound (I), or a tissue sample is contacted with Compound (I), or a negative control for a period of 15 minutes to 1 hour, 1 to 2 hours, 2 to 4 hours, 6 to 12 hours, 12 to 18 hours, 12 to 24 hours, 28 to 24 hours, 24 to 48 hours, 48 to 72 hours.

In certain aspects described herein, the cell(s) is contacted or cultured with a certain concentration of Compound (I), or a tissue sample is contacted with a certain concentration of Compound (I), wherein the certain concentration is 0.0001 μM, 0.0003 μM, 0.001 μM, 0.003 μM, 0.01 μM, 0.05 μM, 1 μM, 2 μM, 5 μM, 10 μM, 15 μM, 20 μM, 25 μM, 50 μM, 75 μM, 100 μM, or 150 μM. In other aspects described herein, the cell(s) is contacted or cultured with a certain concentration of Compound (I), or a tissue sample is contacted with a certain concentration of Compound (I), wherein the certain concentration is 0.0001 μM, 0.0003 μM, 0.0005 μM, 0.001 μM, 0.003 μM, 0.005 μM, 0.01 μM, 0.03 μM, 0.05 μM, 0.1 μM, 0.3 μM, 0.5 μM or 1 μM. In other aspects described herein, the cell(s) is contacted or cultured with a certain concentration of Compound (I), or a tissue sample is contacted with a certain concentration of Compound (I), wherein the certain concentration is 175 μM, 200 μM, 250 μM, 275 μM, 300 μM, 350 μM, 400 μM, 450 μM, 500 μM, 550 μM 600 μM, 650 μM, 700 μM, 750 μM, 800 μM, 850 μM, 900 μM, 950 μM or 1 mM. In some aspects described herein, the cell(s) is contacted or cultured with a certain concentration of Compound (I), or a tissue sample is contacted with a certain concentration of Compound (I), wherein the certain concentration is 5 nM, 10 nM, 20 nM, 24 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, or 950 nM. In certain aspects described herein, the cell(s) is contacted or cultured with a certain concentration of Compound (I), or a tissue sample is contacted with a certain concentration of Compound (I), wherein the certain concentration is between 0.0001 μM to 0.001 μM, 0.0001 μM to 0.01 μM, 0.0003 μM to 0.001 μM, 0.0003 μM to 0.01 μM, 0.001 μM to 0.01 μM, 0.003 μM to 0.01 μM, 0.01 μM to 0.1 μM, 0.1 μM to 1 μM, 1 μM to 50 μM, 50 μM to 100 μM, 100 μM to 500 μM, 500 μM to 1 nM, 1 nM to 10 nM, 10 nM to 50 nM, 50 nM to 100 nM, 100 nM to 500 nM, 500 nM to 1000 nM.

In another aspect, provided herein is a method for determining whether Compound (I) modulates the amount of an RNA transcript (e.g., an mRNA transcript), comprising: (a) administering Compound (I) to a subject (in certain aspects, a non-human animal); and (b) determining the amount of the RNA transcript in a sample obtained from the subject, wherein modulation in the amount of the RNA transcript measured in the sample from the subject administered Compound (I) or form thereof relative to the amount of the RNA transcript in a sample from the subject prior to administration of Compound (I) or form thereof or a sample from a different subject from the same species not administered Compound (I) or form thereof indicates that Compound (I) modulates the amount of the RNA transcript.

In another aspect, provided herein is a method for determining whether Compound (I) modulates the amount of an RNA transcript (e.g., an mRNA transcript), comprising: (a) administering Compound (I) to a first subject (in certain aspects, a non-human animal); (b) administering an inactive control (e.g., a pharmaceutical carrier) to a second subject (in certain aspects, a non-human animal) of the same species as the first subject; and (c) determining the amount of the RNA transcript in a first tissue sample from the first subject and the amount of the RNA transcript in the second tissue sample from the second subject; and (d) comparing the amount of the RNA transcript in the first tissue sample to the amount of the RNA transcript in the second tissue sample, wherein modulation in the amount of the RNA transcript in the first tissue sample relative to the amount of the RNA transcript in the second tissue sample indicates that Compound (I) modulates the amount of the RNA transcript.

In certain aspects, Compound (I) or form thereof is administered to a subject at a dose of about 0.001 mg/kg/day to about 500 mg/kg/day. In some aspects, a single dose of Compound (I) is administered to a subject in accordance with the methods described herein. In other aspects, 2, 3, 4, 5 or more doses of Compound (I) is administered to a subject in accordance with the methods described herein. In specific aspects, Compound (I) is administered in a subject in a pharmaceutically acceptable carrier, excipient or diluent.

In another aspect, provided herein is a method for determining whether Compound (I) modulates the splicing of an RNA transcript (e.g., an mRNA transcript), comprising: (a) administering Compound (I) to a subject (in certain aspects, a non-human animal); and (b) determining the amount of two or more RNA transcript splice variants in a sample obtained from the subject, wherein modulation in the amount of the two or more RNA transcript splice variants measured in the sample from the subject administered Compound (I) or form thereof relative to the amount of the two or more RNA transcript splice variants in a sample from the subject prior to administration of Compound (I) or form thereof or a sample from a different subject from the same species not administered Compound (I) or form thereof indicates that Compound (I) modulates the splicing of the RNA transcript.

In another aspects, provided herein is a method for determining whether Compound (I) modulates the splicing of an RNA transcript (e.g., an mRNA transcript), comprising: (a) administering Compound (I) to a first subject (in certain aspects, a non-human animal); (b) administering a negative control (e.g., a pharmaceutical carrier) to a second subject (in certain aspects, a non-human animal) of the same species as the first subject; (c) determining the amount of two or more RNA transcript splice variants in a first tissue sample from the first subject and the amount of two or more RNA transcript splice variants in the second tissue sample from the second subject; and (d) comparing the amount of the two or more RNA transcript splice variants in the first tissue sample to the amount of the two or more RNA transcript splice variants in the second tissue sample, wherein modulation in the amount of the two or more RNA transcript splice variants in the first tissue sample relative to the amount of the two or more RNA transcript splice variants in the second tissue sample indicates that Compound (I) modulates the splicing of the RNA transcript.

In certain aspects, Compound (I) or form thereof is administered to a subject at a dose of about 0.001 mg/kg/day to about 500 mg/kg/day. In some aspects, a single dose of Compound (I) is administered to a subject in accordance with the methods described herein. In other aspects, 2, 3, 4, 5 or more doses of Compound (I) is administered to a subject in accordance with the methods described herein. In specific aspects, Compound (I) is administered in a subject in a pharmaceutically acceptable carrier, excipient or diluent.

In some aspects, Compound (I) that is contacted or cultured with a cell(s) or a tissue sample or administered to a subject is a Compound (I) described herein.

Techniques known to one skilled in the art may be used to determine the amount of an RNA transcript(s). In some aspects, the amount of one, two, three or more RNA transcripts is measured using deep sequencing, such as ILLUMINA® RNASeq, ILLUMINA® next generation sequencing (NGS), ION TORRENT™ RNA next generation sequencing, 454™ pyrosequencing, or Sequencing by Oligo Ligation Detection (SOLID™), Single Molecule, Real-Time (SMRT) sequencing, Nanopore sequencing. In other aspects, the amount of multiple RNA transcripts is measured using an exon array, such as the GENECHIP® human exon array. In certain aspects, the amount of one, two, three or more RNA transcripts is determined by RT-PCR. In other aspects, the amount of one, two, three or more RNA transcripts is measured by RT-qPCR or digital color-coded barcode technology. Techniques for conducting these assays are known to one skilled in the art.

In some aspects, analysis is performed on data derived from the assay to measure the magnitude of splicing to determine the amount of exons spliced into an mRNA transcript that is produced in the presence of Compound (I) relative to the amount in the absence of Compound (I) or presence of a negative control. In a preferred aspect, the method utilized is calculation of change in Percent Spliced In (ΔPSI). The method utilizes read data from RNAseq (or any other method that can distinguish mRNA splice isoforms) to calculate the ratio (percentage) between reads that either demonstrate inclusion (junctions between the upstream exon and the exon of interest) or exclusion (junction between the upstream and downstream exons, excluding the exon of interest), to demonstrate whether the presence of Compound (I) affects the amount of exon inclusion relative to the amount of inclusion in the absence of Compound (I) or the presence of a negative control. The ΔPSI value is derived from the formula:

ΔPSI(%)=C−U×100

Where “U” represents the value for probability of iExon inclusion (a+b)/2/[(a+b)/2+c] in the absence of Compound (I); and, where “C” represents the value for probability of iExon inclusion (a+b)/2/[(a+b)/2+c] in the presence of. The values for “a” and “b” represent the number of reads supporting inclusion of an iExon in an RNA transcript. In other words, the “a” value is derived from the amount of reads for a first intronic nucleotide sequence comprising, in 5′ to 3′ order: a first exon 5′ splice site operably linked and upstream from a first intronic nucleotide sequence comprising a first branch point further operably linked and upstream from a first intronic 3′ splice site (upstream of the nascent iExon). The “b” value is derived from the amount of reads for a second intronic nucleotide sequence comprising, in 5′ to 3′ order: pseudoexon that when present in an intron can be recognized as a 5′ splice site by the U1 snRNP and/or other components of the pre-mRNA splicing machinery in the presence of Compound (I), wherein gene expression is modulated by inducing alternative splicing of pseudoexons (i.e. iExons) in the transcribed RNA operably linked and upstream from a second intronic nucleotide sequence comprising a second branch point further operably linked and upstream from a second intronic 3′ splice site of a second exon. The value for “C” represents the number of reads supporting exclusion of an iExon. Accordingly, when a Compound (I) enables the splicing machinery to recognize a nascent iExon, the value for “C” in the presence of Compound (I) will differ from the value for “U” in the absence of Compound (I). The statistically significant value for the likelihood of iExon inclusion may be obtained according to statistical analysis methods or other probability analysis methods known to those of ordinary skill in the art.

In some aspects, a statistical analysis or other probability analysis is performed on data from the assay utilized to measure an RNA transcript. In certain aspects, for example, a Fisher's Exact Test statistical analysis is performed by comparing the total number of reads for the inclusion and exclusion of an iExon (or region) based on data from one or more assays used to measure whether the amount of an RNA transcript is modulated in the presence of Compound (I) relative to the amount in the absence of Compound (I) or presence of a negative control. In specific aspects, the statistical analysis results in a confidence value for those modulated RNA transcripts of 10%, 5%, 4%, 3%, 2%, 1%, 0.5%, 0.1%, 0.01%, 0.001% or 0.0001%. In some specific aspects, the confidence value is a p value for those modulated RNA transcripts of 10%, 5%, 4%, 3%, 2%, 1%, 0.5%, 0.1%, 0.01%, 0.001% or 0.0001%. In certain specific aspects, an exact test, student t-test or p value for those modulated RNA transcripts is 10%, 5%, 4%, 3%, 2%, 1%, 0.5% or 0.1% and 10%, 5%, 4%, 3%, 2%, 1%, 0.5%, 0.1%, 0.01%, 0.001% or 0.0001%, respectively.

In certain aspects, a further analysis is performed to determine how Compound (I) is changing the amount of an RNA transcript(s). In specific aspects, a further analysis is performed to determine if modulation in the amount of an RNA transcript(s) in the presence of Compound (I) relative the amount of the RNA transcript(s) in the absence of Compound (I) or a form thereof, or the presence of a negative control is due to changes in transcription, splicing, and/or stability of the RNA transcript(s). Techniques known to one skilled in the art may be used to determine whether Compound (I) changes, e.g., the transcription, splicing and/or stability of an RNA transcript(s).

In certain aspects, the stability of one or more RNA transcripts is determined by serial analysis of gene expression (SAGE), differential display analysis (DD), RNA arbitrary primer (RAP)-PCR, restriction endonuclease-lytic analysis of differentially expressed sequences (READS), amplified restriction fragment-length polymorphism (ALFP), total gene expression analysis (TOGA), RT-PCR, RT-RPA (recombinase polymerase amplification), RT-qPCR, RNA-Seq, digital color-coded barcode technology, high-density cDNA filter hybridization analysis (HDFCA), suppression subtractive hybridization (SSH), differential screening (DS), cDNA arrays, oligonucleotide chips, or tissue microarrays. In other aspects, the stability of one or more RNA transcripts is determined by Northern blot, RNase protection, or slot blot.

In some aspects, the transcription in a cell(s) or tissue sample is inhibited before (e.g., 5 minutes, 10 minutes, 30 minutes, 1 hour, 2 hours, 4 hours, 6 hours, 8 hours, 12 hours, 18 hours, 24 hours, 36 hours, 48 hours, or 72 hours before) or after (e.g., 5 minutes, 10 minutes, 30 minutes, 1 hour, 2 hours, 4 hours, 6 hours, 8 hours, 12 hours, 18 hours, 24 hours, 36 hours, 48 hours, or 72 hours) the cell or the tissue sample is contacted or cultured with an inhibitor of transcription, such as α-amanitin, DRB, flavopiridol, triptolide, or actinomycin-D. In other aspects, the transcription in a cell(s) or tissue sample is inhibited with an inhibitor of transcription, such as α-amanitin, DRB, flavopiridol, triptolide, or actinomycin-D, while the cell(s) or tissue sample is contacted or cultured with Compound (I).

In certain aspects, the level of transcription of one or more RNA transcripts is determined by nuclear run-on assay or an in vitro transcription initiation and elongation assay. In some aspects, the detection of transcription is based on measuring radioactivity or fluorescence. In some aspects, a PCR-based amplification step is used.

In specific aspects, the amount of alternatively spliced forms of the RNA transcripts of a particular gene are measured to see if there is modulation in the amount of one, two or more alternatively spliced forms of the RNA transcripts of the gene. In some aspects, the amount of an isoform(s) encoded by a particular gene is measured to see if there is modulation in the amount of the isoform(s). In certain aspects, the levels of spliced forms of RNA are quantified by RT-PCR, RT-qPCR, RNA-Seq, digital color-coded barcode technology, or Northern blot. In other aspects, sequence-specific techniques may be used to detect the levels of an individual spliceoform. In certain aspects, splicing is measured in vitro using nuclear extracts. In some aspects, detection is based on measuring radioactivity or fluorescence. Techniques known to one skilled in the art may be used to measure modulation in the amount of alternatively spliced forms of an RNA transcript of a gene and modulation in the amount of an isoform encoded by a gene.

### V. Characterization of a Small Molecule-Inducible Intronic Sequence

This disclosure reports on the discovery of pre-mRNA sequences required for alternative splicing of an intronic sequence that is contingent on the presence of a small molecule, e.g., Compound I, as described herein. Thus, in the presence of Compound I, the intronic sequence is converted into an “intron-derived exon” that can be spliced into the mature spliced mRNA, an event that can lead to a frameshift in the mRNA's open reading frame and the appearance of premature stop codons. The ensuing premature termination of translation results in nonsense mediated decay of the mRNA and a concomitent reduction in the amount of protein encoded by the mRNA. Conversely, in the absence of Compound I, the intronic sequence remains dormant and is spliced out of the pre-mRNA without causing a change to the mRNA's reading frame.

### Identification of GA-psiExons in Human Genome

The human genome was searched for potential compound-responsive GA-psiExons having at least one of the following criteria: (1) length between 6-200 nt, 3′ splice site (ss) MAXENT score >2.3 and a 5′ splice site (ss) MAXENT score >−2.1; (2) within intron region of another Refseq annotated gene; (3) 5′ splice site (ss) has AGAgtaag sequence, in which AGA are at positions −3 to −1 and gtaag are at positions +1 to +5.

Methods of determining a 5′ or 3′ splice site's Maxent score are described in Yeo, G. & Burge, C. B. (2004) Journal of computational biology 11, 377-394, the content of which is incorporated by reference herein in its entirety.

In one aspect, a putative psiExon can be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 or 200 nucleotides in length.

In one aspect, a 3′ splice site (ss) of a putative psiExon can have a MAXENT score greater than about 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5, 6, 7, 8 or 9 or more. In one aspect, a 3′ splice site (ss) of a putative psiExon can have a MAXENT score of about 2.3 to about 9.

In one aspect, a 3′ splice site (ss) of a putative psiExon can have a MAXENT score of about 2.3 to about 3.

In one aspect, a 5′ splice site (ss) of a putative psiExon can have a MAXENT score greater than about −2.1, −2.0, −1.9, −1.8, −1.7, −1.6, −1.5, −1.4, −1.3, −1.2, −1.1, −1.0, −0.9, −0.8, −0.7, −0.6, −0.5, −0.4, −0.3, −0.2, −0.1, 0, 0.1, 0.2, 0.3, 0.4, 0.5, 0.6, 0.7, 0.8, 0.9, 1, 1.1., 1.2, 1.3, 1.4, 1.5, 1.6, 1.7, 1.8, 1.9, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5, 6, 7, 8 or 9 or more.

In one aspect, a 5′ splice site (ss) of a putative psiExon can have a MAXENT score of about −2.1 to about 9.

In one aspect, the 5′ splice site (ss) of a putative psiExon can have a MAXENT score of about −2.1 to about 3.

In one aspect, the 5′ splice site (ss) is a noncanonical 5′ splice site having the sequence of NNGAgtrag, in which GA are at positions −2 to −1 and guragu are at positions +1 to +5.

In one aspect, a 5′ splice site (ss) is a noncanonical 5′ splice site having the RNA sequence of ANGAgurngn (SEQ ID NO: 110), CNGAgurngn (SEQ ID NO: 111), GNGAgurngn (SEQ ID NO: 112), UNGAgurngn (SEQ ID NO: 113), NAGAgurngn (SEQ ID NO: 114), NCGAgurngn (SEQ ID NO: 115), NGGAgurngn (SEQ ID NO: 116), NUGAgurngn (SEQ ID NO: 117), AAGAgurngn (SEQ ID NO: 118), ACGAgurngn (SEQ ID NO: 119), AGGAgumgn (SEQ ID NO: 120), AUGAgurngn (SEQ ID NO: 121), CAGAgurngn (SEQ ID NO: 122), CCGAgurngn (SEQ ID NO: 123), CGGAgumgn (SEQ ID NO: 124), CUGAgurngn (SEQ ID NO: 125), GAGAgurngn (SEQ ID NO: 126), GCGAgurngn (SEQ ID NO: 127), GGGAgumgn (SEQ ID NO: 128), GUGAgurngn (SEQ ID NO: 129), UAGAgurngn (SEQ ID NO: 130) or UCGAgurngn (SEQ ID NO: 131), in which GA are at positions −2 to −1 and guragu are at positions +1 to +5, and wherein r is adenine or guanine, and N is any nucleotide.

In one aspect, a 5′ splice site (ss) is a noncanonical 5′ splice site having the RNA sequence of ANGAguragu (SEQ ID NO: 132), CNGAguragu (SEQ ID NO: 133), GNGAguragu (SEQ ID NO: 134), UNGAguragu (SEQ ID NO: 135), NAGAguragu (SEQ ID NO: 136), NCGAguragu (SEQ ID NO: 137), NGGAguragu (SEQ ID NO: 138), NUGAguragu (SEQ ID NO: 139), AAGAguragu (SEQ ID NO: 140), ACGAguragu (SEQ ID NO: 141), AGGAguragu (SEQ ID NO: 142), AUGAguragu (SEQ ID NO: 143), CAGAguragu (SEQ ID NO: 144), CCGAguragu (SEQ ID NO: 145), CGGAguragu (SEQ ID NO: 146), CUGAguragu (SEQ ID NO: 147), GAGAguragu (SEQ ID NO: 148), GCGAguragu (SEQ ID NO: 149), GGGAguragu (SEQ ID NO: 150), GUGAguragu (SEQ ID NO: 151), UAGAguragu (SEQ ID NO: 152), UCGAguragu (SEQ ID NO: 153), UGGAguragu (SEQ ID NO: 154) and UUGAguragu (SEQ ID NO: 155), in which GA are at positions −2 to −1 and guragu are at positions +1 to +5, and wherein r is adenine or guanine, and N is any nucleotide.

Using these criteria, putative psiExons were discovered in introns 1, 8, 40 and 49 of the HTT gene.

Example IV of this disclosure describes the generation of minigene constructs to analyze those intronic sequences in HTT introns 1, 8, 40 and 49 to identify those sequences that are essential for inducing the alternative splicing of an “intron-derived exon” in the presence of Compound I.

The analysis of Example IV shows that only the psiExon in HTT intron 49 is inducible by Compound I. In one aspect, the putative psiExons within HTT introns 1, 8 and 40 are not inducible by Compound I.

Based on these experiments, the sequence elements required for Compound (I) induced splicing to occur are in 5′ to 3′ order: a 5′ exonic splice site, a first intronic branch point, an intronic 3′ splice site, a pseudo-Exonic Splice Enhancer (pseudo-ESE), a noncanonical 5′ intronic splice site, a second intronic branch point, and a 3′ exonic splice site.

As used herein, the term “small molecule-inducible intronic sequence” refers to a sequence having an exon boundary in the presence of Compound (I) defined by the intronic 3′ splice site, the pseudo-Exonic Splice Enhancer (pseudo-ESE) and the 5′ intronic splice site, wherein the 5′ intronic splice site is noncanonical.

Without being bound by any theory, in the presence of Compound (I), the binding affinity of U1 snRNP for the noncanonical 5′ splice site is not increased sufficiently to facilitate the alternative splicing of the pseudoexon. Only in conjunction with the pseudo-ESE proximal to the noncanonical 5′ intronic splice site will the spliceosome then induce a first catalytic step at the noncanonical 5′ intronic splice and an intronic 3′ splice site together with U2 snRNP and associated splicing factors to create a pseudoexon boundary. Excision of a downstream intronic portion by splicing of the noncanonical 5′ splice site and 3′ intronic splice site defines an intron-derived exon (also called herein as pseudoexon or psiExon or small molecule-inducible intronic sequence) and results in the insertion of the intron-derived exon into the mature mRNA.

In one aspect described herein, the pre-mRNA sequence comprises in 5′ to 3′ order: a nucleotide sequence encoding a 5′ exonic splice site, a nucleotide sequence encoding a intronic branch point, a nucleotide sequence encoding an intronic 3′ splice site, a nucleotide sequence encoding a pseudo-ESE (Exonic Splice Enhancer), a nucleotide sequence encoding a 5′ exonic splice site, a nucleotide sequence encoding a second intronic branch point, and a nucleotide sequence encoding a 3′ intronic splice site.

In another aspect described herein, the 5′ exonic splice site

In another aspect described herein, 3′ intronic splice site.

In another aspect described herein, the presence of Compound (I) preferentially increases the binding affinity of U1 snRNP to create an exon boundary defined by a pseudo-ESE, wherein the presence of Compound (I) causes the ISE to act as an ESE, resulting in alternative splicing at the 3′ exonic splice site, wherein an upstream and downstream intronic portion defined by the exon boundary is alternatively spliced, thus inducing retention of an exon.

In another aspect described herein, the presence of Compound (I) preferentially increases the binding affinity of U1 snRNP such that the U1 snRNP remains associated with the 5′ splice site within the exon boundary, wherein the presence of Compound (I) causes the ISE to act as an ESE, resulting in alternative splicing at the 3′ exonic splice site, wherein only an upstream intronic portion defined by the exon boundary is alternatively spliced, thus inducing retention of the downstream remainder of the intron to produce an extended exon.

For example, in SMA, where SMN2 exon 7 is predominantly excluded, the presence of one or more ESEs (Exonic Splicing Enhancers) in proximity to a noncanonical 5′ splice site (within 30-40 nts) (the canonical exon is normally 115-200 nts) in the presence of a small molecule splicing modifier compound induces inclusion of the excluded exon.

In one aspect, splicing of the intronic sequence induced by Compound (I) generates an intron-derived exon that is inserted into the mature mRNA.

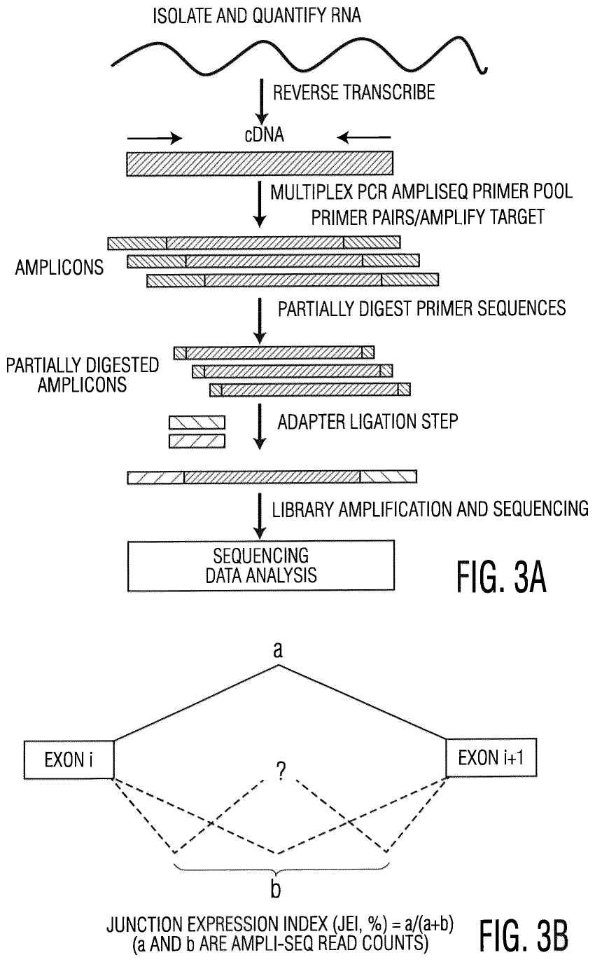

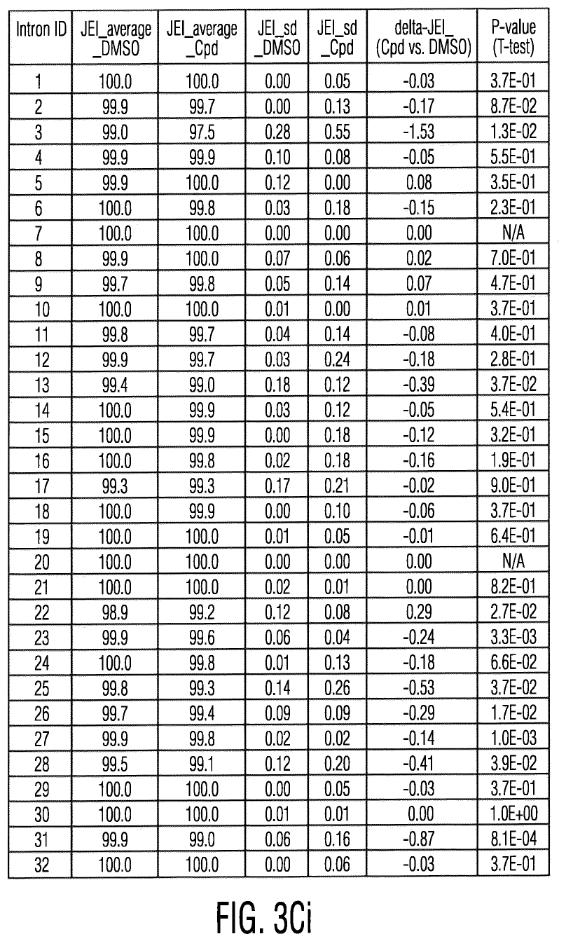

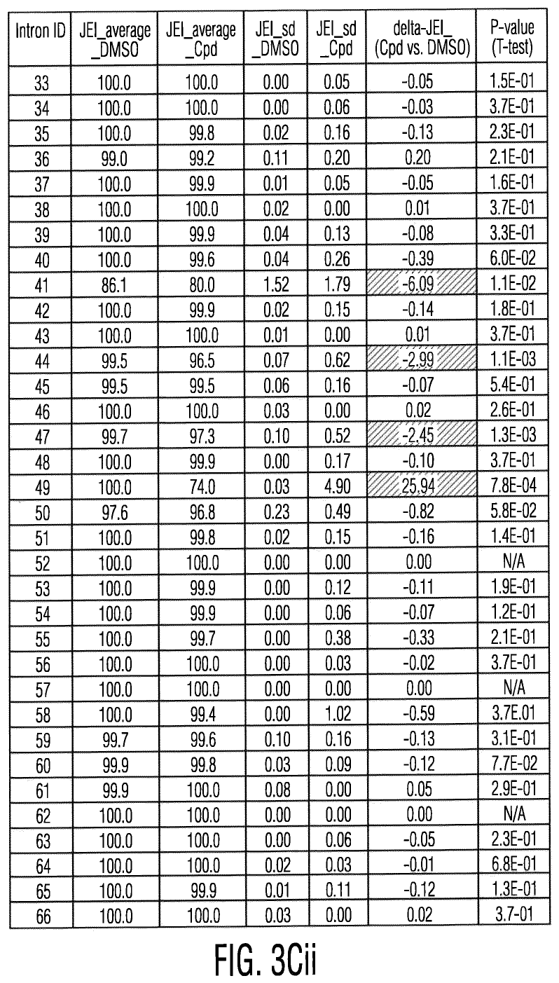

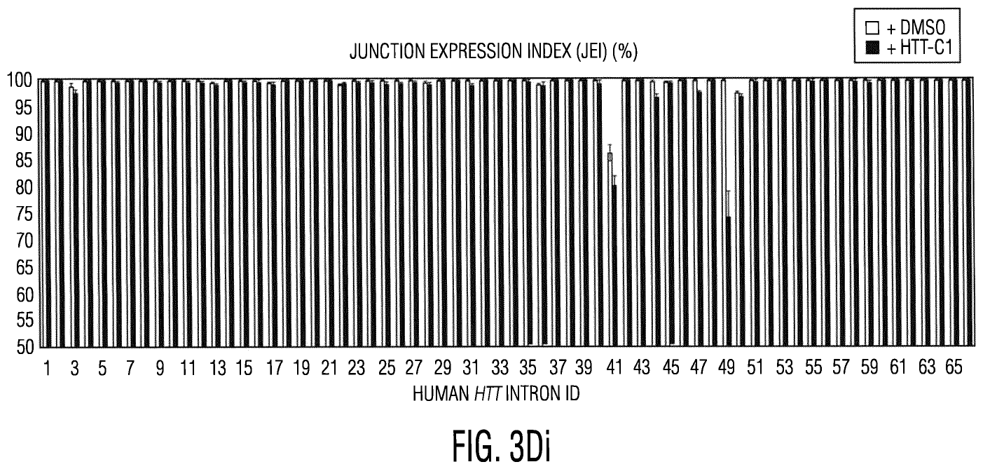

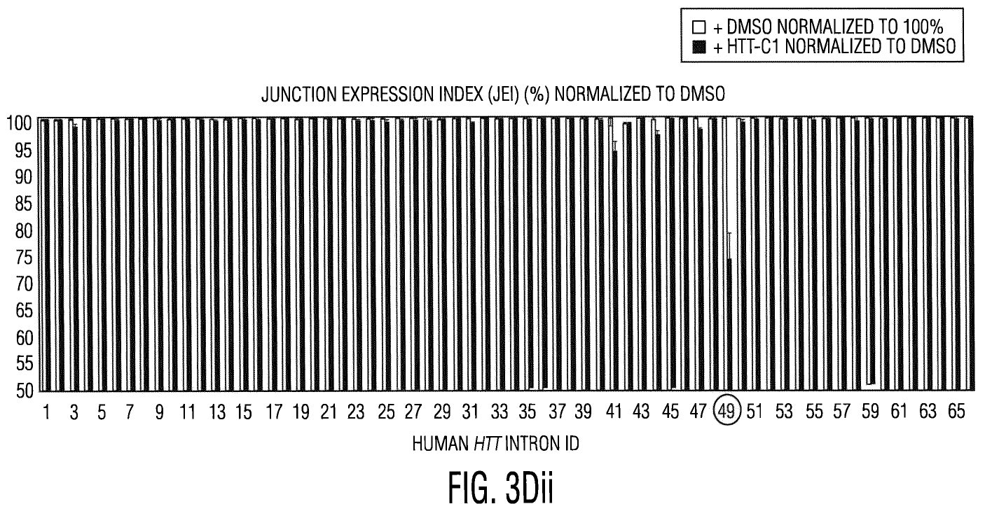

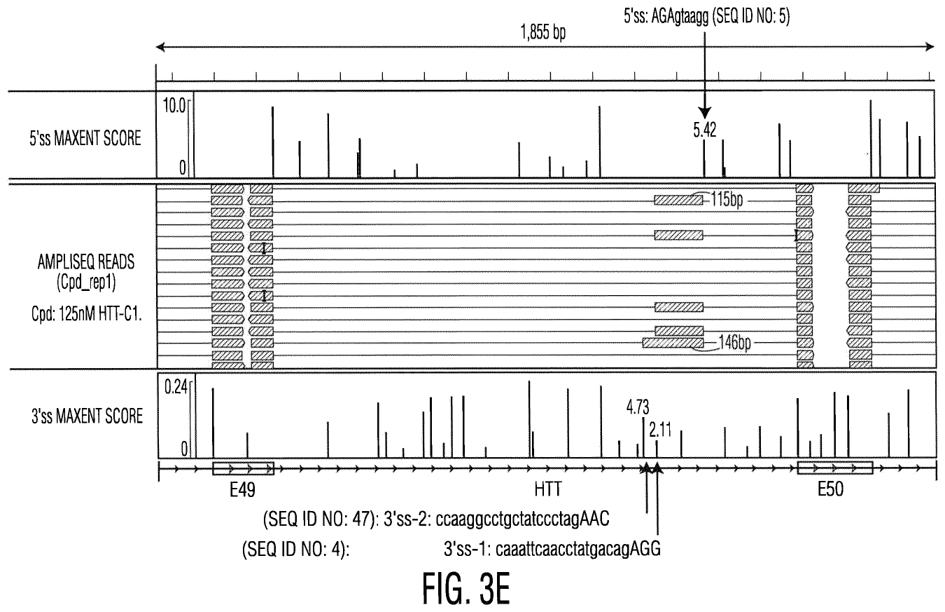

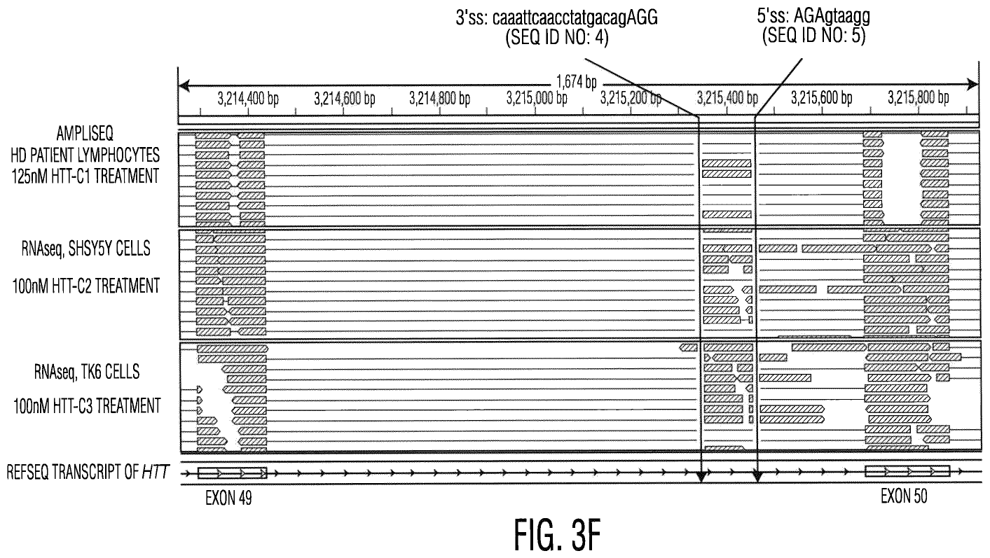

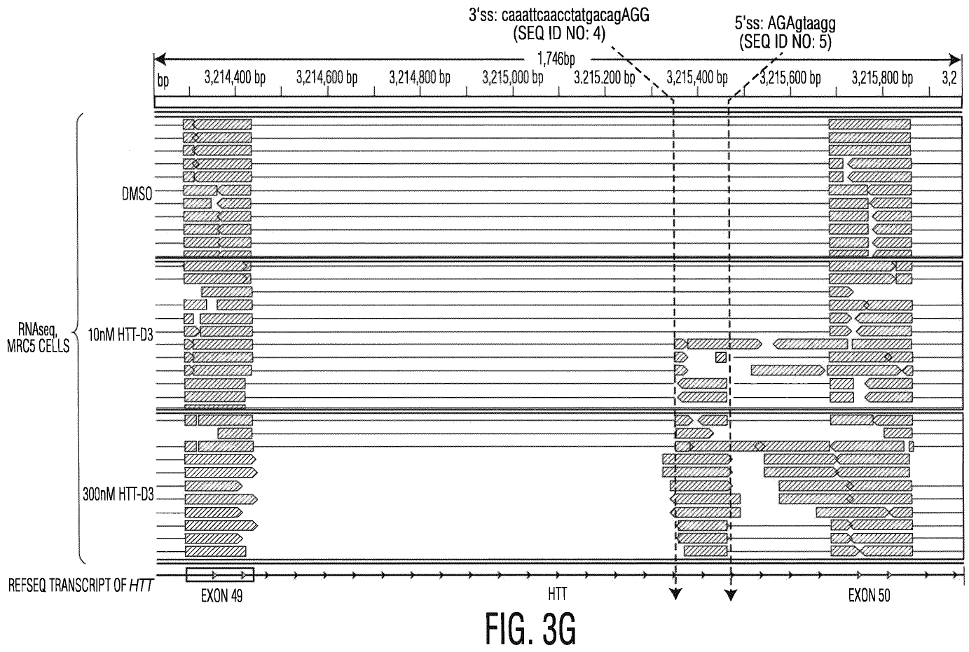

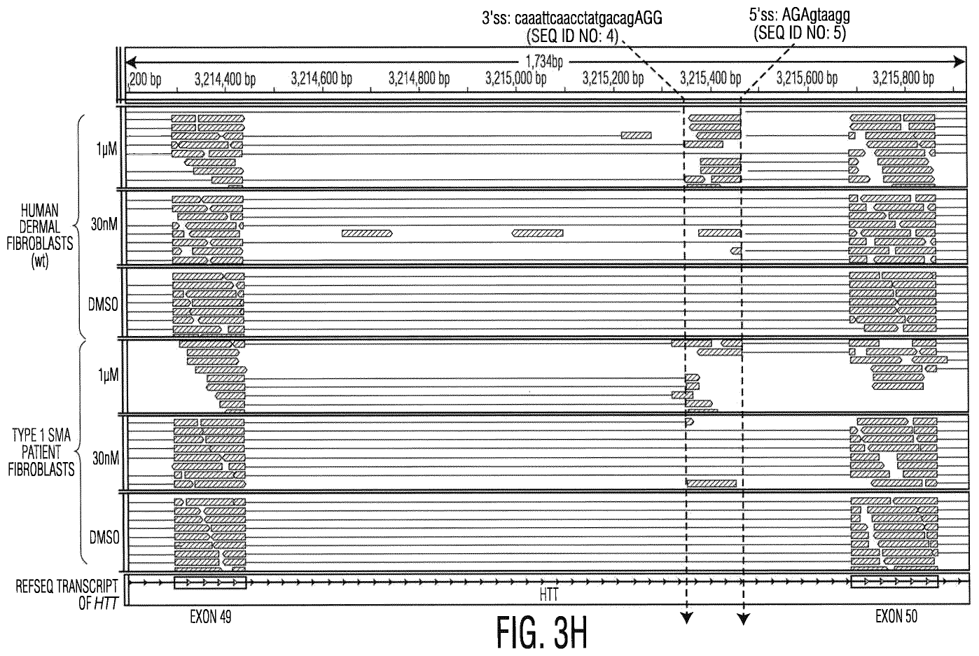

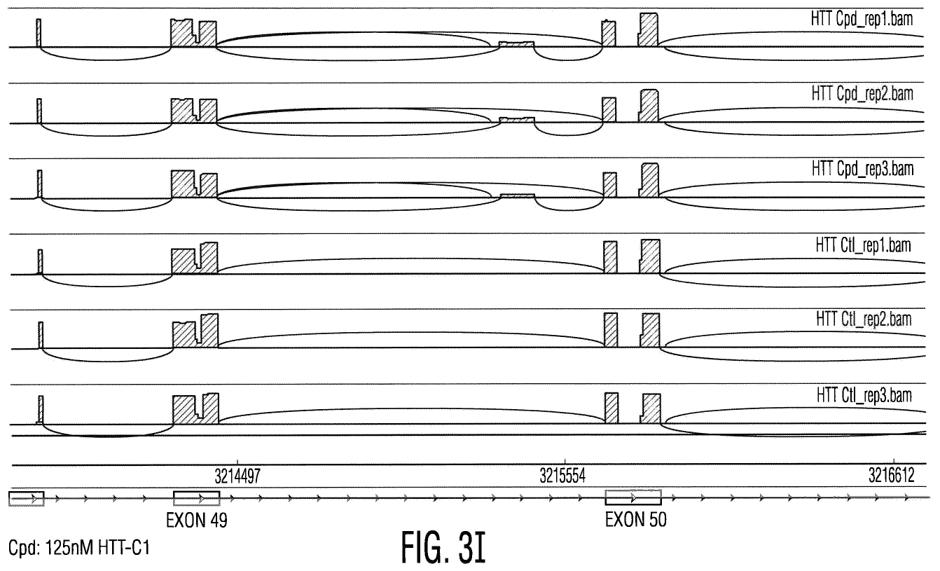

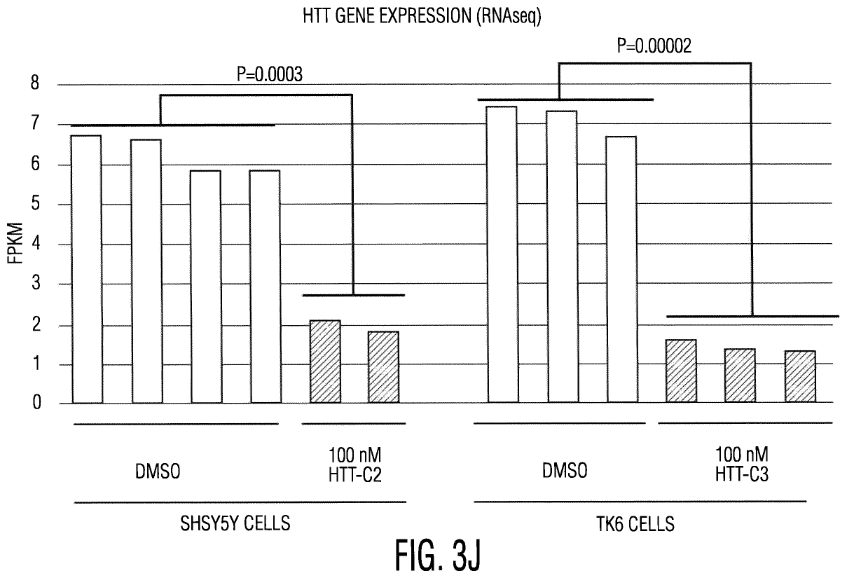

For example, Compound (I) induced splicing of HTT pre-mRNA results in the recognition of two 3′ splice sites which produced several intron-derived exons of 115 nt (SEQ ID NO: 46), and 146 nt (SEQ ID NO: 49) (see, for example, FIGS. 4Ai-iv). In another aspect, Compound (I) induced splicing of HTT pre-mRNA can result in the production other intron-derived exons including, but not limited to, intron-derived exons of 336 nt and 367 nt in length (FIGS. 3E-3G).

For example, in SMA, where SMN2 exon 7 is predominantly excluded, the presence of one or more ESEs (Exonic Splicing Enhancers) in proximity to a noncanonical 5′ splice site in the presence of Compound (I) induces inclusion of the excluded exon.

As described herein, the term “pseudo-ESE” refers to a sequence which enhances splicing, in the presence of Compound (I) and a proximal 5′ splice site and a upstream 3′ splice site, to produce a Compound (I) inducible intronic sequence or pseudoexon.

In one aspect, the 5′ terminal nucleotide of the pseudo-ESE can be about 1-200 nucleotides from the GU sequence within the 5′ splice site.

In one aspect, the 5′terminal nucleotide of the pseudo-ESE can be about 1-150 nucleotides from the GU sequence within the 5′ splice site.

In one aspect, the 5′ terminal nucleotide of the pseudo-ESE can be about 1-100 nucleotides from the GU sequence within the 5′ splice site.

In one aspect, the 5′ terminal nucleotide of the pseudo-ESE can be about 1-50 nucleotides from the GU sequence within the 5′ splice site.

In one aspect, the 5′ terminal nucleotide of the pseudo-ESE and the GU sequence within the 5′ splice site can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 or 200, nucleotides apart.

Thus, splicing of an intronic sequence can be induced by Compound (I) only if the noncanonical 5′ splice site is proximal to a pseudo-ESE.

In the absence of Compound I, the pseudo-ESE and 5′ splice site can not bind to wild type U1 to induce splicing.

In the absence of a pseudo-ESE, there is no splicing at the noncanonical 5′ splice site even in the presence of Compound I.

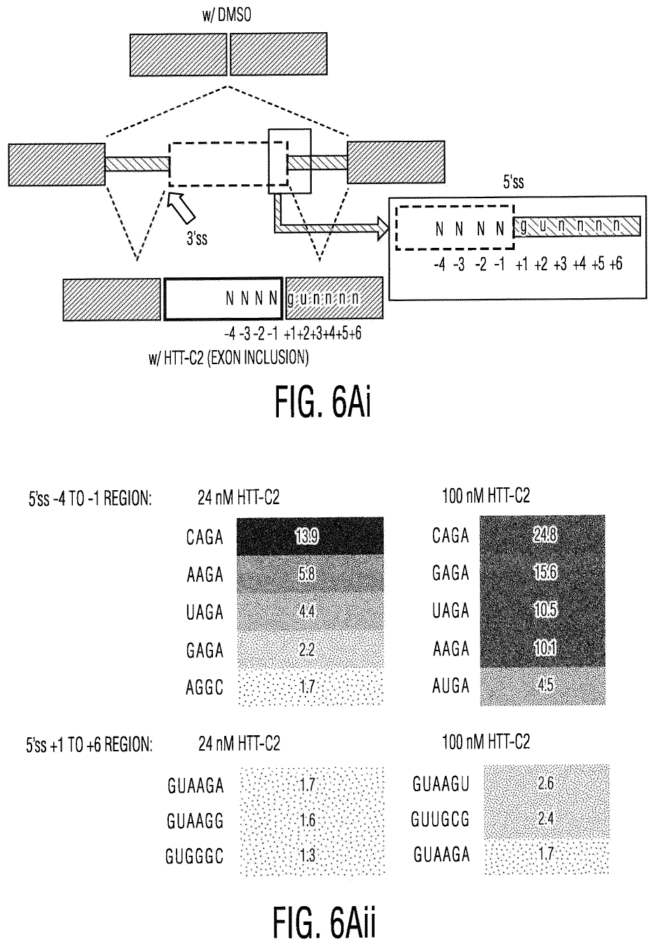

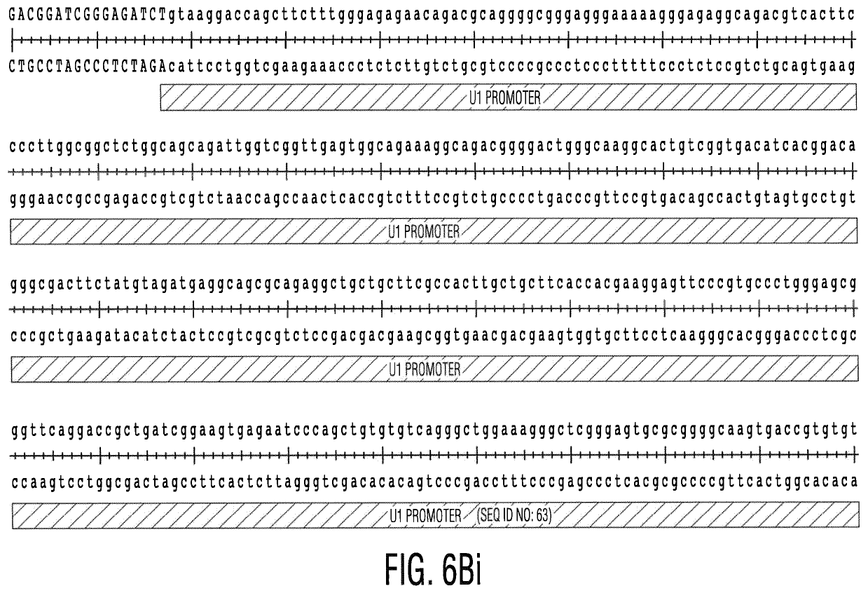

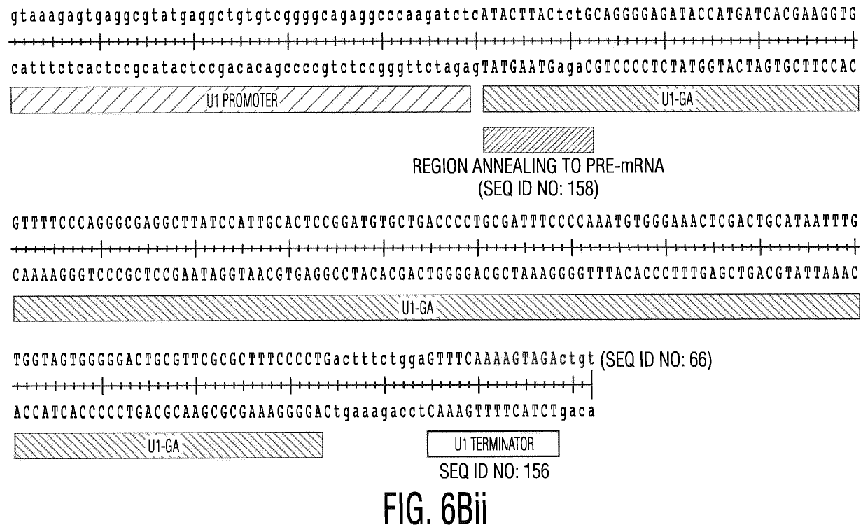

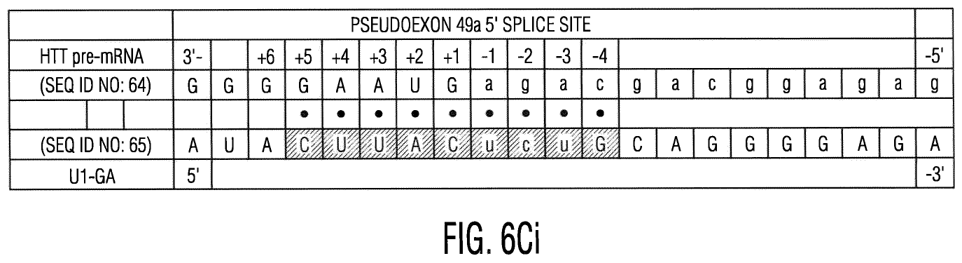

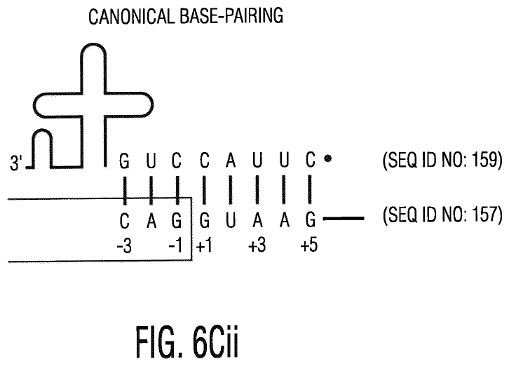

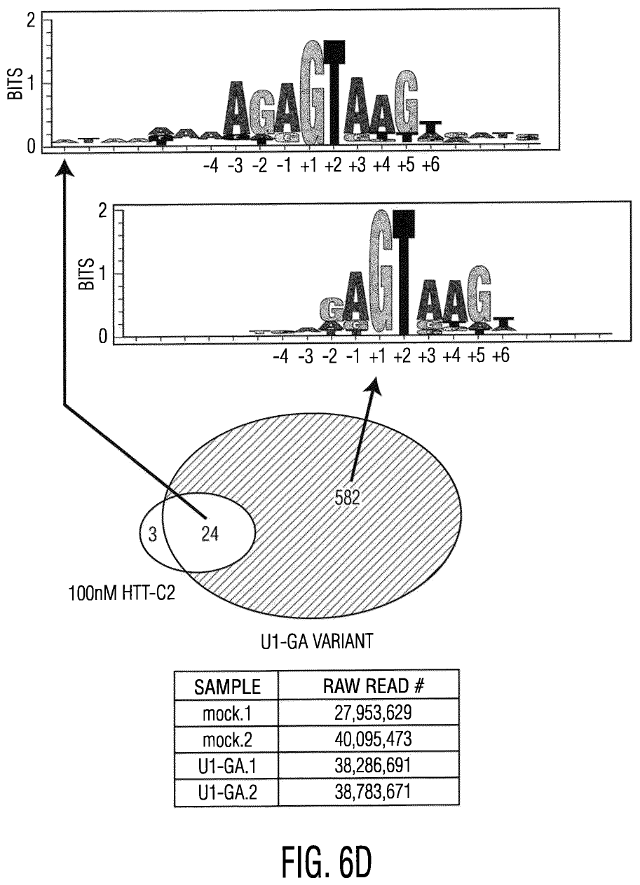

As described herein, the term “U1-variant” refers to a U1 snRNA in which the sequence at the 5′ end that can anneal to the noncanonical 5′ splice site is mutated (i.e., nucleotides between positions +5 and −4 of SEQ ID NO: 64; see FIGS. 6Ci-ii). In one aspect, a variant U1 snRNA is mutated to facilitate the annealing of the 5′ end of U1 snRNA to the noncanonical 5′ splice site.

In one aspect, the terms “canonical splice site” or “consensus splice site” can be used interchangeably and refer to splice sites that are conserved across species. Consensus sequences for the 5′ splice site and the 3′ splice site used in eukaryotic RNA splicing are well known in the art (see, e.g., Gesteland et al. (eds.), The RNA World, 3rd Edition, Cold Spring Harbor Laboratory Press, Cold Spring Harbor, New York, (2006), Watson et al, supra, and Mount, Nucleic Acid Res., 10: 459-472 (1982), the contents of which are incorporated by reference herein in their entirety). These consensus sequences include nearly invariant dinucleotides at each end of the intron: GT at the 5′ end of the intron, and AG at the 3′ end of an intron.

In one aspect, a “canonical 5′ splice site” or splice donor site consensus sequence can be (for DNA) CAG/GTRAG (where A is adenosine, T is thymine, G is guanine, C is cytosine, R is a purine and “/” is the splice site).

In one aspect, a “noncanonical 5′ splice site” can be (for DNA) the sequence NNNN/GTNNN where N can be any one of adenosine, thymine, guanine, cytosine and “/” is the splice site with the exception of a canonical 5′ splice site having the sequence of CAG/GTRAG (where A is adenosine, T is thymine, G is guanine, C is cytosine, R is a purine and “/” is the splice site). In some aspects, a noncanonical 5′ splice site is dormant in the absence of both a proximal pseudo-ESE and Compound (I) as described herein.

In one aspect, the splice acceptor site consists of three separate sequence elements: the branch point or branch site, a polypyrimidine tract and the 3′ splice site consensus sequence. The branch point consensus sequence in eukaryotes is YNYTRAC (where Y is a pyrimidine, N is any nucleotide, and R is a purine; the underlined A is the site of branch formation. The 3′ splice site consensus sequence is YAG (where Y is a pyrimidine) (see, e.g., Griffiths et al, eds., Modern Genetic Analysis, 2nd edition, W.H. Freeman and Company, New York (2002), the contents of which are incorporated by reference herein in their entirety).

### VI. Pharmaceutical Compositions and Modes of Administration

When administered to a patient, Compound (I) is preferably administered as a component of a composition that optionally comprises a pharmaceutically acceptable carrier, excipient or diluent. The composition can be administered orally, or by any other convenient route, for example, by infusion or bolus injection, by absorption through epithelial or mucocutaneous linings (e.g., oral mucosa, rectal, and intestinal mucosa) and may be administered together with another biologically active agent. Administration can be systemic or local. Various delivery systems are known, e.g., encapsulation in liposomes, microparticles, microcapsules, capsules, and can be used to administer the compound.

Methods of administration include, but are not limited to, parenteral, intradermal, intramuscular, intraperitoneal, intravenous, subcutaneous, intranasal, epidural, oral, sublingual, intranasal, intraocular, intratumoral, intracerebral, intravaginal, transdermal, ocularly, rectally, by inhalation, or topically, particularly to the ears, nose, eyes, or skin. The mode of administration is left to the discretion of the practitioner. In most instances, administration will result in the release of a compound into the bloodstream, tissue or cell(s). In a specific aspect, a compound is administered orally.

The amount of Compound (I) that will be effective in the treatment of HD disease resulting from an aberrant amount of mRNA transcripts depends, e.g., on the route of administration, the disease being treated, the general health of the subject, ethnicity, age, weight, and gender of the subject, diet, time, and the severity of disease progress, and should be decided according to the judgment of the practitioner and each patient's or subject's circumstances.

In specific aspects, an “effective amount” in the context of the administration of Compound (I), or composition or medicament thereof refers to an amount of Compound (I) to a patient which has a therapeutic effect and/or beneficial effect. In certain specific aspects, an “effective amount” in the context of the administration of Compound (I), or composition or medicament thereof to a patient results in one, two or more of the following effects: (i) reduces or ameliorates the severity of HD disease; (ii) delays onset of HD disease; (iii) inhibits the progression of HD disease; (iv) reduces hospitalization of a subject; (v) reduces hospitalization length for a subject; (vi) increases the survival of a subject; (vii) improves the quality of life of a subject; (viii) reduces the number of symptoms associated with HD disease; (ix) reduces or ameliorates the severity of a symptom(s) associated with HD disease; (x) reduces the duration of a symptom associated with HD disease associated; (xi) prevents the recurrence of a symptom associated with HD disease; (xii) inhibits the development or onset of a symptom of HD disease; and/or (xiii) inhibits of the progression of a symptom associated with HD disease. In certain aspects, an effective amount of Compound (I) is an amount effective to restore the amount of an RNA transcript of a gene to the amount of the RNA transcript detectable in healthy patients or cells from healthy patients. In other aspects, an effective amount of Compound (I) is an amount effective to restore the amount an RNA isoform and/or protein isoform of gene to the amount of the RNA isoform and/or protein isoform detectable in healthy patients or cells from healthy patients.

In certain aspects, an effective amount of Compound (I) is an amount effective to decrease the aberrant amount of an RNA transcript of a gene which associated with HD disease. In certain aspects, an effective amount of Compound (I) is an amount effective to decrease the amount of the aberrant expression of an isoform of a gene. In some aspects, an effective amount of Compound (I) is an amount effective to result in a substantial change in the amount of an RNA transcript (e.g., mRNA transcript), alternative splice variant or isoform.

In certain aspects, an effective amount of Compound (I) is an amount effective to increase or decrease the amount of an RNA transcript (e.g., an mRNA transcript) of gene which is beneficial for the prevention and/or treatment of HD disease. In certain aspects, an effective amount of Compound (I) is an amount effective to increase or decrease the amount of an alternative splice variant of an RNA transcript of gene which is beneficial for the prevention and/or treatment of HD disease. In certain aspects, an effective amount of Compound (I) is an amount effective to increase or decrease the amount of an isoform of gene which is beneficial for the prevention and/or treatment of HD disease. Non-limiting examples of effective amounts of Compound (I) are described herein.

For example, the effective amount may be the amount required to prevent and/or treat HD disease associated with the aberrant amount of an mRNA transcript of gene in a human subject.

In general, the effective amount will be in a range of from about 0.001 mg/kg/day to about 500 mg/kg/day for a patient having a weight in a range of between about 1 kg to about 200 kg. The typical adult subject is expected to have a median weight in a range of between about 70 and about 100 kg.

Within the scope of the present description, the “effective amount” of Compound (I) for use in the manufacture of a medicament, the preparation of a pharmaceutical kit or in a method for preventing and/or treating HD disease in a human subject in need thereof, is intended to include an amount in a range of from about 0.001 mg to about 35,000 mg.

The compositions described herein are formulated for administration to the subject via any drug delivery route known in the art. Non-limiting examples include oral, ocular, rectal, buccal, topical, nasal, ophthalmic, subcutaneous, intramuscular, intraveneous (bolus and infusion), intracerebral, transdermal, and pulmonary routes of administration.

Aspects described herein include the use of Compound (I) in a pharmaceutical composition. In a specific aspect, described herein is the use of Compound (I) in a pharmaceutical composition for preventing and/or treating HD disease in a human subject in need thereof comprising administering an effective amount of Compound (I) in admixture with a pharmaceutically acceptable carrier, excipient or diluent. In a specific aspect, the human subject is a patient with HD disease associated with the aberrant amount of an mRNA transcript(s).

Compound (I) may optionally be in the form of a composition comprising the compound or a form thereof and an optional carrier, excipient, or diluent. Other aspects provided herein include pharmaceutical compositions comprising an effective amount of Compound (I) and a pharmaceutically acceptable carrier, excipient, or diluent. In a specific aspect, the pharmaceutical compositions are suitable for veterinary and/or human administration. The pharmaceutical compositions provided herein can be in any form that allows for the composition to be administered to a subject.

In a specific aspect and in this context, the term “pharmaceutically acceptable carrier, excipient or diluent” means a carrier, excipient or diluent approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which a therapeutic agent is administered. Such pharmaceutical carriers can be sterile liquids, such as water and oils, including those of petroleum, animal, vegetable, or synthetic origin, such as peanut oil, soybean oil, mineral oil, sesame oil and the like. Water is a specific carrier for intravenously administered pharmaceutical compositions. Saline solutions and aqueous dextrose and glycerol solutions can also be employed as liquid carriers, particularly for injectable solutions.

Typical compositions and dosage forms comprise one or more excipients. Suitable excipients are well-known to those skilled in the art of pharmacy, and non limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a patient and the specific active ingredients in the dosage form. Further provided herein are anhydrous pharmaceutical compositions and dosage forms comprising Compound (I) as described herein. The compositions and single unit dosage forms can take the form of solutions or syrups (optionally with a flavoring agent), suspensions (optionally with a flavoring agent), emulsions, tablets (e.g., chewable tablets), pills, capsules, granules, powder (optionally for reconstitution), taste-masked or sustained-release formulations and the like.

Pharmaceutical compositions provided herein that are suitable for oral administration can be presented as discrete dosage forms, such as, but are not limited to, tablets, caplets, capsules, granules, powder, and liquids. Such dosage forms contain predetermined amounts of active ingredients, and may be prepared by methods of pharmacy well known to those skilled in the art.

Examples of excipients that can be used in oral dosage forms provided herein include, but are not limited to, binders, fillers, disintegrants, and lubricants.

In one aspect, tablets of Compound 1 can be made by direct compression, by admixing Compound 1 with excipients and compressing them to form a tablet. Tablets of Compound 1 can also be made by other methods, including wet granulation or dry granulation. When granulation is used, Compound 1 could be an intergranular and/or extragranular ingredient of a tablet. In one aspect of the present disclosure, Compound 1 is an intragranular ingredient of a tablet. Compound 1 can be mixed with at least one intragranular excipient and wet or dry granulated to form an intragranular blend used in making a tablet. In an aspect of the present disclosure, a tablet is made by a process that includes mixing Compound 1 with at least one intragranular excipient and wet granulating the mixture to form an intragranular blend, mixing the intragranular blend with at least one extragranular excipient, and compressing the resulting mixture to form a tablet.

The term “intragranular” as used herein refers to ingredients that are incorporated into a formulation prior to granulation, i.e., ingredients that are located internally in or part of the granule structure.

The term “extragranular” as used herein, refers to ingredients that are incorporated into a formulation after granulation, i.e., ingredients that are located externally to the granule structure.

In one aspect, a process for making a tablet of the disclosure uses wet granulation in three stages according to the following steps:

### Stage One (Intragranular Stage):

(a) Dissolving the povidone in water,
(b) Passing the remaining intragranular ingredients through a sieve, e.g., a #30 mesh,
(c) Blending the sieved ingredients to form a granulate,
(d) Wetting the granulate with the povidone solution and blending until optimum granules are obtained,
(e) Drying the optimum granules, preferably until a moisture content of about 2% is achieved,
(f) Passing the dried granules through a sieve of a particular size, e.g., a #20 mesh sieve.

### Stage Two (Extragranular Stage)

(a) Passing all the extragranular excipients except for the lubricant (e.g., magnesium stearate) through a sieve, e.g., a #20 mesh,
(b) Adding the sieved extragranular excipients to the milled granules from stage one and blending,
(c) Sieving the lubricant, e.g., using a #30 mesh sieve, and adding it to the blend and blending further,

### Stage Three (Tableting)

Compressing the blend from the final step of Stage two above into tablets using a tablet press, and optionally coating each tablet with a film coating.

In one aspect, tablets of Compound 1 have all of the following characteristics:
Rapid disintegration when dissolved in 0.01 N HCl
Good bioavailability of Compound 1 when administered to a subject
Physical integrity of the tablet, e.g., good friability, and strength.
Stability of Compound 1 in the tablet.

In one aspect, the amount of Compound 1 in a tablet, by weight of the total weight of the tablet, is selected from 5% to 30%, 5% to 25%, 10% to 20%, and 10%.

In one aspect, the amount of Compound 1 in a tablet is in a range from 1 mg to 200 mg.

In another aspect, the amount of Compound 1 in a tablet is in a range from 1 mg to 100 mg.

In another aspect, the amount of Compound 1 in a tablet is selected from 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, and 200 mg.

In another aspect, the amount of Compound 1 in a tablet is selected from 1 mg, 5 mg, 10 mg, 15 mg, 20 mg, 25 mg, 30 mg, 35 mg, 50 mg, 60 mg, 65 mg, 70 mg, 75 mg, 80 mg, 85 mg, 90 mg, 95 mg, 100 mg, 110 mg, 120 mg, 135 mg, and 140 mg.

In another aspect, the amount of Compound 1 in a tablet is selected from 1 mg, 5 mg, 10 mg, 15 mg, 20 mg, 25 mg, 30 mg, 50 mg, 60 mg, 65 mg, 70 mg, 75 mg, 80 mg, 85 mg, 90 mg, 95 mg, 100 mg, 110 mg, 120 mg, 135 mg, and 140 mg.

In another aspect, the amount of Compound 1 in a tablet is selected from 1 mg, 5 mg, 10 mg, 15 mg, 20 mg, 25 mg, 30 mg, 50 mg, 60 mg, 65 mg, 70 mg, and 100 mg.

In another aspect, the amount of Compound 1 in a tablet is selected from 1 mg, 5 mg, 10 mg, 20 mg, 30 mg, and 50 mg.

In another aspect, the amount of Compound 1 in a tablet is selected from 1 mg, 5 mg, 10 mg, 15 mg, 20 mg, 25 mg, 30 mg, 50 mg, 60 mg, 65 mg, 70 mg, and 100 mg.

In another aspect, the amount of Compound 1 in a tablet is selected from 1 mg, 5 mg, 10 mg, 20 mg, 30 mg, and 50 mg.

In another aspect, the amount of Compound 1 in a tablet is selected from 1 mg, 5 mg or 50 mg.

In another aspect, the amount of Compound 1 in a tablet is selected from 5 mg or 50 mg.

In another aspect, the amount of Compound 1 in a tablet is selected from 5 mg, 10 mg, 20 mg, and 30 mg.

In another aspect, the amount of Compound 1 in a tablet is selected from 5 mg, 10 mg, and 20 mg.

The terms “intermittent dosing regimen” or “intermittent dosing schedule”, as used herein, mean a dosing regimen that comprises administering Compound 1, followed by a resting period. For example, Compound 1 is administered according to an intermittent dosing schedule of at least two cycles, each cycle comprising (a) a dosing period and thereafter (b) a resting period.

As used herein, the term “resting period” refers, in particular, to a period of time during which the patient is not given Compound 1 (i.e., a period of time wherein the treatment with Compound 1 is withheld). For example, if Compound 1 is given on a daily basis, there would be rest period if the daily administration is discontinued for some time, e.g., for some number of days, or the plasma concentration of Compound 1 is maintained at sub-therapeutic level for some time e.g., for some number of days. The dosing period and/or the dose of Compound 1 can be the same or different between cycles. The total treatment time (i.e., the number of cycles for treatment) may also vary from patient to patient based, for example, on the particular patient being treated (e.g., Stage I HD patient).

In another aspect, an intermittent dosing schedule comprises at least two cycles, each cycle comprising (a) a dosing period during which a therapeutically effective amount of Compound 1 is administered to said patient and thereafter (b) a resting period. The terms “intermittent dosing regimen” or “intermittent dosing schedule”, as used herein, refer to both a dosing regimen for Compound 1 alone (i.e. monotherapy) or a dosing regimen for administering Compound 1 in combination with at least a further active ingredient (i.e. combination therapy). In another aspect, the terms “intermittent dosing regimen” or “intermittent dosing schedule” refers to repeated on/off treatment, wherein Compound 1 is administered at regular intervals in a periodic manner, for example, once a day, every 2 days, every 3 days, every 4 days, once a week, or twice a week.

The term “once a day” or “once daily” or “QD” in the context of administering a drug means herein administering one dose of a drug once each day, wherein the dose is, for example, administered on the same day of the week.

In one aspect, the terms “administering” or “administration of Compound 1 once a day,” as used herein, refer to the amount of Compound 1 in a tablet in a range of from 1 mg to 100 mg, administered once a day.

In another aspect, the amount of Compound 1 in a tablet is in a range of from 1 mg to 200 mg, administered once a day.

In another aspect, the amount of Compound 1 in a tablet is in a range of from 1 mg to 100 mg, administered once a day.

In another aspect, the amount of Compound 1 in a tablet is selected from 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, and 200 mg, administered once a day.

In another aspect, the amount of Compound 1 in a tablet is selected from 1 mg, 5 mg, 10 mg, 15 mg, 20 mg, 25 mg, 30 mg, 35 mg, 50 mg, 60 mg, 65 mg, 70 mg, 75 mg, 80 mg, 85 mg, 90 mg, 95 mg, 100 mg, 110 mg, 120 mg, 135 mg, and 140 mg, administered once a day.

In another aspect, the amount of Compound 1 in a tablet is selected from 1 mg, 5 mg, 10 mg, 15 mg, 20 mg, 25 mg, 30 mg, 50 mg, 60 mg, 65 mg, 70 mg, 75 mg, 80 mg, 85 mg, 90 mg, 95 mg, 100 mg, 110 mg, 120 mg, 135 mg, and 140 mg, administered once a day.

In another aspect, the amount of Compound 1 in a tablet is selected from 1 mg, 5 mg, 10 mg, 15 mg, 20 mg, 25 mg, 30 mg, 50 mg, 60 mg, 65 mg, 70 mg, and 100 mg, administered once a day.

In another aspect, the amount of Compound 1 in a tablet is selected from 1 mg, 5 mg, 10 mg, 20 mg, 30 mg, and 50 mg, administered once a day.

In another aspect, the amount of Compound 1 in a tablet is selected from 1 mg, 5 mg, 10 mg, 15 mg, 20 mg, 25 mg, 30 mg, 50 mg, 60 mg, 65 mg, 70 mg, and 100 mg, administered once a day.

In another aspect, the amount of Compound 1 in a tablet is selected from 1 mg, 5 mg or 50 mg, administered once a day.

In another aspect, the amount of Compound 1 in a tablet is selected from 5 mg or 50 mg, administered once a day.

In another aspect, the amount of Compound 1 in a tablet is selected from 5 mg, 10 mg, 20 mg, and 30 mg, administered once a day.

In another aspect, the amount of Compound 1 in a tablet is selected from 5 mg, 10 mg, and 20 mg, administered once a day.

The term “once a week” or “once weekly” or “QW” in the context of administering Compound 1 means herein administering one dose of Compound 1 once each week, wherein the dose is, for example, administered on the same day each week.

In one aspect, the terms “administering” or “administration of Compound 1 once a week,” as used herein, refer to Compound 1 administered in an amount selected from a range of from 25 mg to 100 mg once a week, a range of from 25 mg to 200 mg once a week, and a range of from 50 mg to 200 mg once a week.

In another aspect, Compound 1 is administered in an amount selected from 35 mg once a week, 70 mg once a week, and 140 mg once a week.

The term “twice a week” or “twice weekly” or “BIW’ in the context of administering Compound 1 means herein administering one dose of Compound 1 twice each week, wherein each dose is administered on separate days each week at regular intervals in a range of from 48 to 72 hours.

In one aspect, the terms “administering” or “administration of Compound 1 twice a week,” as used herein, refer to Compound 1 administered in an amount selected from a range of from 10 mg to 100 mg twice a week, a range of from 10 mg to 200 mg twice a week, and a range of from 25 mg to 100 mg twice a week.

In another aspect, Compound 1 is administered in an amount selected from a range of from 10 mg to 20 mg twice a week, such as about 15 mg twice a week, a range of from 30 mg to 40 mg twice a week, such as 35 mg twice a week, and a range of from 50 mg to 90 mg twice a week, such as 70 mg twice a week.

In another aspect, the method for modulating the amount of one, two, three or more RNA transcripts of a HD gene described herein, comprising contacting a cell with Compound (I) includes a cell in a cell culture. In other aspects, the cell is contacted with Compound (I) in a subject (e.g., a non-human animal subject or a human subject).

In certain aspects described herein, the cell(s) is contacted or cultured with Compound (I) with Compound (I) for a period of 15 minutes, 30 minutes, 45 minutes, 1 hour, 2 hours, 3 hours, 4 hours, 5 hours, 6 hours, 8 hours, 12 hours, 18 hours, 24 hours, 48 hours, 72 hours or more. In other aspects described herein, the cell(s) is contacted or cultured with Compound (I) with Compound (I) for a period of 15 minutes to 1 hour, 1 to 2 hours, 2 to 4 hours, 6 to 12 hours, 12 to 18 hours, 12 to 24 hours, 28 to 24 hours, 24 to 48 hours, 48 to 72 hours.

In certain aspects described herein, the cell(s) is contacted or cultured with a certain concentration of Compound (I), wherein the certain concentration is 0.01 μM, 0.05 μM, 1 μM, 2 μM, 5 μM, 10 μM, 15 μM, 20 μM, 25 μM, 50 μM, 75 μM, 100 μM, or 150 μM. In other aspects described herein, the cell(s) is contacted or cultured with a certain concentration of Compound (I), wherein the certain concentration is 175 μM, 200 μM, 250 μM, 275 μM, 300 μM, 350 μM, 400 μM, 450 μM, 500 μM, 550 μM 600 μM, 650 μM, 700 μM, 750 μM, 800 μM, 850 μM, 900 μM, 950 μM or 1 mM. In some aspects described herein, the cell(s) is contacted or cultured with a certain concentration of Compound (I), wherein the certain concentration is 5 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, or 950 nM. In certain aspects described herein, the cell(s) is contacted or cultured with a certain concentration of Compound (I), wherein the certain concentration is between 0.01 μM to 0.1 μM, 0.1 μM to 1 μM, 1 μM to 50 μM, 50 μM to 100 μM, 100 μM to 500 μM, 500 μM to 1 nM, 1 nM to 10 nM, 10 nM to 50 nM, 50 nM to 100 nM, 100 nM to 500 nM, 500 nM to 1000 nM. In certain aspects described herein, the cell(s) is contacted or cultured with a certain concentration of Compound (I) that results in a substantial change in the amount of an RNA transcript (e.g., an mRNA transcript), an alternatively spliced variant, or an isoform of a gene (e.g., a gene described herein, infra).

In another aspect, provided herein are methods for modulating the amount of one, two, three or more RNA transcripts of a HTT gene, wherein the precursor RNA transcript transcribed from the HTT gene comprises an intronic sequence comprising a 3′ splice site and a noncanonical 5′ splice site in proximity to a pseudo-ESE, the methods comprising administering to a human or non-human subject Compound (I), or a pharmaceutical composition comprising Compound (I) and a pharmaceutically acceptable carrier, excipient or diluent.

In another aspect, the precursor RNA transcript contains in 5′ to 3′ order: an intronic sequence comprising a 3′ splice site and a noncanonical 5′ splice site in proximity to a pseudo-ESE.

In one aspect, provided herein are methods for modulating the amount of one, two, three or more RNA transcripts of a HTT gene described herein, the methods comprising administering to a human or non-human subject Compound (I), or a pharmaceutical composition comprising Compound (I) and a pharmaceutically acceptable carrier, excipient or diluent.

In certain aspects, Compound (I) contacted or cultured with a cell(s) or administered to a subject is a compound as described herein.

### VII. Methods of Preventing and/or Treating Huntington Disease

In one aspect, provided herein are methods for preventing and/or treating HD disease associated with the aberrant expression of a product of a HD gene (e.g., an mRNA transcript or protein), wherein the precursor RNA transcript transcribed from the gene comprises a small molecule inducible intronic sequence comprising a noncanonical 5′ splice site in proximity to a pseudo-ESE and a 3′ splice site, the methods comprising administering to a human or non-human subject Compound (I), or a pharmaceutical composition comprising Compound (I) and a pharmaceutically acceptable carrier, excipient or diluent.

In one aspect, the precursor RNA transcript comprises in 5′ to 3′ order: a 5′ exonic splice site, a first intronic branch point, a small molecule inducible intronic sequence comprising an intronic 3′ splice site, a pseudo-Exonic Splice Enhancer (pseudo-ESE), a noncanonical 5′ intronic splice site, and, downstream on the intronic sequence, a second intronic branch point, and a 3′ exonic splice site.

In one aspect, the methods described herein prevent the onset or development of one or more symptoms of HD. In another aspect, the methods for preventing HD disease described herein impede the recurrence of the disease or delays the recurrence of the disease. In another aspect, the methods for treating HD disease described herein have one, two or more of the effects: (i) reduce or ameliorate the severity of the disease; (ii) inhibit the progression of the disease; (iii) reduce hospitalization of a subject; (iv) reduce hospitalization length for a subject; (v) increase the survival of a subject; (vi) improve the quality of life of a subject; (vii) reduce the number of symptoms associated with the disease; (viii) reduce or ameliorates the severity of a symptom(s) associated with the disease; (ix) reduce the duration of a symptom(s) associated with the disease; (x) prevent the recurrence of a symptom associated with the disease; (xi) inhibit the development or onset of a symptom of the disease; and/or (xii) inhibit of the progression of a symptom associated with the disease.

The term “rate of progression”, as used herein, refers, for example, to the annual rate of change (e.g., decline) or the rate of change (e.g., decline) per year, for example as assessed according to standard scales, such as clinical scales, or according to neuroimaging measures.

The term “reducing”, as used herein, refers to e.g., 5%, 10%, 20%, 30%, 40%, 50%, 60% or 70% reduction, for example, per year of treatment.

The term “delaying”, as used herein, refers to delay for at least e.g., 0.5, 1, 2, 3, 4, 5, 6, 7, 8, 9, 10, 11, 12, 13, 14 or 15 years.

The terms “slowing progression of HD”, “slowing progression of Huntington's disease”, “to slow the progression of HD” or “to slow the progression of Huntington's disease”, as used herein, refer to delaying the onset of Huntington's disease, e.g., increasing time for the onset of Huntington's disease as defined herein, for example, by at least 25% (e.g., by 25% or more, such as from 25% to 50%).

In another aspect, the terms refer to reducing the rate of progression between stages of Huntington's disease, for example, reducing the rate of progression from an initial stage of HD into a more advanced stage of HD, as assessed, for example, compared to placebo, according to standard scales, such as clinical scales [e.g., according to the UHDRS total functional capacity (TFC) scale, for example, in Neurology, 1979, 29, 1-3]. In another aspect, it refers to reducing the rate of progression from stage 1 of HD into stage 2 of HD (e.g., compared to placebo). In another aspect, the terms refer to reducing the rate of progression from stage 2 of HD into stage 3 of HD (e.g., compared to placebo). In another aspect, the terms refer to reducing the rate of progression from stage 3 of HD into stage 4 of HD (e.g., compared to placebo). In another aspect, the terms refer to reducing the rate of progression from stage 4 of HD into stage 5 of HD (e.g., compared to placebo). In another aspect, the terms refer to reducing the rate of progression from early HD into middle stage HD (e.g., compared to placebo). In another aspect, the terms refer to reducing the rate of progression from middle stage HD into advanced HD (e.g., compared to placebo).

The term “onset of Huntington's disease”, as used herein, refers to clinical diagnosis of HD as generally established [e.g., onset of motor disturbances based on diagnostic confidence score (DCS) of 4, as defined by the Unified Huntington Rating Scale (UHDRS) total motor score (TMS)].

In another aspect, the terms “slowing progression of HD”, “slowing progression of Huntington's disease”, “to slow the progression of HD” or “to slow the progression of Huntington's disease”, as used herein, refer to delaying the onset of symptoms associated with Huntington's disease, e.g., increasing time for the onset of one or more symptom associated with Huntington's disease selected from decline of motor function associated with Huntington's disease, cognitive decline associated with Huntington's disease, psychiatric decline associated with Huntington's disease and decline of functional capacity associated with Huntington's disease, as defined herein. In another aspect, the terms refer to reducing the rate of progression of one or more symptom associated with Huntington's disease selected from decline of motor function associated with Huntington's disease, cognitive decline associated with Huntington's disease, psychiatric decline associated with Huntington's disease and decline of functional capacity associated with Huntington's disease, as defined herein.

As used herein, the term “reducing the rate of” refers, for example, to increasing time for onset or increasing time for a rise of severity (e.g., compared to placebo). In another aspect, the terms “slowing progression of HD”, “slowing progression of Huntington's disease”, “to slow the progression of HD” or “to slow the progression of Huntington's disease”, as used herein, refer to reducing the rate of progression of pre-manifest HD into manifest HD [i.e., delaying the onset of manifest HD; e.g., compared to placebo; e.g., as assessed by a diagnostic confidence score (DCS) of 4, as defined by the Unified Huntington Rating Scale (UHDRS) total motor score (TMS)].

In another aspect, the terms “slowing progression of HD”, “slowing progression of Huntington's disease”, “to slow the progression of HD” or “to slow the progression of Huntington's disease”, as used herein, refer to slowing the progression of Huntington's disease pathaphysiology.

In another aspect, the term “slowing the progression of Huntington's disease pathophysiology”, as used herein, refers to reducing the rate of progression of Huntington's disease pathophysiology, for example, as assessed by magnetic resonance imaging (MRI) [e.g., by neuroimaging measures, such as in Lancet Neural. 2013, 12 (7), 637-649]. For example, it refers to reducing the rate (e.g., reducing the annual rate, for example, versus placebo) of brain (e.g., whole brain, caudate, striatum or cortex) volume loss (e.g., % from baseline volume) associated with Huntington's disease (e.g., as assessed by MRI).

In one aspect, administration of Compound I prevents or mitigates a decline of motor function in HD patients. The term “motor function”, as used herein, refers to motor features of HD comprising, for example, one or more selected from the group consisting of ocular motor function, dysarthria, chorea, postural stability and gait. The term “decline of motor function”, as used herein, refers to decreased motor function (e.g., from normal motor function or from previous clinic visit). Decline of motor function may be assessed, for example, according to standard scales, such as clinical scales (e.g., UHDRS motor assessment scale, as measured by the UHDRS Total Motors Score; e.g., in Movement Disorders, 1996, 11, 136-142). The terms “slowing the decline of motor function” or “to slow the decline of motor function”, as used herein, refer to reducing the rate of decline of motor function (e.g., compared to placebo; e.g., reduction in the annual rate of decline of motor function, for example, versus placebo; e.g., as assessed by the UHDRS Total Motors Score). The term “reducing the rate”, as used herein, refers to increasing time for onset or increasing time for a rise of severity (e.g., compared to placebo; e.g., reduction in the annual rate of decline, for example, versus placebo).

In one aspect, administration of Compound I prevents or mitigates cognitive decline associated with HD. The term “cognitive decline”, as used herein, refers to decreased cognitive abilities (e.g., from normal cognition function or from previous clinic visit). In one aspect, the term refers to, for example, decline of one or more cognition functions selected from the group consisting of attention, processing speed, visuospatial processing, timing, emotion processing, memory, verbal fluency, psychomotor function, and executive function. Cognitive decline may be assessed, for example, according to standard scales, such as clinical scales [e.g., as assessed by the Symbol Digit Modalities Test, the Stroop Word Reading Test, the Montreal Cognitive Assessment or the HD Cognitive Assessment Battery (comprising the Symbol Digit Modalities Test, Trail Making Test B, One Touch Stockings, Paced Tapping, Emotion Recognition Test, Hopkins Verbal Learning Test); e.g., in Movement Disorders, 2014, 29 (10), 1281-1288). The terms “slowing cognitive decline” or “to slow cognitive decline”, as used herein, refer to reducing the rate of cognitive decline (e.g., compared to placebo; e.g., reduction in the annual rate of cognitive decline versus placebo; e.g., as assessed by the Symbol Digit Modalities Test, by the Stroop Word Reading Test, by the Montreal Cognitive Assessment or by the HD Cognitive Assessment Battery). The term “reducing the rate”, as used herein, refers to increasing time for onset or increasing time for a rise of severity (e.g., compared to placebo; e.g., reduction in the annual rate of decline, for example, versus placebo).

In one aspect, psychiatric decline is prevented or mitigated in HD patients treated with Compound I. The term “psychiatric decline”, as used herein, refers to decreased psychiatric function (e.g., from normal psychiatric function or from previous clinic visit). In one aspect, the term refers to, for example, one or more psychiatric functions selected from the group consisting of apathy, anxiety, depression obsessive compulsive behavior, suicidal thoughts, irritability and agitation. Psychiatric decline may be assessed, for example, according to standard scales, such as clinical scales (e.g., as assessed by the Apathy Evaluation Scale or by the Hospital Anxiety and Depression Scale; e.g., in Movement Disorders, 2016, 31 (10), 1466-1478, Movement Disorders, 2015, 30 (14), 1954-1960). The terms “slowing psychiatric decline” or “to slow psychiatric decline”, as used herein, refer to reducing the rate of psychiatric decline (e.g., compared to placebo; e.g., reduction in the annual rate of psychiatric decline versus placebo; e.g., as assessed by the Apathy Evaluation Scale or by the Hospital Anxiety and Depression Scale). The term “reducing the rate”, as used herein, refers to increasing time for onset or increasing time for a rise of severity (e.g., compared to placebo; e.g., reduction in the annual rate of decline, for example, versus placebo).

The term “functional capacity”, as used herein, refers, for example, to the ability to work, handle financial affairs, manage domestic chores, perform activities of daily living, and level of care needed. Functional capacity comprises, for example, one or more selected from the group consisting of capacity to work, capacity to handle financial affairs, capacity to manage domestic chores, capacity to perform activities of daily living, and level of care needed.

The term “decline of functional capacity”, as used herein, refers to decreased functional capacity (e.g., from normal functional capacity or from previous clinic visit). Decline of functional capacity may be assessed, for example, according to standard scales, such as clinical scales (e.g., UHDRS functional assessment scale and independence scale, and UHDRS Total Functional Capacity Scale e.g., in Movement Disorders, 1996, 11, 136-142).

The terms “slowing the decline of functional capacity” or “to slow the decline of functional capacity”, as used herein, refer to reducing the rate of decline of functional capacity (e.g., compared to placebo; e.g., reduction in the annual rate of decline of functional capacity versus placebo; e.g., as assessed by the UHDRS functional assessment scale and independence scale or by the UHDRS Total Functional Capacity Scale). The term “reducing the rate”, as used herein, refers to increasing time for onset or increasing time for a rise of severity (e.g., compared to placebo; e.g., reduction in the annual rate of decline, for example, versus placebo).

The term “decline”, as used herein, refers, for example, to worsening over time (e.g., annually or per year) of a condition or of a particular feature of a condition, for example, as assessed according to standard scales, such as clinical scales.

The term “Unified Huntington Disease Rating Scale” or “UHDRS” as used herein, refers to the clinical rating scale developed by the Huntington Study Group (e.g., in Movement Disorders, 1996, 11, 136-142, which is incorporated fully herein by reference), which assesses domains of clinical performance and capacity in HD. The UHDRS comprises rating scales for motor function, cognitive function, and functional capacity. It yields scores assessing primary features of HD (e.g., motor, and cognitive) and overall functional impact of these features.

The term “cHDRS” refers to the composite Unified Huntington Disease Rating Scale, which provides composite measure of motor, cognitive and global functioning (e.g., in Neurology 2017, 89, 2495-2502).

The terms “HD stage 1”, “HD stage 1”, “Huntington's disease stage 1”, “Huntington's disease stage I”, “stage 1 of Huntington's disease” or “stage I of Huntington's disease”, as used herein, refer to a disease stage of HD as clinically stablished [e.g., as assessed according to standard scales, for example, clinical scales, such as on the basis of the UHDRS total functional capacity (TFC) scale, wherein the TFC score is from 11 to 13]. At HD stage 1, typically, the patient has been clinically diagnosed with HD, is fully functional at home and at work and maintains independence as regards functional capacities; typically, 0 to 8 years from onset of Huntington's disease.

The terms “HD stage 2”, “HD stage II”, “Huntington's disease stage 2”, “Huntington's disease stage II”, “stage 2 of Huntington's disease” or “stage II of Huntington's disease”, as used herein, refer to a disease stage of HD as clinically stablished [e.g., as assessed according to standard scales, for example, clinical scales, such as on the basis of the UHDRS total functional capacity (TFC) scale, wherein the TFC score is from 7 to 10]. At HD stage 2, typically, the patient is still functional at work, however at lower capacity, is mostly able to carry out daily activities, despite some difficulties, and usually requires only slight assistance; typically, 3 to 13 years from onset of Huntington's disease.

The terms “HD stage 3”, “HD stage Ill”, “Huntington's disease stage 3”, “Huntington's disease stage Ill”, “stage 3 of Huntington's disease” or “stage III of Huntington's disease”, as used herein, refer to a disease stage of HD as clinically stablished [e.g., as assessed according to standard scales, for example, clinical scales, such as on the basis of the UHDRS total functional capacity (TFC) scale, wherein the TFC score is from 4 to 6]. At HD stage 3, typically, the patient can no longer conduct work or manage household chores, requires substantial help for daily financial affairs, domestic responsibilities, and activities of daily living; typically, 5 to 16 years from onset of Huntington's disease.

The terms “HD stage 4”, “HD stage IV”, “Huntington's disease stage 4”, “Huntington's disease stage IV”, “stage 4 of Huntington's disease” or “stage IV of Huntington's disease”, as used herein, refer to a disease stage of HD as clinically stablished [e.g., as assessed according to standard scales, for example, clinical scales, such as on the basis of the UHDRS total functional capacity (TFC) scale, wherein the TFC score is from 1 to 3]. At HD stage 4, typically, the patient is not independent, but still can reside at home with help from either family or professionals, however, requiring substantial assistance in financial affairs, domestic chores, and most activities of daily living; typically, 9 to 21 years from onset of Huntington's disease.

The terms “HD stage 5”, “HD stage V”, “Huntington's disease stage 5”, “Huntington's disease stage V”, “stage 5 of Huntington's disease” or “stage V of Huntington's disease”, as used herein, refer to a disease stage of HD as clinically stablished [e.g., as assessed according to standard scales, for example, clinical scales, such as on the basis of the UHDRS total functional capacity (TFC) scale, wherein the TFC score is 0]. At HD stage 5, typically, the patient needs total support in daily activities from professional nursing care; typically 11 to 26 years from onset of Huntington's disease.

The terms “early HD”, “early Huntington's disease”, “early stage of HD” or “early stage of Huntington's disease”, as used herein, refer to a disease stage of HD, wherein the patient is largely functional and may continue to work and live independently, despite suffering from, for example, one or more selected from the group consisting of minor involuntary movements, subtle loss of coordination and difficulty thinking through complex problems. In another aspect, the terms “early HD”, “early Huntington's disease”, “early stage of HD” or “early stage of Huntington's disease”, refer to “HD stage 2”, as defined herein.

The terms “moderate HD”, “moderate Huntington's disease”, “moderate stage of HD”, “moderate stage of Huntington's disease”, “middle stage HD”, “middle stage Huntington's disease”, “middle stage of HD” or “middle stage of Huntington's disease”, as used herein, refer to a disease stage of HD, wherein the patient may no be able to work, manage own finances or perform own household chores, but will be able to eat, dress, and attend to personal hygiene with assistance. Typically, at this stage, for example, chorea may be prominent, as well as problems with swallowing, balance, falls, weight loss, and problem solving. In another aspect, the terms “moderate HD”, “moderate Huntington's disease”, “moderate stage of HD”, “moderate stage of Huntington's disease”, “middle stage HD”, “middle stage Huntington's disease”, “middle stage of HD” or “middle stage of Huntington's disease” refer to “HD stage 3”, as defined herein.

The terms “advanced HD”, “advanced Huntington's disease”, “advanced stage of HD”, “advanced stage of Huntington's disease”, “late HD” or “late Huntington's disease”, “late stage of HD” or “late stage of Huntington's disease”, as used herein, refer to a disease stage of HD, wherein the patient requires assistance in all activities of daily living. Typically, at this stage, for example, chorea may be severe, but more often it is replaced by rigidity, dystonia, and bradykinesia. In another aspect, the terms “advanced HD”, “advanced Huntington's disease”, “advanced stage of HD”, “advanced stage of Huntington's disease”, “late HD” or “late Huntington's disease”, “late stage of HD” or “late stage of Huntington's disease” refers to “HD stage 4” or “HD stage 5”, as defined herein.

The terms “juvenile HD” or “juvenile Huntington's disease”, as used herein, refer to diagnosis of HD as clinically stablished (e.g., on the basis of confirmed family history or positive genetic test (i.e. confirmation of CAG repeat expansion 2-36); and onset of symptoms by age <21 years).

The terms “pediatric HD” or “pediatric Huntington's disease”, as used herein, refer to a patient affected by HD (e.g., on the basis of: confirmed family history or positive genetic test (i.e. confirmation of CAG repeat expansion 2-36) and clinical diagnosis) and who is aged <18 years.

In another aspect, provided herein are methods for preventing and/or treating a subject with Huntington's disease (HD), wherein huntingtin pre-mRNA comprises a small molecule inducible intronic sequence comprising a noncanonical intronic 5′ splice site in proximity to a pseudo-ESE and an intronic 3′ splice site. Administration of Compound (I), or a pharmaceutical composition comprising Compound (I) and a pharmaceutically acceptable carrier, excipient or diluent to the subject induces alternative splicing of the small molecule inducible intronic sequence (pseudoexon) into the mature huntingtin mRNA. Insertion of the intron-derived exon into the mature huntingtin mRNA causes a frameshift that disrupts the open reading frame and introduces one or more premature stop codons. This ensuing premature termination of translation earmarks the mRNAs for nonsense mediated decay which results in a decrease in the amount of huntingtin protein.

In one aspect, Compound (I) is therapeutically effective if the amount of Compound (I) decreases huntingtin protein expression by about 30% to about 50% relative to a control thereby alleviating one or more symptoms of HD.

In one aspect, Compound (I) is therapeutically effective if the amount of Compound (I) decreases huntingtin protein expression by about 20%, 30%, 40%, 50% or 60% and alleviates one or more symptoms of HD including, but not limited to, involuntary movements of the limbs and body, impaired speech, difficulty swallowing and breathing and limited mobility.

In another aspect, treating or ameliorating Huntington's Disease with Compound 1, or a pharmaceutically acceptable salt thereof, has one or more of the following effects: (i) a favorable therapeutic profile, such as a favorable safety profile or metabolic profile; or, (ii) a favorable off-target effect profile, such as a favorable psychiatric adverse event profile, a favorable toxicity (e.g. genotoxicity) or cardiovascular adverse event (e.g. blood pressure, heart rate, electrocardiography parameters) profile.

In one aspect, a patient in need thereof is orally administered a tablet of the disclosure, containing a therapeutically effective amount of Compound 1.

In another aspect, the tablet contains the therapeutically effective amount of Compound I is in a range of from 1 mg to 200 mg of Compound 1.

In another aspect, the tablet contains the therapeutically effective amount in a range of from 1 mg to 100 mg of Compound 1.

In another aspect, the tablet contains the therapeutically effective amount selected from 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, and 200 mg.

In another aspect, the tablet contains the therapeutically effective amount selected from 1 mg, 5 mg, 10 mg, 15 mg, 20 mg, 25 mg, 30 mg, 35 mg, 50 mg, 60 mg, 65 mg, 70 mg, 75 mg, 80 mg, 85 mg, 90 mg, 95 mg, 100 mg, 110 mg, 120 mg, 135 mg, and 140 mg.

In another aspect, the tablet contains the therapeutically effective amount selected from 1 mg, 5 mg, 10 mg, 15 mg, 20 mg, 25 mg, 30 mg, 50 mg, 60 mg, 65 mg, 70 mg, 75 mg, 80 mg, 85 mg, 90 mg, 95 mg, 100 mg, 110 mg, 120 mg, 135 mg, and 140 mg.

In another aspect, the tablet contains the therapeutically effective amount selected from 1 mg, 5 mg, 10 mg, 15 mg, 20 mg, 25 mg, 30 mg, 50 mg, 60 mg, 65 mg, 70 mg, and 100 mg.

In another aspect, the tablet contains the therapeutically effective amount selected from 1 mg, 5 mg, 10 mg, 20 mg, 30 mg, and 50 mg.

In another aspect, the tablet contains the therapeutically effective amount selected from 1 mg, 5 mg, 10 mg, 15 mg, 20 mg, 25 mg, 30 mg, 50 mg, 60 mg, 65 mg, 70 mg, and 100 mg.

In another aspect, the tablet contains the therapeutically effective amount selected from 1 mg, 5 mg or 50 mg.

In another aspect, the tablet contains the therapeutically effective amount selected from 5 mg or 50 mg.

In another aspect, the tablet contains the therapeutically effective amount selected from 5 mg, 10 mg, 20 mg, and 30 mg.

In another aspect, the tablet contains the therapeutically effective amount selected from 5 mg, 10 mg, and 20 mg.

In one aspect, a patient in need thereof is orally administered a tablet of the disclosure, containing a therapeutically effective amount of Compound 1, administered once a day.

In another aspect, the tablet contains the therapeutically effective amount in a range of from 1 mg to 200 mg of Compound 1, administered once a day.

In another aspect, the tablet contains the therapeutically effective amount in a range of from 1 mg to 100 mg of Compound 1, administered once a day.

In another aspect, the tablet contains the therapeutically effective amount selected from 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, and 200 mg, administered once a day.

In another aspect, the tablet contains the therapeutically effective amount selected from 1 mg, 5 mg, 10 mg, 15 mg, 20 mg, 25 mg, 30 mg, 35 mg, 50 mg, 60 mg, 65 mg, 70 mg, 75 mg, 80 mg, 85 mg, 90 mg, 95 mg, 100 mg, 110 mg, 120 mg, 135 mg, and 140 mg, administered once a day.

In another aspect, the tablet contains the therapeutically effective amount selected from 1 mg, 5 mg, 10 mg, 15 mg, 20 mg, 25 mg, 30 mg, 50 mg, 60 mg, 65 mg, 70 mg, 75 mg, 80 mg, 85 mg, 90 mg, 95 mg, 100 mg, 110 mg, 120 mg, 135 mg, and 140 mg, administered once a day.

In another aspect, the tablet contains the therapeutically effective amount selected from 1 mg, 5 mg, 10 mg, 15 mg, 20 mg, 25 mg, 30 mg, 50 mg, 60 mg, 65 mg, 70 mg, and 100 mg, administered once a day.

In another aspect, the tablet contains the therapeutically effective amount selected from 1 mg, 5 mg, 10 mg, 20 mg, 30 mg, and 50 mg, administered once a day.

In another aspect, the tablet contains the therapeutically effective amount selected from 1 mg, 5 mg, 10 mg, 15 mg, 20 mg, 25 mg, 30 mg, 50 mg, 60 mg, 65 mg, 70 mg, and 100 mg, administered once a day.

In another aspect, the tablet contains the therapeutically effective amount selected from 1 mg, 5 mg or 50 mg, administered once a day.

In another aspect, the tablet contains the therapeutically effective amount selected from 5 mg or 50 mg, administered once a day.

In another aspect, the tablet contains the therapeutically effective amount selected from 5 mg, 10 mg, 20 mg, and 30 mg, administered once a day.

In another aspect, the tablet contains the therapeutically effective amount selected from 5 mg, 10 mg, and 20 mg, administered once a day.

In another aspect, the tablet contains the therapeutically effective amount of 1 mg of Compound 1.

In another aspect, the tablet contains the therapeutically effective amount of 5 mg of Compound 1.

In another aspect, the tablet contains the therapeutically effective amount of 10 mg of Compound 1.

In another aspect, the tablet contains the therapeutically effective amount of 15 mg of Compound 1.

In another aspect, the tablet contains the therapeutically effective amount of 20 mg of Compound 1.

In another aspect, the tablet contains the therapeutically effective amount of 25 mg of Compound 1.

In another aspect, the tablet contains the therapeutically effective amount of 30 mg of Compound 1.

In another aspect, the tablet contains the therapeutically effective amount of 35 mg of Compound 1.

In another aspect, the tablet contains the therapeutically effective amount of 40 mg of Compound 1.

In another aspect, the tablet contains the therapeutically effective amount of 45 mg of Compound 1.

In another aspect, the tablet contains the therapeutically effective amount of 50 mg of Compound 1.

In another aspect, the tablet contains the therapeutically effective amount of 55 mg of Compound 1.

In another aspect, the tablet contains the therapeutically effective amount of 60 mg of Compound 1.

In another aspect, the tablet contains the therapeutically effective amount of 65 mg of Compound 1.

In another aspect, the tablet contains the therapeutically effective amount of 70 mg of Compound 1.

In another aspect, the tablet contains the therapeutically effective amount of 75 mg of Compound 1.

In another aspect, the tablet contains the therapeutically effective amount of 80 mg of Compound 1.

In another aspect, the tablet contains the therapeutically effective amount of 85 mg of Compound 1.

In another aspect, the tablet contains the therapeutically effective amount of 90 mg of Compound 1.

In another aspect, the tablet contains the therapeutically effective amount of 95 mg of Compound 1.

In another aspect, the tablet contains the therapeutically effective amount of 100 mg of Compound 1.

In another aspect, the tablet contains the therapeutically effective amount of 105 mg of Compound 1.

In another aspect, the tablet contains the therapeutically effective amount of 110 mg of Compound 1.

In another aspect, the tablet contains the therapeutically effective amount of 115 mg of Compound 1.

In another aspect, the tablet contains the therapeutically effective amount of 120 mg of Compound 1.

In another aspect, the tablet contains the therapeutically effective amount of 125 mg of Compound 1.

In another aspect, the tablet contains the therapeutically effective amount of 130 mg of Compound 1.

In another aspect, the tablet contains the therapeutically effective amount of 135 mg of Compound 1.

In another aspect, the tablet contains the therapeutically effective amount of 140 mg of Compound 1.

In another aspect, the tablet contains the therapeutically effective amount of 145 mg of Compound 1.

In another aspect, the tablet contains the therapeutically effective amount of 150 mg of Compound 1.

In another aspect, the tablet contains the therapeutically effective amount of 155 mg of Compound 1.

In another aspect, the tablet contains the therapeutically effective amount of 160 mg of Compound 1.

In another aspect, the tablet contains the therapeutically effective amount of 165 mg of Compound 1.

In another aspect, the tablet contains the therapeutically effective amount of 170 mg of Compound 1.

In another aspect, the tablet contains the therapeutically effective amount of 175 mg of Compound 1.

In another aspect, the tablet contains the therapeutically effective amount of 180 mg of Compound 1.

In another aspect, the tablet contains the therapeutically effective amount of 185 mg of Compound 1.

In another aspect, the tablet contains the therapeutically effective amount of 190 mg of Compound 1.

In another aspect, the tablet contains the therapeutically effective amount of 195 mg of Compound 1.

In another aspect, the tablet contains the therapeutically effective amount of 200 mg of Compound 1.

In another aspect, a tablet containing the therapeutically effective amount of Compound 1 is administered once per day.

In another aspect, the tablet containing the therapeutically effective amount of Compound 1 is administered twice per day.

In another aspect, a tablet containing the therapeutically effective amount of Compound 1 is administered three times per day.

In another aspect, a tablet containing the therapeutically effective amount of Compound 1 is administered once per week.

In another aspect, a tablet containing the therapeutically effective amount of Compound 1 is administered once every two weeks.

In one aspect, a use of a tablet containing a therapeutically effective amount of Compound 1, or a pharmaceutically acceptable salt thereof, in treating or ameliorating Huntington's Disease as a disease-modifying therapy, includes Huntington's disease selected from the group consisting of Huntington's Disease genetically characterized by CAG repeat expansion of from 36 to 39 in the HTT gene on chromosome 4; and, Huntington's disease genetically characterized by CAG repeat expansion of from >39 in the HTT gene on chromosome 4.

In one aspect, a use of a tablet containing a therapeutically effective amount of Compound 1, or a pharmaceutically acceptable salt thereof, in treating or ameliorating Huntington's Disease as a disease-modifying therapy, includes Huntington's disease selected from the group consisting of manifest Huntington's disease, juvenile Huntington's disease, pediatric Huntington's disease, early stage of Huntington's disease, middle stage of Huntington's disease, advanced stage of Huntington's disease, stage I of Huntington's disease, stage II of Huntington's disease, stage Ill of Huntington's disease, stage IV of Huntington's disease, stage V of Huntington's disease, and pre-manifest Huntington's disease.

In one aspect, a tablet containing a therapeutically effective amount of Compound 1, or a pharmaceutically acceptable salt thereof, is administered according to an intermittent dosing schedule.

In another aspect, a tablet containing a therapeutically effective amount of Compound 1, or a pharmaceutically acceptable salt thereof, is administered once a week or twice a week.

In another aspect, a tablet containing a therapeutically effective amount of Compound 1, or a pharmaceutically acceptable salt thereof, is administered orally.

In another aspect, a tablet containing a therapeutically effective amount of Compound 1, or a pharmaceutically acceptable salt thereof, is provided in the form of a pharmaceutical composition.

In another aspect, a tablet containing a therapeutically effective amount of Compound 1, or a pharmaceutically acceptable salt thereof, is provided in the form of a pharmaceutical combination.

In another aspect, a tablet containing a therapeutically effective amount of Compound 1, or a pharmaceutically acceptable salt thereof, is administered following gene therapy or treatment with an antisense compound.

In one aspect, a method of treatment for slowing progression of Huntington's disease in a subject in need thereof, comprising administering to the subject one or more tablets containing a therapeutically effective amount of Compound 1.

In another aspect, a method of treatment for slowing the decline of motor function associated with Huntington's disease in a subject in need thereof, comprising administering to the subject one or more tablets containing a therapeutically effective amount of Compound 1.

In another aspect, a method of treatment for slowing cognitive decline associated with Huntington's disease in a subject in need thereof, comprising administering to the subject one or more tablets containing a therapeutically effective amount of Compound 1.

In another aspect, a method of treatment for slowing psychiatric decline associated with Huntington's disease in a subject in need thereof, comprising administering to said subject one or more tablets containing a therapeutically effective amount of Compound 1.

In another aspect, a method of treatment for slowing the decline of functional capacity associated with Huntington's disease in a subject in need thereof, comprising administering to the subject one or more tablets containing a therapeutically effective amount of Compound 1.

In another aspect, a method of treatment for slowing the progression of Huntington's disease pathophysiology [e.g. reducing the rate of brain (e.g. whole brain, caudate, striatum or cortex) volume loss (e.g. % from baseline volume)] associated with Huntington's disease (e.g. as assessed by MRI)] in a subject in need thereof, comprising administering to the subject one or more tablets containing a therapeutically effective amount of Compound 1.

In another aspect, a method of treatment for slowing the decline of motor function associated with Huntington's disease in a subject in need thereof, comprising administering to the subject one or more tablets containing a therapeutically effective amount of Compound 1; wherein, motor function is selected from the group consisting of ocular motor function, dysarthria, dystonia, chorea, postural stability and gait.

In another aspect, a method of treatment for slowing cognitive decline associated with Huntington's disease in a subject in need thereof, comprising administering to the subject one or more tablets containing a therapeutically effective amount of Compound 1; wherein, cognitive decline is selected from the group consisting of attention, processing speed, visuospatial processing, timing, emotion processing, memory, verbal fluency, psychomotor function, and executive function.

In another aspect, a method of treatment for slowing psychiatric decline associated with Huntington's disease in a subject in need thereof, comprising administering to said subject one or more tablets containing a therapeutically effective amount of Compound 1; wherein, psychiatric decline is selected from the group consisting of apathy, anxiety, depression, obsessive compulsive behavior, suicidal thoughts, irritability, and agitation.

In another aspect, a method of treatment for slowing the decline of functional capacity associated with Huntington's disease in a subject in need thereof, comprising administering to the subject one or more tablets containing a therapeutically effective amount of Compound 1; wherein, functional capacity comprises one or more selected from the group consisting of capacity to work, capacity to handle financial affairs, capacity to manage domestic chores, capacity to perform activities of daily living, and level of care needed.

In another aspect, a method of treatment for slowing the progression of Huntington's disease pathophysiology [e.g. reducing the rate of brain (e.g. whole brain, caudate, striatum or cortex) volume loss (e.g. % from baseline volume)] associated with Huntington's disease (e.g. as assessed by MRI)] in a subject in need thereof, comprising administering to the subject one or more tablets containing a therapeutically effective amount of Compound 1.

In one aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of Compound 1 in a range of from 1 to 200 mg.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of Compound 1 in a range of from 1 to 100 mg.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 1 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 5 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 10 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 15 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 20 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 25 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 30 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 35 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 40 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 45 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of contains 50 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 55 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 60 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 65 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 70 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 75 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 80 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 85 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 90 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 95 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 100 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 105 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 110 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 115 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 120 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 125 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 130 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 135 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 140 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 145 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 150 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 155 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 160 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 165 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 170 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 175 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 180 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 185 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 190 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 195 mg of Compound 1.

In another aspect, a method of treating or ameliorating Huntington's Disease in a subject in need thereof, comprising administering to the subject one or more tablets containing a therapeutically effective amount of Compound 1, or a pharmaceutically acceptable salt thereof, wherein each tablet contains a therapeutically effective amount of 200 mg of Compound 1.

In another aspect, a method of treating or ameliorating Huntington's Disease in a subject in need thereof, comprises administering to the subject one or more tablets containing a therapeutically effective amount of Compound 1, or a pharmaceutically acceptable salt thereof, once per day.

In another aspect, a method of treating or ameliorating Huntington's Disease in a subject in need thereof, comprises administering to the subject one or more tablets containing a therapeutically effective amount of Compound 1, or a pharmaceutically acceptable salt thereof, twice per day.

In another aspect, a method of treating or ameliorating Huntington's Disease in a subject in need thereof, comprises administering to the subject one or more tablets containing a therapeutically effective amount of Compound 1, or a pharmaceutically acceptable salt thereof, three times per day.

In another aspect, a method of treating or ameliorating Huntington's Disease in a subject in need thereof, comprises administering to the subject one or more tablets containing a therapeutically effective amount of Compound 1, or a pharmaceutically acceptable salt thereof, once per week.

In another aspect, a method of treating or ameliorating Huntington's Disease in a subject in need thereof, comprises administering to the subject one or more tablets containing a therapeutically effective amount of Compound 1, or a pharmaceutically acceptable salt thereof, once every two weeks.

In one aspect, a method of treating or ameliorating Huntington's Disease in a subject in need thereof, comprises administering to the subject one or more tablets containing a therapeutically effective amount of Compound 1, or a pharmaceutically acceptable salt thereof, wherein Huntington's disease is selected from the group consisting of Huntington's Disease genetically characterized by CAG repeat expansion of from 36 to 39 in the HIT gene on chromosome 4; and, Huntington's disease genetically characterized by CAG repeat expansion of from >39 in the HTT gene on chromosome 4.

In one aspect, a method of treating or ameliorating Huntington's Disease in a subject in need thereof, comprises administering to the subject one or more tablets containing a therapeutically effective amount of Compound 1, or a pharmaceutically acceptable salt thereof, wherein Huntington's disease is selected from the group consisting of manifest Huntington's disease, juvenile Huntington's disease, pediatric Huntington's disease, early stage of Huntington's disease, middle stage of Huntington's disease, advanced stage of Huntington's disease, stage I of Huntington's disease, stage II of Huntington's disease, stage Ill of Huntington's disease, stage IV of Huntington's disease, stage V of Huntington's disease, and pre-manifest Huntington's disease.

In one aspect, a method of treating or ameliorating Huntington's Disease in a subject in need thereof, comprises administering to the subject one or more tablets containing a therapeutically effective amount of Compound 1, or a pharmaceutically acceptable salt thereof, according to an intermittent dosing schedule.

In another aspect, a method of treating or ameliorating Huntington's Disease in a subject in need thereof, comprises administering to the subject one or more tablets containing a therapeutically effective amount of Compound 1, or a pharmaceutically acceptable salt thereof, once a day, once a week or twice a week.

In another aspect, a method of treating or ameliorating Huntington's Disease in a subject in need thereof, comprises administering to the subject one or more tablets containing a therapeutically effective amount of Compound 1, or a pharmaceutically acceptable salt thereof, orally.

In another aspect, a method of treating or ameliorating Huntington's Disease in a subject in need thereof, comprises administering to the subject one or more tablets containing a therapeutically effective amount of Compound 1, or a pharmaceutically acceptable salt thereof, in the form of a pharmaceutical composition.

In another aspect, a method of treating or ameliorating Huntington's Disease in a subject in need thereof, comprises administering to the subject one or more tablets containing a therapeutically effective amount of Compound 1, or a pharmaceutically acceptable salt thereof, in the form of a pharmaceutical combination.

In another aspect, a method of treating or ameliorating Huntington's Disease in a subject in need thereof, comprises administering to the subject one or more tablets containing a therapeutically effective amount of Compound 1, or a pharmaceutically acceptable salt thereof, following gene therapy or treatment with an antisense compound.

In another aspect, a method of treating or ameliorating Huntington's Disease in a subject in need thereof, comprises administering to the subject one or more tablets containing a therapeutically effective amount of Compound 1, or a pharmaceutically acceptable salt thereof, to produce an inframe stop codon between exons 49 and 50 in the HTT mRNA.

In another aspect, a method of slowing progression of Huntington's Disease in a subject in need thereof, comprises administering to the subject one or more tablets containing a therapeutically effective amount of Compound 1, or a pharmaceutically acceptable salt thereof, to produce an inframe stop codon between exons 49 and 50 in the HTT mRNA.

### VIII. Kits

The term “kit” as used herein refers to a packaged product or article of manufacture comprising components. The kit preferably comprises a box or container that holds the components of the kit. The box or container is affixed with a label or a Food and Drug Administration approved protocol. The box or container holds components of the disclosure which are preferably contained within plastic, polyethylene, polypropylene, ethylene, or propylene vessels. The vessels can be capped tubes or bottles. The kit can also include instructions for use of the reagents.

In another aspect, provided herein are kits comprising, in a container, a Compound (I) described herein, and instructions for use. In some aspects, the kits further comprise a negative control, such as phosphate buffered saline or a Compound (I) that does not recognize an inducible pseudoexon, in a separate container.

In one aspect, the kits further comprise primers and/or antibodies, in one or more separate containers, for assessing the production of an mRNA transcript from a modulated endogenous gene and/or protein production therefrom.

In one aspect, the kits comprise the small molecule 2-[3-(2,2,6,6-tetramethylpiperidin-4-yl)-3H-[1,2,3]triazolo[4,5-c]pyridazin-6-yl]-5-(2H-1,2,3-triazol-2-yl)phenol having the structure of:

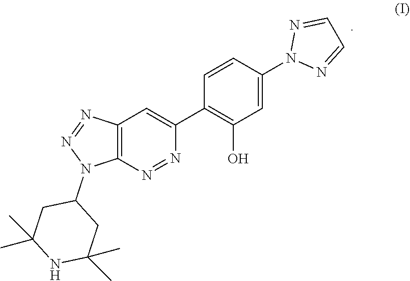 [SMILES: CC1(C)CC(n2nnc3cc(-c4ccc(-n5nccn5)cc4O)nnc32)CC(C)(C)N1]

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EXAMPLES

Examples have been set forth below for the purpose of illustration and to describe certain specific aspects of the disclosure. However, the scope of the claims is not to be in any way limited by the examples set forth herein. Various changes and modifications to the disclosed aspects will be apparent to those skilled in the art and such changes and modifications may be made without departing from the spirit of the disclosure and the scope of the appended claims.

The practice of the disclosure employs, unless otherwise indicated, conventional molecular biological and immunological techniques within the skill of the art. Such techniques are well known to the skilled worker and are explained fully in the literature. See, e.g., Bailey, J. E. and Ollis, D. F., Biochemical Engineering Fundamentals, McGraw-Hill Book Company, NY, 1986; Current Protocols in Immunology, John Wiley & Sons, Inc., NY, N.Y. (1991-2015), including all supplements; Current Protocols in Molecular Biology, John Wiley & Sons, Inc., NY, N.Y. (1987-2015), including all supplements; Sambrook, et al., Molecular Cloning: A Laboratory Manual, 2nd Edition, Cold Spring Harbor, N.Y. (1989); and Harlow and Lane, Antibodies, a Laboratory Manual, Cold Spring Harbor, N.Y. (1989), all the contents of which are incorporated by reference herein in their entireties.

### Example I: Identification of Compounds with Huntingtin Lowering Activity

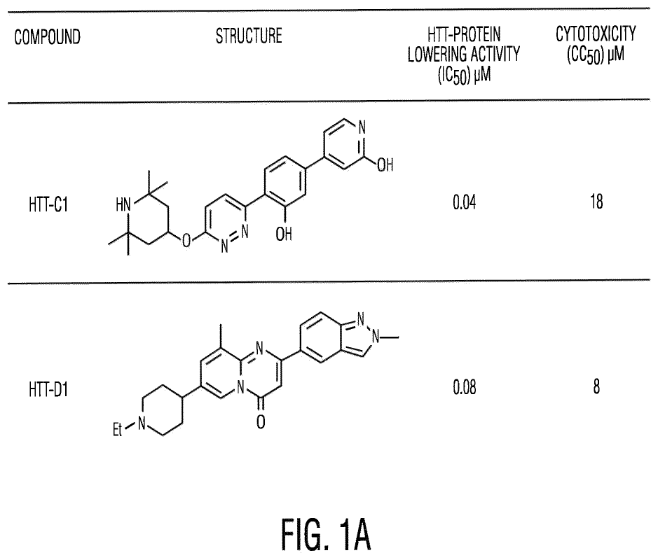

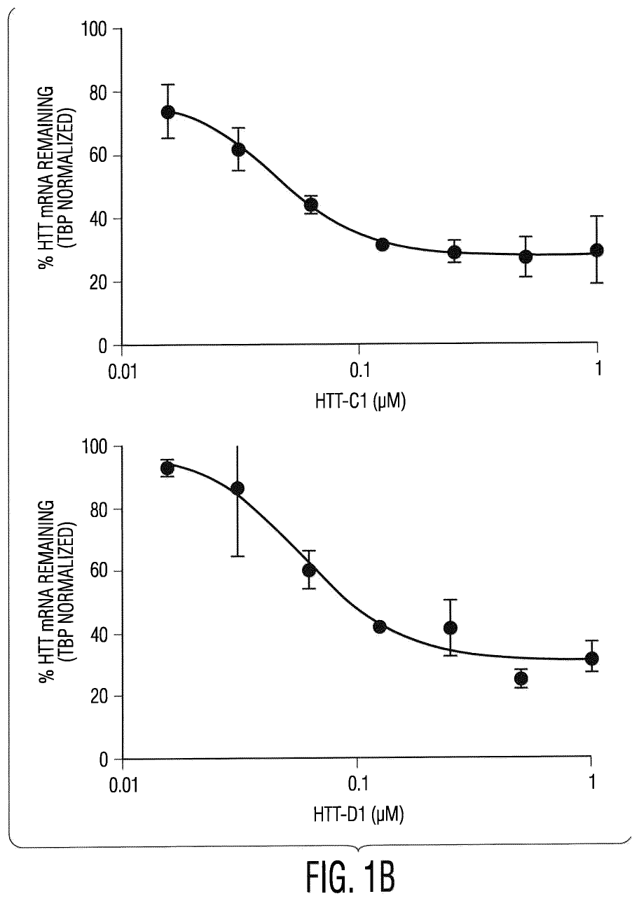

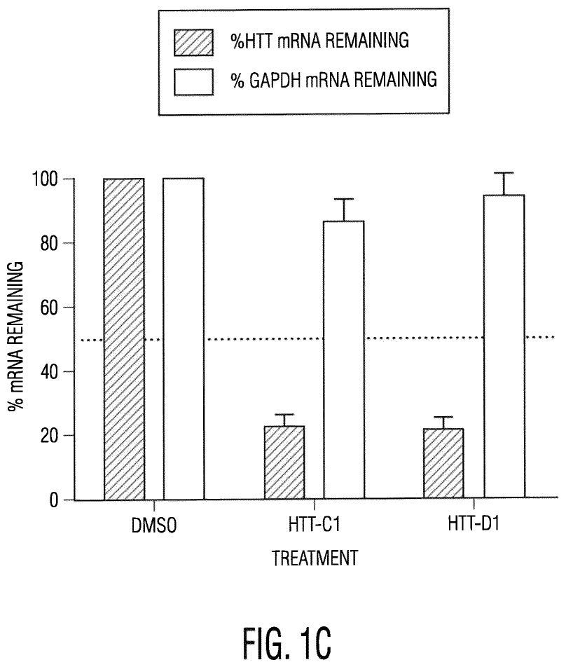

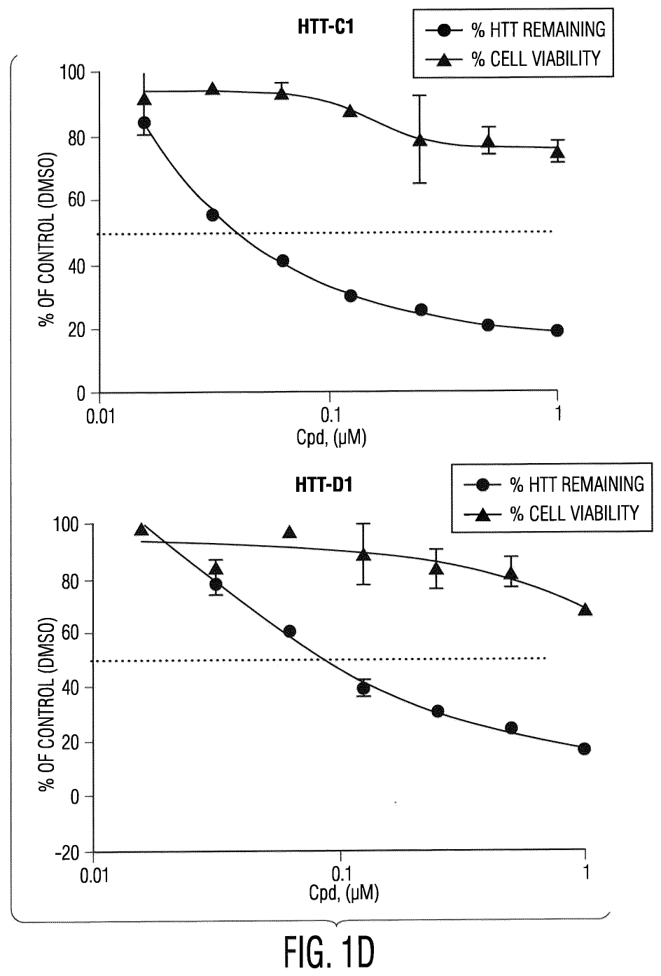

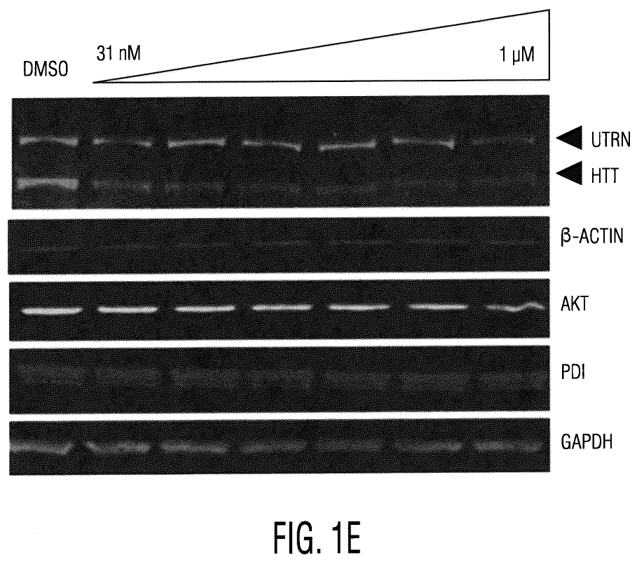

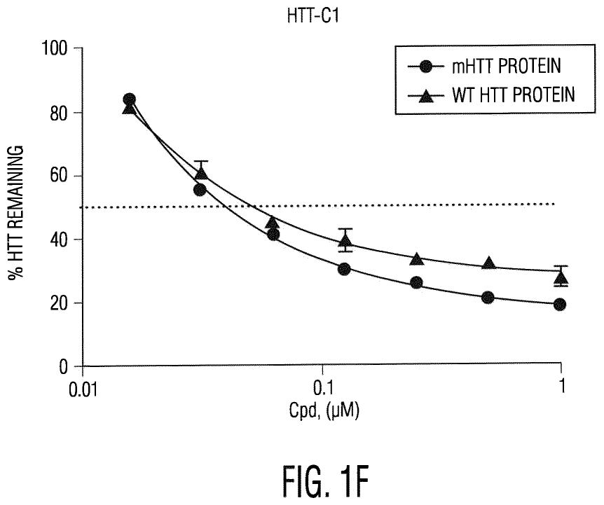

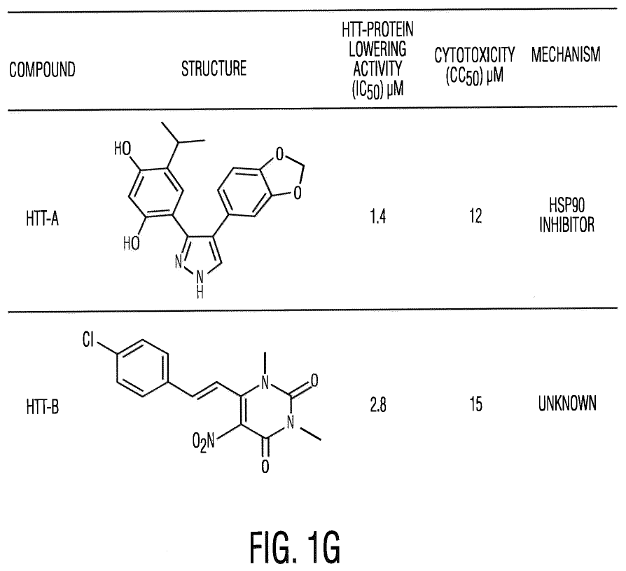

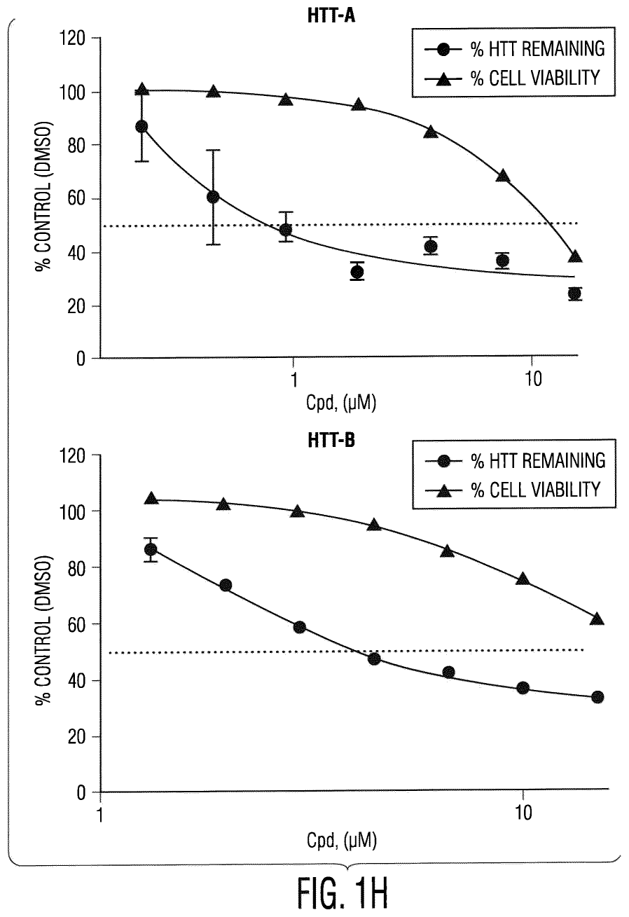

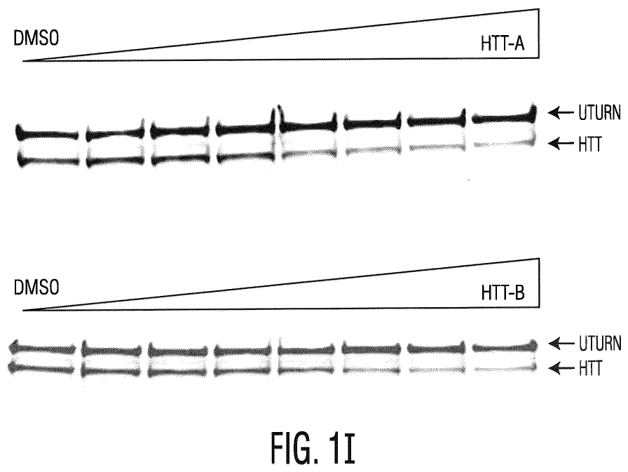

A highly sensitive and robust HTT protein detection assay was developed to screen a proprietary library of ˜300,000 compounds for molecules that can lower the level of HTT protein in fibroblasts derived from patients with HD. Various classes of active compounds (hits) were identified by screening large numbers of diverse chemical compounds. The hits included heat shock protein 90 inhibitors (HTT-A) previously shown to reduce mutant HTT levels (Baldo et al. (2012) J. Biol. Chem. 287, 1406-1414) and HTT-B (FIG. 1G) that were also found to lower HTT protein levels (FIGS. 1H-1I). Of particular interest were compounds belonging to a class of small molecule splicing modifiers, HTT-C1 and HTT-D1 (FIG. 1A), originally discovered in a spinal muscular atrophy drug screen (Naryshkin et al. (2014) Science 345, 688-693; Palacino. et al. (2015) Nat. Chem. Biol. 11, 511-517 (the contents of both publications are hereby incorporated by reference herein in their entireties). These compounds were further tested to determine if they induced a dose-dependent decrease in HTT mRNA and protein levels.

### Cell Cultures

Human B lymphocytes and fibroblasts derived from the same homozygous patient with HD (GM04856/GM04857) and a healthy donor (GM07492/GM07491) (Coriell Institute for Medical Research, Camden, NJ), human neuroblastoma (SH-SY5Y) cells (ATCC®), human embryonic kidney 293 (HEK293) cells (ATCC); Madin-Darby Canine Kidney (MDCK) cells (ATCC); MDCK cells expressing multidrug resistance mutation 1 (MDCK-MDR1) (Absorption Systems); mouse CT26 cells (ATCC) were cultured at 37° C. in a humidified 5% CO2 atmosphere. Fibroblasts were maintained in Dulbecco's Modified Eagle's Medium (DMEM) with 10% (v/v) fetal bovine serum (Thermo Fisher Scientific) and 1% penicillin-streptomycin (Thermo Fisher Scientific).

### High Throughput Library Screening

Human fibroblasts derived from a homozygous patient with HD (GM4857) were cultured for 96 hours in the presence of test compounds (in 0.5% DMSO) or controls at 37° C. in a humidified 5% C02 atmosphere. After 96 hours, cells were lysed and frozen. HTT protein levels were measured in lysates as described below. Compounds that decreased HTT protein levels relative to DMSO control were further tested in a dose response assay.

### Quantification of HTT Protein

For analysis in the electrochemiluminescence (ECL) assays, test compounds were serially diluted 3-fold in 100/DMSO (Sigma) to generate a 7-point concentration curve. A solution of test compound (500 nL, 200× in DMSO) was added to each test well with Acoustic Transfer System (EDC Biosystems); final concentration of DMSO was 0.5%. Fibroblasts were seeded in 96-well flat-bottomed plates (Thermo Fisher) at 4×10³ cells/well in 100 μl of culture medium containing the test compound or DMSO vehicle control and incubated for 96 hours (37° C., 5% CO2, 100% relative humidity). After removal of the supernatant, cells were lysed in 50 μL of 1×LB11 extraction buffer (50 mM Tris (pH 7.4), 300 mM NaCl, 10% [w/v] glycerol, 3 mM EDTA, 1 mM MgCl2, 20 mM glycerophosphate, 25 mM NaF, 1% Triton X-100), containing a Complete™ protease inhibitor cocktail (Roche Diagnostics) with shaking at 4° C. for 30 minutes; the plates were then stored at −20° C.

### Electrochemiluminescence Protein Assay

Meso Scale Discovery® 96-well plates (MSD®) were coated overnight at 4° C. with primary antibodies in phosphate-buffered saline (PBS; 30 μl/well). The plates were washed three times with 0.05% Tween-20 in 1×PBS (PBS-T; 200 μl/well) then blocked (100 μl/well; 5% bovine serum albumin [BSA] in PBS-T) for 5-6 hours at room temperature (RT) with shaking. Plates were then washed three times with PBS-T. Cell lysates were transferred to the antibody coated plates (25 μl/well) and incubated with shaking overnight at 4° C. After removal of the lysates, the plates were washed three times with PBS-T, and 25 μl of detection antibody in 1% BSA, PBS-T was added to each well and incubated with shaking for 1 hour at room temperature. After three washes with PBS-T, 25 μl of Sulfo-Tag secondary antibody (MSD®; 0.25 μg/ml in 1% BSA, PBS-T) was added to each well and incubated with shaking for 1 hour at RT. After washing three times with PBS-T, 150 μl of read buffer T with surfactant (MSD®) was added to each empty well and the plate was imaged on the SI 6000 imager (MSD®) according to manufacturers' instructions for 96-well plates. Primary capture antibodies included: anti-polyglutamine-expanded HTT mouse monoclonal antibody (mAb) clone MW1 (1 μg/mL; Developmental Studies Hybridoma Bank); anti-HTT MAB2166 mAb (1 sg/mL; Millipore); anti-human KRAS rabbit polyclonal antibody (1 pg/mL; Thermo Fisher Scientific). Detection antibodies included: Huntingtin (D7F7) XP® Rabbit mAb (0.25 μg/ml; Cell Signalling Technology®); anti-hKRAS mouse mAb (0.25 μg/ml; LSBIO).

### Western Blot Analysis

For western blot analysis, the fibroblast cell line GM04857 (from CCR) were plated at 5×10⁴ cells/well in 1 mL 10% FBS/DMEM with GlutaMAX™ supplement (Thermo Fisher) in 24-well plates (Thermo Fisher) and incubated for 3-4 hours (37° C., 5% CO2, 100% relative humidity). Cells were treated with test compounds at different concentrations (0.5% DMSO) in triplicate wells for 96 hours. Cells were then lysed in 75 μL Laemmli buffer (Bio-Rad Laboratories, Inc.). Lysates could then be frozen.

Samples were mixed with loading buffer, boiled for 10 min and 45 ul was loaded per well and electrophoresed in a 3-8% Tris-Acetate gel @ 130V for 5-6 hrs. (12+2 wells; Invitrogen™ NuPAGE™ 3 to 8%, Tris-Acetate, 1.0 mm, Midi Protein Gel, 12+2-well). The protein MW ladder used was Invitrogen Himark HMW ladder (10 uL per well). Following electrophoresis, protein was electro transferred to a 0.45 FM Nitrocellulose membrane (Bio-Rad) at 150 mA for 90 min and then incubated with Li-Cor Blocking buffer overnight at 4° C. with agitation.

Antibodies used: anti-HTT (Millipore, cat. #AB2166; dilution: 1:1000), anti-UTRN (Vector Laboratories, cat. #VP-U579; dilution: 1:250), anti-PDI (Santa Cruz, cat. #SC20132; dilution 1:10,000), anti-β-actin (Sigma, cat. #A2228; dilution: 1:10,000), anti-GAPDH (Thermo Fisher, cat. #PAI-987; dilution: 1:1000), anti-AKT (Cell Signaling, cat. #9272; dilution: 1:1000).

Secondary antibodies used: Alexa Fluor® 680 goat anti-mouse IgG (Thermo Fisher Scientific) and IRDye® 800CW donkey anti-Rabbit IgG (LI-COR; dilution 1:10,000)+0.1% Tween at RT for 1 hr. Blots were washed 4-5 min in PBS-T, washed again in PBS-T for 10 minutes and rinsed in PBS prior to scanning in an Odyssey Imager.

Quantification of HTT mRNA

Test compounds were serially diluted 3-fold in 100% DMSO to generate a 7-point concentration curve. A solution of test compound (500 nl, 200× in DMSO) was added to each test well with Acoustic Transfer System. Fibroblasts were seeded in 96-well flat-bottomed plates (Thermo Fisher Scientific) at 1×10⁴ cells/well in 100 μl of culture medium containing the test compound or DMSO vehicle control and incubated for 24 hours (37° C., 5% CO2, 100% relative humidity). After removal of the supernatant, cells were lysed in RNA lysis buffer (1 M Tris-HCL pH 7.4, 5M NaCl, 10% IGEPAL®CA-630; 50 μL/well) for 1 minute at RT, before 50 μL of chilled nuclease free water was added to each well; plates were then transferred immediately onto ice before storing at −80° C. overnight.

RT-qPCR Quantification of HTT mRNA in Cells

Cell lysates were assayed by quantitative reverse transcriptase polymerase chain reaction (RT-qPCR) to measure mRNA levels of HTT and glyceraldehyde 3-phosphate dehydrogenase (GAPDH) in the presence or absence of the test compound. TaqMan-based RT-qPCR primers and probes (Thermo Fisher Scientific) are shown in TABLE III.

A reverse transcriptase quantitative polymerase chain reaction (RT-qPCR) reaction mixture of primers and probe sets for HTT and GAPDH was prepared according to TABLE III. RNA samples were transferred (2 μL/well) to an Armadillo 384-Well PCR plate (Thermo Fisher Scientific) containing 8 μL/well of the AgPath-ID™ one-step RT-PCR reaction mixture (Thermo Fisher Scientific) in a final volume of 20 μL (see TABLE III). The plate was then sealed with MicroAmp™ Optical Adhesive Film (Thermo Fisher Scientific) and placed in the CFX384 Touch™ Real-Time PCR thermocycler (Bio-Rad Laboratories, Inc.). RT-qPCR was carried out at the following temperatures for indicated times: Step 1: 48° C. (30 min); Step 2: 95° C. (10 min); Step 3: 95° C. (15 sec); Step 4: 60° C. (1 min); then, repeated Steps 3 and 4 for a total of 40 cycles

TABLE III
PREPARATION OF PCR MIX

| | | | SEQ | Final |
|---|---|---|---|---|
| Assay | Primer/ | | ID | concen- |
| mix | probe | Sequence | NO.: | tration |
| HTT | Forward | Thermo Fisher | Not publicly | |
| | primer | Scientific | available | |
| | Reverse | Hs00918174_ml | | |
| | primer | (Catalog # 4331182) | | |
| | | Taqman Gene | | |
| | | Expression: 60X | | |
| GAPDH | Forward | CAACGGATTTGGTCGTATTGG | 1 | 100 nM |
| | primer | | | |
| | Reverse | TGATGGCAACAATATCCACTT | 2 | 100 nM |
| | primer | TACC | | |
| | Probe | CGCCTGGTCACCAGGGCTGCT | 3 | 75 nM |

HTT, Huntingtin;
GAPDH, glyceraldehyde 3-phosphate dehydrogenase

| Reagent | Volume (μL) | Final Concentration |
|---|---|---|
| RT-PCR buffer (2X) | 5 | 1X |
| RT-PCR enzyme mixture (25X) | 0.4 | 1X |
| HTT Primer/Probe (60X) | 0.16667 | 1X |
| GAPDH assay (20X) | 0.5 | 1X |
| H₂O | 1.94 | — |

### Compounds Having HIT Lowering Activity

The library screen identified the following compounds as having HTT protein and mRNA lowering activity are described in TABLE IV below.

TABLE IV
CHEMICAL STRUCTURES OF COMPOUNDS WITH HTT LOWERING
ACTIVITY

| COMPOUND | STRUCTURE | NET MDR1 ER | AVERAGE KP, UU |
|---|---|---|---|
| HTT-A | 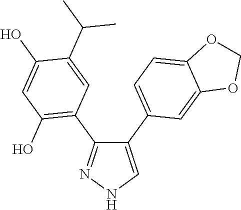 [SMILES: CC(C)c1cc(-c2n[nH]cc2-c2ccc3c(c2)OCO3)c(O)cc1O] | — | — |
| HTT-B | 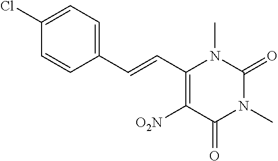 [SMILES: Cn1c(/C=C/c2ccc(Cl)cc2)c([N+](=O)[O-])c(=O)n(C)c1=O] | — | — |
| HTT-C1 | 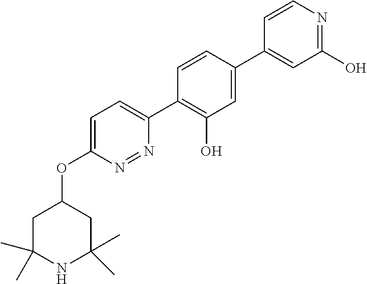 [SMILES: CC1(C)CC(Oc2ccc(-c3ccc(-c4ccnc(O)c4)cc3O)nn2)CC(C)(C)N1] | >62.7 | 0.084 |
| HTT-C2 | 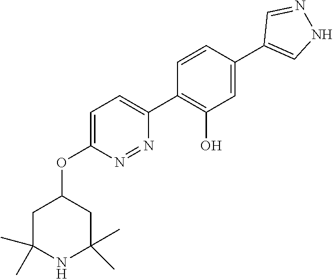 [SMILES: CC1(C)CC(Oc2ccc(-c3ccc(-c4cn[nH]c4)cc3O)nn2)CC(C)(C)N1] | 25.3 | 0.179 |
| HTT-C3 | 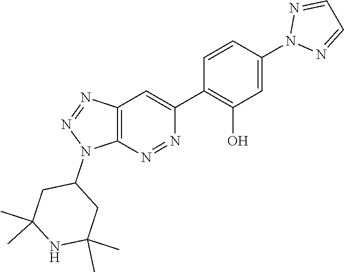 [SMILES: CC1(C)CC(n2nnc3cc(-c4ccc(-n5nccn5)cc4O)nnc32)CC(C)(C)N1] | 2.0 | 0.848 |
| HTT-D1 | 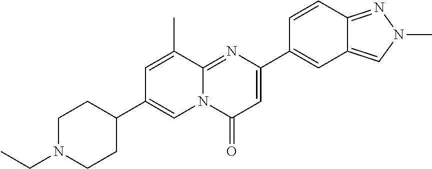 [SMILES: CCN1CCC(c2cc(C)c3nc(-c4ccc5nn(C)cc5c4)cc(=O)n3c2)CC1] | 11.8 | 0.242 |
| HTT-D2 | 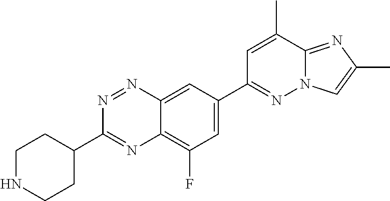 [SMILES: Cc1cn2nc(-c3cc(F)c4nc(C5CCNCC5)nnc4c3)cc(C)c2n1] | 21.8 | 0.089 |
| HTT-D3 | 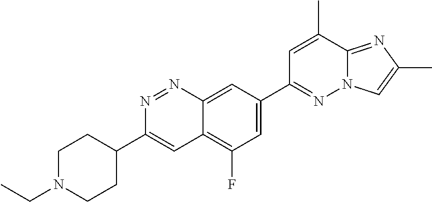 [SMILES: CCN1CCC(c2cc3c(F)cc(-c4cc(C)c5nc(C)cn5n4)cc3nn2)CC1] | 7.10 | 0.472 |
| RG7916 | 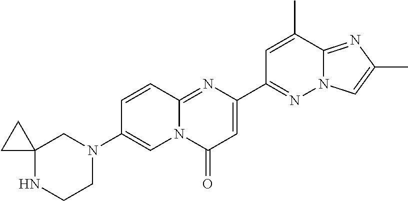 | | |

The compounds HTT-C1 and HIT-D1 (FIG. 1A) were tested to determine if they induced a dose-dependent decrease in HTT expression. Both HTT-C1 and HTT-D1 induced a dose-dependent decrease in the amount of HTT mRNA (FIGS. 1B-1C) and HTT protein (FIGS. 1D-1E) in fibroblasts derived from HD patients. HTT-C1 lowered HTT protein expression from both alleles in a dose dependent manner, i.e., both wild type and mutant HTT protein gene expression (see FIG. 1F). HTT-C1 had no effect on mouse HTT protein expression at concentrations that reduce human HTT protein expression.

### Example II: HTT-C1 Modifies the Splicing of Human HTT mRNA

To determine if the compound HTT-C1 altered the splicing of HTT pre-mRNA, primer walking was first used to evaluate all HTT splice junctions.

### Primer Walking Assay and Endpoint RT-PCR Analysis

B lymphocytes (GM04856 cells) were plated in 6-well plates at 5×10⁵ cells/well in 2 mL of 10% FBS, DMEM and incubated for 6 hours (37° C., 5% CO2, 100% relative humidity). Cells were then treated with HTT-C1 at 125 nM (in 0.5% DMSO) in triplicate for 24 hours. RNA was purified with the RNeasy Mini Kit (Qiagen) according to the manufacturer's protocol. Samples were prepared for RT-PCR (described previously) using 0.04 μL of each primer (at 100 μM). For reverse transcription and PCR, the following steps were performed: RT step: 48° C. (15 min); PCR steps: Step 1: 95° C. (10 min), Step 2: 95° C. (30 sec), Step 3: 55° C. (30 sec), Step 4: 68° C. (1 min); Steps 2 to 4 were repeated for 34 cycles, then held at 4° C. PCR products were separated on 2% agarose E-gels, stained with ethidium bromide and visualized using a UVP gel imager.

Primer sets used for primer walking can be found in TABLE V below:

TABLE V
PRIMERS USED IN HTT PRIMER WALKING ASSAY

| SEQ | | SEQ | | | EXPECTED |
|---|---|---|---|---|---|
| ID | | ID | | HTT EXON | AMPLICON |
| NO: | FORWARD PRIMER | NO: | REVERSE PRIMER | BOUNDARY | SIZE (bp*) |
| 14 | CGGCTGTGGCTGAGGAG | 15 | CCAAGGTCTCCTGGACTGAT | Exon 1-Exon 6 | 453 |
| 16 | CTGGATCAGCAGTGAGCATCT | 17 | TTGAAAGGACAGGGCTGCAT | Exon 7-Exon 10 | 498 |
| 18 | TGACTCTGAATCGAGATCGGATGT | 19 | CAGAAGGCTGCCTGCAGT | Exon 11-Exon 14 | 563 |
| 20 | GACTCTGCACCTCTTGTCCATT | 21 | CTGTTCCTCAGAGTCAGCACAT | Exon 15-Exon 19 | 548 |
| 22 | GGTGAGCTTTTTGGAGGCAAA | 23 | GGTCAGAATCATTGTGGCCATC | Exon 20-Exon 25 | 580 |
| 24 | ACCTGCTGAAGGTGATTAACATTTGT | 25 | GGGTTGGAAGATAAGCCATCAAA | Exon 26-Exon 31 | 574 |
| 26 | CAGAAAGTGTCTACCCAGTTGAAGA | 27 | AGACAGTCGCTTCCACTTGTC | Exon 32-Exon 37 | 640 |
| 28 | TCCGTCCGGTAGACATGCT | 29 | AAGTCAGAATCCTCCTCTTCTCC | Exon 38-Exon 42 | 709 |
| 30 | CAGCGGCCTGTTCATCCA | 31 | CAGAAATTTCACTCATCCCTAGG | Exon 43-Exon 48 | 625 |
| | | | CTTA | | |
| 32 | TGCCCAGTCATTTGCACCTT | 33 | TCTCCTCCTGCTCCATCA | Exon 49-Exon 54 | 756 |
| 34 | CCAGCTGTAAGCTGCTTGGA | 35 | GTGCACCCTTCGCAGTTC | Exon 55-Exon 60 | 630 |
| 36 | CACTGCCAAGCAGCTCATC | 37 | GTTGGAGAGGGACAGCATGAC | Exon 61-Exon 66 | 736 |

*bp: base pairs

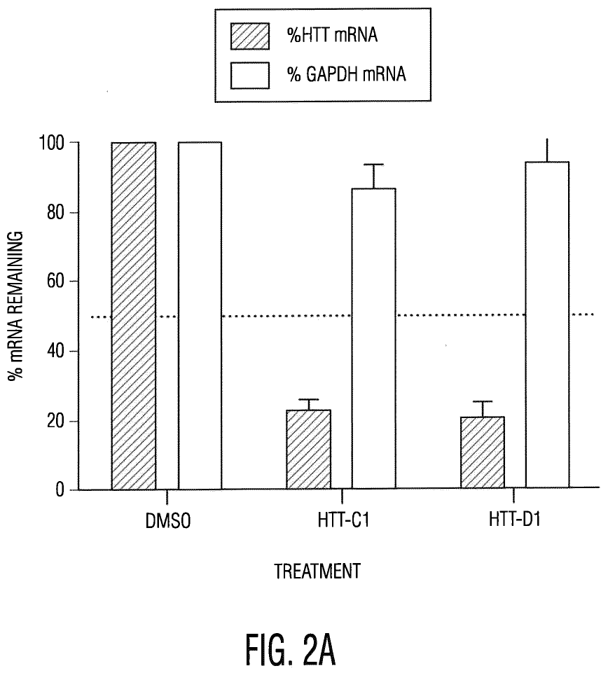

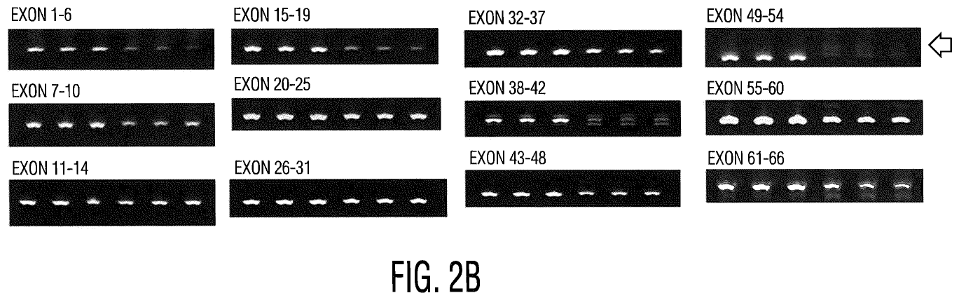

As shown in FIG. 2A, treatment of B lymphocytes (GM04856 cells) derived from HD patients with 125 nM HTT-C1 or HTT-D1 compound significantly reduced the amount of HTT mRNA as compared to a DMSO control. In parallel, total RNA from the HTT-C1 treated B lymphocytes was also probed by primer walking to determine if the observed decrease in HTT mRNA was due to an alteration in splicing of the HTT pre-mRNA. The RT-PCR analysis of FIG. 2B demonstrated that HTT-C1 induced differential splicing between exons 49-54 (see arrow).

### Targeted Next-Generation Sequencing

Novel splicing events originating from HTT pre-mRNA upon compound treatment were further analyzed using AmpliSeq technology. The GM04856 cells were plated in 6-well plates at 500,000 cells/well in 2000 μl DMEM/10% FBS and incubated for 6 hours in a cell culture incubator (37° C., 5% CO2, 100% relative humidity). Cells were then treated in triplicate with HTT-C1 at 125 nM (in 0.5% DMSO) for 24 hours. Following treatment, RNA was purified using an RNeasy Mini Kit (Qiagen) according to the manufacturer's protocol. Novel splice variants induced by the HTT-C1 compound were detected using Ion AmpliSeq technology (Life Technologies), a PCR-based target enrichment and next-generation sequencing platform. PCR enrichment of HTT exon targets was accomplished by applying a custom HTT AmpliSeq panel. The panel consisted of two separate PCR primer pools, each producing 33 amplicons. The complete HTT assay had 66 amplicons (mean size, 135 bp) covering all 67 exons of the HTT gene. The Ampliseq workflow included: a) RNA reverse transcription, b) target amplification, c) partial primer digestion, d) adapter ligation, e) library amplification, f) sequencing and finally, g) sequencing data analysis (see FIG. 3A).

For data analysis, Ampliseq reads (Fastq format) were mapped to human genome (hg19) using tophat2 which allowed identification of both known and novel splice junctions. For each one of the 66 introns of HTT gene, a Junction Expression Index (JEI) was calculated using the percent of reads supporting the splicing of the exact annotated intron among all reads supporting the splicing isoforms using either the 5′ splice or/and 3′ splice site of that intron (FIG. 3B). A JEI value of 100% indicates full splicing of the intron. A JEI value less than 100% indicates alternative splicing paths exist (e.g., inclusion of a cryptic exon or use of alternative 5′ or 3′ splice sites). Samples were tested in triplicate for 125 nM HTT-C1 or DMSO treatment group and compared using the Student's t-test. A decrease of JEI between DMSO and compound treated samples of >5% and a T-test P-value<0.05 indicated a significant change in HTT splicing.

Consistent with the primer walking data, subsequent Ion Ampliseq™ (Life Technologies) analysis of all 66 introns of the full-length HTT transcript, found that the junction expression index (JEI) in intron 49 was significantly reduced (>25%; P<0.05, see FIGS. 3Cii, 3Di-ii and FIG. 3H). These results demonstrate that Compound (I) induced a splicing event that resulted in the inclusion of a novel exon within intron 49 of the HTT pre-mRNA.

Potential 5′ and 3′ splice sites within intron 49 were evaluated using MaxEntScan. This program models the sequences of short sequence motifs such as those involved in RNA splicing while simultaneously accounting for non-adjacent as well as adjacent dependencies between positions. This method is based on the ‘Maximum Entropy Principle’ and generalizes most previous probabilistic models of sequence motifs such as weight matrix models and inhomogeneous Markov models (publicly available at on the web site of the Burge lab at MIT; hollywood.mit.edu/burgelab/maxent/Xmaxentscan_scoreseq).

The identified intronic exon is not conserved across species, and contrary to the known exons 49 and 50, it has a weak 5′ splice site (MaxEnt scores <6; FIG. 3E), and multiple alternate 3′ splice sites. Furthermore, the 5′ splice site is noncanonical with the nucleotide sequence, guanine (G) adenine (A), at the −2 to −1 position, i.e., distinct from the canonical AG sequence. Thus, the small molecule-induced exonic sequence is a pseudoexon (psiExon). Ampliseq analysis of FIG. 3E identified two HTT-C1 inducible pseudoexon sequences having 115 and 146 nucleotides in length ((SEQ ID NOs: 46 and 49, respectively).

This analysis also demonstrated that the several candidate splice modifiers (HTT-C1, HTT-C2, HTT-C3, and HTT-D3) induced the inclusion of an Intron 49 pseudoexon in a variety of different cell types (HD fibroblasts, SH-SY5Y (ATCC® CRL-2266™) neuroblastoma cells, TK6 (ATCC® CRL-8015™) lymphoblast cells and MRC-5 (ATCC® CCL-171™) fibroblasts (FIGS. 3F-3H). In FIG. 3J, cells treated with HTT-C2 or HTT-C3 reduced HTT gene expression as quantified using RNAseq analysis. The normalized gene expression values are reported as Fragment Per Kb per Million total reads (FPKM). P-values are based on two tailed Student's t-test.

As shown in FIG. 4C, the small molecules induced splicing of the pseudoexon to the Exon 49 caused a frameshift mutation that introduced premature stop codons downstream of Exon 49. Splicing to the 115 nucleotide pseudoexon 49a-1 (SEQ ID NO: 46) results in two premature stop codons within the pseudoexon 49a-1 (see FIG. 4Bi), whereas splicing of the 146 nucleotide pseudoexon 49a-2 (SEQ ID NO: 49) results in a premature stop codon, not in the pseudoexon, but further downstream in Exon 50 (FIG. 4Ei-ii). These frameshift mutations in the HTT mRNA triggers the observed degradation of HTT mRNA through translation-linked mRNA decay and a commensurate reduction in HTT protein levels.

In one example, the noncanonical GAgu signature nucleotides at the HTT pseudoexon 49a-1 5′ splice site is reminiscent of the 5′ splice site sequence of the SMN exon 7 that was recently shown to function in the presence of the splicing modulator risdiplam (Ratni et al., (2018) J. Med. Chem. 9; 61(15):6501-6517).

### Example III: Selectivity of Small Molecule-Induced Splicing

RNA-Seq Library Preparation from SHY5Y and U) Transfected HEK293 Cells

SHY5Y cells were seeded in 6-well plates at 6×10⁵ cells/well in 2 mL 10% FBS, DMEM and incubated for 4 hours. Cells were then treated with two biological replicates of HT-C1 at 24 nM or 100 nM (in 0.1% DMSO), or four biological replicates of vehicle control (DMSO) for 24 hours (37° C., 5% CO2, 100% relative humidity). HEK293 cells transfected with U1 snRNA minigene constructs treated with either duplicates of 1 μM HTT-C1 or 0.5% DMSO control and incubated for 48 hours (37° C., 5% CO2, 100% relative humidity).

Total RNA was extracted from the treated SHY5Y cells and U1 snRNA minigene transfected HEK293 cells using the RNeasy plus mini kit. RNA concentration and quality were assessed using a NanoDrop spectrophotometer (ThermoFisher Scientific).

For library preparation and sequencing, mRNA was enriched from about 3 μg of total RNA using oligo(dT) beads (ThermoFisher Scientific). The mRNA was fragmented randomly using fragmentation buffer followed by cDNA synthesis using the mRNA template and random hexamers primer. Second-strand synthesis buffer (Illumina), deoxynucleotides, ribonuclease H and DNA polymerase I were added to initiate second-strand synthesis. After a series of terminal repair, A-ligation and sequencing adaptor ligation, the double-stranded cDNA library was size selected and enriched by PCR. RNA libraries were sequenced in a HiSeq sequencer (Illumina).

### RNA-Seq Analysis

RNA sequencing reads were mapped to human genome (hg19) using Spliced Transcripts Alignment to a Reference (STAR) software (version 2.5) (Dobin et L., (2013) Bioinformatics 1; 29(1):15-21). Uniquely mapped reads (with MAPQ>10) having <5 nt/100 nt mismatches were used for analysis.

For gene expression analysis, the number of reads in a coding sequence (CDS) region of protein-coding genes and exonic regions of non-coding genes were counted and analyzed using DESeq2 (Love et al., (2014) Genome Biology, 15:550). For splicing analysis, reads were counted for different exons or exonic regions. For each exon, a Percent-Spliced-In (PSI) value was calculated using the percent of average read number supporting the inclusion of the exon (include both the upstream and downstream junctions) among all reads supporting either the inclusion or the exclusion of an exon. A minimal of 20 for the denominator of PSI calculation was required. Otherwise a “NA” value would be generated. PSI values for biological replicates were averaged and the PSI difference between two treatment groups was calculated. For statistical test, a 2×2 read counts table was made for each exon with rows for reads supporting inclusion or exclusion and columns for the two comparing sample groups (biological replicates were combined). Fisher's Exact Test was used for statistical test. PSI change of >20% (or <−20%) and P-value <0.001 was used to select exons being regulated by the treatment.

k-Mer Analysis

For comparing sequence difference of a particular region for two groups of exons (e.g., UP vs. NC), the k-mer (k=4 to 6) frequencies of the two groups were compared by Fisher's Exact Test (one k-mer vs. all other k-mers, group 1 vs. group 2). The resulting P-value was converted to a significance score (SS=−S×Log 10 P-value), in which S is the sign indicating enrichment (1) or depletion (−1) of the k-mer in group 1.

### Sequence Logo

Sequence logos were generated using WebLogo.

### Analysis of the RNA-Seq Library

To investigate if small molecules can modify splicing of other genes, RNA-seq analysis of transcriptome changes was analyzed in human SH-SY5Y cells treated with a close analog of HTT-C1, HTT-C2 (24 nM and 100 nM) or control (0.1% DMSO) (FIG. 5Ai-ii). HTT-C2 (24 nM; ˜2× the IC₅₀) selectively and potently downregulated expression of HTT demonstrating the compound's selectivity for HTT splicing. Downregulation of the expression of other genes by HTT-C2 was accentuated as the concentration of HTT-C increased (100 nM HTT-C2 (˜10×IC50), suggesting a dose-dependent effect.

The RNA-seq data from SHSY5Y cells treated with HTT-C2 (24 nM and 100 nM) or control (0.1% DMSO) were further analyzed to determine if HTT-C2 modified the splicing of these other mRNA targets in a similar manner as with HTT pre-mRNA, i.e. through the inclusion of an intronic pseudoexon (psiExon). HTT-C2 treatment altered 165 and 215 splicing events in the 24 nM and 100 nM treatment groups, respectively. Most of the regulated alternative splicing events were cassette exons (CE) (FIG. 5Bi), with the majority exon inclusion events (up regulation of an exon or exonic region ([UP]) representing most changes.

For example, 100 nM HTT-C2 induced 3.3 times more UP events than exon skipping events (down regulation of an exon or exonic region [DN]) (FIG. 5Bii). Since the HTT pseudoexon 49a-1 is a novel exon without any public transcript database annotations of the 5′ splice site and 3′ splice site, the other compound-regulated UP CEs were reviewed for annotations and found that 22% and 44% of the UP CEs in the 24 nM and 100 nM treatment groups, respectively, had no annotations for at least one of the splice sites, representing additional novel pseudoexon (psiExon) splicing events (FIG. 5Biii-iv). Thirty-one pseudoexons were induced at either concentration of HTT-C2. 15 pseudoexon (psiExon) inclusion events were tested and all of them were validated using endpoint RT-PCR (FIG. 5Ci-ii). These 31 pseudoexons (psiExon) demonstrate an extremely low basal inclusion rate (median percent spliced in index [PSI]: 0.7%) compared with annotated exons that were unaffected by compound (FIG. 5D), indicating that they are not spliced in normal conditions. Located within intronic regions with low sequence conservation (FIG. 5E), the pseudoexons' (psiExon) lengths are shorter (median size of 64 base pairs) with significantly weaker 5′ splice sites than annotated unaffected exons (FIG. 5Fi-ii). Like with HTT small molecule induced splicing, these pseudoexon-containing genes were significantly downregulated in HTT-C2 treated cells (P<0.05) because of premature termination codons or frameshifts introduced by pseudoexon inclusion (FIG. 5G).

### Analysis of Nonsense-Mediated Decay

To test if the small molecule-induced spliced transcripts are unstable due to nonsense-mediated decay (NMD), GM04856 lymphoblast cells were treated with cycloheximide (CHX). First cells were treated with DMSO or 250 nM HTT-C1 for 18 h. DMSO or 10 uM CHX was then added, and cells harvested for RNAs after 2 h, 4 h, and Sh. Compound treatment results in ˜80% reduction in HTT mRNA (measured by RT-qPCR) as shown in FIG. 5H.

### Sequence Specificity

HTT pseudoexon 49a-1 has a 5′ splice site sequence GAguaag, in which GA is at the −2 to −1 position (FIG. 6Ai). Sequence logo and k-mer analysis confirmed a significant enrichment of GA sequence for exons activated by HTT-C2 (FIG. 6Aii). Additionally, the sequence motif also demonstrates enrichment of 5′ splice sites with A at the −3 and +3 position; represented by the enriched 5′ splice site AGAguaag. This differs from risdiplam data, which identifies GGAguaag as the sequence motif indicating differences in the target sequence preference of the HTT class of splicing modifiers such as HTT-C2.

### U1-GA Variant Recruitment to Noncanonical 5′Splice Sites

To understand if HTT splicing modifiers function to stabilize U1 interaction with 5′ splice sites, HEK293 cells were transfected with a variant U1 snRNA (U1-GA variant; FIG. 6Bi-ii) that forms a strong base pairing with noncanonical 5′ splice sites (FIG. 6Ci-ii). HTT-C1 induced HTT mRNA splicing was then compared to mock-transfected cells using RNAseq analysis.

Wild-type and mutant HTT and U1-GA snRNA minigene constructs (FIG. 6Bi-ii) were synthesized at GenScript®. For U1-GA snRNA constructs, 5×10⁶ HEK293 cells were transfected with 2 μg of plasmid DNA or mock control in 6-well plates, using 6 μl Fugene6® (Promega) according to the manufacturer's instructions; after incubating for 24 hours (37° C., 5% CO2, 100% relative humidity), cells were treated with either 1 μM HTT-C1 or 0.5% DMSO control and incubated for 48 hours. For HTT constructs, 5×10⁶ HEK293 cells were transfected with 50 ng of plasmid DNA in 24-well plates, using 6 μl Fugene6® according to the manufacturer's instructions. After incubating overnight (37° C., 5% CO₂, 100% relative humidity), cells were treated with varying concentrations of compounds in a final concentration 0.05% DMSO and incubated for 24 hours.

Of the 27 pseudoexons activated by HTT-C2, 24 were also activated by the U1-GA variant and displayed a strong AGA sequence feature at the −3 to −1 position of the 5′ splice site. 582 pseudoexons were only activated by the U1-GA variant and not by HTT-C2 (FIG. 6D). These pseudoexons have a strong preference for GA at −2 to −1 position at 5′ splice sites, but do not show any preference for A at the −3 or +3 position. These data indicate that both HTT-C2 and variant U1-GA can enhance U1 recruitment at 5′ splice sites having a GA at −2 to −1 position and demonstrate the specificity of HTT-C2 for sequences with −3 A sequence. In conclusion, HTT-C2 activates a set of pseudoexons with preference for AGAguaag 5′ splice site sequences and triggers target gene downregulation by the nonsense-mediated decay (NMD) pathway.

### Example IV: Exonic Enhancers are Required for Pseudoexon Inclusion

In view of the limited number of pseudoexon inclusion events identified by treatment with either HTT-C2 or the U1-GA variant (n=700; FIG. 6C), a search for putative pseudoexons in the genome and specifically within the HTT gene was initiated based on the presence of a noncanonical GAgu 5′ splice site and a 3′ splice site within approximately 150 nucleotides upstream of the 5′ splice site.

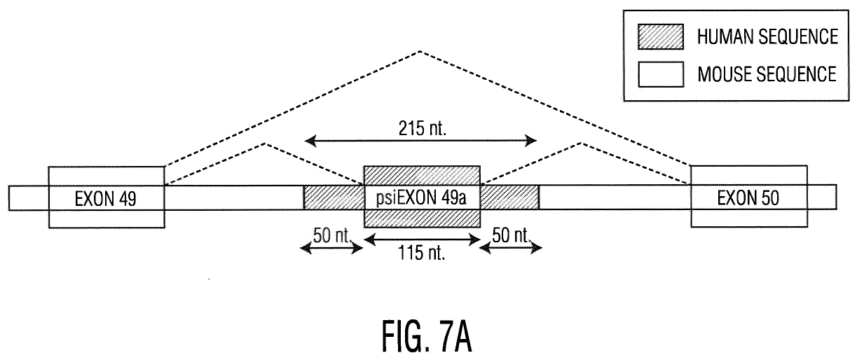

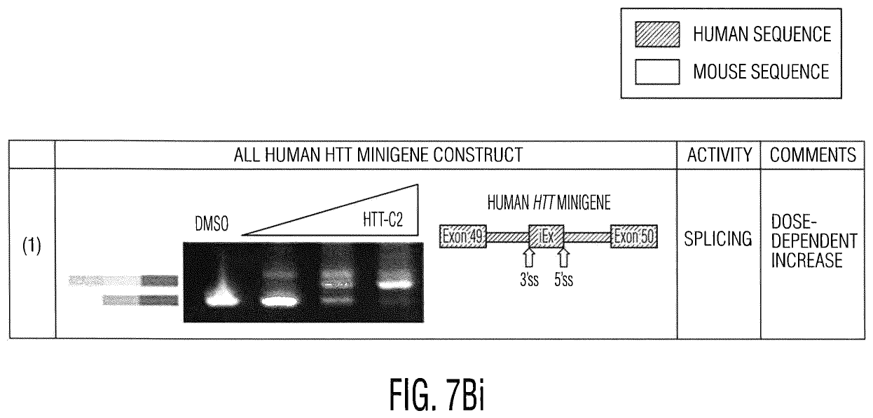

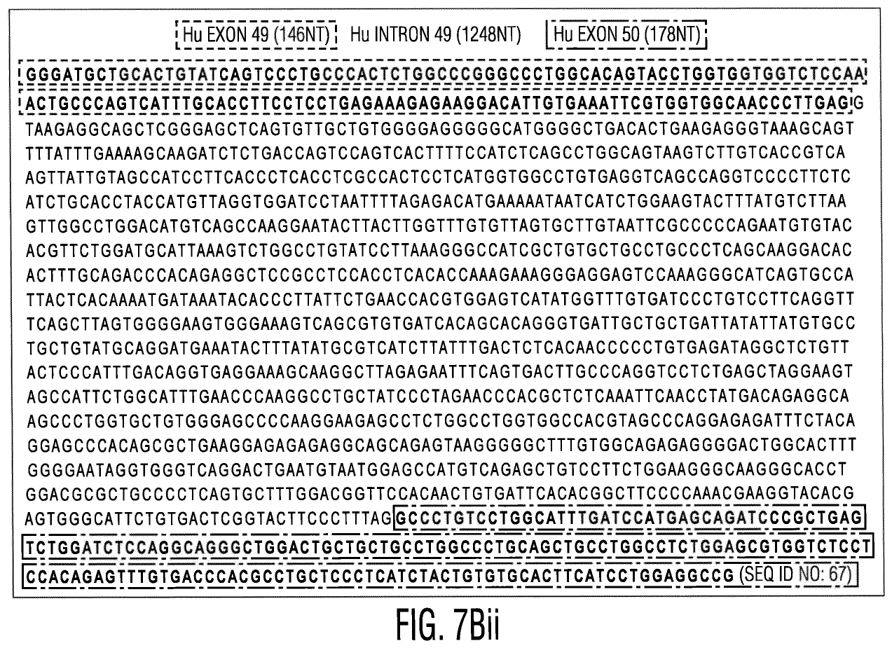

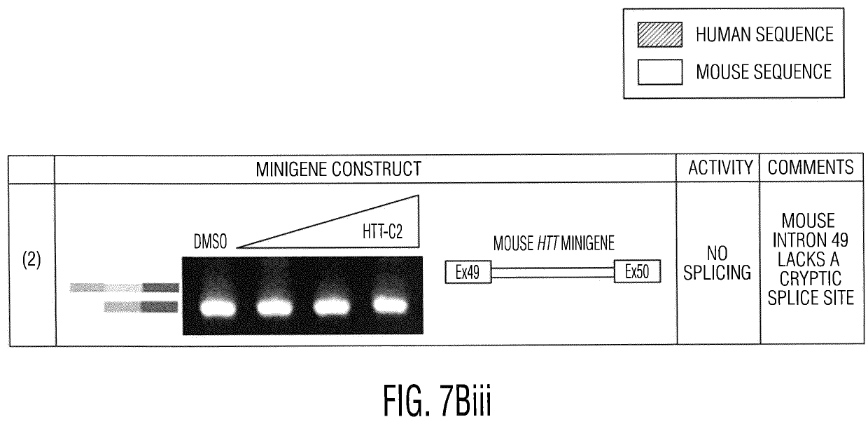

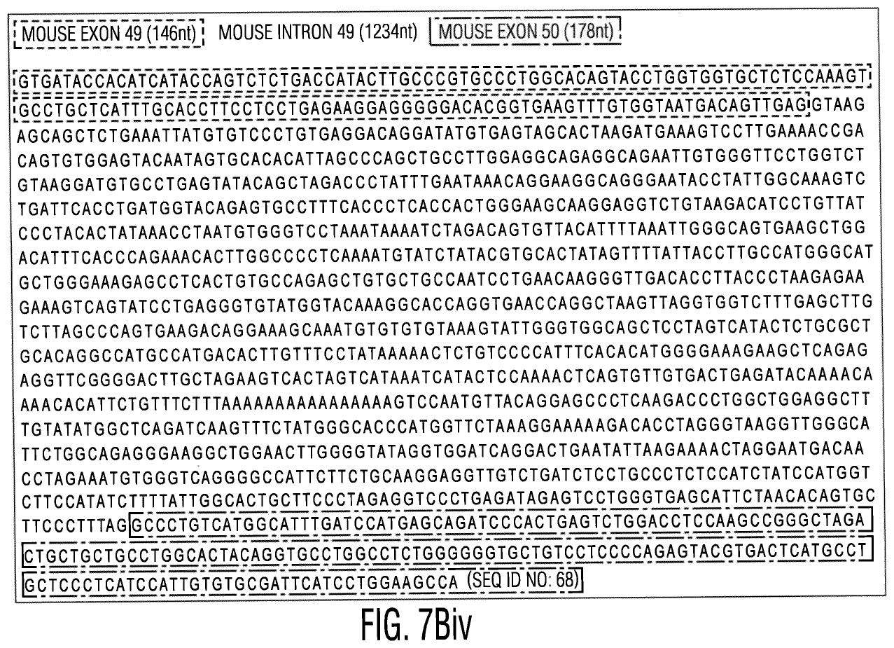

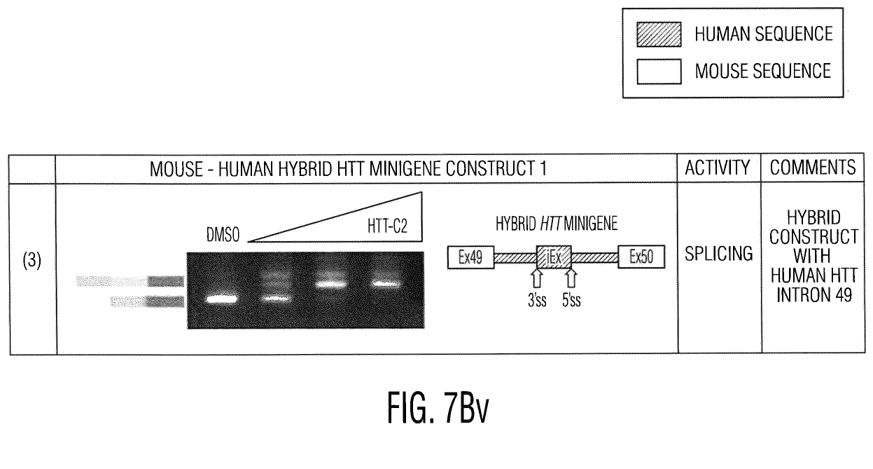

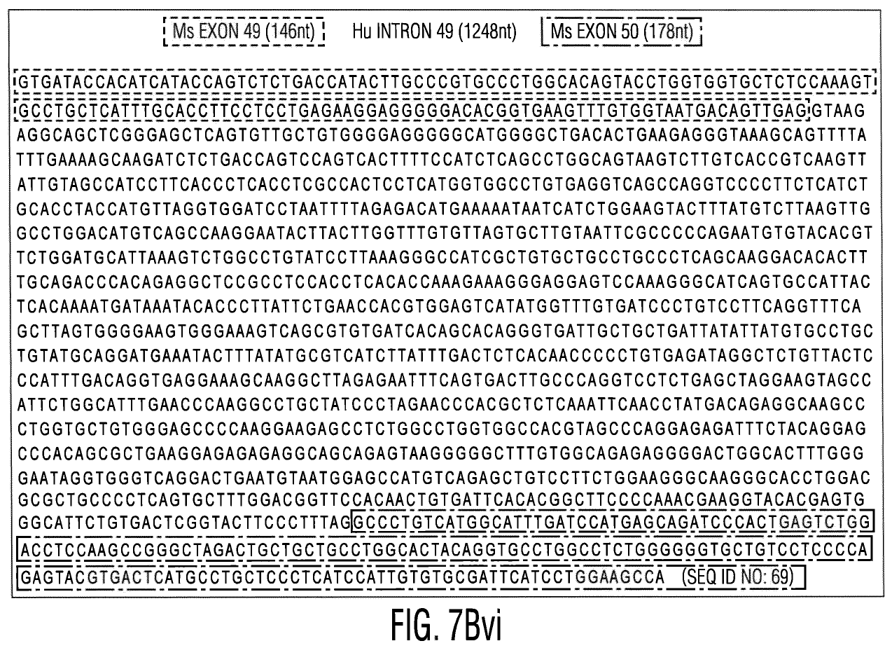

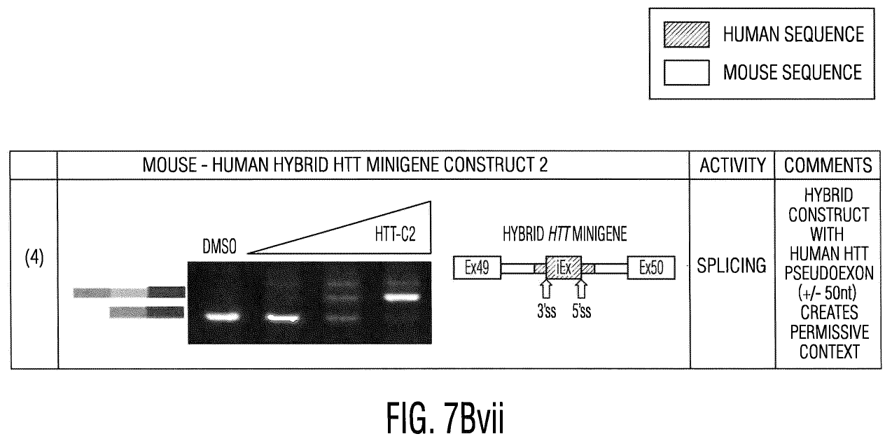

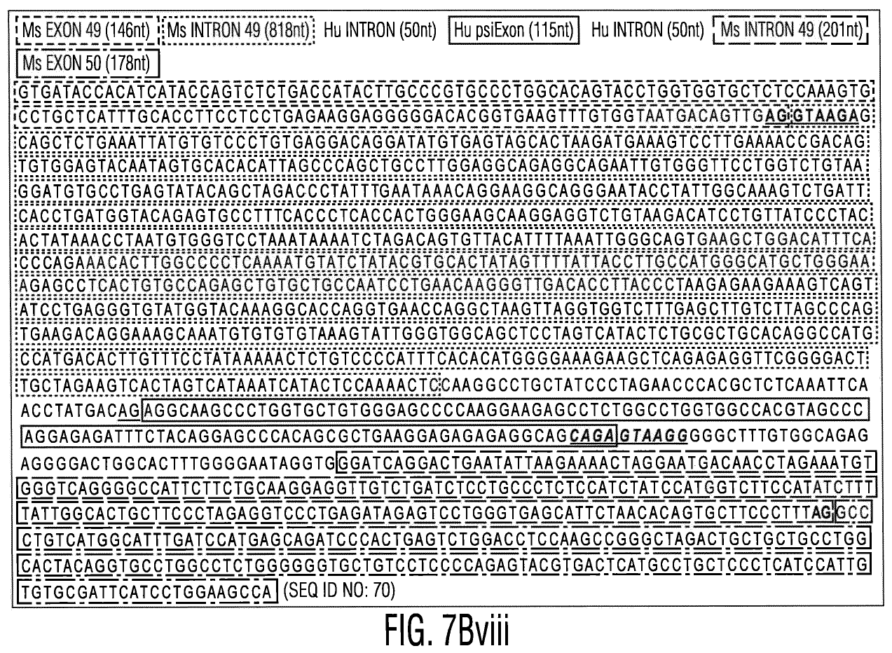

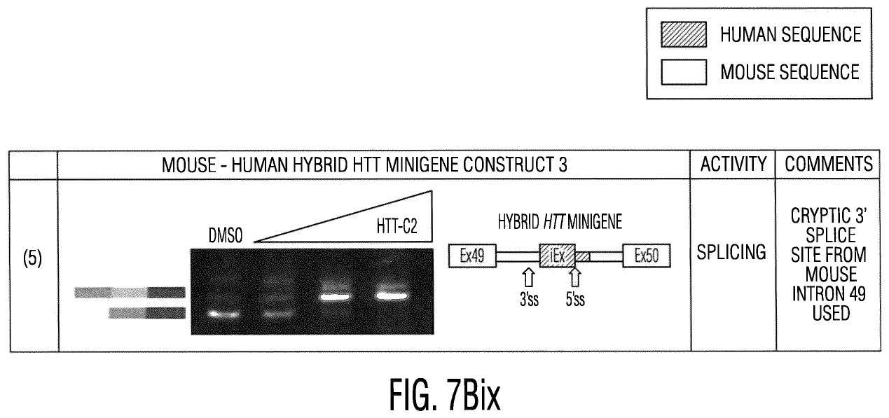

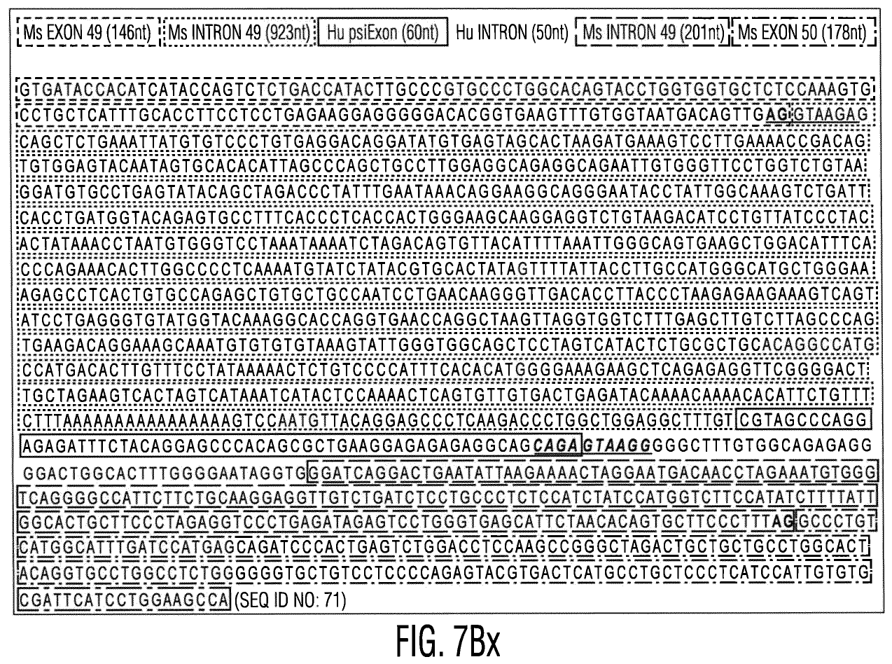

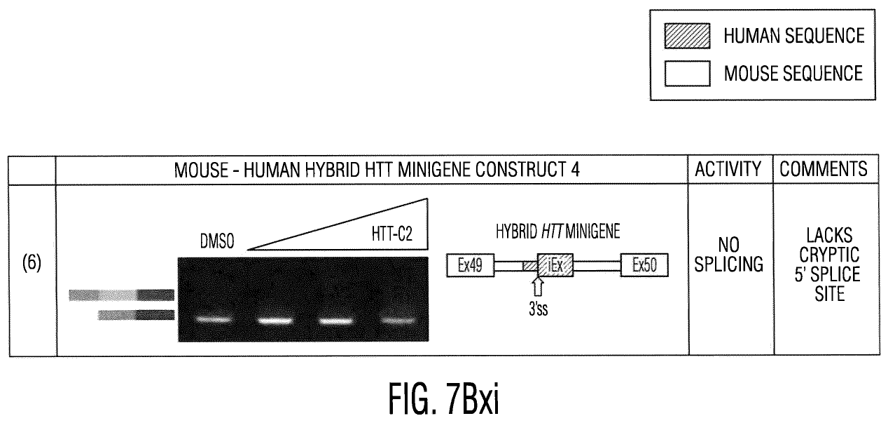

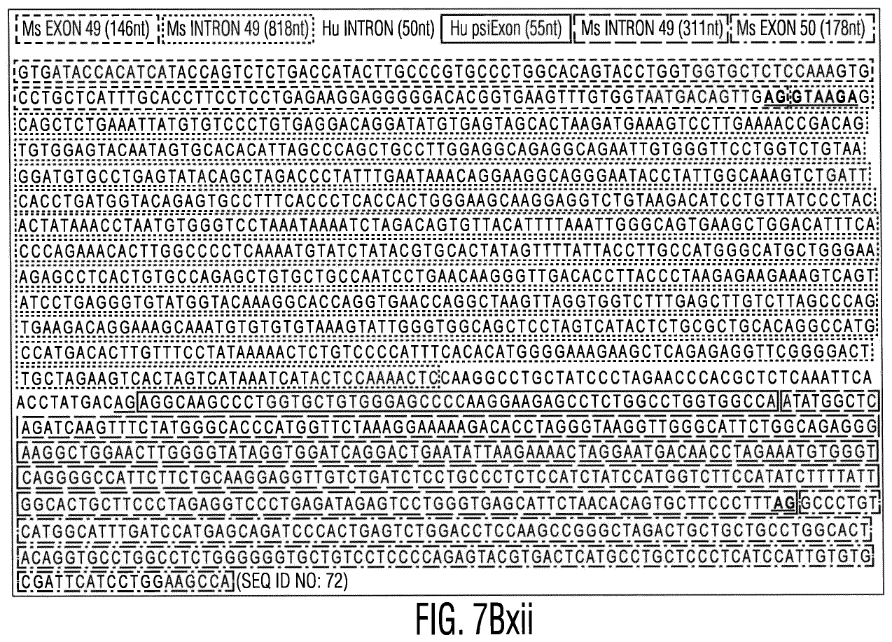

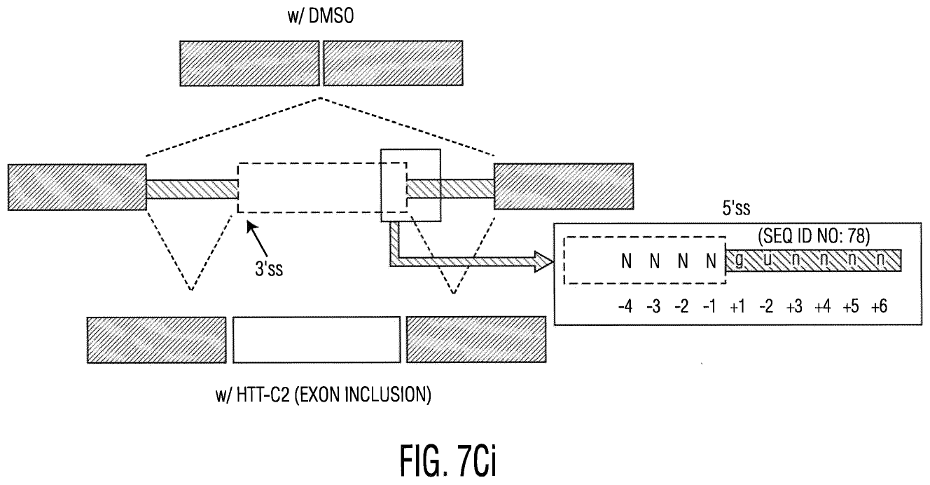

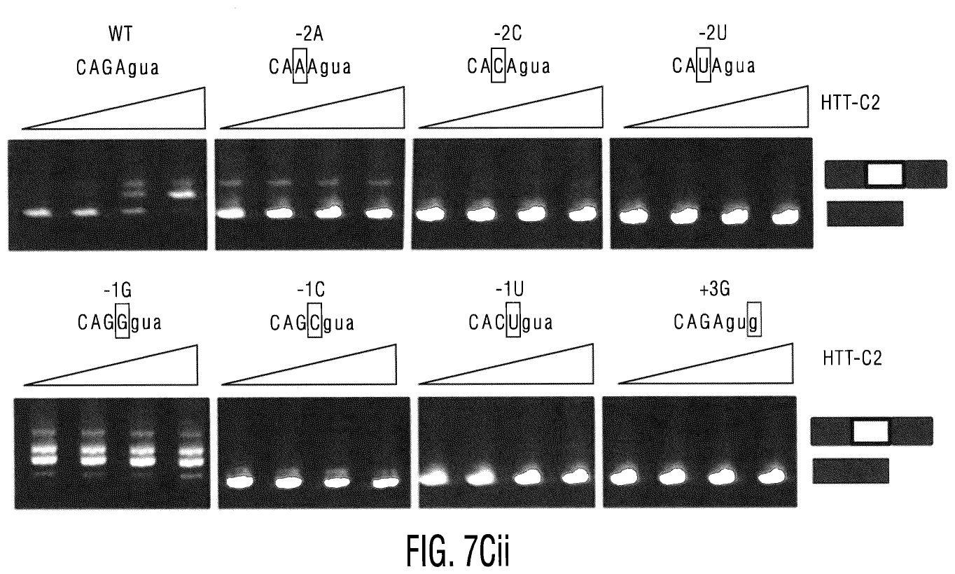

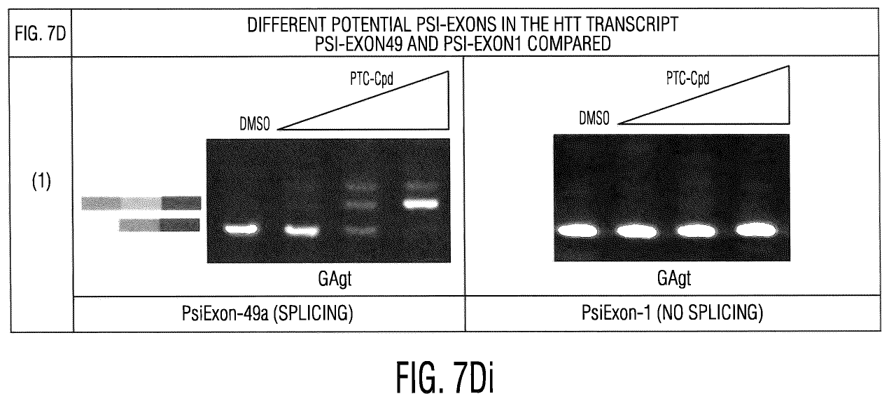

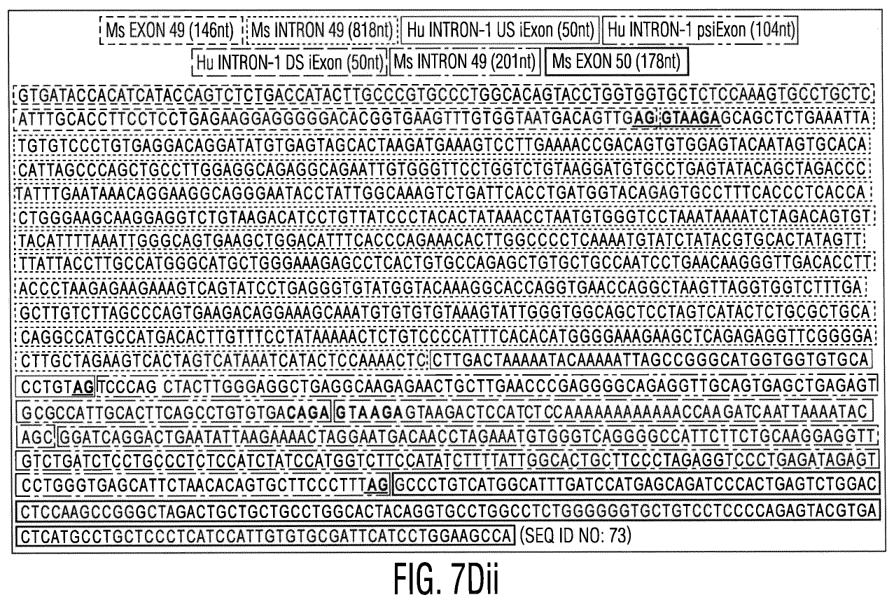

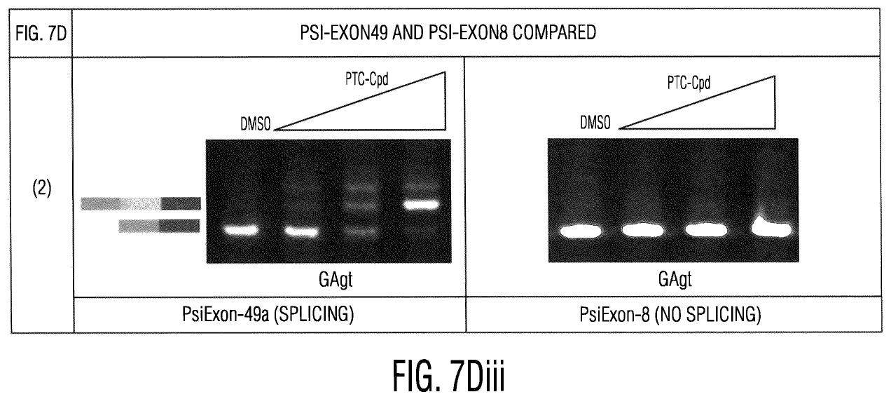

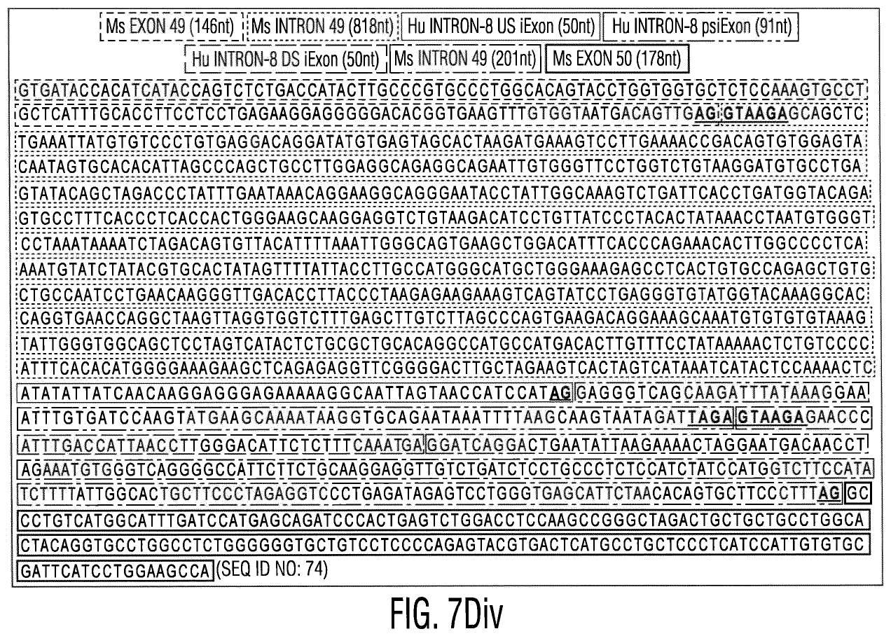

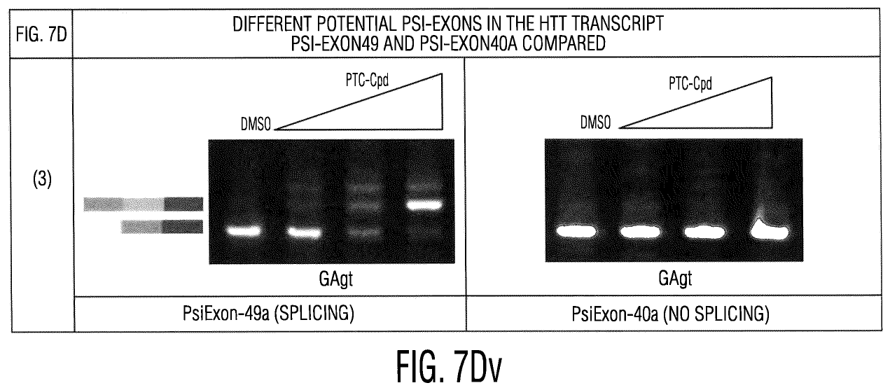

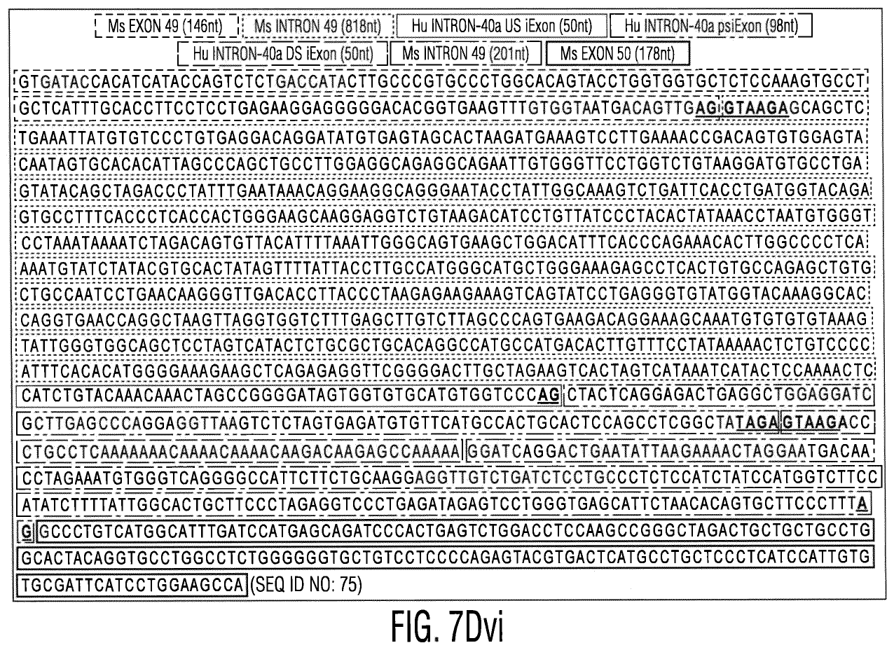

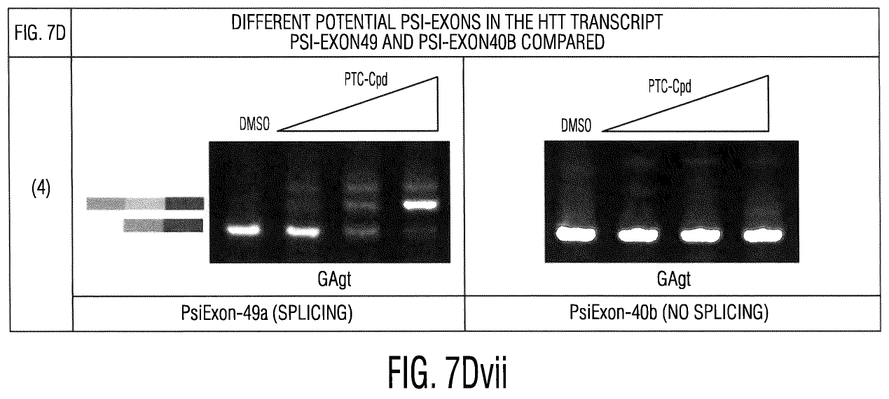

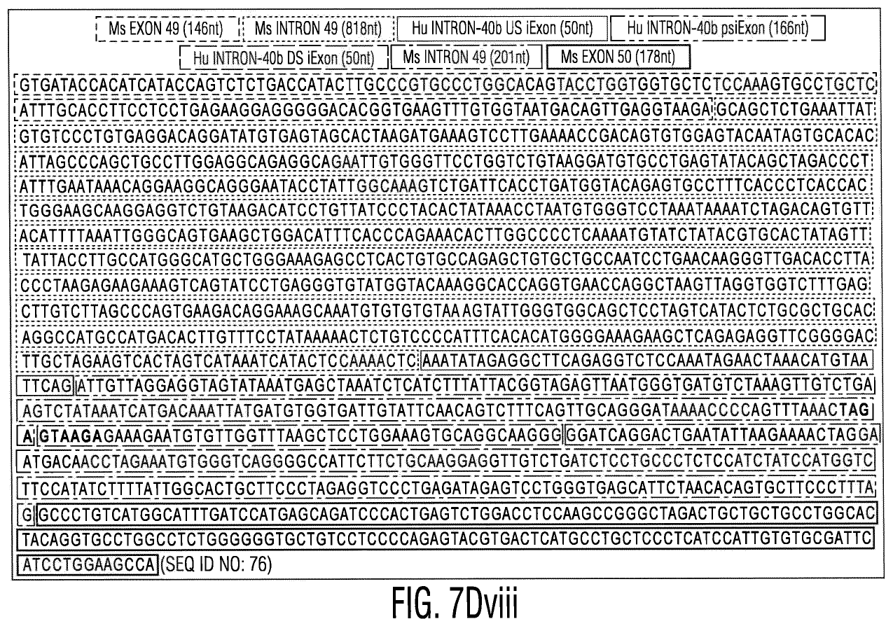

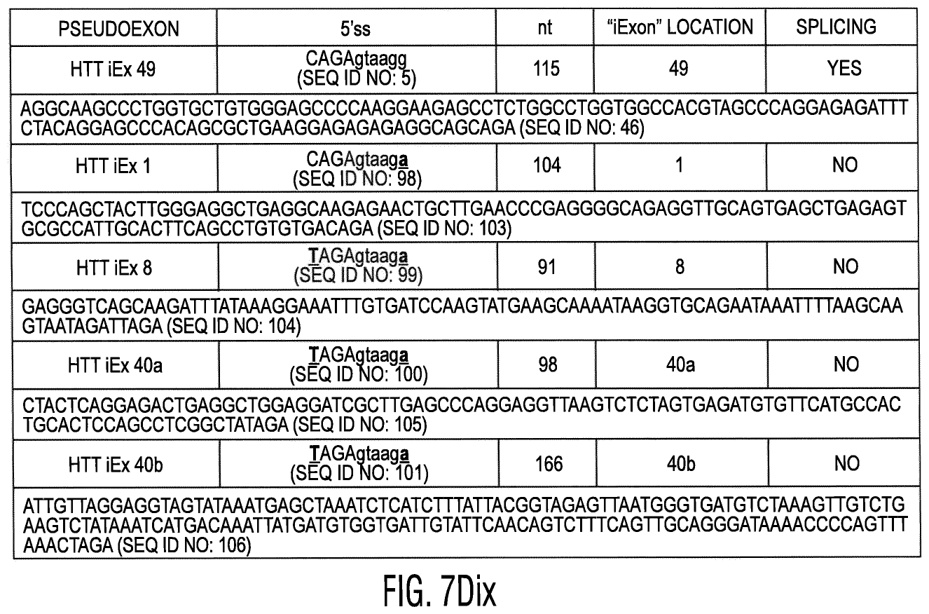

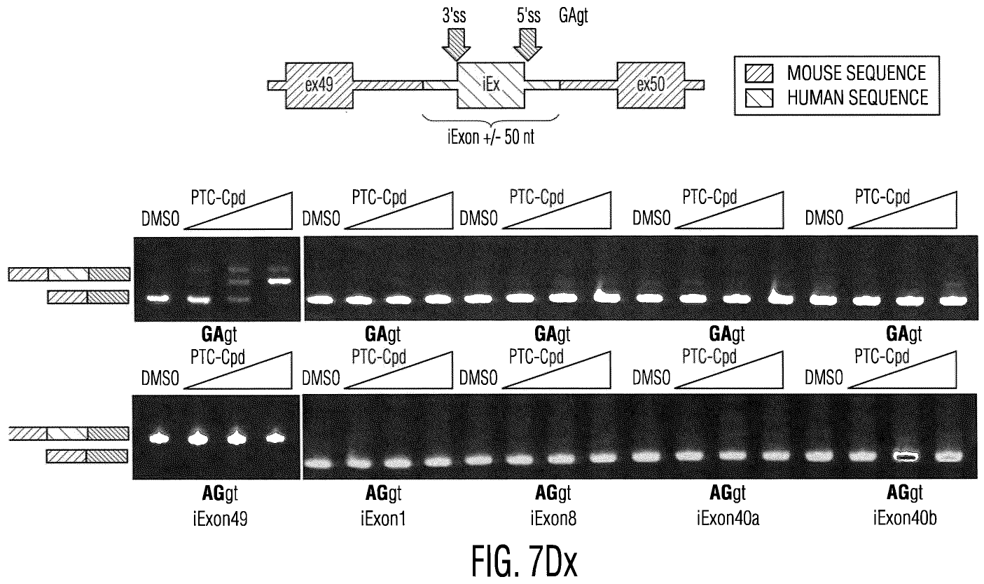

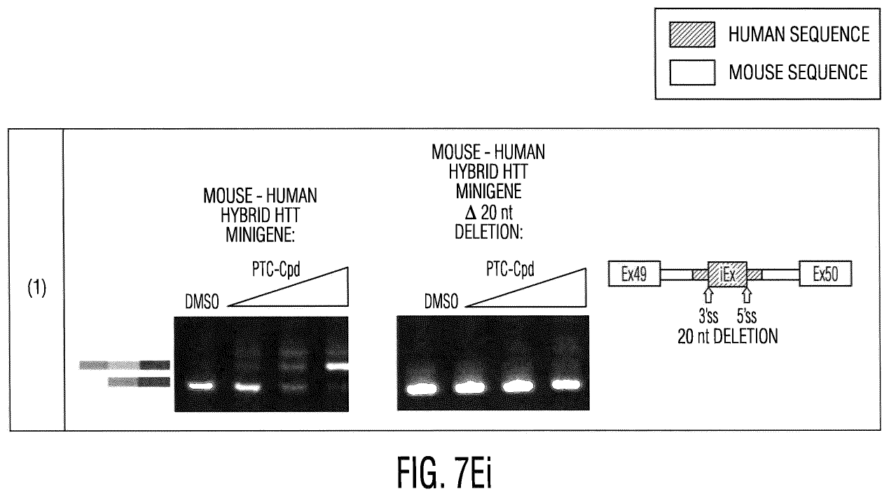

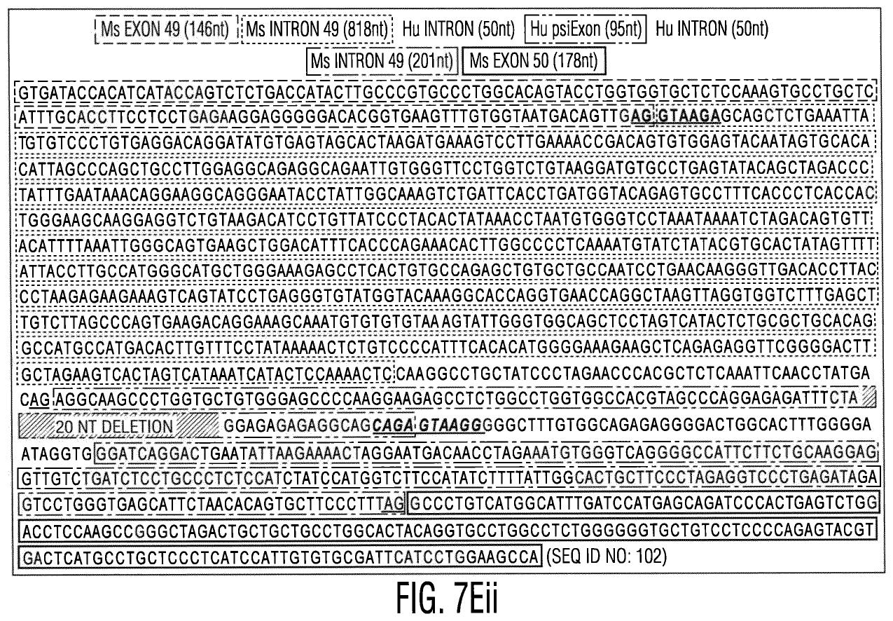

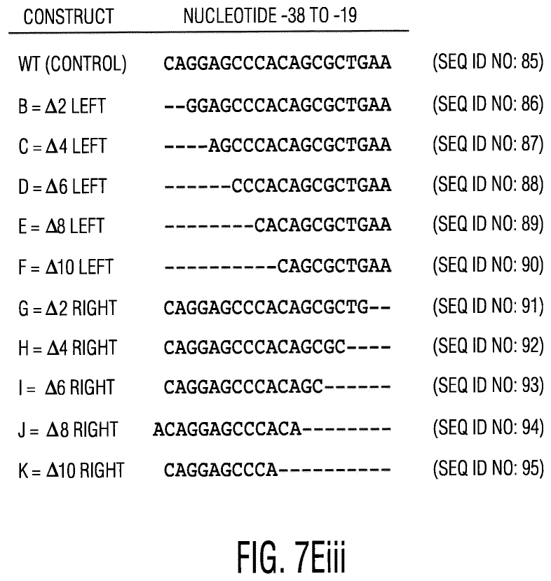

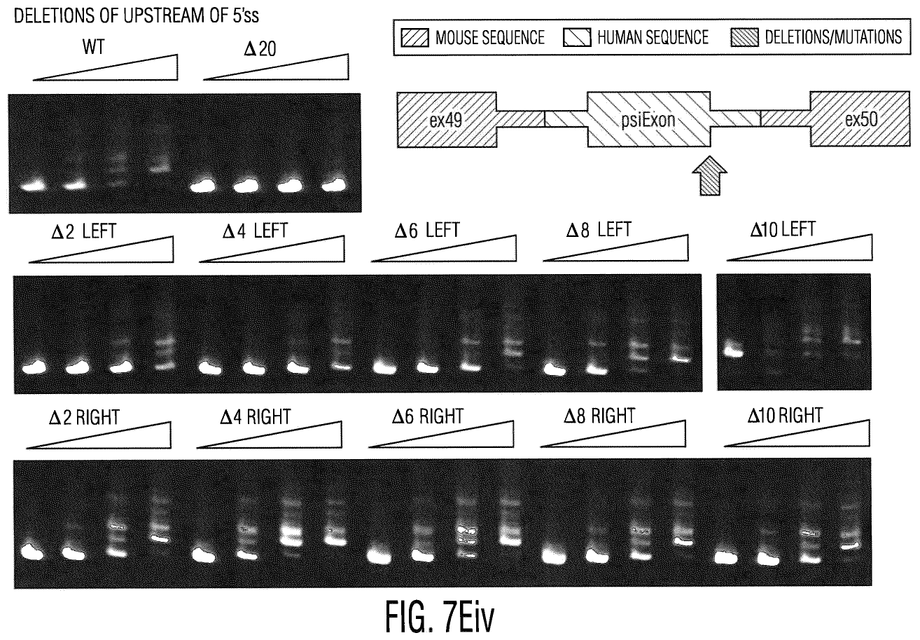

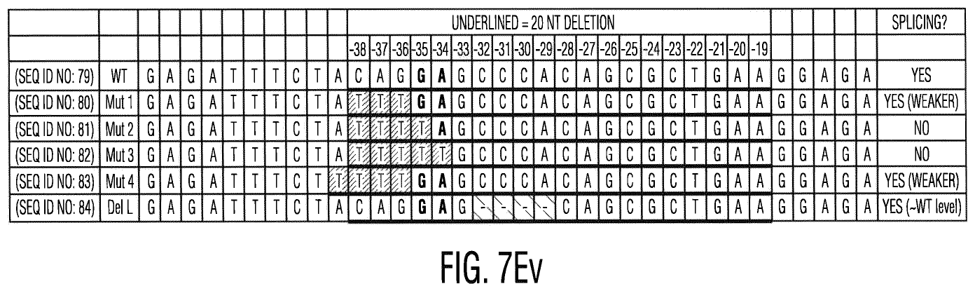

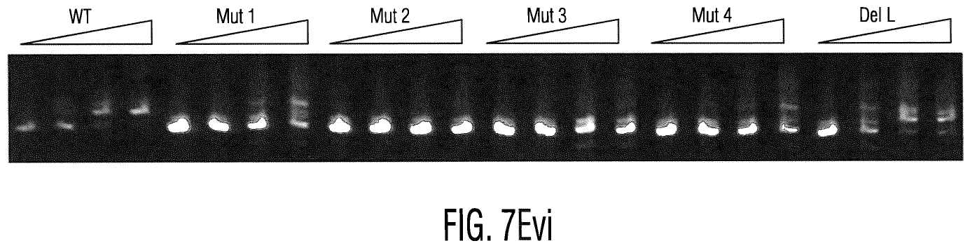

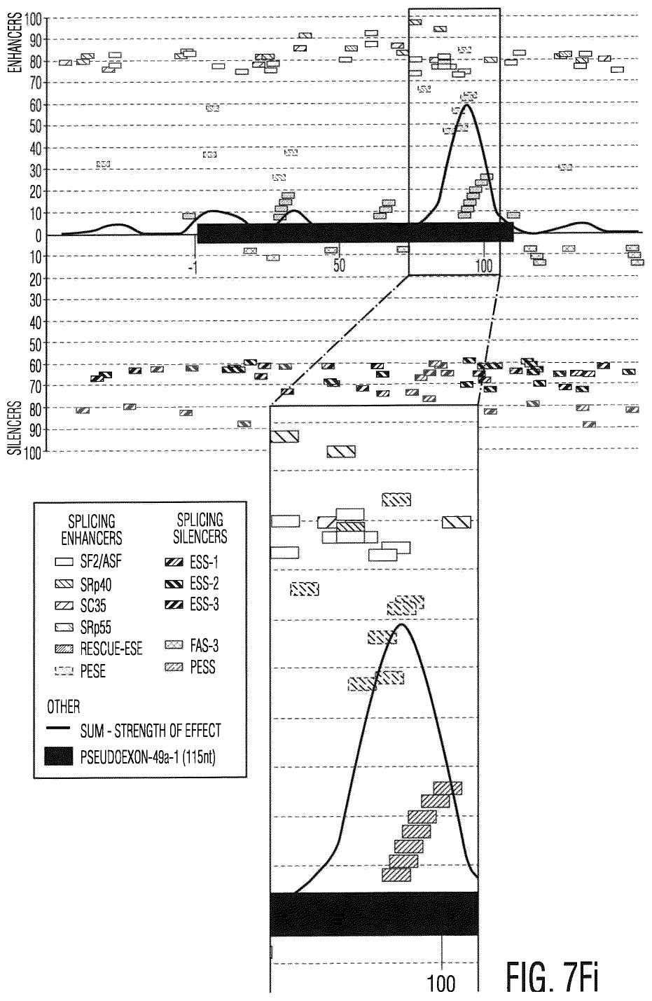

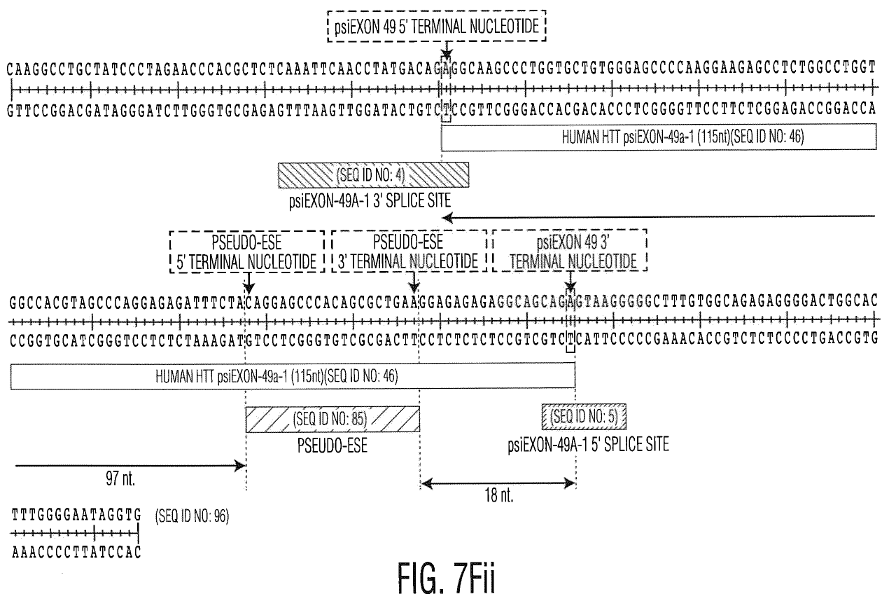

Greater than fifty thousand pseudoexons were identified that are not targeted by either HTT-C2 or U1-GA variant. Four examples were identified within introns 1, 8 and 40 of the human HTT gene. The lack of activity in the presence of either HTT-C2 or U1-GA variant indicated that additional sequence elements are required to promote intronic pseudoexon inclusion (see, for example, FIGS. 7Bxi).

### Minigene Constructs

To identify potential sequence elements, human, mouse, and hybrid mouse/human HTT intron 49 minigene constructs were generated (FIGS. 7A, 7Bi-xii, 7Di-viii and 7Ei-vi). HEK293 cells transfected with the minigene constructs were treated with varying concentrations of test compounds in a final concentration 0.05% DMSO and incubated for 24 hours. Total RNA was isolated from the cells using the RNeasy plus mini kit (Qiagen) and RNA concentration and quality were assessed using a NanoDrop spectrophotometer (Thermo Fisher Scientific). cDNA was synthesized using an iScript™ cDNA synthesis kit (Bio-rad Laboratories) according to the manufacturer's instructions. Endpoint PCRs were set up using Platinum™ PCR SuperMix High Fidelity (Invitrogen) and the resulting PCR products separated on 2% eGels (Invitrogen). Primers were directed against common sequences in the minigene constructs:

| | T7 Forward: |
|---|---|
| (SEQ ID NO: 56) | |
| | 5′-TAATACGACTCACTATAGGG-3′; |
| | BGH Reverse, |
| (SEQ ID NO: 59) | |
| | 5′-TAGAAGGCACAGTCGAGG-3′. |

The human HTT intron 49 minigene responded in a dose dependent manner with HTT-C2 (10 nM, 100 nM, 1 μM) (FIG. 7Bi-ii) but not the mouse Hit construct (FIG. 7Biii-iv). Hybrid constructs containing either the human HTT intron 49 (FIG. 7Bv-vi) or the human HTT pseudoexon (+/−50 nucleotides; see FIG. 7Bvii-viii) were spliced, indicating that the pseudoexon likely contains the responsive sequence. Interestingly, a construct lacking the 3′ splice site of the human HTT 49a pseudoexon was sensitive to compound-induced splicing (FIG. 7Bix-x). Splicing of this truncated pseudoexon utilised a cryptic 3′ splice site present in the mouse Hit intron. No compound-induced pseudoexon inclusion occurred within the construct lacking the 5′ splice site (FIG. 7Bxi-xii). These data indicate that the human 5′ splice site is required for compound-induced splicing of human HTT.

### Mutational Analysis of the Noncanonical 5′ Splice Site

Additional, point mutations at the noncanonical 5′ splice site confirmed that GA is required at the −2 and −1 position (FIG. 7Ci) for compound-induced insertion of the pseudoexon (FIG. 7Cii). Furthermore, a single nucleotide change from A to G at the −1 position, changing the 5′ splice site into a canonical 5′ splice site, resulted in full inclusion of the pseudoexon in the absence of compound (FIG. 7Cii). Unlike with pseudoexon 49a, minigene constructs with the HTT pseudoexons 1, 8, 40a or 40b within HTT were not spliced (FIG. 7Di-viii). Splicing was absent even when the −2 and −1 position were changed to canonical A and G, respectively (FIG. 7Dx). Thus, additional sequence elements within the HTT pseudoexon 49a are required for splicing.

Exon definition is critical for recognition and inclusion of an exon during pre-mRNA splicing, A bioinformatic analysis of the HTT pseudoexon using Human Splicing Finder at INSERM/University of Marseilles; web page: umd.be/Redirect.html) identified a number of potential sequences that could be required for HTT-C2 induced pseudoexon inclusion (see FIG. 7Fi).

### Identification of a Pseudo Exonic Splicing Enhancer (Pseudo-ESE)

Deletion of 20 nucleotides from within the region predicted from bioinformatic analysis to enhance splicing (outlined in FIG. 7Fi), close to the 5′ splice site in the HTT minigene, resulted in a loss of compound-induced splicing (FIG. 7Ei-ii). To narrow down the region responsible for the small molecule-induced splicing at the 5′ splice site, 5′ and 3′ deletions of the 20 nt nucleotide region within a mouse/human HTT minigene were generated (FIG. 7Eiii) and tested for pseudoexon inclusion in HEK293 cells. Deletion of 2 or 4 nucleotides at the left-hand side of this region reduced the level of pseudoexon inclusion compared with wild-type HTT, indicating that this region was also important for regulating splicing events (FIG. 7Eiv). Additional mutations within the 20 nt enhancer sequence also abolished pseudoexon inclusion (FIG. 7v-vi). Compound (I) induced inclusion of the HTT intron 49 pseudoexon into spliced HTT mRNA therefore requires both the upstream pseudo exonic splicing enhancer (pseudo-ESE) and a proximal noncanonical GAgu 5′ splice site. Thus, each of these elements while necessary for Compound (I) induced pseudoexon inclusion are not by themselves sufficient for small molecule-induced splicing at the noncanonical 5′ splice site. A map of the different elements in relation to pseudoexon 49 can be found in FIG. 7Fii.

### Example V: Small Molecule-Induced Htt Pseudoexon Splicing In Vivo

The splicing modifiers lowered HTT mRNA and protein levels with high potency in cultured cells. To determine if this also applies to HTT mRNA and protein in vivo, HTT-C1 was administered to an HD mouse model.

### Animal Studies

BACHD mice were obtained from The Jackson Laboratory (ME, USA).

Mice hemizygous for the BACHD transgene are viable and fertile. Under the control of endogenous human htt regulatory machinery, BACHD mice have relatively high expression levels of a neuropathogenic, full-length human mutant Huntingtin (fl-mhtt) modified to harbor a loxP-flanked human mutant htt exon 1 sequence (containing 97 mixed CAA-CAG repeats encoding a continuous polyglutamine (polyQ) stretch). Prior to Cre recombinase exposure, BACHD mice exhibit progressive motor deficits, neuronal synaptic dysfunction, and selective late-onset neuropathology without somatic polyQ repeat instability in the aged brain.

The Hu97/18 mouse is a humanized mouse model of HD obtained by intercrossing BACHD mice with YAC18 mice having a knockout of the endogenous mouse HD homolog (Hdh). Hu97/18 mice recapitulate the genetics of HD, having two full-length, genomic human HTT transgenes heterozygous for the HD mutation and polymorphisms associated with HD in populations of Caucasian descent (described in Southwell et al. Hum Mol Genet. (2017) 15; 26(6):1115-1132, the content of which is incorporated by reference here in its entirety).

The genotype of each animal was confirmed by an in-house polymerase chain reaction assay prior to enrolment in the study.

### Quantification of HTT Protein in Animal Tissues

Test mice were euthanised and brain, muscle (quadriceps), and blood samples were harvested 2 hours after the last dose on Day 20. Prior to analysis, crude total protein from brain and muscle tissue samples was prepared by sample lysis in MSD® assay buffer 1 (MSD®) with Complete™ Protease Inhibitor Cocktail added (Roche Diagnostics). Tissues were then homogenised using TissueLyser II (Qiagen) plus a 5 mm stainless steel bead. The lysate was clarified by centrifugation at 16,000×g for 20 minutes at 4° C. and the total protein concentration quantified with the Pierce™ BCA Protein Assay Kit (Thermo Scientific) according to the manufacturer's instructions. Whole blood was collected by cardiac puncture into EDTA collection tubes. An aliquot (100-200 μL) was added to 1.5 mL of eBioscience™ 1× Red Blood Cell Lysis Buffer (Thermo Fisher) and mixed well for 5 minutes before collecting the white blood cells (WBC) by centrifugation at 400×g. The supernatant was discarded, and the pellet of WBCs was frozen in liquid nitrogen and stored at −70° C. Brain and muscle sample lysates were analysed for hHTT and KRAS protein expression using the ECL protein assay (described previously); using the same method WBC samples were also analysed for hHTT expression but not KRAS.

Each tissue sample was tested in duplicate using the ECL protein assay and the average hHTT and KRAS readouts were calculated. The ratio of the mean hHTT signal to the mean KRAS signal (×1000) was determined for each test animal. The hHTT/KRAS ratio grand mean for the vehicle group of five test animals was calculated and the fold change relative to the vehicle grand mean was determined for each test animal in each group. Percent hHTT (% hHTT) lowering normalised to KRAS was determined for each test animal by subtracting the fold change from one and multiplying the difference by 100. Each experiment was performed twice yielding ten % hHTT lowering values for each treatment group. For each treatment group, the mean % hHTT lowering plus the standard error of the mean (SEM) was plotted as a bar graph. The % hHTT lowering in white blood cell samples was determined without KRAS using the grand hHTT vehicle mean instead of the grand hHTT/KRAS ratio vehicle mean. In vivo pharmacokinetic studies

Oral pharmacokinetics (PK) of compounds was evaluated in WT littermates from the BACHD colony (FVB background). Mice were treated with test compounds (10 mg/kg) by oral gavage in 0.5% hydroxypropylmethyl cellulose (HPMC) with 0.1% Tween 80. Blood was collected by terminal cardiac puncture at specified time points (3 mice per time point) and centrifuged to generate plasma. Brain tissue was collected at the time of blood collection and homogenized in water. Protein was precipitated from plasma and brain homogenates with acetonitrile, methanol mixture (5:1, v:v) containing an internal standard that is a close analog of the test compounds. The mixture was filtered through an EMD Millipore MultiScreen™ Solvinert Filter Plate (MSRLN04, Millipore, Burlington, MA). Calibration standards were prepared in the same matrix and processed with the testing samples. Filtrates were analysed using Acquity ultra performance liquid chromatography (UPLC) system (Waters Corporation) tandem with Xevo TQ-s Spectrometer (Waters Corporation). Samples were injected on to a Waters UPLC Acquity BEH C18 Column (2.1*50 mm, 1.7 μm) maintained at 50° C. The injection volume was 3 μL and the mobile phase flow rate was 0.45 mL/min. The mobile phase consisted of 2 solvents: A) 0.1% formic acid in water and B) 0.1% formic acid in acetonitrile. The initial mobile phase started with 5% solvent B for 0.4 min, which was changed to 98% solvent B over 0.8 min with linear gradients and then maintained at 95% solvent B for another 0.4 min. The drug concentrations were acquired and processed with MassLynx 4.1 software. PK parameters were estimated using the non-compartment method within Phoenix® WinNonlin® Build 8.1 (Certara USA, Inc., Princeton, NJ).

### In Vivo Pharmacodynamic (PD) Studies

BACHD: Pharmacodynamic (PD) evaluations were performed in BACHD mice aged 6-10 weeks. Compound or vehicle (HPMC/0.1% Tween 80) was administered to BACHD mice (5 female mice per group) once daily for 21 doses (QDx21) by oral gavage; dosing volumes were 10 mL/kg. Each animal was regularly observed for mortality or signs of pain, distress, or overt toxicity and findings were recorded. Body weights were recorded at the start and at least once a week during the course of the study. As described previously, tissue samples were obtained and prepared for ECL protein assay analysis from each animal as described previously.

Hu97/Hu18: Both sexes of 2-4 month old Hu97/181 mice were used. Mice were maintained under a 12 h light:12 h dark cycle in a clean facility with free access to food and water. Experiments were performed with the approval of the Institute Animal Care and Use Committee of the University of Central Florida. Mice were treated with vehicle control or 2, 6, or 12 mg/kg of compound daily by oral gavage for 21 consecutive days. Mice were weighed 3× weekly and observed daily for general health and neurological signs, including gait, head tilt, and circling. No adverse events were observed, and no mice were removed from the study.

### Hu97/Hu18 Terminal Tissue and Sample Collection

Mice were anesthetized with Avertin (2,2,2-tribromoethanol, Sigma Aldrich, catalog #T48402) and secured in a stereotaxic frame (Stoelting). The ear bars were raised and the nose piece used to position the mice in a manner that would allow for a near 90° tilt of the head to access the cistema magna. A 1 cm2 section of dorsal neck skin was removed and muscle layers were completely dissected away to expose the cisterna magna, which was then cleaned with PBS and 70% ethanol and dried using compressed air. A 50 cc Hamilton® syringe with point style 2 with a 12o bevel was then lowered carefully into the cistema magna. CSF was slowly withdrawn at a rate of 10 μl/min using an UltraMicroPump with Micro4 controller (World Precision Instruments). CSF samples were collected in pre-chilled tubes, centrifuged, then flash frozen in liquid N2 prior to storage at −80° C.

Whole blood was then collected by cardiac puncture into EDTA coated tubes and divided into 3 aliquots. One was immediately snap frozen, while plasma was isolated from another and crude PBMCs from the third. Mice were then decapitated and the brain removed and placed in ice for −1 min to increase tissue rigidity. During this interval, liver, heart and quadriceps muscle were isolated and snap frozen. Brains were them microdissected into cortex, hippocampus, striatum, cerebellum, and midbrain/brain stem.

Immunoprecipitation and flow cytometry (IP-FCM) mtHTT quantitadon

Approximately 10,000 5 μm caboxylate-modified latex beads (Invitrogen, catalog #C37255) were coupled with capture antibody, HDB4E10 anti-HTT, in 50 μl of NP40 lysis buffer (150 mM NaCl, 50 mM Tris pH 7.4, Halt phosphatase (Thermo Scientific, catalog #78420) and Halt protease inhibitor cocktails (Thermo Scientific, catalog #78429), 2 mM sodium orthovanadate, 10 mM sodium fluoride NaF, 10 mM Iodoacetamide, and 1% NP40). Capture antibody coupled beads were then combined with 10 μl of CSF, or 20 μl of plasma in triplicate in a 96-well V-bottom plate (Thermo Scientific, catalog #249944), brought to a total volume of 50 μl in NP40 lysis buffer, mixed well, and incubated overnight at 4° C. The next day, the plate was spun down for 1 min at 650 RCF and supernatant was removed. Beads were washed 3 times in IP-FCM wash buffer (100 mM NaCl, 50 mM Tris pH 7.4, 1% bovine serum albumin, 0.01% Sodium Azide). MW1 anti-expanded polyglutamine probe antibody was biotinylated using EZ-Link Sulfo-NHS-Biotin (Thermo Scientific, catalog #21217), and 50 μl of the diluted antibody was incubated with the HDB4E10 beads bound to mtHTT for 2 hr at 4° C. Beads were washed 3 times with 200 μl of IP-FCM wash buffer. Streptavidin-PE (BD Biosciences, catalog #554061) was prepared at 1:200 and 50 μl added to each well and incubated at room temperature protected from light for 30 min. Beads were washed 3 times with 200 μl of IP-FCM buffer, resuspended in 200 μl of IP-FCM wash buffer, and fluorescence intensity of approximately 2000 beads per sample, HDB4E10/MW1 mtHTT bead complexes, were measured using an Acuri C6 flow cytometer (BD Biosciences). Median fluorescent intensity of PE measured for each sample to determine relative mtHTT protein levels.

### MDCK-MDR1 Efflux Assay

The MDR1 efflux assay was conducted at Absorption System LLC (Exton, PA). Briefly, MDCK-MDR1 and MDCK-WT cell monolayers were grown to confluence on collagen-coated, microporous membranes in 12-well assay plates (Thermofisher). Compound solutions (10 μM) in permeability assay buffer (Hanks' balanced salt solution (HBSS), 10 mM HEPES, 15 mM glucose; pH of 7.4) were placed in the donor chamber. The receiver chamber was filled with assay buffer plus 1% BSA. Cell monolayers were dosed on the apical side (A-to-B) or basolateral side (B-to-A) and incubated at 37° C. (5% CO2, 100% relative humidity). Sampling from the donor chambers was performed at 0 and 1 hour; and from the receiver chambers at 1 hour. Each determination was performed in duplicate. The flux of lucifer yellow was also measured post-experimentally for each monolayer to ensure no damage was inflicted to the cell monolayers during the flux period. All samples were assayed by LC-MS/MS using electrospray ionisation. The apparent permeability (Papp) and percent recovery was determined using the following equation:

$$Papp = ( dCr / dt ) \times Vr / ( A \times C 0 )$$

dCr/dt represents the slope of the cumulative receiver concentration versus time in μM/s; Vr is the volume of the receiver compartment (cm3); Vd is the volume of the donor compartment in (cm3); A is the area of the insert (1.13 cm2 for 12-well); CO is the average measured concentration of the donor chamber at time zero in μM; Net Efflux ratio (ER) is defined as Papp(B-to-A)—Papp(A-to-B).

### Unbound Brain Partition Coefficient (Kp,Uu)

The unbound brain partition coefficient (Kp,uu) is defined as the ratio between unbound brain free drug concentration and unbound plasma concentration. It was calculated using the following equation:

$$Kp , uu = {C}_{brain} \times fu , b / ( {C}_{plasma} * fu , p )$$

C_{brain} and Cₚₗₐₛₘₐ represent the compound concentrations in brain and plasma, respectively. fu,b and fu,p are the unbound fraction of each testing article in brain and plasma, respectively. Both fu,b and fu,p were determined in vitro using Pierce Rapid Equilibrium Dialysis (RED) device at Absorption System LLC (Exton, PA). Kp,uu was calculated individually for each animal from multiple mouse PK studies and the average values are reported here.

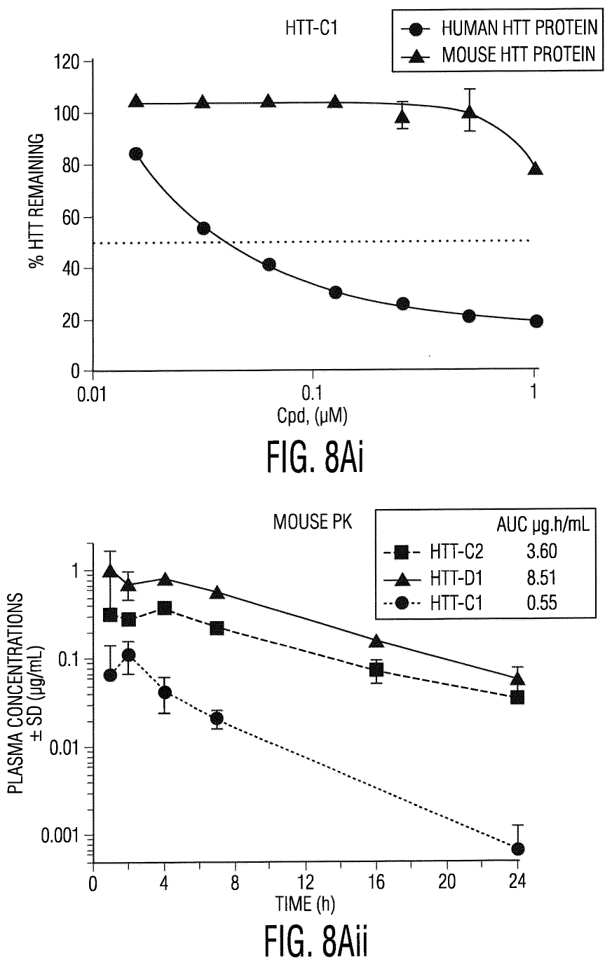

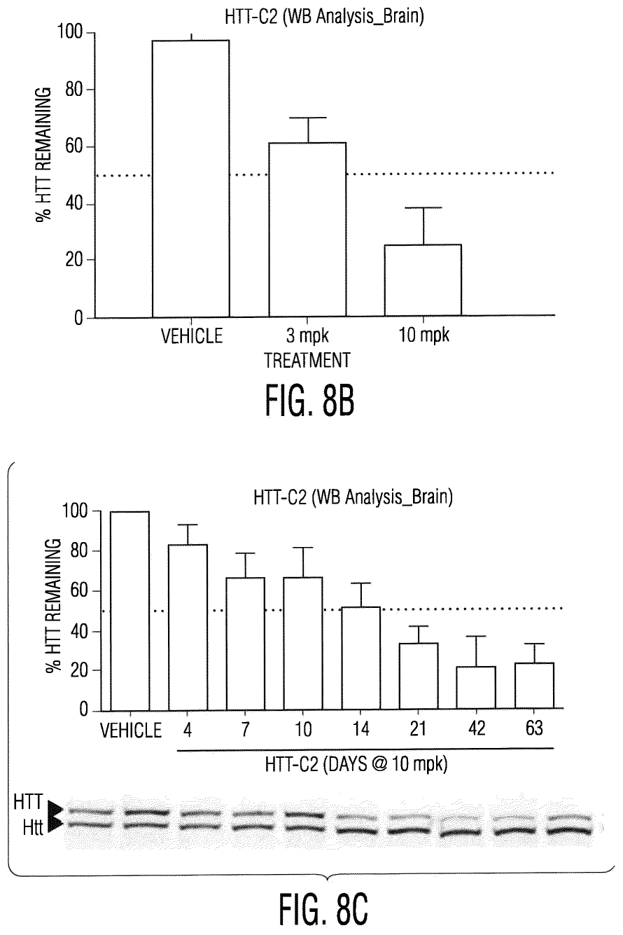

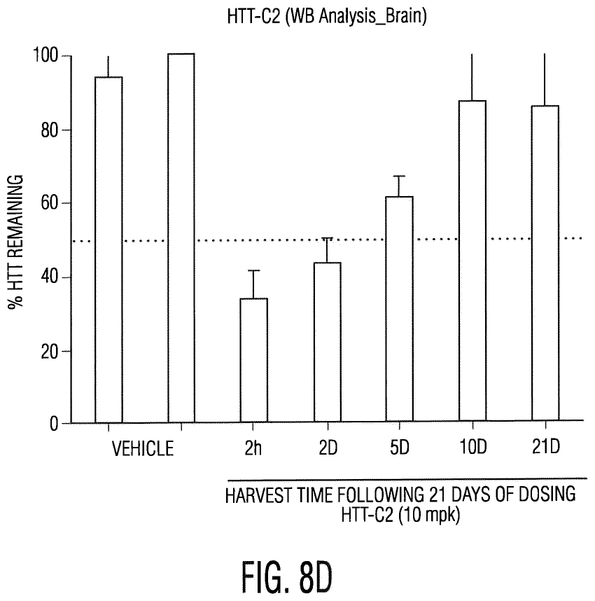

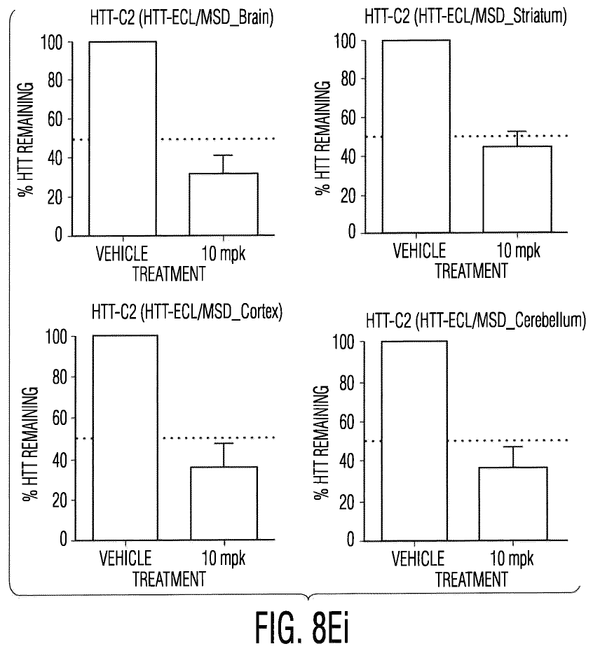

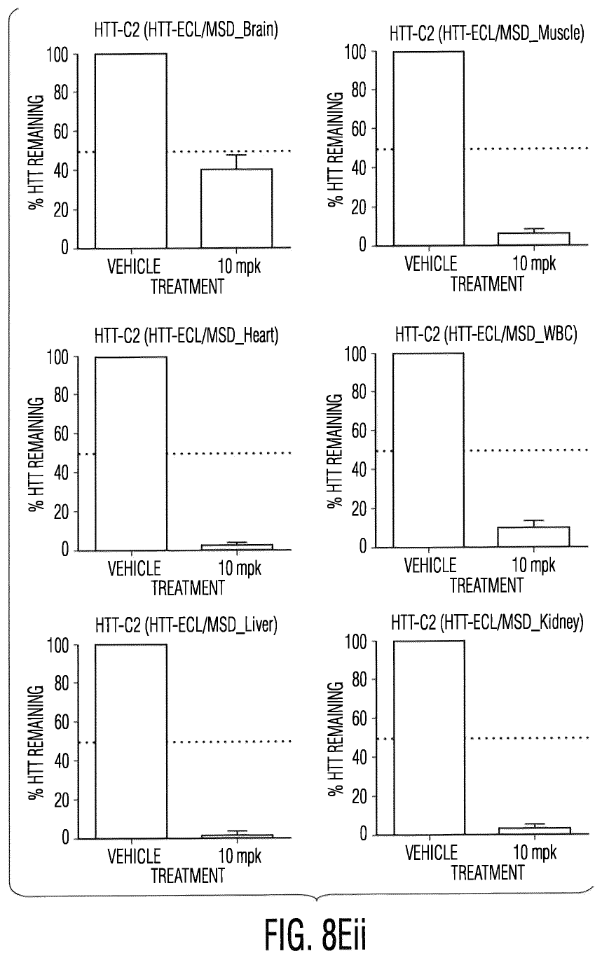

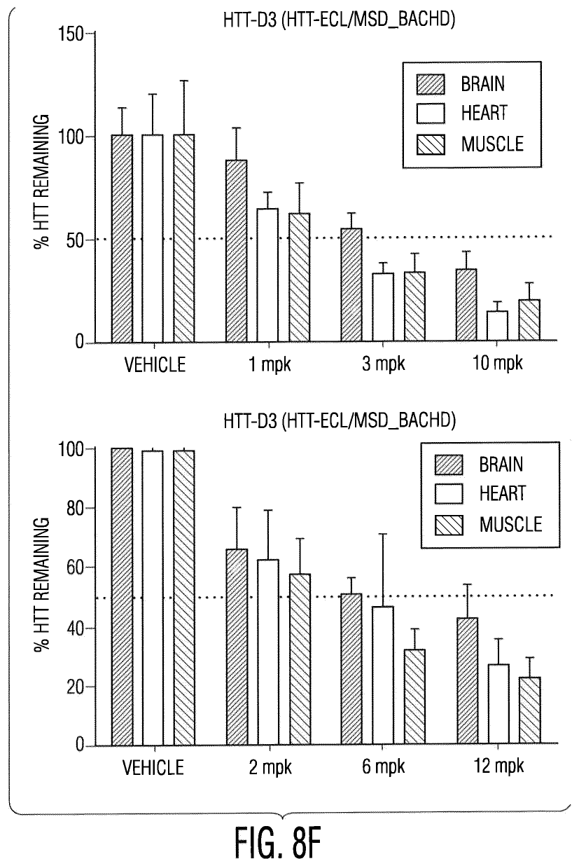

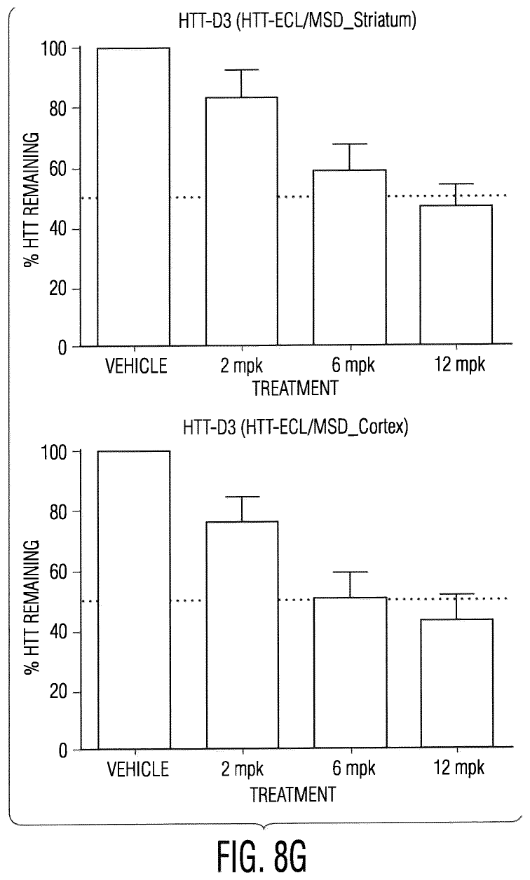

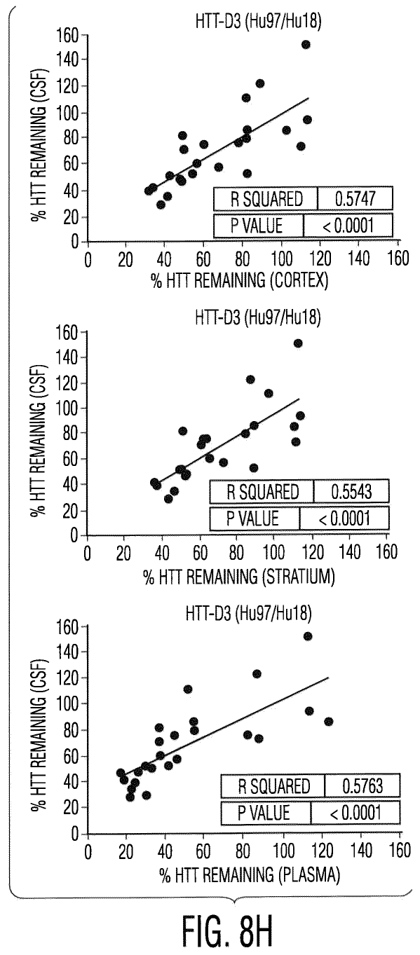

As shown in FIG. 8Ai, mouse Htt protein levels were minimally affected by HTT-C1 treatment in contrast to human HTT levels in human cells, which suggests that HTT-C1 induced splicing activity is not conserved in mice and a mouse model expressing full-length human HTT would be needed to explore this activity in vivo. As the splicing modifiers required the presence of a specific human HTT region, target engagement and pharmacodynamic effects of the compounds were assessed using the HD transgenic mouse model (BACHD) which expresses a full-length human mutant HTT gene. BACHD mice display mild pathology and late onset HD phenotype, progressing gradually over many months, with no signs of striatal degeneration.

In vivo studies in BACHD mice were undertaken to evaluate whether splicing modifiers could lower HTT levels in the brain. Compound HTT-C2 demonstrated superior exposure to HTT-C1 after a single oral dose of 10 mg/kg FIG. 8Aii(2)) and was prioritised for further evaluation.

Daily oral administration of HTT-C2 resulted in a dose-dependent reduction of HTT levels within brain tissue (FIG. 8B). Time course experiments using HTT-C2 revealed that maximal reductions in HTT levels were achieved by Day 21 of treatment, with no further reduction observed beyond this time point (FIG. 8C). These effects were reversible, as protein expression levels returned to control levels within 10 days of treatment cessation (FIG. 8D). Uniform lowering of HTT protein by >50% was achieved throughout the whole brain following treatment with HTT-C2, most importantly within the striatum and cortex (FIG. 8Ei). Administration of HTT-C2 also dose dependently lowered HTT protein within peripheral tissues (FIG. 8Eii). Target engagement by the compound for effective HTT lowering in the brain correlated well with the free compound exposure (fAUC) in the plasma, provided the compound showed minimal efflux in the BBB permeability assay (data not shown).

HTT was reduced in all tissues evaluated after treatment with HTT-C2, although attenuated lowering in the brain was observed when compared to the periphery (FIG. 8Eii). Studies suggest that a 50% global reduction in HTT is predicted to be well tolerated, however, greatly exceeding the level is not desirable. Therefore, HTT-C2 would not be a suitable candidate as an HTT lowering therapeutic, because, as observed in the mouse, the doses required to achieve 50% HTT lowering in the brain, lead to a reduction in excess of 90% in the blood cells, muscle, heart, liver and kidney (FIG. 8Eii). In addition, diagnostic sampling of HTT lowering in blood would greatly underestimate the amount of lowering achieved in the CNS. The disproportionate HTT lowering observed in the periphery versus the brain with HTT-C2 treatment can largely be attributed to P-gp efflux, as observed in an in vitro MDCK-MDR1 permeability assay (data not shown).

This data prompted a reevaluation of HTT-D1, which demonstrated reduced efflux over HTT-C1 and HTT-C2. While improving potency for the series produced HTT-D2 (IC₅₀, 10 nM), additional optimisation led to HTT-D3 with much reduced MDR1 efflux compared with HTT-D2. As a result, HTT-D3 has much better brain penetration as compared to other compounds, indicated by the higher unbound free drug ratio between brain and plasma (Kp,uu; see TABLE IV). Administration of HTT-D3 achieved dose dependent and more equitable lowering of HTT protein within the brain and peripheral tissues of two humanised HD mouse models, BACHD and Hu97/18 mice (FIG. 8F). In the Hu97/Hu18 model, uniform HTT protein reduction was observed in two critical brain sections, striatum, and cortex (FIG. 8G). These results demonstrate that reduction of brain HTT protein by HTT-D3 results in correlative reduction of CSF HTT protein. In addition, similar correlation was observed between plasma and CSF HTT protein levels upon HTT-D3 treatment (FIG. 8H). Small molecules with reduced efflux, such as HTT-D3, are potential HTT lowering therapeutics for HD with the added benefit that measuring HTT levels in an accessible and non-invasive peripheral tissue (blood or plasma) could reliably predict the level of HTT lowering in the CNS.

### Nanostring Analysis

Pre-mRNA splicing changes triggered by splicing-modulating compounds was quantified with Nanostring technology (Naryshkin et al., (2014) Science, 345, 688-693; Palacino et al., (2015) Nat. Chem. Biol., 11, 511-517, the contents of which are incorporated by reference herein in their entireties). The drug dose-response was analyzed using NanoString-Splice web service.

To generate RNA for the NanoString experiments, 510⁵ cells (SHSY5Y or HEK293) were seeded in 6-well plates. Cells were then treated with various concentrations of compound or DMSO. For compound HTT-C2, the final compound concentrations were 4.8 nM, 24 nM, 120 nM, 600 nM, and 3 μM. For compound HTT-C3, the final compound concentrations were 3.22 nM, 9.65 nM, 28.94 nM, 86.81 nM, 260.42 nM, 781.25 nM, 1.5625 μM, 3.125 μM, 6.25 μM, 12.5 μM, and 25 μM. The final DMSO concentration was 0.5% or less. Cells were incubated for ˜20 h (37° C., 5% CO2, 100% relative humidity). Total RNA was isolated using the RNeasy plus mini kit (Qiagen), according to the manufacturer's manual. RNA concentration and quality were assessed by using a NanoDrop spectrophotometer (ThermoFisher).

To quantify splicing changes of selected targets, specific probes were designed by the NanoString Bioinformatics team and synthesized at IDT. They were used in combination with nCounter Element Tagsets (NanoString) and 500 ng isolated total RNA to set up 16-20 h hybridization reactions according to the manufacturer's manual, using a T100 Thermal Cycler (BioRad). NanoString nCounter cartridges were set up using a nCounter Prep Station (NanoString) following the protocol provided by the manufacturer. Cartridges were then analyzed using a nCounter Digital Analyzer (NanoString), following the protocol provided by the manufacturer.

### NanoString Probe Design for Splicing Profiling and Counts Data Normalization

For each alternative exon, two probe sets were designed for targeting the inclusion (I) and skipping (S) isoforms. The toehold exchange technology (Zhang et al. (2012) Nat Chem 4(3): 208-214, the content of which is incorporated by reference herein in its entirety) to was used to increase the specificity of probe targeting. The counts data generated from an nCounter MAX instrument were normalized by spike-in positive controls and a set of reference genes using nSolver 3.0 software.

### Adjusted Percent-Spliced-In (PSI) Value Using I-Probe Relative Hybridization Efficiency Factor (Ei)

Ei was used to represent the relative hybridization efficiency of 1-probe over S-probe. Total mRNA amount for the two isoforms was assumed not to change in different conditions (samples). To estimate Ei for an I- and S-probe pair, the R function “optimize” was used to find the Ei which minimizes the coefficient of variation (CV) of the total adjusted counts T=I/Ei+S (I and S are the normalized counts for I- and S-probe respectively) for multiple samples. Only Ei between 0.01 and 100 was searched, assuming the difference of hybridization efficiency for the two probes is <100 fold. The CV was compared to the estimated Ei and CV values when Ei=1 (no adjustment), and set Ei to 1 if the difference of the two CVs was less than 0.1 (which means that Ei-adjustion did not decrease the CV significantly). PSI, which reflects the percent of inclusion isoforms, was calculated using the formula: raw-PSI=I/(I+S), adjusted-PSI=(I/Ei)/(I/Ei+S).

### Estimate Effective Dose of a Splicing-Modulating Compound Using Adjusted-PSI or Normalized Counts

R-package dre (Ritz et al., (2015) PLoS One 10(12): e0146021) was used to perform the dose-response analysis. The four-parameter log-logistic model (LL.4) was used as the fitting function.

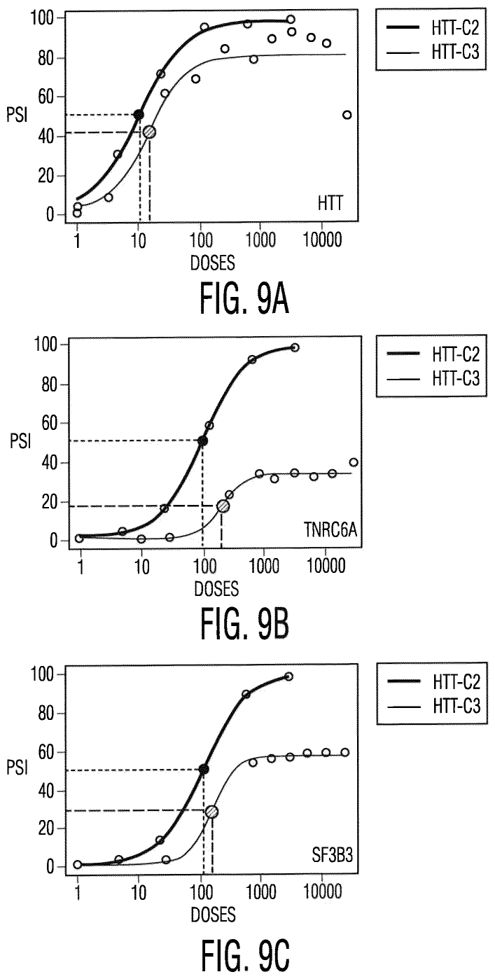

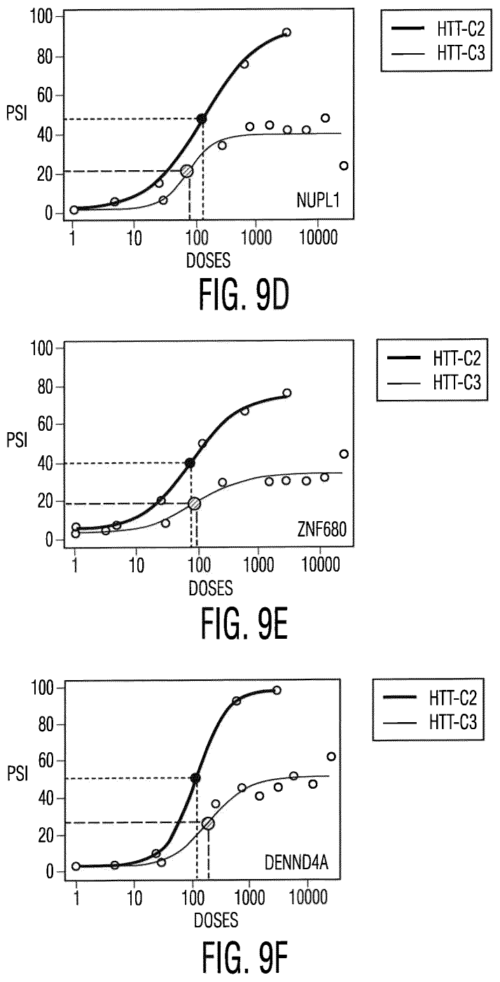

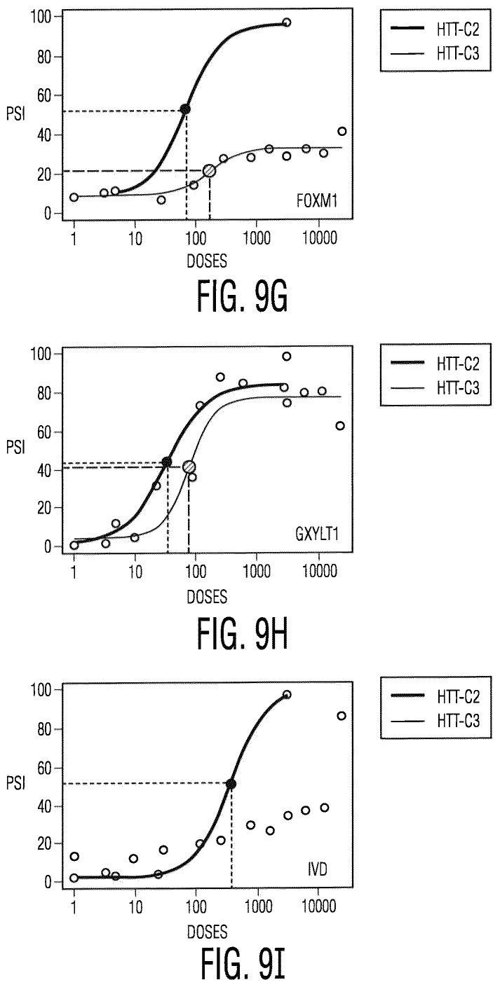

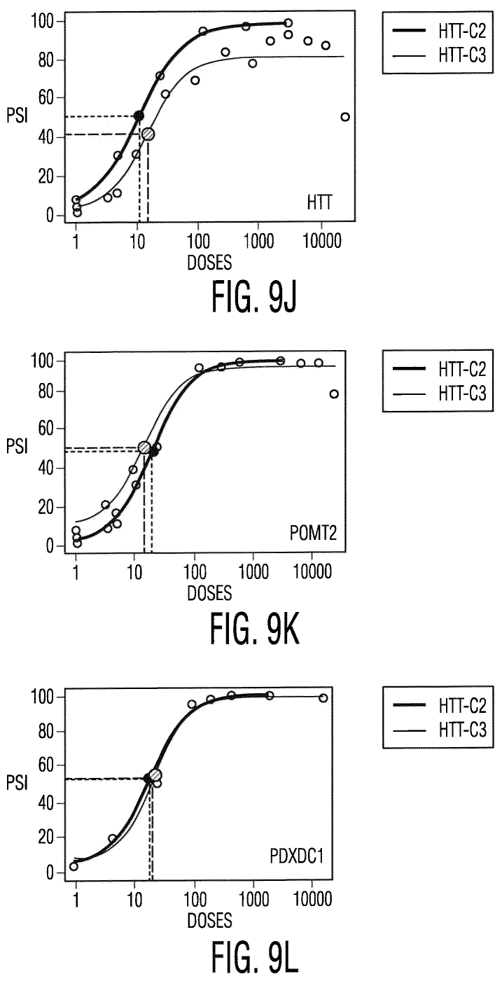

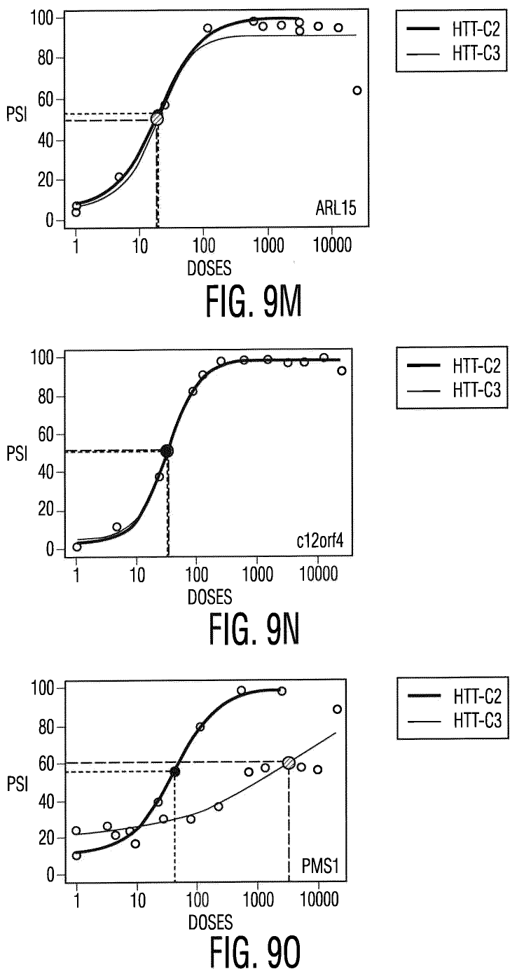

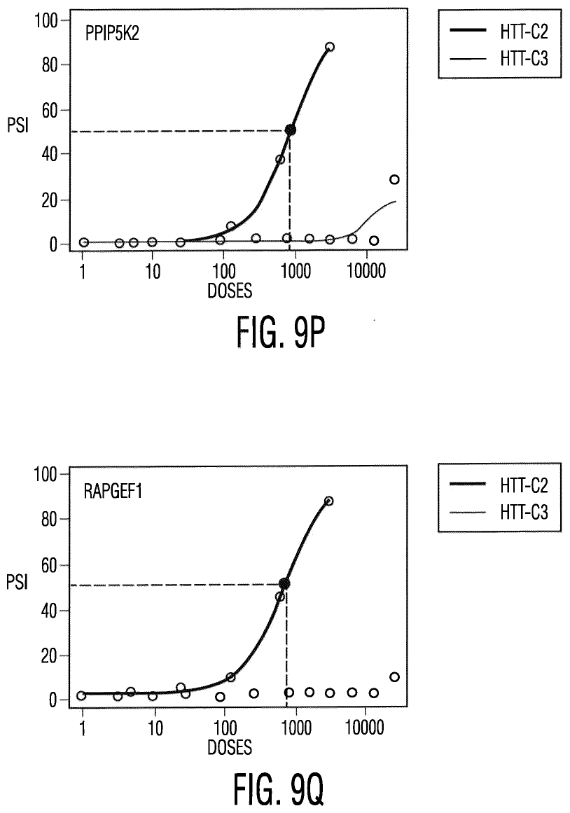

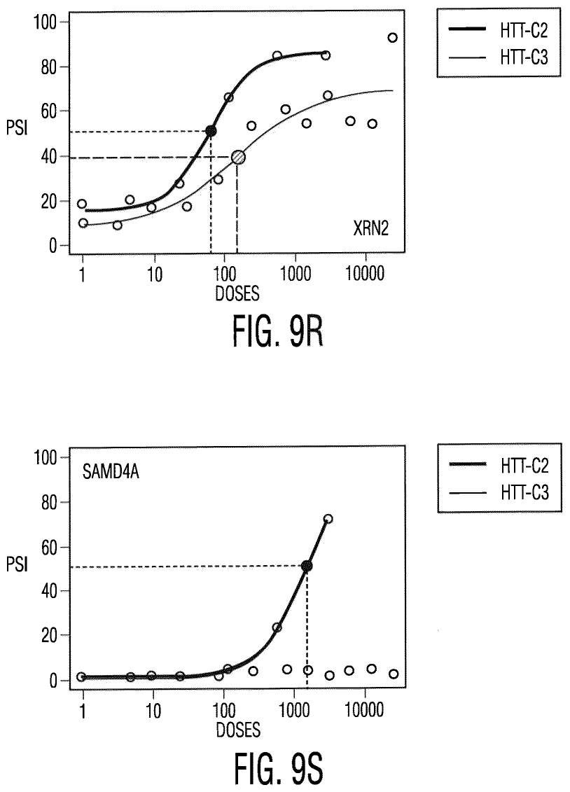

### Graphical Representations of Percent Spliced in (PSI) Transcripts Effected by HTT-C2 Versus HTT-C3 are Shown in FIGS. 9A-9S for the Following Genes:

| | | |
|---|---|---|
| HTT: | Huntingtin | (Entrez Gene: 3064) |
| GXYLT1: | Glucoside Xylosyltransferase | (Entrez Gene: 283464) |
| POMT2: | Protein O-Mannosyltransferase 2 | (Entrez Gene: 29954) |
| PDXDC1: | Pyridoxal Dependent Decarboxylase | |
| | Domain Containing 1 | (Entrez Gene: 23042) |
| ARL15: | ADP Ribosylation Factor Like GTPase 15 | (Entrez Gene: 54622) |
| c12orf4: | Chromosome 12 Open Reading Frame 4 | (Entrez Gene: 57102) |
| TNRC6A: | Trinucleotide Repeat Containing | |
| | Adaptor 6A | (Entrez Gene: 27327) |
| SF3B3: | Splicing Factor 3b Subunit 3 | (Entrez Gene: 23450) |
| FOXM1: | Forkhead Box M1 | (Entrez Gene: 2305) |
| NUPL1: | Nucleoporin 58 | (Entrez Gene: 9818) |
| ZNF680: | Zinc Finger Protein 680 | (Entrez Gene: 340252) |
| DENND4A: | DENN Domain Containing 4A | (Entrez Gene: 10260) |
| PPIP5K2 | Diphosphoinositol Pentakisphosphate | (Entrez Gene: 23262) |
| | Kinase 2 | |
| RAPGEF1 | Rap Guanine Nucleotide Exchange Factor 1 | (Entrez Gene: 2889) |
| SAMD4A | Sterile Alpha Motif Domain Containing 4A | (Entrez Gene: 23034) |
| XRN2 | 5′-3′ Exoribonuclease 2 | (Entrez Gene: 22803) |
| PMS1 | PMS1 Homolog 1, Mismatch Repair | |
| | System Component | (Entrez Gene: 5378) |
| IVD | Isovaleryl-CoA Dehydrogenase | (Entrez Gene: 3712) |

### Example VI: Compound I Tablet Formulations

Six development batches (1-6) of tablets of Compound 1 were prepared using direct compression, by weighing and sieving components through mesh #35 followed by low shear mixing and compressing into tablets. However, the flow was not adequate and sticking to tablet punches was observed while tableting. Two batches of placebo tablets (7 and 8) were prepared the same way without Compound 1. The compositions of the batches are illustrated in Table VI, below.

TABLE VI

| Blend # | 1 | 2 | 3 | 4 | 5 | 6 |
|---|---|---|---|---|---|---|
| Excipient | % w/w | % w/w | % w/w | % w/w | % w/w | % w/w |
| Compound 1 | 5 | 5 | 2 | 10 | 2 | 10 |
| Lactose monohydrate | 38 | | 78 | 70 | 50 | 50 |
| Pearlitol ® SD100 | | 38 | | | | |
| Pregelatinized starch | | | 18 | 18 | | |
| STARX1500 | | | | | | |
| Microcrystalline cellulose | 50 | 50 | | | 41 | 33 |
| Sodium Starch Glycolate | 5 | 5 | | | | |
| Croscarmellose Sodium | | | | | 5 | 5 |
| Colloidal Silicon Dioxide | 1 | 1 | 1 | 1 | 1 | 1 |
| Magnesium Stearate | 1 | 1 | 1 | 1 | 1 | 1 |
| Total | 100 | 100 | 100 | 100 | 100 | 100 |

To resolve poor flow and sticking issues with direct compression, dry granulation using roller compaction was introduced to prepare another three batches (9, 10, and 11) by weighing and sieving the components through mesh #35, mixing with a turbula mixer followed by roller compaction. That was followed by crushing the ribbons and passing through mesh #20 and using a turbula mixer to mix the extra granular components, followed by compression in a tablet press. Good blend uniformity was obtained. The composition of all three batches was the same and similar to batch 6, above, except that it contained equal percent by weight of lactose monohydrate and mcc (41.5% each). However, different roller compaction parameters (roll speed, screw speed, and pressure) were used for each batch. Batch 9 produced the best ribbons on roller compaction. Batch 11 stuck to the roll and the ribbons were brittle, while batch 12 produced a discontinuous ribbon. Dissolution tests were conducted on dry granulation batch 9, but there were granules floating on the surface of the dissolution media due to a wetting issue with Compound 1.

To mitigate the wetting issue a decision was made to introduce surfactants into the formulations. Direct compression batches 13 and 14 were prepared with 5% w/w sodium lauryl sulfate (SLS) and 1% Poloxamer 188, respectively, and with 50 mg strength of Compound 1. Batch 13 with SLS showed even more undissolved granules of Compound 1 floating around with minimal drug release than Batch 9, tested in Example 2. Batch 14 with 1% Poloxamer 188 showed better dissolution performance with no granules floating on the surface of the dissolution media. Thus, inclusion of Poloxamer seemed to mitigate the wetting of the Compound 1 granules. However, a mounding phenomenon observed during the dissolution experiment at 50 revolutions per minute (rpm), with complete drug release only observed when the paddle speed was increased to 150 rpm at 75 minutes. A direct compression batch 17 was also prepared in a similar way with a lower concentration of microcrystalline cellulose and Poloxamer 407 as a surfactant. There were issues with poor processability with this batch.

The composition of batches 13, 14, and 17 are summarized in Table VII, below:

TABLE VII

| Blend # | 13 | 14 | 17 |
|---|---|---|---|
| Excipient | % w/w | % w/w | % w/w |
| Compound 1 | 10 | 10 | 10 |
| Lactose monohydrate | 39 | 41 | 60 |
| Pregelatinized starch | | | 10 |
| STARX1500 | | | |
| Microcrystalline cellulose | 39 | 41 | 13 |
| Croscarmellose sodium | 5 | 5 | 4 |
| Sodium Lauryl Sulfate | 5 | | |
| Poloxamer 188 | | 1 | |
| Poloxamer 407 | | | 1 |
| Colloidal silicon dioxide | 1 | 1 | 1 |
| Magnesium stearate | 1 | 1 | 1 |
| Total | 100 | 100 | 100 |

To minimize the mounding phenomenon in the dissolution vessel, wet granulation batches 15 and 16 were prepared with a lower amount of Avicel PH102 and 0.5% w/w and 2.5% w/w polyvinylpyrrolidone (PVP) K30, respectively and 1% Poloxamer 407. Wet granulation was performed using a mortar and pestle. Intragranular ingredients were passed through #20 mesh sieve and blended. Povidone K30 was dissolved in water to obtain granulation fluid. Then, the preblend was wet granulated with the povidone K30 solution using the mortar and pestle to obtain optimum granules. The wet mass was dried in a tray oven at 60° C. until achieving a moisture content of about 2%. The dried granules were passed through #20 sieve and blended with #20 mesh screened extragranular excipients. The unlubricated blend was mixed with #35 mesh screened magnesium stearate to obtain the final blend.

Batch 15 was found to be an optimal formulation with little mounding upon dissolution at 50 rpm. Batch 16 with 2.5% w/w PVP K30 was found to be inferior to Batch 15 in terms of dissolution performance, most likely due to more compact granules due to a higher level of PVP K30 binder.

A wet granulation batch 18 was also prepared in the same way as above with a lower amount % of Avicel PH 2 (10% w/w) to test whether the mounding in dissolution could be reduced further. However, upon dissolution testing, that batch failed to release Compound 1 completely.

A wet granulation batch 19 was also prepared in a similar way as described above but with 30% mcc in both the intragranular and extragranular blends. This batch also had issues with mounding and poor integrity.

An additional dry granulation batch 20 was prepared with 41% microcrystalline cellulose (mcc) and lactose monohydrate in the intragranular blend but no mcc or lactose monohydrate in the extragranular components.

The composition of batches 15, 16, 18, 19, and 20 are shown in Table VIII, below.

TABLE VIII

| Blend # | 15 | 16 | 18 | 19 | 20 |
|---|---|---|---|---|---|
| Excipient | % w/w | % w/w | % w/w | % w/w | % w/w |
| Intragranular | | | | | |
| Components | | | | | |
| Compound 1 | 10 | 10 | 10 | 10 | 10 |
| Lactose monohydrate, | 20 | 20 | 10 | 30 | 41 |
| FlowLac90 | | | | | |
| Microcrystalline | 20 | 20 | 10 | 30 | 41 |
| Cellulose | | | | | |
| Povidone K30 | 1 | 2.5 | 1 | 1 | |
| Croscarmellose Sodium | 2.5 | 2.5 | 2 | 2 | 3 |
| Colloidal Silicon Dioxide | | | | | 0.5 |
| Magnesium Stearate | | | | | 0.5 |
| Extragranular | | | | | |
| Components | | | | | |
| Lactose monohydrate, | 41.5 | 50 | 61.5 | 11.5 | |
| FlowLac90 | | | | | |
| Croscarmellose Sodium | 2.5 | 2.5 | 3 | 3 | 2 |
| Poloxamer 407 micro | 1 | 1 | 1 | 1 | 1 |
| Kolliphor ® P407 micro | | | | | |
| Colloidal Silicon Dioxide | 0.5 | 0.5 | 0.5 | 0.5 | 0.5 |
| Magnesium Stearate | 1 | 1 | 1 | 1 | 0.5 |
| Total | | | | | |

Dissolution performance of batches 9, 14, 15, 16, 17, 18, and 20 were tested in 500 ml 0.01N HCl while stirring with paddles at 50 rpm to 60 min, increased to 75 rpm to 75 min, increased to 150 rpm until 90 min, removing 5 ml at 5, 10, 15, 20, 30, 45, 60, 75, and 90 min.

Batches 15, 16, and 17 were also tested for degradation at 7 and 14 days after storage at 80° C. at 5% relative humidity and at 80° C. and 75% relative humidity. No degradation was found in any of the samples at the lower humidity, and degradation was minimal and comparable in all three batches tested at the higher humidity level.

Dissolution stability of Batch 15 was tested at various paddle speeds (50, 65, and 75 revolutions per minute) and at accelerated temperature conditions. It was found that the % of drug released at each time point was higher at faster speeds, and 81%, 88%, and 95% of the initial release respectively was released at each speed, respectively in the first 5 minutes. Release rates were even faster when tested in 0.01N HCl at a paddle speed of 75 rpm at room temperature, 40° C., or 65° C., where the % of initial release was 95.4, 92.6, and 96.4, respectively.

Based on the results above, Batches 15 (wet granulation) and 20 (dry granulation) were selected for further testing, with pharmokinetics.

### Example VII: Oral (Po) Administration of Compound I Tablet Formulations

A study was conducted, as follows, to evaluate the exposure of Compound 1 following oral (PO) administration of three formulations of the compound in fasted male Cynomolgus monkeys. One of the formulations (Batch 21) was a suspension of 6% w/w Compound 1 in 0.5% w/w hydroxypropylmethyl cellulose (HPMC). The other two formulations tested were tablets from the wet granulation Batch 15 and from the dry granulation batch 20 prepared as described above.

The monkeys were separated into three groups of four animals each. Monkeys were fed in the afternoon prior to the day of dosing and the remaining food was removed at 7 pm. Food was returned at four hours post dosing. Each monkey received an oral dose of 30 mg of Compound 1 via rubber oral gavage tube or tablet (5 ml of 6 mg/ml of Compound 1 in suspension Batch 21, 2 tablets/animal of 15 mg per tablet of wet granulation Batch 15 or dry granulation Batch 20), and each dose was followed by a 3 ml flush using deionized water. Blood samples were drawn from each monkey at the following time points: pre-dose (0), 0.5, 1, 2, 3, 4, 6, 8, 12, 24, and 48 hours. Each sample was centrifuged for at a temperature of to 8° C. at 3,000×g for 5 minutes, plasma was collected, and frozen on dry ice until testing. Plasma concentrations were determined by LC-MS/MS. Pharmacokinetics parameters were determined.

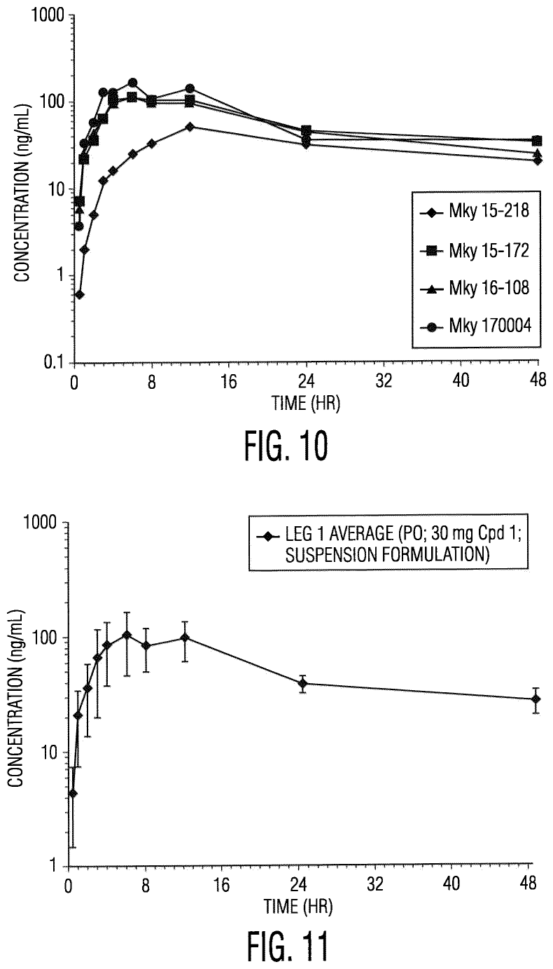

A plot of the individual plasma concentrations of Compound 1 after oral administration of the oral Compound 1 suspension formulation (Batch 21) in 0.5% HPMC in water at 30 mg in the male Cynomolgus Monkeys (Leg 1) is provided in FIG. 10. The four monkeys in the study are identified as “Mky 15-218,” “Mky 15-172,” “Mky 16-108,” and “Mky 170004” in FIG. 10 and other figures below. A plot of mean plasma concentrations at each time point in Leg 1 is provided in FIG. 11. The results are summarized in Table IX, below:

TABLE IX

| Animal ID | 15-218 | 15-172 | 16-108 | 170004 | Mean (n = 4) | SD |
|---|---|---|---|---|---|---|
| Animal Weight (kg) | 4.20 | 4.18 | 4.07 | 4.96 | 4.35 | 0.41 |
| Dosed (mg) | 30 | 30 | 30 | 30 | 30 | 0 |
| Dose (mg/kg) | 7.14 | 7.18 | 7.37 | 6.05 | 6.93 | 0.60 |
| Cₘₐₓ (ng/mL) | 50.6 | 113 | 113 | 167 | 111 | 47.6 |
| tₘₐₓ (hr) | 12 | 6.0 | 6.0 | 6.0 | 7.5 | 3.0 |
| t_{1/2} (hr) | ND | 22.6 | 18.7 | ND | 20.7 | ND |
| AUCₗₐₛₜ (hr · ng/mL) | 1388 | 2845 | 2623 | 3203 | 2515 | 788 |

As shown in Table IX, following PO dosing of the suspension formulation (Batch 21) at 30 mg/animal (Leg 1), maximum plasma concentrations (average of 111±47.6 ng/mL) were observed between 6 and 12 hours post dosing. The average half-life following oral dosing was 20.7 hours. The average total exposure for Compound 1 (Leg 1) at 30 mg/animal was 2515±788 hr*ng/mL and based on the dose normalized AUC last was 369 138 hr*kg*ng/mL/mg.

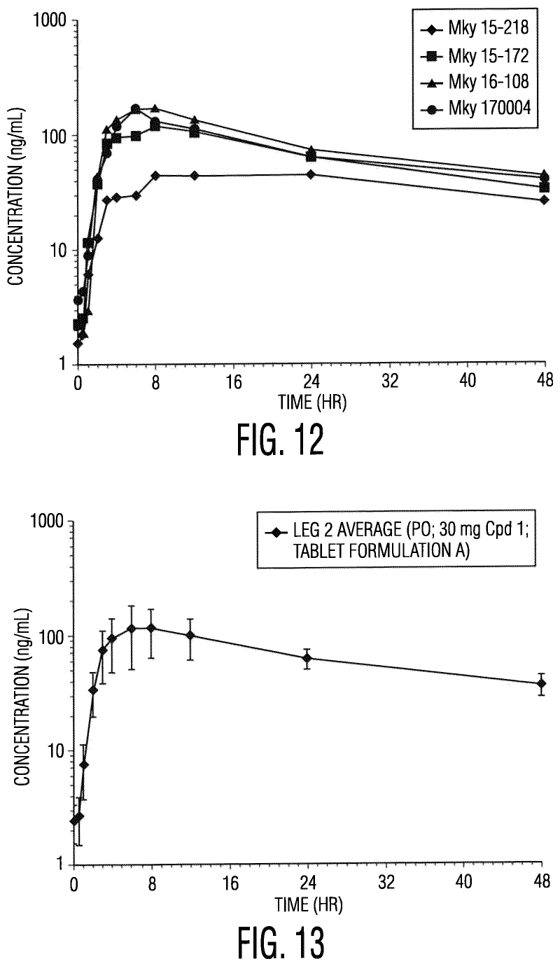

A plot of the individual plasma concentrations obtained from each monkey after oral administration of 30 mg/animal (Leg 2) of Tablet Formulation A (wet granulation Batch 15) is provided in FIG. 12. A plot of mean plasma concentration at each time point is provided in FIG. 13. The results of Leg 2 of the study are summarized in Table X:

TABLE X

| Animal ID | 15-218 | 15-172 | 16-108 | 170004 | Mean (n = 4) | SD |
|---|---|---|---|---|---|---|
| Animal Weight (kg) | 4.21 | 4.36 | 4.00 | 4.95 | 4.38 | 0.41 |
| Dosed (mg) | 30 | 30 | 30 | 30 | 30 | 0 |
| Dose (mg/kg) | 7.13 | 6.88 | 7.50 | 6.06 | 6.89 | 0.61 |
| Cₘₐₓ (ng/mL) | 43.7 | 118 | 170 | 166 | 124 | 58.8 |
| tₘₐₓ (hr) | 8.0 | 8.0 | 8.0 | 6.0 | 7.5 | 1.0 |
| t_{1/2} (hr) | 43.4 | 22.0 | 22.8 | 23.7 | 28.0 | 10.3 |
| AUCₗₐₛₜ (hr · ng/mL) | 1718 | 1364 | 4052 | 3506 | 3110 | 997 |
| Relative F (%) | 124% | 107% | 157% | 110% | 124% | 23% |

As shown in Table X, following PO dosing of tablet formulation A (wet granulation Batch 15) at 30 mg/animal (Leg 2), maximum plasma concentrations (average of 124±58.8 ng/mL) were observed between 6 and 8 hours post dosing. The average half-life following oral dosing was 28.0±10.3 hours. The average total exposure for Compound 1 (Leg 2) at 30 mg/animal was 3110±997 hr*ng/mL and based on the dose normalized AUCₗₐₛₜ was 455±151 hr*kg*ng/mL/mg.

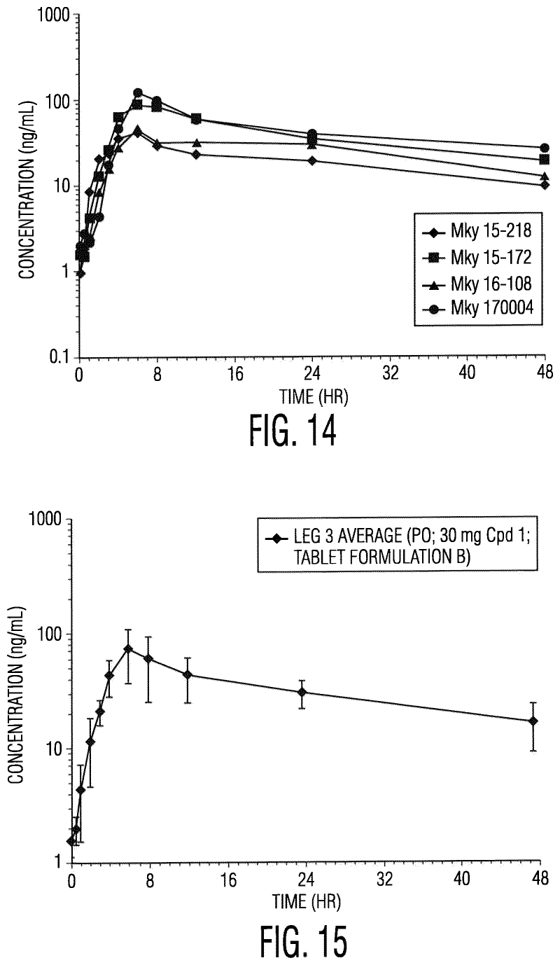

A plot of the individual plasma concentrations obtained from each monkey after oral administration of 30 mg/animal (Leg 3) of Tablet Formulation B (dry granulation Batch 20) is provided in FIG. 14. A plot of mean plasma concentration at each time point is provided in FIG. 15. Results of Leg 4 of the study are summarized in Table XI, where * indicates p<0.05 when compared to AUCₗₐₛₜ from suspension formulation.

TABLE XI

| Animal ID | 15-218 | 15-172 | 16-108 | 170004 | Mean (n = 4) | SD |
|---|---|---|---|---|---|---|
| Animal Weight (kg) | 4.49 | 4.46 | 4.32 | 5.14 | 4.60 | 0.37 |
| Dosed (mg) | 30 | 30 | 30 | 30 | 30 | 0 |
| Dose (mg/kg) | 6.68 | 6.73 | 6.94 | 5.84 | 6.55 | 0.49 |
| Cₘₐₓ (ng/mL) | 41.5 | 87.1 | 45.5 | 120 | 73.5 | 37.2 |
| tₘₐₓ (hr) | 6.0 | 6.0 | 6.0 | 6.0 | 6.0 | 0.0 |
| t_{1/2} (hr) | 26.0 | 22.4 | 24.8 | 32.2 | 26.4 | 4.20 |
| AUCₗₐₛₜ (hr · ng/mL) | 920 | 1883 | 1185 | 2107 | 1524 | 562 |
| Relative F (%) | 62% | 62% | 42% | 63% | 58% | 10% |

As one can see from Table XI, following PO dosing of tablet formulation B (dry granulation Batch 20) at 30 mg/animal (Leg 3), maximum plasma concentrations (average of 73.5±37.2 ng/mL) were observed at 6 hours post dosing. The average half-life following oral dosing was 26.4±4.20 hours. The average total exposure for Compound 1 (Leg 3) at 30 mg/animal was 1524±562 hr*ng/mL and based on the dose normalized AUCₗₐₛₜ was 237±102 hr*kg*ng/mL/mg.

Based on the average dose normalized AUCₗₐₛₜ values, Tablet formulation A (wet granulation Batch 15) had an exposure of 455 hr*kg*ng/mL/mg, which is 124±23% of the exposure from suspension formulation (369 hr*kg*ng/mL/mg). Tablet formulation B (dry granulation Batch 20) had an exposure of 237 hr*kg*ng/mL/mg, which is 58±10% of the exposure from suspension formulation. Thus, the AUC from solid formulation A was found to be very comparable to the one from the suspension formulation. However, the AUC from solid formulation B was significantly lower (P<0.05) compared to the value from the suspension formulation. In other words, these studies show that Compound 1 was significantly more bioavailable in the tablets produced by dry granulation (formulation Batch 20) than in the suspension formulation or in the tablets produced by wet granulation (formulation Batch 15).

Using the tablets of wet granulation formula Batch 20 as a starting point, additional studies were conducted to identify excipients and concentrations of each excipient that could be scaled up and readily processed during the tableting process, and which had superior physical characteristics, including rapid dissolution characteristics. Grades of lactose and cellulose were selected that are particularly suitable for wet granulation processes and the total amount of intragranular excipients was increased. The concentration of Povidone was increased from 1% to 2% to 5% in three different batches. The total amount of lactose monohydrate used in the formulation was also increased, and the ratio of mcc to lactose monohydrate was reduced. Examples of three formulations prepared and tested are provided in Table XII, below. 500 gram batches of 50 g of Compound 1 per batch were prepared of each formulation below.

TABLE XII

| Batch No | 22 | 23 | 24 | 25 |
|---|---|---|---|---|
| Ingredient name | % w/w | % w/w | % w/w | % w/w |
| Intragranular | | | | |
| Compound 1 | 10.0 | 10.0 | 10.0 | 10.0 |
| Microcrystalline cellulose 101 | 25.0 | 20.0 | 20.0 | 20.0 |
| Lactose monohydrate 310 | 15.0 | 40.0 | 40.0 | 40.0 |
| Povidone K30 | 1.5 | 2.0 | 5.0 | 3.0 |
| Croscarmellose sodium | 2.5 | 2.5 | 2.5 | 2.5 |
| Poloxamer 407 | 1.0 | | | |
| Water USP | 30% IP | 25% IP | 40% IP | 40% IP |
| Subtotal (dry basis) | 55.0 | 74.5 | 77.5 | 77.5 |
| Extragranular | | | | |
| Lactose monohydrate 316 | 41.0 | 20.0 | 17.0 | 19.0 |
| Croscarmellose sodium | 2.5 | 2.5 | 2.5 | 2.5 |
| Poloxamer 407 | | 1.0 | 1.0 | 1.0 |
| Colloidal silicon dioxide | 0.5 | 0.5 | 0.5 | 0.5 |
| Magnesium stearate MF-3-V | 1.0 | 1.5 | 1.5 | 1.5 |
| Total (dry basis) | 100.0 | 100.0 | 100.0 | 100.0 |

Tablets produced by wet granulation with the compositions described above were coated, but the coating did not affect the disintegration time. Table XIII below, shows the results from testing tablet cores produced from batches 23-25, above, some with 5 mg (A) and others with 50 mg (B) of Compound 1. PGP-T

TABLE XIII

| | Weight | Thickness | Hardness | | |
|---|---|---|---|---|---|
| Cores | (mg) | (mm) | (kp) | Friability | Disintegration |
| (Strength/ | n-10/Reported as Average | | | (%) | (min) |
| Tooling) | (Minimum-Maximum) | | | wt: 6.5 g | N = 6 |
| Batch 23A | 52.1 | 2.50 | 3.5 | 00.07 | First: 5.2 |
| (5 mg/5 mm) | (51.7-52.6) | (2.47-2.53) | (3.1-3.9) | | Last: 6.1 |
| Batch 23B | 509 | 5.08 | 9.9 | 0.17 | First: 3.5 |
| (50 mg/11 mm) | (507-510) | (5.05-5.10) | (8.9-10.6) | | Last: 4.0 |
| Batch 24B | 508 | 4.99 | 9.9 | 0.17 | First: 17.3 |
| (50 mg/11 mm) | (504-512) | (4.95-5.02) | (9.6-11.1) | | Last: 18.8 |
| Batch 25A | 51.7 | 2.54 | 2.3 | 0.08 | First: 6.0 |
| (5 mm/11 mm) | (50.7-52.6) | (2.52-2.56) | (2.0-2.5) | | Last: 6.8 |
| Batch 25B | 510 | 5.14 | 10.6 | 0.14 | First: 11.3 |
| (50 mg/11 mm) | (508-515) | (5.12-5.17) | (10.0-11.5) | | Last: 12.6 |

Sticking occurred in batch 22, with a PVP level of 1% and the formulation exhibited a large amount of fines when the formulation was scaled up to 500 g. Due to such issues, the compression of this lot was aborted. Batch 23, with a PVP level of 2%, was found to compress into tablets with no sticking issues. For Batch 24, with a PVP level of 5%, disintegration time of produced tablets increased considerably to 17 min. The final blend of Batch 24 also showed segregation between granules and powdered extra-granular excipients. Batch 25, with PVP level of 3% showed a disintegration time between that of batches 23 and 24, indicative of the role of PVP as a binder.

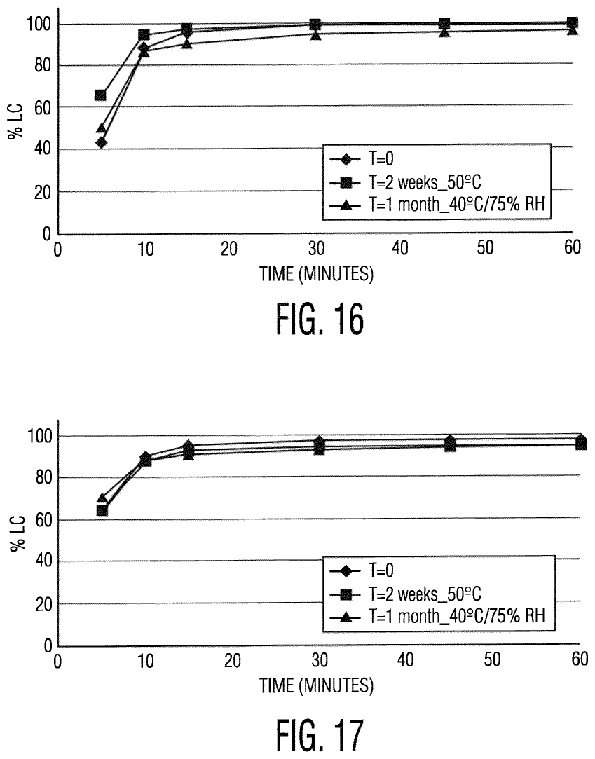

Tablets produced from Batch 23 (with 2% PVP) containing 5 mg and 50 mg of Compound 1, respectively, as described above were tested for stability after storage for 2 weeks at 50° C. and after 1 month at 40° C./75% relative humidity. Dissolution was carried out in 500 ml of 0.01N HCl, Apparatus II, stirred at 75 revolutions per min. The tablets showed chemical stability by no increase in related substances observed. The resulting dissolution profiles are illustrated in FIG. 16 (5 mg tablet) and FIG. 17 (50 mg tablet). The dissolution profiles show immediate release of Compound 1 from each tablet, and comparable profiles to initial, even after storage at the higher temperature and humidity.

### Example VIII: Phase 1 Clinical Study Protocol

Phase 1 Dose Escalation Study was initiated to assess the safety and pharmacokinetics of Compound 1 Oral Tablets (5 mg and 50 mg) compared to placebo in healthy subjects.

### Primary Study Objectives:

(i) To characterize the safety and tolerability of single ascending doses of Compound 1 in healthy subjects; (ii) To characterize the safety and tolerability of Compound 1 administered for 14 or up to 21 days in healthy subjects; (iii) To characterize the pharmacokinetics in plasma and cerebrospinal fluid (CSF) after administration of Compound 1 for 7 days in healthy subjects; (iv) To characterize the food effect on the pharmacokinetics (PK) in plasma of Compound 1 after administration of a single dose of Compound 1 in healthy subjects; and, (v) To characterize the safety and tolerability of Compound 1 administered for up to 28 days in healthy subjects.

### Secondary Study Objectives:

(i) To characterize the pharmacokinetics of single doses of Compound 1 in healthy subjects;

(ii) To characterize the pharmacokinetics of Compound 1 administered for 14 or up to 21 days in healthy subjects; (iii) To assess the QTc and drug concentration effect of Compound 1 after repeated ascending doses; (iv) To assess safety and tolerability of Compound 1 after administration for 7 days in healthy subjects; (v) To characterize the safety and tolerability of single doses of Compound 1 administered in the fed state in healthy subjects; and, (vi) To characterize the pharmacokinetics of Compound 1 administered for up to 28 days in healthy subjects.

### Exploratory Study Objectives:

(i) To explore the effect of single dose of Compound 1 administered on huntingtin (HTT) premRNA splicing in the blood of healthy subjects; (i) To explore the effect of Compound 1 administered for 14 or up to 21 days on HTT pre-mRNA splicing and HTT protein levels in the blood of healthy subjects; (ii) To explore the effect of single dose of Compound 1 administered (with food) on HTT pre-mRNA splicing in the blood of healthy subjects; and, (iii) To explore the effect of Compound 1 administered for up to 28 days on HTT premRNA splicing and HTT protein levels in the blood of healthy subjects.

### Study Design:

The Phase 1 study was conducted in 5 parts: single ascending doses (SAD)(Part 1), multiple ascending doses (MAD)(Part 2), CSF and blood sampling after 7 days of Compound 1 administration (Part 3), food effect (Part 4), and multiple dosing for up to 28 days (Part 5). Part 1, Part 2, and Part 5 are doubleblind; Part 3 and Part 4 are open-label. Note that Part 3, Part 4, and Part 5 may be conducted concurrently.

### Study Methodology:

The study was monitored by a Safety Review Committee (SRC). The intent of the SRC was to ensure that treatment does not pose undue risk to subjects. Safety and tolerability was assessed by the SRC between each cohort prior to ascending from one dose level to the next higher dose level in Part 1 (single ascending dose [SAD]) and Part 2 (multiple ascending dose [MAD]), and prior to initiating Part 3 (CSF), Part 4 (FE), and Part 5.

The SRC was composed of the following personnel: Principal Investigator or delegate (delegation only when the Principal Investigator is not available); Sponsor medical monitor or delegate (must be a physician); Other internal or external experts may be invited to participate in the review or may be consulted.

The parts of the study were not necessarily be conducted in numerical sequence and may run concurrently. The SRC met prior to the initiation of Part 5 to determine the doses to be used in this portion of the study. Doses (which may include loading and maintenance doses) were selected prior to initiation of Part 5 based on the available SAD and MAD data. The SRC did not plan to meet between cohorts within Part 5.

### Part 1 (SAD):

The single ascending dose (SAD) part of the study was randomized, double-blinded, and placebo controlled in healthy male and female subjects.

Five dose levels are planned to be tested in 5 cohorts of 8 subjects each (Cohort 1.1 to 1.5). However, the Sponsor may elect to evaluate an additional cohort(s) as long as the stopping criteria have not been met.

The initial dose in the first cohort was 51/10 of the human equivalent dose (HED) estimated from the NOAEL (no observed adverse effect level) of the (male) rat, which is the most sensitive species, following the FDA guidance on the maximum recommended starting dose (MRSD) and EMA guidelines. The NOAEL of the rat is 6 mg/kg. This was set by the observation in male rats of germ cell exfoliation in epididymis and testes. The HED of 0.97 mg/kg was calculated; this scaled in a 70 kg human to 68 mg. Adjusting this dose to 1/10, the dose of the first cohort was 6.8; the actual administered dose will be 5 mg.

In Cohort 1.1, sentinel dosing was performed in 2 subjects (1 subject with Compound 1 and 1 subject with placebo). The remaining subjects in this cohort were dosed at least 24 hours later, if no clinically significant safety issues are observed. The remaining 6 subjects (5 subjects with Compound 1 and 1 subject with placebo) may be dosed as a group. Cohort 1.1 was the only cohort in which sentinel dosing was performed. In subsequent cohorts, all 8 subjects may be dosed as a group.

After each cohort completed dosing, a dose escalation meeting was to take place. The dose level for the next cohort would be based upon the PK and safety from the previous cohort. The incremental increase in dose was determined by the relationship of mean exposure in the cohort to that of the NOAEL.

If the mean area under the curve (AUC) was <1/10 of that at the NOAEL, the dose may be increased by up to 200%. That is, the subsequent dose may be up to three times the prior dose.

If the mean AUC was between ≥1/10 and <1/5 of the AUC at the NOAEL, the dose may be increased by up to 100%. That is, the subsequent dose may be up to two times the prior dose.

If the mean AUC was between ≥1/5 and <1/2 of the AUC at the NOAEL, the dose may be increased by up to 50%. That is, the subsequent dose may be up to one- and one-half times the prior dose.

The highest dose level was that associated with a mean exposure not exceeding 1/2 of the AUC at the NOAEL; no additional escalations were to be performed. The dose escalation was to continue unless dose escalation stopping criteria were met.

Eligibility was to be assessed during a screening period of up to 28 days. Subjects were to into the clinic 1 day before dosing (Day −1). On the morning of Day 1, Compound 1 or placebo were orally administered after an overnight fast of at least 10 hours. Subjects were released from the clinic on Day 8 after all required study procedures are completed and if medically appropriate. A follow-up safety phone call was to occur 4 weeks (±1 week) after discharge on Day 8.

### Part 2 (MAD):

The multiple ascending (MAD) part of the study was randomized, double-blind, and placebo controlled in healthy male and female subjects. Up to five regimens are planned to be tested in up to 5 cohorts of 8 subjects each (Cohort 2.1 to 2.5). Within each cohort, 6 subjects were to receive Compound 1 and 2 subjects were to receive placebo. Subjects in Cohort 2.1 and 2.2 were to be dosed for 14 days, subjects in Cohort 2.3 to 2.5 were to be dosed for up to 21 days.

Part 2 may be initiated once at least 2 cohorts in Part 1 have been dosed, safety parameters have been reviewed, the respective SAD PK parameters have been calculated, and MAD dosing simulations of corresponding SAD doses have been performed. Selection of specific multiple dose levels were to be informed by available SAD PK data, simulations and general safety observed in Part 1. After the dose levels have been evaluated in once-daily format, a pharmacokinetic simulation was to be performed to determine the fluctuation within the dosing interval. In Cohort 2.3, dosing on Day 1 and 2 were to be with a loading dose that was to be higher than the dose selected for the remainder of the scheduled doses. A similar dosing schedule may be selected for Cohort 2.4 and 2.5. Alternative dosing schedules may be considered for all cohorts in Part 2 if data collected and analyzed during the study warrant it.

Eligibility was to be assessed during a screening period of up to 28 days. Subjects were to check into the clinic 1 day before dosing (Day −1). On each morning of the scheduled dosing period (ie, Day 1 up to Day 21), Compound 1 or placebo were to be orally administered after an overnight fast of at least 10 hours. Subjects were to be released from the clinic 7 days after the last dose (ie, Day 21 or up to Day 28) and after all required study procedures are completed and if medically appropriate. Subjects were to return to the clinic for an ambulant visit 7 days after release (ie, Day 28 or up to Day 35) for the collection of PK and PD (mRNA and HTT protein) samples. A follow-up safety phone call or ambulant visit was to occur on Day 49 (±7 days).

### Part 3 (CSF):

The concentrations of Compound 1 in plasma and CSF were to be assessed in an open-label design in healthy male and female subjects. A single dose of Compound 1 was to be administered daily for 7 days in 1 cohort of 6 subjects (Cohort 3.1). The dose level of Part 3 was to be determined based upon a review of the safety, tolerability, and PK data of Part 1 and Part 2 of the study. While the MAD dose was to be determined further in development, that dose and schedule was to be applied to this part of the study.

Eligibility was to be assessed during a screening period of up to 28 days. Subjects were to check into the clinic 1 day before dosing (Day −1). On the morning of Day 1 to Day 7, Compound 1 was to be orally administered after an overnight fast of at least 10 hours each day. Serial sampling of CSF and sampling of plasma for drug concentrations was to be performed on Day 7. The exact timing of the CSF and blood samples was to be determined based on the results of Part 1 and Part 2. Subjects were to be released from the clinic on Day 9 after all required study procedures are completed and if medically appropriate. A follow-up safety phone call was to occur 4 weeks (f1 week) after discharge on Day 9.

### Part 4 (FE):

The food effect (FE) part was a parallel, open-label part in healthy male and female subjects in up to 3 cohorts of 6 subjects each. Up to 3 dose levels of Compound 1 was to be administered 30 minutes after the start of a high-fat, high calorie breakfast. Part 4 may be initiated when sufficient data of Part 1 are available. The dose levels for this part were to be chosen based upon a review of available safety, tolerability and PK data as determined in Part 1 and Part 2.

Eligibility was to be assessed during a screening period of up to 28 days. Subjects were to check into the clinic 1 day before dosing (Day −1). On the morning of Day 1, Compound 1 was to be orally administered after ingestion of a standardized, high-fat, high calorie breakfast. Subjects are released from the clinic on Day 8 after all required study procedures are completed and if medically appropriate. A follow-up safety phone call was to occur 4 weeks (+1 week) after discharge on Day 8.

Part 5 (Multiple Dosing for up to 28 days [MD28D]):

Part 5 was a randomized, double-blind, and placebo-controlled assessment of multiple doses for up to 28 days in healthy male and female subjects. Up to 3 cohorts of 8 subjects each are planned. Prior to the initiation of Part 5, the SRC was to meet for selection of dose (which may include loading and maintenance doses), dosing regimen (including fed or fasted condition), and duration (up to 28 days) for this part of the study based upon available data from the completed cohorts of Part 1 and Part 2. Within each cohort, 6 subjects were to receive Compound 1, and 2 subjects were to receive placebo. The total dose on any day was not to exceed doses that were established as well tolerated in Part 1 (SAD).

Eligibility was to be assessed during a screening period of up to 28 days. Subjects were to check into the clinic 1 day before dosing (Day −1). On each day of dosing, Compound 1 or placebo was to be orally administered in the morning either after an overnight fast or following a standard highfat meal, per the SRC determined regimen selected for a given cohort. Subjects were to be released from the clinic 7 days after the final dose and after all required study procedures are completed and if medically appropriate. Subjects were to return to the clinic for an ambulant visit 7 days after being released from the clinic for the collection of PK and PD (mRNA and HTT protein) samples, and safety assessments. On Day 1 and on the day of anticipated maximum exposure (ie, either Day 2 or, if loading doses are not used, Day 29) patients were to be monitored with a 24-hour Holter monitor device.

### Study Population:

Part 1: Up to 48 male and female subjects between 18 and 65 years of age, inclusive.

Part 2: Up to 40 male and female subjects between 18 and 65 years of age, inclusive.

Part 3: 6 male and female subjects between 50 and 65 years of age, inclusive.

Part 4: Up to 18 male and female subjects between 18 and 65 years of age, inclusive.

Part 5: Up to 24 male and female subjects between 18 and 65 years of age, inclusive.

### Inclusion Criteria:

The following criteria must be met by all subjects to be considered for study participation:

For Part 1, Part 2, Part 4, and Part 5: Healthy male or female subjects aged from 18 to 65 years old, inclusive, at Screening. For Part 3: healthy male of female subjects aged 50 to 65 years old, inclusive, at Screening.

Subjects must understand the nature of the study and must provide signed and dated written informed consent before the conduct of any study-related procedures.

Body Mass Index (BMI) of ≥18.5 kg/m² and ≤30.0 kg/m² with a body weight ≤50.0 kg for male subjects and a body weight ≥45.0 kg for female subjects at Screening.

Healthy as determined by the Investigator, based upon a medical evaluation including medical history, physical examination, laboratory test results, ECG recording (eg, QTcF ≤450 msec for males and QTcF ≤470 ms for females) and vital signs. Out of range values can be repeated once.

Male subjects and female subjects of childbearing potential must be willing to use 2 methods of birth control for the duration of the study and for 30 days after the last dosing.

Postmenopausal female subjects must have had ≥12 months of spontaneous amenorrhea (with follicle-stimulating hormone (FSH) ≥30 mIU/mL at Screening). Surgically sterile women are defined as those who have had a hysterectomy, bilateral ovariectomy, or bilateral tubal ligation ≥6 months prior to Screening.

All female subjects of childbearing potential must have a negative serum pregnancy test result at Screening and a negative urine pregnancy test on Day −1.

Male subjects must agree to not donate sperm for the duration of the study and for at least 3 months after the last dosing.

Part 3 only: Subject must be willing to undergo lumbar puncture for CSF sampling.

Part 4 only: Subject must be willing and able to consume the entire high-fat breakfast in the designated timeframe.

### Exclusion Criteria:

Subjects will be excluded when they meet any of the following criteria:

Subjects that participated in any drug or device clinical investigation within 60 days prior to Screening or who anticipate participating in any drug or device clinical investigation within the duration of this study.

Prior or ongoing medical condition (eg, concomitant illness, psychiatric condition), medical history, physical findings that, in the Investigator's opinion, could adversely affect the safety of the subject or could impair the assessment of study results.

An abnormal general neurological examination.

Presence of any clinically significant abnormality during Screening.

Any psychological, emotional problems, any disorders or resultant therapy that are likely to invalidate informed consent or limit the ability of the subject to comply with the protocol requirements.

A positive Hepatitis B surface antigen, positive Hepatitis C antibody or human immunodeficiency virus (HIV) antibody result at Screening.

Donation of plasma within 7 days prior to dosing. Donation or loss of blood (excluding volume drawn at screening or menses) of 50 mL to 499 mL of blood within 30 days, or more than 499 mL within 56 days prior to the dosing.

Excessive alcohol consumption (regular alcohol intake ≥21 units per week for male subjects and >14 units per week for female subjects) within 6 months prior to Screening. One unit (8 g) is equivalent to a % pint (280 mL) of beer, 1 measure (25 mL) of spirits or 1 small glass (125 mL) of wine.

The subject is a smoker or uses other nicotine-containing products. Ex-smokers must have ceased smoking >3 months prior to Screening.

A positive urine drug screen, cotinine screen or alcohol breath test at Screening or on Day 1 of each treatment period.

Females who are pregnant or nursing.

Subject has previously received Compound 1.

Part 3 only: Contraindication to lumbar puncture, eg, low platelet count, abnormal prothrombin time international normalized ratio (PT-INR), spinal deformities or other spinal conditions that in the judgment of the Investigator would preclude a lumbar puncture.

### Duration of Treatment:

Part 1: 1 day; Part 2: 14 days (Cohort 2.1 and 2.2) or up to 21 days (Cohort 2.3 to 2.5); Part 3: 7 days; Part 4: 1 day; Part 5: up to 28 days.

### Criteria for Evaluation:

### Efficacy:

The following PK parameters were assessed wherever feasible on Part Day 1 (Single Dose) PK [Part 1 (SAD), Part 2 (MAD, Day 1), Part 4 (FE), and Part 5 (MD28D, Day 1)]: Cₘₐₓ; the maximum observed plasma concentration, Cₘₐₓ/D; Dose normalized Cₘₐₓ (Part 1 only); Tₘₐₓ; the time to reach Cₘₐₓ; AUC_{0.24} (Area under the concentration-time curve from 0 to 24 hours); AUC₀₋₇₂ (Area under the concentration-time curve from 0 to 72 hours); AUC₀₋ₜₐᵤ (Area under the concentration-time curve within dosing interval, calculated by linear up/log down trapezoidal method, for Part 2 only); AUC₀₋ₜ (Area under the concentration-time curve from time zero to time t, where t is the time of the last measured (or measurable) concentration (Cₜ), calculated by linear up/log down trapezoidal method (Parts 1 and 4 only); AUC₀₋ₜ/D (Dose normalized AUC from time zero to the last quantifiable concentration, Part 1 only); AUC_{0-inf}; (Area under the concentration-time curve from time zero to infinity, AUC_{0-inf}=AUC₀₋ₜ+Cₜ/λ_{z}, where λ_{Z} is the terminal elimination rate constant, calculated by linear up/log down trapezoidal method (Parts 1 and 4 only); AUC_{0-inf}/D (Dose normalized AUC from time zero to infinity, Part 1 only); λ_{z} (Apparent terminal rate constant calculated by linear regression of the terminal linear portion of the log concentration vs. time curve, Parts 1 and 4 only); t_{1/2} (Apparent terminal half-life calculated as ln(2)/λ_{z}, Parts 1 and 4 only); CL/F (Total body clearance, calculated as Dose/AUC_{0-inf}, Parts 1 and 4 only); and, V_{z}/F (Apparent volume of distribution, calculated as Dose/(λ_{z}*AUC_{0-inf})).

The following PK parameters were assessed wherever feasible on Day 14, Day 21, or Day 28 (Multiple Dose) PK [Part 2 (MAD) Cohort 2.1 and 2.2 (Day 14), Cohort 2.3 to 2.5 (Day 21), and Part 5 (MAD) Cohort 5.1 to 5.3 (Day 28)]: Cₘₐₓ (The maximum observed plasma concentration over a dosing interval); Tₘₐₓ (The time to reach Cₘₐₓ over a dosing interval); Cₘᵢₙ (The minimum concentration over a dosing interval); C_{avg} (Average concentration over a dosing interval); AUC₀₋ₜₐᵤ (Area under the concentration-time curve within dosing interval, calculated by linear up/log down trapezoidal method); AUC₀₋ₜₐᵤ/D (Dose normalized AUC₀₋ₜₐᵤ); λ_{z} (Apparent terminal rate constant calculated by linear regression of the terminal linear portion of the log concentration vs. time curve); t_{1/2} (Apparent terminal half-life calculated as ln(2)/λ_{z}); CL/F (Total body clearance, calculated as Dose/AUC₀₋ₜₐᵤ); V_{z}/F (Apparent volume of distribution, calculated as Dose/(λ_{z}*AUC₀₋ₜₐᵤ)); AUCR_{auc} (Accumulation ratio based on AUC₀₋ₜₐᵤ:AUC₀₋ₜₐᵤ on Last Dose*/AUC₀₋ₜₐᵤ on Day 1); and, AUCR_{cmax} (Accumulation ratio based on Cₘₐₓ:Cₘₐₓ on Last Dose*/Cₘₐₓ on Day 1).

The following PK parameters were assessed wherever feasible for *Last Dose on Day 14 for Part 2 (Cohort 2.1 and 2.2) or Day 21 for Part 2 (Cohort 2.3 to 2.5) or Day 28 for Part 5 (Cohort 5.1 to 5.3) and Day 7 (Multiple Dose) PK [Part 3 (Day 7)]:

Cₘₐₓ (The maximum observed plasma concentration); Tₘₐₓ (The time to reach Cₘₐₓ); AUC_{0.5-12} (Area under the concentration-time curve from time 0.5 to 12 hours, calculated by linear up/log down trapezoidal method); and, CSF/Plasma ratio (Concentration ratios in CSF over plasma (Part 3 only).

### Safety:

The following parameters were defined as parameters regarding safety and tolerability:

Change from baseline to each scheduled time point up to EOS for vital signs; Change from baseline to each scheduled time point up to EOS for ECG parameters; Change from baseline to each scheduled time point up to EOS for clinical laboratory tests; Changes from baseline in C-SSRS scores (Part 2, Part 3, and Part 5 only); Treatment-emergent adverse events (AEs) up to EOS; Treatment-emergent AEs leading to premature discontinuation of study drug; Treatment-emergent serious adverse events (SAEs) up to EOS; and, Abnormalities in physical examination.

### Statistical Methods:

### Pharmacokinetics:

Individual subject listings were provided. Mean and individual plasma concentration-time profiles for Compound 1 were presented graphically for each group.

PK variables were to be summarized using arithmetic mean, standard deviation, geometric mean, median, minimum, maximum, and CV %.

Attainment of steady state conditions were to be determined by visual inspection of the trough plasma concentrations.

To assess the effect of food, the PK parameters of Compound 1 in fasted (Part 1) and fed (Part 4) condition were to be graphically displayed, and descriptive statistics were to be prepared. Statistical analysis per dose level were to be performed in 6 subjects for Compound 1 using treatment in fed condition as test (Part 4) and the treatment with the same dose in fasted condition as reference (Part 1).

The primary PK parameters were to be Cₘₐₓ, AUC₀₋ₜ, and AUC_{0-inf}. The PK parameters of Cₘₐₓ, AUC₀₋ₜ, and AUC_{0-inf} were to be naturally log-transformed first, and the means of these logtransformed parameters were to be estimated by the linear model with treatment (Compound 1 administered under fed conditions over that of Compound 1 administered under fasting conditions) as the only fixed factor. The difference of these means (in log scale) and its 90% confidence interval (CI) were to be exponentiated to form the ratio of geometric means (GMR) and corresponding CI for the ratio. Absence of food effect were to be concluded if all 90% CI results of the GMRs for the Cₘₐₓ, AUC₀₋ₜ, and AUC_{0-inf} are contained within the interval 80.00%-125.00%.

### Safety:

All safety parameters were to be summarized by dose level in Part 1 through Part 5.

Summary statistics (mean, median, standard deviation, minimum, maximum, and number of available observations) were to be provided for continuous demographic variables (eg, age, height, and weight). Individual subject listings of demographic data were to be provided.

Qualitative demographic characteristics (gender, race) were to be summarized by counts and percentages. Other baseline subject characteristics (eg, medical history, physical examination clinical findings, previous medications, and inclusion/exclusion checklist) were to only be listed.

ECG variables, vital sign measurements and laboratory measurements were to be summarized at each time point using mean, median, standard deviation, min, max, number of available observations, and change from baseline. C-SSRS parameters were to be analyzed using descriptive statistics where appropriate. Individual subject listings of ECG data, vital signs data, laboratory measurements and C-SSRS (Part 2, Part 3, and Part 5 only) were to be provided.

Distributions of these parameters were to be compared between the treatment groups (fasted or fed) only descriptively. No statistical inference were to be performed.

Holter analysis/Compound 1 plasma concentration-QTc effects may be performed, and results were to be provided in a separate report.

### Phase 1 Study Results

The key objectives of the Phase 1 healthy volunteer trial were to establish a target dose range of Compound 1 for lowering HTT mRNA and protein. The trial consisted of single (SAD) ascending dose (SAD) and multiple (MAD) ascending dose (MAD) cohorts. The dosing in all cohorts was well-tolerated with no safety-related findings, exhibiting dose-dependent splicing of HTT mRNA. The study duration for the MAD cohort was of a longer duration, enabling longer-term evaluation of HTT mRNA splicing and HTT protein lowering. The MAD cohort demonstrated that Compound 1 showed a long drug half-life, with maintenance of splicing up to 72 hours following the last dose.

The CSF sampling enabled the evaluation of pharmacokinetics of Compound 1 in the CSF wherein Compound 1 levels in the CSF were compared with Compound 1 levels in plasma. The Phase 1 Study results demonstrated that Compound 1 levels in the CSF were equal to or greater than levels observed in plasma. The food effect portion enabled the evaluation of pharmacokinetics of Compound 1 in plasma after administration of a single dose of Compound 1 in healthy subjects.

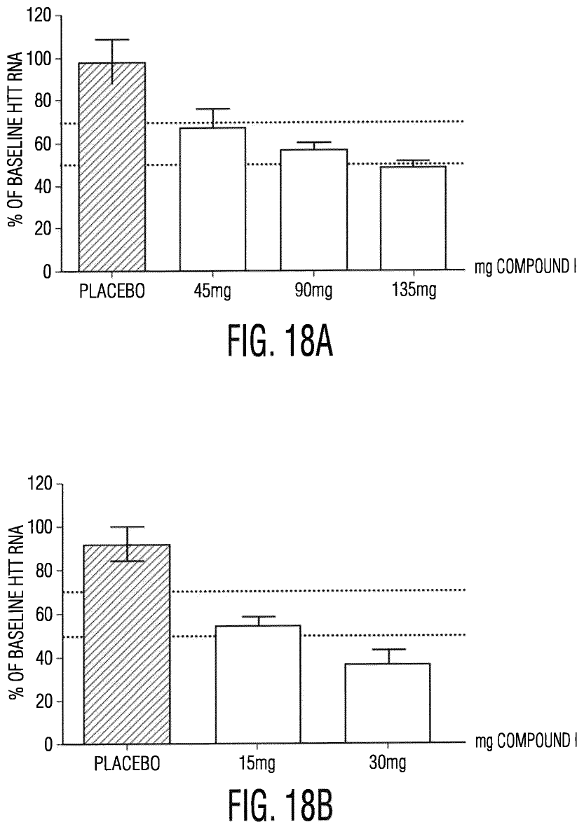

As shown in FIG. 18A, the SAD cohort resulted in a dose-dependent lowering of HTT mRNA in whole blood taken from healthy volunteers 24 hours after they were administered with either placebo, 5 mg, 15 mg, 45 mg, 90 mg, or 135 mg of Compound 1.

Similarly, the MAD cohort (FIG. 18B) also showed a dose-dependent lowering of HTT mRNA in whole blood taken from healthy volunteers dosed with either placebo, 15 mg or 30 mg of Compound 1 for 14 days. The amount of HTT mRNA was then evaluated by RT-PCR 6 hours after administration of Compound 1 on day 14.

The target level of 30-50% lowering was achieved with the lowest dose tested both in the SAD and MAD cohorts. The half-life of HTT mRNA was estimated to be about 24 hours. Thus, after one day, if no HTT mRNA was synthesized, the total amount of HTT mRNA would be predicted to be about 50% of baseline. The administration of Compound 1 Compound in the SAD cohort essentially inhibited all de novo HTT mRNA synthesis. Thus, even with higher concentrations of Compound 1, the total amount of HTT mRNA remained at about 50% of baseline representing the amount of HTT mRNA synthesized prior to the administration of Compound 1.

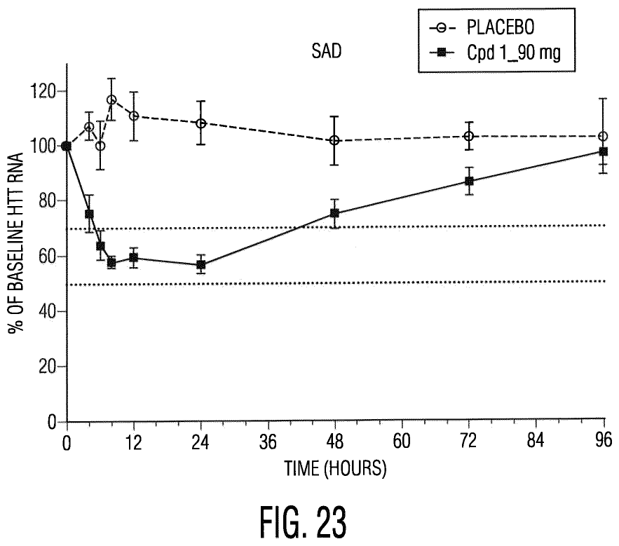

Results from measurement of HTT mRNA in the whole blood of subjects in the SAD cohorts are illustrated in FIG. 23. The results also show that the HTT splicing effect of Compound 1 is reversible and persists for 72 hours post cessation of treatment.

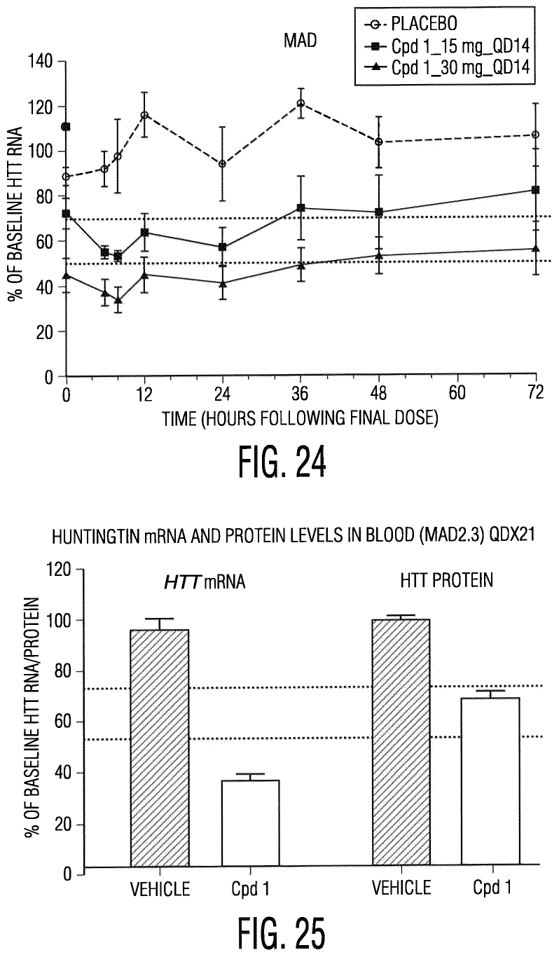

Results from measurement of HTT RNA in the whole blood of human subject administered a placebo or 15 or 30 mg of Compound 1, as described in the Multiple Ascending Dose (MAD) study above are illustrated in FIG. 24. HTT splicing was monitored after the final dose at day 14, calculated as % HTT remaining from baseline (pre-dose day 0).

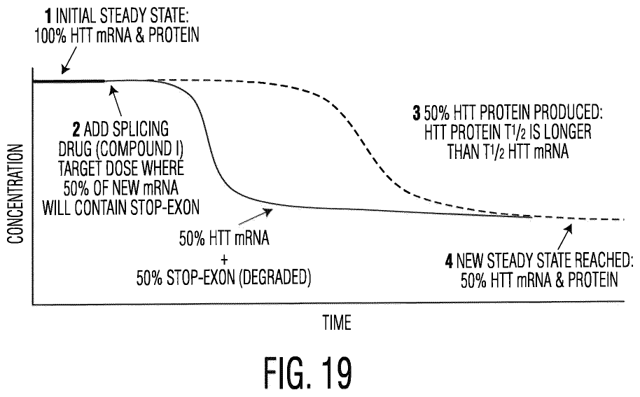

FIG. 19 is an exemplary depiction of HTT mRNA and protein degradation kinetics that leads to a steady-state levels of RNA and protein.

In untreated cells, there is a steady state level of mRNA and protein because the amount of mRNA or protein being synthesized matches the amount that is being degraded, so that the mRNA and protein levels are the same over time. The addition of Compound 1 triggers the inclusion of the HTT pseudoexon into the transcript which results in the rapid decay of the HTT mRNA and a reduction of HTT mRNA to −50% of baseline. The half-life of the HTT mRNA is about 24 hours. Hence, a day after drug treatment, the amount of HTT mRNA present is regulated by the dose of Compound 1. In this example, ˜50% of newly synthesized mRNA was inhibited. Of the HTT mRNA synthesized prior to treatment, about 50% is degraded after 24 hours. The HTT protein level depends on how much mRNA is produced. Thus, a reduction by 50% would cause a 50% reduction of the HTT protein. However, HTT protein has a half-life of about 5-7 days, so it takes longer to get to the new steady state level. Finally, a new steady state is reached where 50% of the mRNA is present, and the new level of protein has fallen to 50% of the original amount. Changes in HTT protein levels were assessed in MAD cohort over a longer period of time. Accordingly, healthy subjects were treated for 21 days before the amount of HTT mRNA and protein was measured in blood samples taken from each subject.

FIG. 25 shows the huntingtin mRNA and protein levels measured in whole blood from MAD cohort 2.3 (30 mg administered for 21 days with 100 mg LD for 2 days), as described above, as a percent of baseline, after administration of vehicle or compound 1 to a human, 24 hours after the last dose. The results show HTT mRNA reduction reached steady state. Longer dosing was required for HTT protein levels to reach maximal steady state reduction. It is anticipated that the observed HTT mRNA changes in blood will result in similar decreases in HTT protein levels in Huntington's disease patients when steady state decrease in HTT is attained over time with continued treatment with Compound 1.

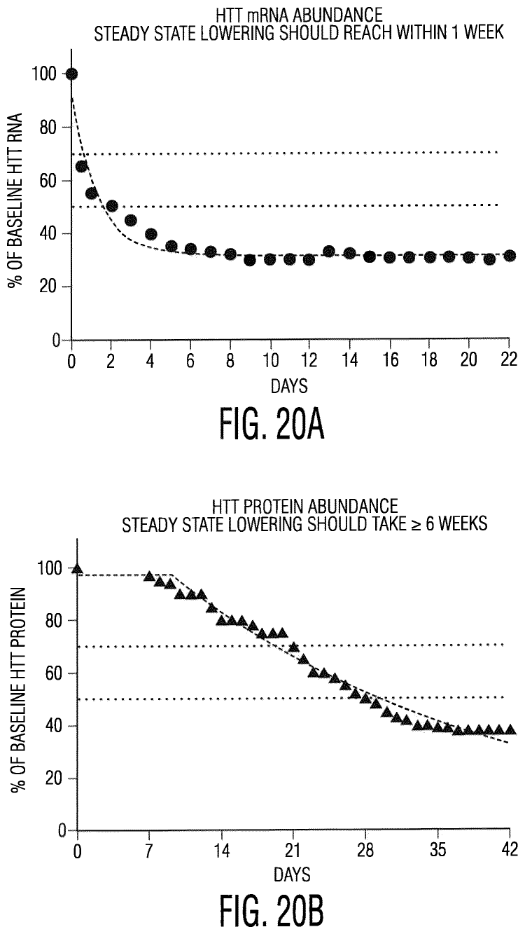

FIG. 20 shows graphs that model the rate of HTT mRNA (FIG. 20A) and HTT protein (FIG. 20B) decay based on their half-lives and predict the time to reach steady state after Compound 1 treatment at 30 mg daily dose. For HTT mRNA, the half-life was estimated to be about 24 hours. HTT mRNA reaches steady state after approximately 5 days. For HTT protein, the half-life was estimated to be 5-7 days and consequently HTT protein steady state levels would only be attained about 6 weeks from the beginning of treatment.

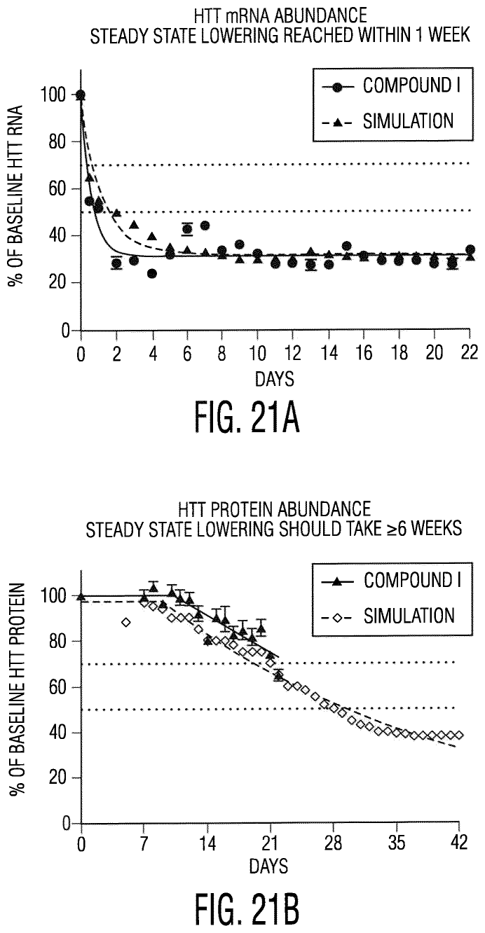

FIG. 21 compares the trajectory of HTT mRNA (FIG. 21A) and protein (FIG. 21B) lowering seen in the Multiple Ascending Dose Study with those values predicted from the half-life of HTT mRNA and protein as shown in FIG. 20. The results show that HTT mRNA levels rapidly decreased and reached steady state at about 4-5 days of treatment. As predicted, the rate of protein lowering was much slower, but after 21 days of treatment there was approximately 40% lowering in the amount of HTT protein. Equivalent steady state levels of HTT mRNA and protein could therefore be reached after about 4-5 weeks from the onset of treatment.

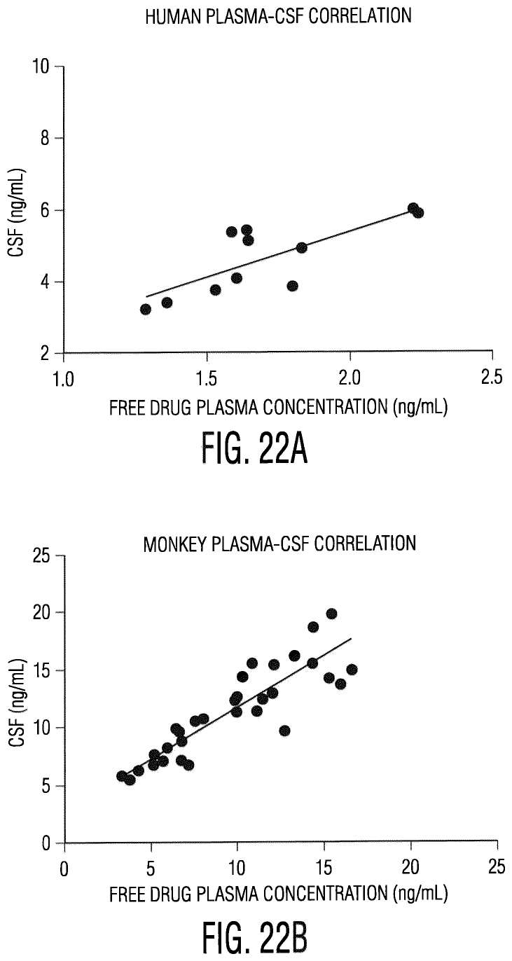

As shown in FIG. 22, the level of Compound 1 in the cerebrospinal fluid (CSF) demonstrated that Compound 1 therefore crossed the blood brain barrier and was in direct correlation with the level of Compound 1 in free plasma both in humans (FIG. 22A) and non-human primates (FIG. 22B). The two subjects in this cohort received 30 mg daily dose. Compound 1 therefore crossed the blood brain barrier. The levels of Compound 1 found in the CSF were at least equivalent or greater than levels observed in the plasma, thus demonstrating in humans that Compound 1 was not subject to efflux.

In the food effect cohort, Compound 1 showed similar exposures regardless of whether the subjects were fasted or fed.

In conclusion, the Phase I study demonstrated Compound 1 penetrated the blood brain barrier and selectively reduced HTT mRNA and protein in both the CNS and periphery in a dose dependent manner. These results confirm that exposure to Compound 1 in human patients leads to demonstrable reduction for both HTT mRNA and HTT protein.

### Example VIII: Phase 2 Clinical Study Protocol

A 12 week Phase 2, Randomized, Placebo-Controlled, Dose-Finding Study to Evaluate the Safety and Efficacy of Compound 1 in Subjects with Huntington's Disease.

Prior to the development of this Phase 2 study, Compound 1 was extensively evaluated in in vivo and in vitro preclinical pharmacology models, in a comprehensive toxicology program, and in an ongoing Phase 1 study in healthy volunteers. Together, the resulting data validate that Compound 1 treatment results in dose-dependent pre-mRNA splicing and reduced protein transcription and that Compound 1 treatment is safe and well tolerated in the clinic at single doses as high as 135 mg and multiple doses as high as 30 mg for 21 days.

The present 12-week double-blind study will allow for the quantification of the effect of Compound 1 on total HTT (tHTT) protein reduction in subjects with HD and evaluation of the safety of two doses over 12 weeks of Compound 1 treatment.

A parallel-group design was selected because it allows recruitment of patients for all treatment arms in the same timeframe. The time course in untreated patients for HTT protein, mRNA, and other indicators of drug response in the blood are not available. The use of a parallel arm design with concurrent placebo control allows a direct assessment comparison to determine the effect of active treatment.

The patient population was selected to reduce variability in an otherwise heterogeneous disease population by identifying subjects with active disease who have not yet experienced functional decline. In this study, at randomization, subjects will thus be enrolled in the trial based upon CAG repeat length and Baseline measures of the Symbol Digit Modality Test (SDMT), Total Motor Score (TMS), Independence Scale (IS) and Total Functional Capacity (TFC). These factors will be used to identify and enroll subjects with active disease who have not yet experienced functional decline, which may indicate a disease progression amenable to intervention. The Huntington's disease prognostic index (PIHD) or its normed version (PINHD) score can be used to predict likelihood of HD progression. The PIN score will be calculated at Baseline to identify subjects eligible for participation in the study.

Based on the kinetics of Compound 1-mediated HTT lowering in humans, the maximal extent of tHTT protein lowering in HD patients is expected to be achieved between 4 and 6 weeks. The 12-week dosing regimen may further demonstrate that a steady state decrease in tHTT is maintained over time with continued Compound 1 treatment in the Phase 2 Study, followed by a one year, open label extension. In addition to the primary endpoints of tHTT protein change from Baseline and safety, the Phase 2 study includes exploratory clinical outcome endpoints to assess the effect of Compound 1 on subjects' cognition and motor function as measured by the Unified Huntington's Disease Rating Scale (UHDRS). The UHDRS has been extensively studied and developed to assess disease progression in multiple domains. Cognitive impairment, motor function loss, and accelerated brain volume loss in the caudate and putamen are key features of this disorder and have a notable impact on quality of life. The assessment of more sensitive and early motor changes via wearable devices will also be included in the Phase 2 study as an exploratory endpoint. Studying these endpoints over 12 weeks will provide insight into the rate of change in earlier stages of disease and identify key measurements which may be early indicators of HD progression.

### Risk/Benefit Assessment

As described, HD is a relentlessly progressive, neurodegenerative disorder. Early in the course of the disease, patients exhibit subtle symptoms; as the disease progresses, involuntary writhing movements become more pronounced, voluntary motor capabilities decline, and speech and swallowing are increasingly impaired, while aggressive and disinhibited behavior become more frequent. Late-stage disease is marked by severe inability to walk, speak, swallow, or care for oneself, culminating in the need for full-time care and ultimately death, typically 15 to 18 years after the onset of symptoms (see, Caron, N, Wright, G and Hayden, M; (2020a), Huntington Disease; Seattle, WA; University of Washington).

There are currently no disease modifying interventions approved for use in HD and, without intervention, the patient population to be included in this trial will face continued disease progression, loss of function, and inevitably, death. The inexorable disease progression and inevitable mortality of the disease indicate that HD represents a high unmet medical need. Reduction of mHTT has been confirmed as an important therapeutic target.

As described above, in the Phase 1 study, multiple doses of Compound 1 were associated with marked reductions in HTT mRNA and protein. Pharmacokinetic-pharmacodynamic (PKPD) modeling based on interim data from the Phase I study determined that exposures at the 10 mg and 20 mg QD doses were associated with decreases in full-length HTT mRNA levels that precisely bookend the established mean 30% to 50% target range for HTT protein reduction. The 10 mg and 20 mg QD doses are thus anticipated to be associated with therapeutic benefit and the eventual slowing of disease progression in this Phase 2 study.

The Phase 1 study results provided evidence of Compound 1 safety and tolerability at single doses ranging from 5 mg to 135 mg and multiple doses of 15 mg and 30 mg for durations of up to 21 days. In this study, Compound 1 was safe and generally well tolerated. In both the single ascending dose (SAD) and multiple ascending dose (MAD) portions of the Phase 1 study, the overall incidence of AEs was comparable between subjects who received placebo and those who received Compound 1. There were no events considered to be dose-limiting toxicities, and all Adverse Events (AEs) were resolved at the time of the interim analysis cut-off date. There were also no clinically significant laboratory abnormalities or electrocardiogram (ECG) findings at any dose in either portion of the study.

The Phase 1 study will have a Data and Safety Monitoring Board (DSMB) that will closely monitor the safety of subjects. Based on the preclinical and clinical data to date, Compound 1 has a favorable risk/benefit profile in subjects with HD.

### Primary Study Objectives:

Evaluate the safety and pharmacodynamic effects of 2 treatment regimens of Compound 1 and placebo in subjects with Huntington's disease (HD) as assessed by: (i) Occurrence of treatment-emergent adverse events (TEAEs) and abnormalities in laboratory values, electrocardiogram (ECG), vital signs, slit lamp eye examination, and physical examination; and,

(ii) Reduction in blood total huntingtin protein (HTT) levels. This aspect is intended to demonstrate the safety, tolerability and pharmacology of Compound 1 and reduction of HTT mRNA and HTT protein in HD patients.

### Secondary Study Objectives:

(i) Determine the effect of Compound 1 on HTT mRNA in blood and mHTT protein in cerebrospinal fluid (CSF); and, (ii) Reduction in blood mutant huntingtin protein (mHTT) levels. This aspect is intended to demonstrate the effect of Compound 1 on blood based, CSF-based and radiographic biomarkers of Huntington's disease.

### Exploratory Study Objectives:

(i) Assess the effect of Compound 1 on change in whole brain, caudate, and putamen volume via volumetric magnetic resonance imaging (vMRI); (ii) Assess the effect of change in ventricular volume via vMRI; (iii) Assess the effect of Compound 1 on plasma and CSF neurofilament light chain (NfL) protein concentrations; (iv) Assess change after 12 weeks of treatment in relevant scales, which will include an Assessment using the Unified Huntington's Disease Rating Scale (UHDRS) and each of its subcomponents, including (a) Symbol Digit Modalities Test (SDMT), (b) Total Motor Score (TMS), (c) Independence Scale, (d) Total Functional Capacity (TFC); (e) Gait and motor assessment via a wearable accelerometer; (f) Clinical Global Impression of Change (CGI-C); and, (g) Huntington's Disease Quality of Life questionnaire (HDQoL).

### Pharmacokinetic Objective:

Evaluate the concentration of Compound 1 in subjects with HD.

### Clinical Endpoints:

### Primary Safety Endpoints:

Evaluate the safety profile as characterized by TEAEs, laboratory abnormalities, ECG, vital signs, slit lamp eye examination, and physical examination.

### Primary Efficacy Endpoint:

Change from Baseline in blood total HTT protein at Visit 5.

### Biomarker Endpoints:

(i) Percent reduction in HTT protein in CSF; (ii) Changes in neurofilament light chain (NfL) in plasma and CSF; and (iii) Change in caudate, putamenal, ventricular volume on volumetric MRI imaging.

### Secondary Endpoints:

(i) Change from Baseline in blood HTT mRNA at Visits 3, 4, and 5; (ii) Change from Baseline in CSF mHTT at Visit 5; and, (iii) Change from Baseline in blood mHTT protein at Visit 5.

### Exploratory Endpoints:

(i) Change from Baseline in whole brain, caudate, putamen, and ventricular volume (as assessed by vMRI); (ii) Change from Baseline in plasma and CSF NfL protein concentrations; (iii) Change from Baseline in UHDRS scores for each subscale, including the SDMT, TMS, Independence Scale, and TFC; (iv) Change from Baseline in total UHDRS; (v) Change from Baseline in wearable accelerometer assessment of gait and motor function; (vi) Assessment of change via the CGI-C; and, (vii) Change from Baseline in the HDQoL questionnaire.

### Pharmacokinetic Endpoint

(i) Plasma trough concentration (Ctrough) and accumulation ratio of plasma of Compound 1 on Visits 3, 4, and 5; and, (i) Accumulation ratio of Compound 1 in CSF on Visit 5.

### Biomarker Endpoints:

(i) Percent reduction in HTT protein in CSF; (ii) Changes in neurofilament light chain (NfL) in plasma and CSF; and (iii) Change in caudate, putamenal, ventricular volume on volumetric MRI imaging.

### Study Design/Methodology:

The Phase 2 Study is a randomized, placebo-controlled, parallel arm, dose-finding study to evaluate the safety and efficacy of 10 and 20 mg of Compound 1 and to determine the HTT protein lowering effect of these doses after 12 weeks of treatment in subjects with HD.

Individuals who sign an informed consent will enter screening to determine eligibility for the study. At Screening, potential subjects will have their gene mutation status confirmed by the Investigator (either via historical gene sequencing or through an in-study gene sequencing assessment) and undergo additional evaluation to confirm they meet the enrollment criteria. Subjects who satisfy all enrollment criteria at Screening will undergo baseline evaluations and be randomized to either 10 or 20 mg of study drug or placebo in a 1:1:1 randomization for a total of 12 weeks on treatment (plus or minus visit windows). Once assigned treatment, subjects will take their assigned dose of study medication, once a day, in the morning, at least 2 hours before their first meal of the day. Subjects will be asked to return to the clinic every 28 days after randomization (approximately Days 29, 57 and Day 85) or receive home care services in lieu of in-person visits to undergo study assessments. On Day 85, subjects will take their final dose of study medication and complete the end of study assessments. There will be a follow-up safety visit on Day 113 via telephone/telehealth to collect AEs.

### Sample Size Justification:

The sample size calculation is based on mean change from Baseline in blood total HTT protein at Visit 5 (primary endpoint). Using effect size of 0.85 (i.e., the magnitude of treatment difference is 85% of one standard deviation), achievement of 90% power at 2-sided alpha level 0.05 would require 31 subjects. Assuming a 10% dropout rate, approximately 35 subjects will be randomized to each dose.

### Planned Number of Patients:

Approximately 200 adult male and female subjects will be enrolled.

### Inclusion Criteria:

Individuals eligible to participate in this study include those who meet all of the following inclusion criteria: (i) Ambulatory male or female patient aged 25 years and older, inclusive; (ii) Subject (or legally authorized representative) is willing and able to provide informed consent and comply with all protocol requirements; (iii) Genetically confirmed HD diagnosis with a cytosine-adenine-guanine (CAG)repeat length from 42 to 50, inclusive; (iv) A UHDRS-Independence Scale score of 100; (v) A TFC score of 13; (vi) A normed prognostic index for HD score between 0.18 to 4.93, inclusive; (vii) Women of childbearing potential (WOCBP): must agree to use highly effective methods of contraception during dosing and for 6 months after stopping the study medication.

WOCBP are defined as women who are fertile, following menarche and until becoming postmenopausal unless permanently sterile. Permanent sterilization methods include hysterectomy, bilateral salpingectomy, and bilateral oophorectomy. A postmenopausal state is defined as no menses for 12 months without an alternative medical cause. A high follicle stimulating hormone (FSH) level in the postmenopausal range may be used to confirm a postmenopausal state in women not using hormonal contraception or hormonal replacement therapy. However, in the absence of 12 months of amenorrhea, a single FSH measurement is insufficient. Highly effective contraception methods are defined as those that can achieve a failure rate of less than 1% per year when used consistently and correctly and include those selected from (a) combined (estrogen and progestogen containing) hormonal contraception associated with inhibition of ovulation, including contraception that is administered orally (WOCBP using oral contraception should have been stable on the same pill for a minimum of 3 months prior to Screening), intravaginally, or transdermally; (b) progestogen-only hormonal contraception associated with inhibition of ovulation, including contraception that is administered orally (WOCBP using oral contraception should have been stable on the same pill for a minimum of 3 months prior to Screening), via injectable, implantable, intrauterine device or intrauterine hormone-releasing system; or, (c) contraception associated with bilateral tubal occlusion, vasectomized partner or sexual abstinence.

(viii) Sexually active and fertile males must use a condom during intercourse while taking study drug and for 6 months after stopping study drug, and should neither father a child nor donate sperm in this period. A condom is required to be used also by vasectomized men in order to prevent potential delivery of the drug via seminal fluid.

### Main Criteria for Exclusion:

Individuals are not eligible to participate in this study if they have met or meet any of the following exclusion criteria: (i) Inability or unwillingness to swallow oral tablets; (ii) Receipt of an experimental agent within 90 days or 5 half-lives prior to Screening or anytime over the duration of this study, including RNA- or DNA-targeted HD specific investigational agents, such as antisense oligonucleotides, cell transplantation, or any other experimental brain surgery; (iii) Any history of gene therapy exposure for the treatment of HD; (iv) Participation in an investigational trial or investigational paradigm (such as exercise/physical activity, cognitive therapy, brain stimulation, etc.) within 90 days prior to Screening or anytime over the duration of this study; (v) Presence of an implanted deep brain stimulation device; (vi) Family history of early onset cataracts or presence of cataracts at Baseline using a cataract grading system (Lens Opacities Classification System III) exam; (vii) Brain and spinal pathology that may interfere with CSF homeostasis and circulation, increased intracranial pressure (including presence of a shunt for the drainage of CSF or an implanted CNS catheter), malformations, and/or tumors; (viii) Hospitalization for any major medical or surgical procedure involving general anesthesia within 12 weeks of Screening or planned during the study; (ix) At significant risk of suicide as measured by the Columbia Suicide Severity Rating Scale (C-SSRS) with a moderate risk rating or higher score; (x) Risk of a major depressive episode, psychosis, confusional state, or violent behavior as assessed by the Investigator; (xi) Any medical history of brain or spinal disease that would interfere with the lumbar puncture process or safety assessments; (xii) History of malignancy of any organ system (other than localized basal cell carcinoma of the skin or in situ cervical cancer), treated or untreated, within the past 5 years, regardless of whether there is evidence of local recurrence or metastases; (xiii) Any medical history or condition that would interfere with the ability to complete the protocol-specified assessments (eg, implanted shunt, conditions precluding MRI scans); (xiv) Antidepressant or benzodiazepine use, unless receiving a stable dose for at least 6 weeks prior to Screening and with a dose regimen that is not anticipated to change during the study; (xvi) Lifetime history of drug or alcohol use in the high risk category of risk drinking levels according to the World Health Organization for a duration of 1 month or longer as assessed by the Investigator; (xvii) Clinically significant medical condition, which in the opinion of the Investigator could adversely affect the safety of the subject or impair the assessment of study results; (xviii) Current significant renal impairment defined as estimated glomerular filtration rate <60 mL/min at Screening; (xvix) Current hepatic impairment resulting in elevated liver function test (aspartate transaminase, alanine transaminase, alanine phosphatase) at 3 times the upper limit of normal at Screening; (xx) Pregnancy, planning on becoming pregnant during the course of the trial, or currently breastfeeding; (xxi) Use of medications that are moderate or strong inhibitors of CYP3A4 within 1 week of Screening or medications that are moderate or strong inducers of CYP3A4 within 2 weeks of Screening or planned use of moderate or strong CYP3A4 inhibitor or inducer medications during the study period.

### Investigational and Reference Product, Dosage and Mode of Administration:

Compound 1 tablets will be administered orally QD. The two investigation product dosing arms will be 10 mg for 12 weeks and 20 mg for 12 weeks.

Compound 1 active investigational product and matching placebo reference product tablets will be administered orally QD. Compound 1 investigational drug product is a film-coated tablet dosage form for oral administration. The white to off-white round coated tablets will be provided in 2 dosage strengths of 10 mg and 20 mg tablets which each contain Compound 1 drug substance and excipients selected from microcrystalline cellulose, lactose monohydrate, povidone K30, croscarmellose sodium, poloxamer 407, and magnesium stearate. The 10 mg and 20 mg tablets will be provided in 2 different sizes. The placebo tablet contains the same compendial excipients and is manufactured in the same tablet sizes with the same appearance to match the 10 mg and 20 mg Compound 1 tablets.

Evidence for the safety of the selected doses is provided by the ongoing Phase 1 study and the results of the comprehensive preclinical toxicology program to date. In the Phase 1 study, single doses ranging from 5 mg to 135 mg and multiple doses for 14 days of 15 mg and 30 mg have been safe and generally well tolerated.

A target 30% to 50% decrease in mHTT is the range associated with decreased pathology and anticipated therapeutic benefit in patients. In the Phase 1 study, Compound 1-mediated HTT pre-mRNA splicing was dose dependent across all cohorts in both the SAD and MAD portions of the study. Mean decreases in full-length HTT mRNA levels of 40% and 60% were observed after 14 days of treatment with Compound 1 at 15 mg and 30 mg, respectively. On the basis of these clinical data, a PK-PD compartment model was used to simulate percentage of mRNA decreases (and thus the anticipated magnitude of HTT protein lowering) at additional potential clinical doses.

At the selected doses of 10 mg QD and 20 mg QD, the predicted percent full-length HTT mRNA decreases are within the target range of 30 to 50% reduction from baseline. Preclinical data in a bacterial artificial chromosome transgenic mouse model of HD, mice showed a strong correlation between levels of HTT pre-mRNA splicing and the degree of protein lowering following Compound 1 administration. Therefore, the observed preclinical HTT mRNA changes are anticipated to result in similar decreases in HTT protein levels in HD patients. Thus, based upon the totality of the clinical and preclinical safety data to date, and the anticipated reduction in HTT mRNA and protein derived from clinical data and pharmacokinetic-pharmacodynamic modeling, the doses of 10 mg and 20 mg are expected to be safe, well tolerated, and beneficial to subjects with HD.

### Reference Product, Dosage and Mode of Administration:

Matching placebo tablets will be administered orally QD.

### Safety Criteria:

Safety assessments will include observed TEAEs, clinical labs, vital signs, ECG, C-SSRS, slit lamp eye examination, and physical examination.

### Efficacy Criteria:

Assessment of efficacy will include analysis of: (i) blood HTT protein and CSF NfL, (ii) UHDRS, (iii) CGI-C, (iv) wearable accelerometer for motor function, and (v) neuroimaging (vMRI).

### Enrichment Criteria

Enrichment is defined as the prospective use of any patient characteristic to select a study population in which detection of a drug effect (if one is in fact present) is more likely than it would be in an unselected population. Due to the highly variable population of patients with HD, the enrichment strategy for this Phase 2 study is intended to select for subjects who have preserved capacity for activities of daily living, work, finances, and self-care, but have reduced performance on motor and cognitive tests and are predicted to experience functional impact on activities of daily living within 3 years. The TMS and SDMT from the UHDRS will be assessed at Screening (along with CAG repeat length and age) and used to identify this population via a validated HD prognostic index for pre-manifest HD patients.

The Huntington's disease prognostic index (PIHD) or its normed version (PINHD) can be used to predict likelihood of HD progression, with higher scores indicating greater risk of functional decline. Natural history survival curves generated using the PIHD show the disease trajectory in patients with a particular PIHD score. The PINHD score allows researchers to predict disease progression in a studied population with a high degree of certainty. Historically, disease progression was commonly indexed by the CAG-Age Product (CAP), which is a type of burden score of age and CAG expansion that has several variants. When CAP is supplemented with the TMS and SDMT from the UHDRS, predictive likelihood of HD progression increases. Using these enrichment criteria, a group of subjects with HD and no functional decline (measured via the TFC and IS) can be identified and changes in blood HTT levels after treatment can be measured. This group is likely to experience decline without HTT lowering treatment as it has been found that earlier stages of HD are marked by increases in mHTT levels in CSF compared to controls.

At Baseline in this study, subjects' cognitive and motor function will be assessed by SDMT and TMS scores, respectively. Enrolled subjects will present with no functional decline as assessed by the TFC and IS. Subjects will be included in the study based on the calculation of PINHD scores as calculated by the IRT prior to randomization. Subjects with baseline PINHD scores between 0.18 to 4.93 inclusive will be eligible for enrollment in the trial. The following formula will be utilized to calculate the PINHD score:

$$PIHD = 51 \times ( TMS ) + ( - 34 ) \times SDMT + 7 \times ( age ) \times ( CAG - 34 ) .$$

The PIHD score is converted to a normalized score using the following conversion:

$$PINHD = ( PIHD - 883 ) / 1044$$

The ENROLL HD database (periodic data update 5) was utilized to identify the 0.18 to 4.93 range of PINHD scores for inclusion in the study.

### Pharmacokinetics:

Pharmacokinetic assessment will include plasma Ctrough (at Visits 3, 4, and 5). Accumulation ratio will be calculated and reported in plasma (Visits 3, 4, and 5) and CSF (Visit 5).

### Statistical Methods:

A repeated measure analysis model (repeat on visit) will be used to compare each dose with placebo for blood total HTT protein. The model will include dose, visit, dose by visit interaction and baseline. Nominal p-values and 95% confidence interval for each pairwise comparison at Visit 5 (active versus placebo) will be provided. The model will include PINHD as a stratification factor. The same analysis used for blood HTT protein will be used for blood HTT mRNA. Dose-response relationships will be explored. Demographic and baseline characteristics, disposition, safety, and efficacy endpoints will be summarized descriptively by dose group. Statistical models will be applied to understand the relationship between UHDRS and its components to blood and CSF assessments.

### Phase 2 Study Results

The primary objective of the 12 week Phase 2a, randomized, placebo-controlled, dose-finding study is to evaluate the safety and pharmacodynamic effects of two treatment regimens of Compound 1 and placebo in subjects with Huntington's Disease. The primary objective assesses the occurrence of treatment-emergent adverse events (TEAEs); abnormalities in laboratory values, electrocardiogram (ECG), vital signs, slit lamp eye examination, and physical examination; and reduction in blood total huntingtin protein (HTT) levels.

The secondary objectives of the study determine the effect of Compound 1 on HTT mRNA in blood and mHTT protein in cerebrospinal fluid (CSF); and reduction in blood mutant huntingtin protein (mHTT) levels.

The exploratory objectives of the study assess the effect of Compound 1 on change in whole brain, caudate, and putamen volume via volumetric magnetic resonance imaging (vMRI); assess the effect of change in ventricular volume via vMRI; assess the effect of Compound 1 on plasma and CSF neurofilament light chain (NfL) protein concentrations; assess change after 12 weeks of treatment in relevant scales, which will include an assessment using the Unified Huntington's Disease Rating Scale (UHDRS) and each of its subcomponents. The UHDRS subcomponents are used to assess qualitative efficacy including, (a) Symbol Digit Modalities Test (SDMT), (b) Total Motor Score (TMS), (c) Independence Scale; (d) Total Functional Capacity (TFC); (e) Gait and motor assessment via a wearable accelerometer; (f) Clinical Global Impression of Change (CGI-C); and (g) Huntington's Disease Quality of Life questionnaire (HDQoL).

The pharmacokinetic objectives of the study evaluate the concentration of Compound 1 in subjects with HD.

It will be appreciated that, although specific aspects of the disclosure have been described herein for purposes of illustration, the disclosure described herein is not to be limited in scope by the specific aspects herein disclosed. These aspects are intended as illustrations of several aspects of the disclosure. Any equivalent aspects are intended to be within the scope of this disclosure. Indeed, various modifications of the disclosure in addition to those shown and described herein will become apparent to those skilled in the art from the foregoing description, which modification also intended to be within the scope of this disclosure.

All references cited herein are incorporated herein by reference in their entirety and for all purposes to the same extent as if each individual publication or patent or patent application was specifically and individually indicated to be incorporated by reference in its entirety for all purposes.

The invention claimed is:

1.-35. (canceled)

36. A small molecule-inducible alternatively spliced huntingtin (HTT) mRNA, wherein the small molecule-inducible mRNA is inducible only in the presence of a small molecule composition and results in premature termination of translation and truncation of HTT polypeptide, and wherein cDNA of the alternatively spliced mRNA comprises a noncanonical 5′ splice site and a 3′ splice site and having a splice junction nucleotide sequence of SEQ ID NO: 51.

37. The alternatively spliced mRNA of claim 36, wherein cDNA of the alternatively spliced mRNA comprises the nucleotide sequence of SEQ ID NO: 46 or SEQ ID NO: 49.

38. The alternatively spliced mRNA of claim 36, having a splice junction nucleotide sequence of SEQ ID NO:53, SEQ ID NO: 55, or SEQ ID NO: 50.

39. The alternatively spliced mRNA of claim 36, wherein the small molecule-inducible RNA is not inducible in the absence of a pseudo-exonic splicing enhancer (pseudo-ESE) proximal to the 5′ splice site.

40. The alternatively spliced mRNA of claim 39, wherein the pseudo-ESE is within 100 nucleotides upstream of the 5′ splice site.

41. The alternatively spliced mRNA of claim 39, wherein the pseudo-ESE comprises at least 10 nucleotides of the nucleotide sequence of SEQ ID NO: 85.

42. The alternatively spliced mRNA of claim 36, wherein the 5′ splice site comprises the nucleotide sequence of SEQ ID NO: 5.

43. The alternatively spliced mRNA of claim 36, wherein the 3′ splice site comprises the nucleotide sequence of SEQ ID NO: 4 or 47.

44. The alternatively spliced mRNA of claim 36, wherein the small molecule composition comprises an effective amount of a compound selected from the group consisting of: 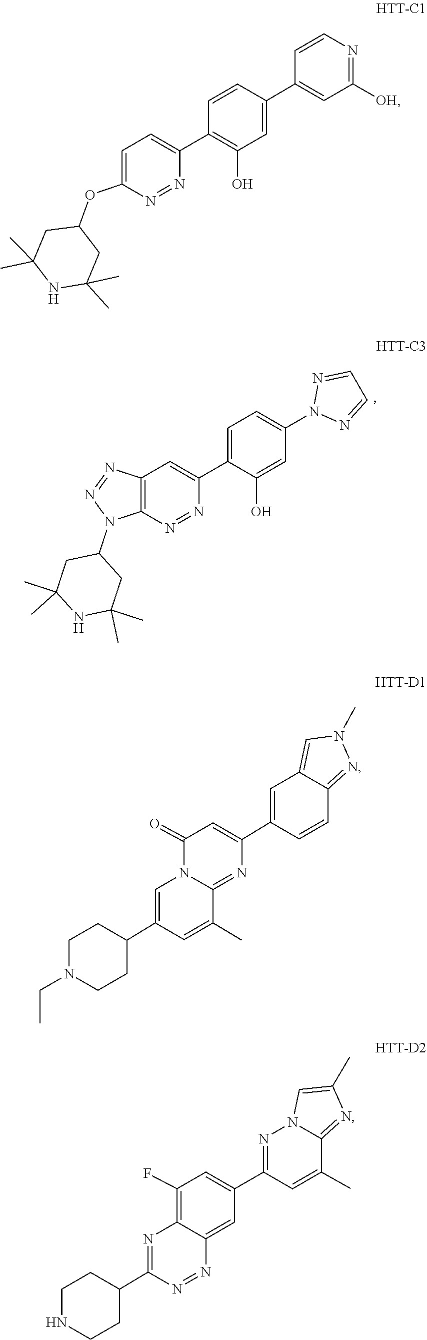 [SMILES: CC1(C)CC(Oc2ccc(-c3ccc(-c4ccnc(O)c4)cc3O)nn2)CC(C)(C)N1 ; CC1(C)CC(n2nnc3cc(-c4ccc(-n5nccn5)cc4O)nnc32)CC(C)(C)N1 ; CCN1CCC(c2cc(C)c3nc(-c4ccc5nn(C)cc5c4)cc(=O)n3c2)CC1 ; Cc1cn2nc(-c3cc(F)c4nc(C5CCNCC5)nnc4c3)cc(C)c2n1] 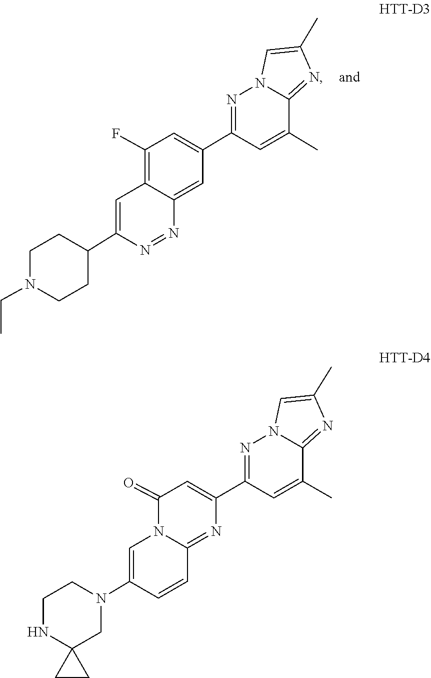 [SMILES: CCN1CCC(c2cc3c(F)cc(-c4cc(C)c5nc(C)cn5n4)cc3nn2)CC1 ; Cc1cn2nc(-c3cc(=O)n4cc(N5CCNC6(CC6)C5)ccc4n3)cc(C)c2n1]
or a pharmaceutically acceptable salt thereof,
effective at inducing the alternatively spliced huntingtin (HTT) mRNA.

45. The alternatively spliced mRNA of claim 36, wherein the small molecule composition comprises an effective amount of the compound having the structure of: 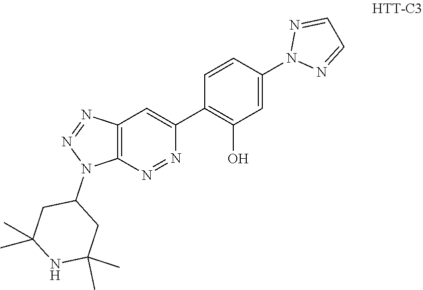 [SMILES: CC1(C)CC(n2nnc3cc(-c4ccc(-n5nccn5)cc4O)nnc32)CC(C)(C)N1]
or of a compound having the structure of 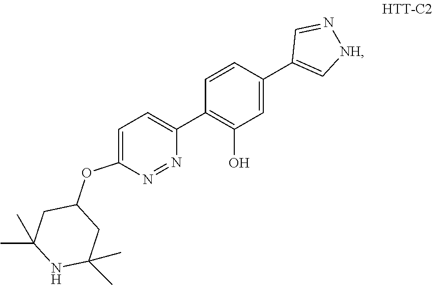 [SMILES: CC1(C)CC(Oc2ccc(-c3ccc(-c4cn[nH]c4)cc3O)nn2)CC(C)(C)N1]
or a pharmaceutically acceptable salt thereof,
effective at inducing the alternatively spliced huntingtin (HTT) mRNA.

46. The alternatively spliced mRNA of claim 36, wherein the small molecule-inducible mRNA is inducible only in the presence of a small molecule composition, wherein precursor RNA transcript comprises in 5′ to 3′ order:
a 5′ exonic splice site,
a first intronic branch point,
an intronic 3′ splice site,
a pseudo-ESE (Exonic Splice Enhancer),
a noncanonical 5′ splice site,
a second intronic branch point, and
a 3′ exonic splice site.

47. The alternatively spliced mRNA of claim 46, wherein
the pseudo-ESE comprises at least 10 nucleotides of the nucleotide sequence of SEQ ID NO: 85;
the noncanonical 5′ splice site comprises a nucleotide sequence of SEQ ID NO: 5; and
the intronic 3′ splice site comprises a nucleotide sequence of SEQ ID NO: 4 or 47.

48. The alternatively spliced mRNA of claim 46, wherein the sequence between the intronic 3′ splice site and the noncanonical 5′ splice site comprises the nucleotide sequence of SEQ ID NO: 46 or 49.

49. The alternatively spliced mRNA of claim 36, wherein the HIT mRNA comprises a CAG repeat mutant HIT mRNA.

50. The alternatively spliced mRNA of claim 49, wherein the HIT mRNA comprises a CAG repeat mutant HIT mRNA having more than 36 CAG repeats.

51. A method for reducing the expression of a gene in a subject, whereby small molecule-inducible alternatively spliced huntingtin (HTT) mRNA is induced, comprising a therapeutically effective amount of a small molecule composition comprising a compound having the structure of 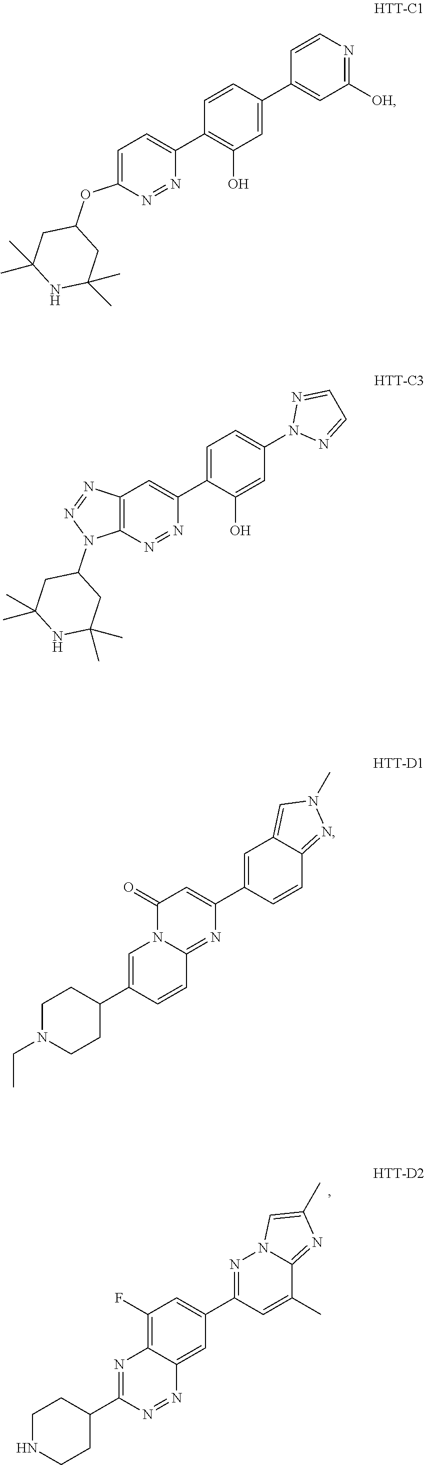 [SMILES: CC1(C)CC(Oc2ccc(-c3ccc(-c4ccnc(O)c4)cc3O)nn2)CC(C)(C)N1 ; CC1(C)CC(n2nnc3cc(-c4ccc(-n5nccn5)cc4O)nnc32)CC(C)(C)N1 ; CCN1CCC(c2ccc3nc(-c4ccc5nn(C)cc5c4)cc(=O)n3c2)CC1 ; Cc1cn2nc(-c3cc(F)c4nc(C5CCNCC5)nnc4c3)cc(C)c2n1] 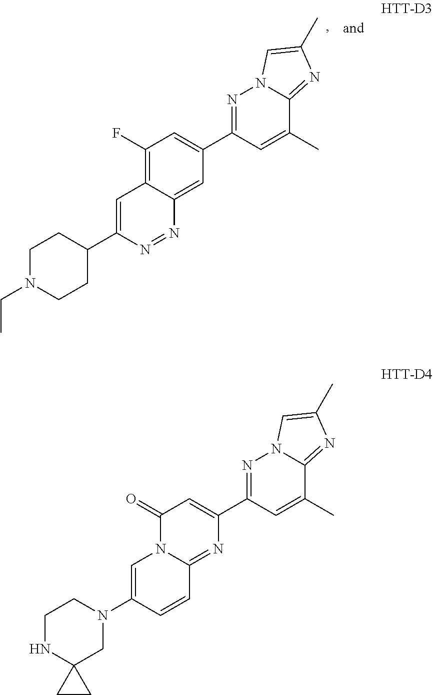 [SMILES: CCN1CCC(c2cc3c(F)cc(-c4cc(C)c5nc(C)cn5n4)cc3nn2)CC1 ; Cc1cn2nc(-c3cc(=O)n4cc(N5CCNC6(CC6)C5)ccc4n3)cc(C)c2n1]
or a pharmaceutically acceptable salt thereof.

52. The method of claim 51, wherein the subject has Huntington's disease.

53. The method of claim 51, wherein the amount of the small molecule composition is therapeutically effective if it decreases huntingtin (HTT) protein expression by about 30% to about 50% relative to a control.

54. The method of claim 51, wherein the small molecule composition comprises an effective amount of the compound having the structure of: 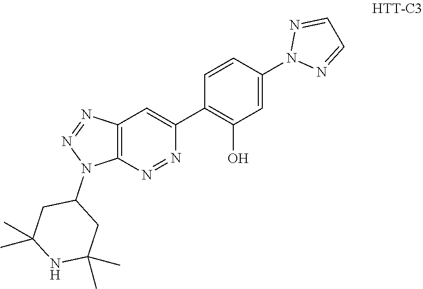 [SMILES: CC1(C)CC(n2nnc3cc(-c4ccc(-n5nccn5)cc4O)nnc32)CC(C)(C)N1]
or a pharmaceutically acceptable salt thereof,
effective at inducing the alternatively spliced huntingtin (HTT) mRNA.

55. A method for determining a therapeutically effective amount of a small molecule composition effective at reducing the amount of protein in a subject comprising measuring the amount of the small molecule-inducible alternatively spliced mRNA of claim 36 in a sample taken from the subject before and after administration of the small molecule composition.

56. The method of claim 55, wherein the small molecule composition comprises an effective amount of the compound having the structure of:

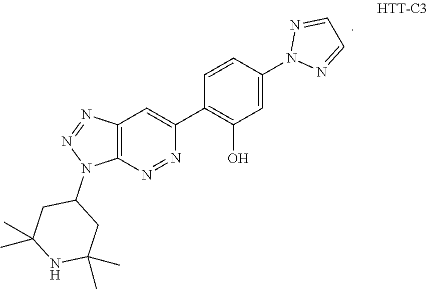 [SMILES: CC1(C)CC(n2nnc3cc(-c4ccc(-n5nccn5)cc4O)nnc32)CC(C)(C)N1]

57. The method of claim 55, wherein the mRNA comprises a CAG repeat mutant HTT mRNA.

58. The method of claim 55, wherein the subject has Huntington's disease.
